(19) United States
(12) Patent Application Publication
(10) Pub. No.: US 2026/0027208 A1
(43) Pub. Date: Jan. 29, 2026

(54) HOST CELLS BEARING KRAS BINDING PROTEIN AND KNOCKOUT OF ENDOGENOUS TCR AND METHODS OF USE THEREOF

(71) Applicant: Affini-T Therapeutics, Inc., Watertown, MA (US)

(72) Inventors: Ankit GUPTA, Waertown, MA (US); Joshua FRANCIS, Watertown, MA (US); Santosh NARAYAN, Watertown, MA (US); Nathaniel SWANSON, Watertown, MA (US); Nicholas ROUILLARD, Watertown, MA (US); Cheryl BLACK, Watertown, MA (US); Gary SHAPIRO, Watertown, MA (US); Xingyue HE, Watertown, MA (US); Allison DRAIN, Watertown, MA (US)

(73) Assignee: Affini-T Therapeutics, Inc., Watertown, MA (US)

(21) Appl. No.: 19/288,396

(22) Filed: Aug. 1, 2025

Related U.S. Application Data
(63) Continuation of application No. PCT/US2024/014303, filed on Feb. 2, 2024.
(60) Provisional application No. 63/483,231, filed on Feb. 3, 2023.
(60) Provisional application No. 63/492,146, filed on Mar. 24, 2023.
(60) Provisional application No. 63/496,356, filed on Apr. 14, 2023.
(60) Provisional application No. 63/503,403, filed on May 19, 2023.
(60) Provisional application No. 63/545,883, filed on Oct. 26, 2023.

(51) Int. Cl. A61K 40/11; A61K 35/15; A61K 35/17; A61K 40/15; A61K 40/17; A61K 40/32; A61K 40/42; A61P 35/00; C07K 14/705; C07K 14/725; C12N 5/0783; C12N 5/0786; C12N 5/10; C12N 15/11; C12N 15/63

(52) U.S. Cl. CPC A61K 40/11; A61K 35/15; A61K 35/17; A61K 40/15; A61K 40/17; A61K 40/32; A61K 40/42; A61P 35/00; C07K 14/7051; C07K 14/70517; C12N 5/0636; C12N 5/0645; C12N 5/0646; C12N 5/10; C12N 15/11; C12N 15/63; C12N 2510/00

(57) ABSTRACT

The present disclosure provides methods and compositions for adoptive T cell therapy, particularly extracellular binding proteins targeting KRAS peptides, host cells comprising the binding proteins, and methods of use and manufacture thereof.

### CROSS-REFERENCE TO RELATED APPLICATIONS

The present application is a continuation under 35 U.S.C. § 111(a) of PCT International Patent Application No. PCT/US2024/014303, filed Feb. 2, 2024, designating the United States and published in English, which claims priority to and the benefit of the following U.S. Provisional Application Nos. 63/483,231, filed Feb. 3, 2023; 63/492,146, filed Mar. 24, 2023; 63/496,356, filed Apr. 14, 2023; 63/503,403, filed May 19, 2023; and 63/545,883, filed Oct. 26, 2023, the entire contents of each of which are incorporated herein by reference.

### SEQUENCE LISTING

The present application contains a Sequence Listing which has been submitted electronically in XML format. The entire contents of the electronic XML Sequence Listing (Date of creation: Feb. 27, 2024; Size: 288,915 bytes; Name: 218378-040105PCT_SL.xml) are incorporated by reference herein.

### BACKGROUND

Adoptive T cell therapy (ACT) has demonstrated activity in solid tumors but requires further optimization to become more reproducibly effective. T cells engineered with T Cell Receptors (TCRs) recognizing intracellular oncogenic drivers like mutant KRAS, the most frequently altered gene in human cancers, have the potential to induce durable responses in patients with solid tumors.

### SUMMARY

There is a need for improved engineered host cells and compositions for adoptive cell therapy against solid tumors. Without wishing to be bound by theory, it is understood that competition between transgenic and endogenous TCRs for the available CD3 pool in T cells and the potential of mispairing of transgenic and endogenous TCR chains can lead to decreased surface expression of the engineered TCR, and thus diminished sensitivity when engineered TCRs are used in adoptive cell therapies.

Accordingly, some aspects of the present disclosure related to host cells bearing an exogenous transgenic mutant KRAS-targeted TCR where the endogenous TCRs have been edited to eliminate endogenous TCR expression and mispairing with the transgenic TCR. In some cases, this engineering involves use of a Type V-A CRISPR nuclease alongside compatible gRNAs that resulted in TCR knockout in >90% of human primary T cells. This editing, in some embodiments, has extremely high specificity for knockout of TCR-associated loci, improves expression of exogenous TCR, and improves the sensitivity of the engineered host cells to therapeutically targeted cells (e.g., enhanced in vitro cytotoxicity against tumor cells expressing the mutant KRAS G12D peptide).

In an aspect, the present disclosure provides a host cell. The host cell includes a heterologous extracellular binding protein, where the extracellular binding protein is capable of binding to a peptide:HLA complex, where the peptide comprises a KRAS G12D mutant peptide and a genomic mutation that decreases expression of an endogenous T cell receptor α constant (TRAC), T cell receptor β constant 1 (TRBC1), or T cell receptor β constant 2 (TRBC2).

In another aspect, the present disclosure provides a host cell. The host cell includes a polynucleotide encoding a heterologous extracellular binding protein inserted at a TRAC, TRBC1, or TRBC2 locus, where the extracellular binding protein is capable of binding to a peptide:HLA complex, where the peptide comprises a KRAS G12D mutant peptide, and where the host cell has decreased expression of TRAC, TRBC1, or TRBC2.

In another aspect, the present disclosure provides a polynucleotide encoding an extracellular binding protein. The extracellular binding protein includes a TCR α chain variable (Vα) domain including an amino acid sequence with at least 80% sequence identity to any one of SEQ ID NOs: 9, 25, 41, 57, 73, 89, 105, 123, 139, 155, 171, 187, 229, 230, 239, 240, 249, 250, 259, 260, 269, and 270; a TCR β chain variable (Vβ) domain including an amino acid sequence with at least 80% sequence identity to any one of SEQ ID NOs: 1, 17, 33, 49, 65, 81, 97, 131, 147, 163, 179, 195, 234, 235, 244, 245, 254, 255, 264, 265, 274, and 275; or a TCRα FR1, CDR1, FR2, CDR2, FR3, CDR3, or FR4 region or a TCRβ FR1, CDR1, FR2, CDR2, FR3, CDR3, or FR4 region including an amino acid sequence with at least 80% sequence identity to any one of SEQ ID NOs: 2-8, 10-16, 18-24, 26-32,34-40, 42-48, 50-56, 58-64, 66-72, 74-80, 82-88, 90-96, 98-104, 106-112, 124-130, 132-138, 140-146, 148-154, 156-162, 164-170, 172-178, 180-186, 188-194, 196-202, 231-233, 236-238, 241-243, 246-248, 251-253, 25-258, 261-263, 266-268, 271-273, and 276-278.

In another aspect, the present disclosure provides a vector. The vector includes the polynucleotide of any one of the above aspects, or embodiments thereof.

In another aspect, the present disclosure provides a cell. The cell includes the polynucleotide or the vector of any of the above aspects, or embodiments thereof.

In another aspect, the present disclosure provides a pharmaceutical composition. The pharmaceutical composition includes the host cell of any of the above aspects, or embodiments thereof, and a pharmaceutically acceptable carrier, excipient, or diluent.

In another aspect, the present disclosure provides a method of treating a disease or disorder associated with a KRAS G12 mutation in a subject. The method involves administering to the subject an effective amount of the host cell or the pharmaceutical composition of any one of the above aspects, or embodiments thereof.

In any of the above aspects, or embodiments thereof, the peptide:HLA complex includes an HLA protein encoded by an HLA-A*11 allele. In any of the above aspects, or embodiments thereof, the peptide:HLA complex includes an HLA protein encoded by an HLA-A*11:01 allele.

In any of the above aspects, or embodiments thereof, the extracellular binding protein includes a T cell receptor (TCR) α chain variable (Vα) region, a TCR β chain variable (Vβ) region, a T cell receptor (TCR) α chain constant (Cα) region, or a T cell receptor (TCR) β chain constant (Cβ) region.

In any of the above aspects, or embodiments thereof, the KRAS G12D mutant peptide includes an amino acid sequence of VVVGADGVGK (SEQ ID NO: 295).

In any of the above aspects, or embodiments thereof, the Vα domain or the VP domain are human, humanized, or chimeric.

In any of the above aspects, or embodiments thereof, the extracellular binding protein is human, humanized, or chimeric.

In any of the above aspects, or embodiments thereof, the extracellular binding protein includes: a TCR α chain variable (Vα) domain including an amino acid sequence with at least 80% sequence identity to any one of SEQ ID NOs: 9, 25, 41, 57, 73, 89, 105, 123, 139, 155, 171, 187, 229, 230, 239, 240, 249, 250, 259, 260, 269, and 270; a TCR β chain variable (Vβ) domain including an amino acid sequence with at least 80% sequence identity to any one of SEQ ID NOs: 1, 17, 33, 49, 65, 81, 97, 131, 147, 163, 179, 195, 234, 235, 244, 245, 254, 255, 264, 265, 274, and 275; or a TCRα FR1, CDR1, FR2, CDR2, FR3, CDR3, or FR4 region or a TCRβ FR1, CDR1, FR2, CDR2, FR3, CDR3, or FR4 region including an amino acid sequence with at least 80% sequence identity to any one of SEQ ID NOs: 2-8, 10-16, 18-24, 26-32,34-40, 42-48, 50-56, 58-64, 66-72, 74-80, 82-88, 90-96, 98-104, 106-112, 124-130, 132-138, 140-146, 148-154, 156-162, 164-170, 172-178, 180-186, 188-194, 196-202, 231-233, 236-238, 241-243, 246-248, 251-253, 25-258, 261-263, 266-268, 271-273, and 276-278.

In any of the above aspects, or embodiments thereof, the extracellular binding protein specifically binds the KRAS G12D mutant peptide.

In any of the above aspects, or embodiments thereof, the extracellular binding protein is at least 5-, 10-, 25-, 50-, 100-, 200-, 500-, or 1000-fold selective for the KRAS G12D mutant peptide versus other 10-mer peptides encoded by a genome of the cell.

In any of the above aspects, or embodiments thereof, the host cell further includes (i) a transgenic polynucleotide encoding a polypeptide that includes an extracellular portion of a CD8 co-receptor α (CD8α) chain or (ii) a transgenic polynucleotide encoding a polypeptide that includes an extracellular portion of a CD8 co-receptor β (CD8β) chain.

In any of the above aspects, or embodiments thereof, the extracellular binding protein has a log₁₀ EC50 for the KRAS G12 mutant peptide of about −6.0 or less, about −6.1 or less, about −6.2 or less, about −6.3 or less, about −6.4 or less, about −6.5 or less, about −6.6 or less, about −6.7 or less, about −6.8 or less, about −6.9 or less, about −7.0 or less, about −7.1 or less, about −7.2 or less, about −7.3 or less, about −7.4 or less, about −7.5 or less, about −7.6 or less, about −7.7 or less, about −7.8 or less, about −7.9 or less, about −8.0 or less, about −8.1 or less, about −8.2 or less, about −8.3 or less, about −8.4 or less, about −8.5 or less, about −8.6 or less, about −8.7 or less, about −8.8 or less, about −8.9 or less, about −9 or less, about −9.1 or less, or about −9.2 or less.

In any of the above aspects, or embodiments thereof, when the host cell is in the presence of a tumor cell that expresses a KRAS G12D mutant peptide, CD137 expression of the host cell is elevated as compared to: (i) CD137 expression by a reference human T cell not expressing the binding protein, when the reference human T cell is in the presence of the tumor cell; or (ii) CD137 expression by the human T cell expressing the binding protein when not in the presence of the tumor cell or when not in the presence of an antigen-presenting cell expressing a peptide:HLA complex.

In any of the above aspects, or embodiments thereof, the genomic mutation that causes or contributes to decreased expression of the endogenous T cell receptor α constant (TRAC), T cell receptor β constant 1 (TRBC1), or a T cell receptor β constant 2 (TRBC2) includes an indel in the TRAC, TRBC1, or TRBC2 locus. In any of the above aspects, or embodiments thereof, the genomic mutation that causes or contributes to decreased expression of the endogenous T cell receptor α constant (TRAC), T cell receptor β constant 1 (TRBC1), or T cell receptor β constant 2 (TRBC2) is a missense mutation that causes or contributes to reduced function or stability of a T cell receptor α or T cell receptor β polypeptide encoded by a genome of the cell. In any of the above aspects, or embodiments thereof, the genomic mutation that decreases expression of the endogenous T cell receptor α constant (TRAC), T cell receptor β constant 1 (TRBC1), or a T cell receptor β constant 2 (TRBC2) results in premature termination of a T cell receptor α or T cell receptor β polypeptide encoded by the endogenous TRAC, TRBC1, or TRBC2. In any of the above aspects, or embodiments thereof, the host cell includes genomic mutations that decreases expression of both (i) TRAC; and (ii) TRBC1 or TRBC2. In any of the above aspects, or embodiments thereof, the host cell includes genomic mutations that decreases expression of TRAC, TRBC1, and TRBC2.

In any of the above aspects, or embodiments thereof, the host cell includes an immune cell or a precursor thereof. In any of the above aspects, or embodiments thereof, the immune cell includes a T cell, a NK cell, a NK-T cell, a dendritic cell, a macrophage, a monocyte, or any combination thereof. In any of the above aspects, or embodiments thereof, the immune cell includes a T cell, where the T cell includes a CD4⁺ T cell, a CD8⁺ T cell, a CD4⁻ CD8⁻ double negative T cell, a γδ T cell, or any combination thereof.

In any of the above aspects, or embodiments thereof, the polynucleotide encoding a heterologous extracellular binding protein is inserted at the TRAC locus, and where the host cell has decreased expression of TRAC.

In any of the above aspects, or embodiments thereof, the host cell further includes a recombinant protein including a IL-7 receptor alpha (IL7RA) intracellular domain and a IL7RA transmembrane domain. In any of the above aspects, or embodiments thereof, the IL7RA intracellular domain has at least 80% sequence identity to SEQ ID NO: 224. In any of the above aspects, or embodiments thereof, the IL7RA transmembrane domain has at least 80% sequence identity to SEQ ID NO: 225.

In any of the above aspects, or embodiments thereof, the recombinant protein further includes a CD34 or CD58 extracellular domain. In any of the above aspects, or embodiments thereof, the CD58 extracellular domain has at least 80% sequence identity to SEQ ID NO: 227. In any of the above aspects, or embodiments thereof, the recombinant protein has at least 80% sequence identity to SEQ ID NO: 223.

In any of the above aspects, or embodiments thereof, the binding protein is capable of binding to a peptide:HLA complex, where the peptide comprises a KRAS G12 mutant peptide. In any of the above aspects, or embodiments thereof, the KRAS G12 mutant peptide is a KRAS G12D mutant peptide. In any of the above aspects, or embodiments thereof, the KRAS G12D mutant peptide includes an amino acid sequence of VVVGADGVGK (SEQ ID NO: 295).

In any of the above aspects, or embodiments thereof, the nucleic acid sequence is codon optimized.

In any of the above aspects, or embodiments thereof, the extracellular binding protein is human, humanized, or chimeric.

In any of the above aspects, or embodiments thereof, the extracellular binding protein is selective for the KRAS G12D mutant peptide.

In any of the above aspects, or embodiments thereof, the extracellular binding protein has a log₁₀ EC50 for the KRAS G12 mutant peptide of about −6.0 or less, about −6.1 or less, about −6.2 or less, about −6.3 or less, about −6.4 or less, about −6.5 or less, about −6.6 or less, about −6.7 or less, about −6.8 or less, about −6.9 or less, about −7.0 or less, about −7.1 or less, about −7.2 or less, about −7.3 or less, about −7.4 or less, about −7.5 or less, about −7.6 or less, about −7.7 or less, about −7.8 or less, about −7.9 or less, about −8.0 or less, about −8.1 or less, about −8.2 or less, about −8.3 or less, about −8.4 or less, about −8.5 or less, about −8.6 or less, about −8.7 or less, about −8.8 or less, about −8.9 or less, about −9 or less, about −9.1 or less, or about −9.2 or less.

In any of the above aspects, or embodiments thereof, the extracellular binding protein includes an amino acid sequence of any one of SEQ ID NOs: 1, 9, 17, 25, 33, 41, 49, 57, 65, 73, 81, 89, 97, 105, 123, 131, 139, 147, 155, 163, 171, 179, 187, 195, 229, 230, 234, 235, 239, 240, 244, 245, 249, 250, 254, 255, 259, 260, 264, 269, 265, 270, 274, and 275.

In any of the above aspects, or embodiments thereof, the polynucleotide further includes a promoter. In any of the above aspects, or embodiments thereof, the promoter is an elongation factor-1 alpha (EF-1α) promoter.

In any of the above aspects, or embodiments thereof, the polynucleotide includes RNA, DNA, or a combination thereof.

In any of the above aspects, or embodiments thereof, the vector is a lentiviral vector, a γ-retroviral vector, or an adeno-associated virus (AAV) vector.

In any of the above aspects, or embodiments thereof, the pharmaceutical composition includes both CD4+ cells and CD8+ cells bearing: (i) the extracellular binding protein and (ii) the genomic mutation that decreases expression of the endogenous T cell receptor α constant (TRAC), T cell receptor β constant 1 (TRBC1), or T cell receptor β constant 2 (TRBC2).

In any of the above aspects, or embodiments thereof, the pharmaceutical composition further includes either or both of (i) a transgenic polynucleotide encoding a polypeptide that includes an extracellular portion of a CD8 co-receptor α (CD8α) chain, or (ii) a transgenic polynucleotide encoding a polypeptide that includes an extracellular portion of a CD8 co-receptor β (CD8β) chain.

In any of the above aspects, or embodiments thereof, the composition includes about a 1:1 ratio of CD4+ T cells to CD8+ T cells.

In any of the above aspects, or embodiments thereof, the subject is positive for an HLA-A*11 allele. In any of the above aspects, or embodiments thereof, the subject is positive for an HLA-A*11:01 allele.

In any of the above aspects, or embodiments thereof, the KRAS G12 mutation is a KRAS G12D mutation.

In any of the above aspects, or embodiments thereof, the disease or disorder includes a cancer. In any of the above aspects, or embodiments thereof, the cancer is a solid cancer. In any of the above aspects, or embodiments thereof, the cancer is a hematological malignancy.

In any of the above aspects, or embodiments thereof, the disease or disorder is a bile duct tumor, cholangiocarcinoma, colon adenocarcinoma, pancreas cancer, a pancreatic ductal adenocarcinoma (PDAC); a colorectal cancer; a lung cancer, a non-small-cell lung carcinoma; a biliary cancer; an endometrial cancer; a cervical cancer; an ovarian cancer; a bladder cancer; a liver cancer; a myeloid leukemia, myeloid leukemia, acute myeloid leukemia; a myelodysplastic syndrome; a lymphoma, Non-Hodgkin lymphoma; Chronic Myelomonocytic Leukemia; Acute Lymphoblastic Leukemia (ALL); a cancer of the urinary tract; a cancer of the small intestine; a breast cancer; a melanoma, a cutaneous melanoma, an anal melanoma, or a mucosal melanoma; a glioma; a poorly differentiated thyroid gland carcinoma; a neuroblastoma; a histiocytic and dendritic cell neoplasm; neurofibromatosis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; or thyroid gland papillary carcinoma.

In any of the above aspects, or embodiments thereof, the subject is determined to carry a KRAS G12D allele prior to the administering. In any of the above aspects, or embodiments thereof, the subject has been genotyped for an HLA-A allele prior to the administering.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description or the claims, such terms are intended to be inclusive in a manner similar to the term “comprising”.

By “agent” is meant a polypeptide, nucleic acid molecule, small compound, or cell comprising a heterologous polynucleotide. In some embodiments, the cell is an immune cell (e.g., T cell) that is autologous or heterologous to a subject.

By “alteration” is meant a change (increase or decrease) in the expression levels, structure, or activity of a gene or polypeptide as detected by standard art known methods such as those described herein. As used herein, an alteration includes a 10% change in expression levels, a 25% change, a 40% change, or a 50% or greater change in expression levels.

By “ameliorate” is meant decrease, suppress, attenuate, diminish, arrest, or stabilize the development or progression of a disease.

As used herein, the term “CD8 co-receptor” or “CD8” generally refers to the cell surface glycoprotein CD8. CD8 is present at the cell surface as either as an CD8alpha subunit-CD8alpha subunit homodimer or a CD8alpha subunit-CD8beta subunit heterodimer. The CD8 co-receptor assists in the function of cytotoxic T cells (CD8+) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). There are five (5) documented human CD8 beta chain isoforms (see UniProtKB identifier P10966) and a single documented human CD8 alpha chain isoform (see UniProtKB identifier P01732).

By “CD8alpha” or “CD8α” is meant a polypeptide having at least about 85% amino acid sequence identity to NCBI Accession Nos. NP_001759.3 or NP_741969.1, or a fragment thereof having co-receptor activity for ligand recognition by T cell receptors when dimerized with another CD8α or a CD8β. Exemplary CD8α amino acid sequences are provided below:

>NP_001759.3 T-cell surface glycoprotein CD8 alpha chain isoform 1
precursor [Homo sapiens]
(SEQ ID NO: 219)
MALPVTALLLPLALLLHAARPSQFRVSPLDRTWNLGETVELKCQVLLSNPTSGCSWLFQPRGAAASPTFL
LYLSQNKPKAAEGLDTQRFSGKRLGDTFVLTLSDFRRENEGYYFCSALSNSIMYFSHFVPVFLPAKPTTT
PAPRPPTPAPTIASQPLSLRPEACRPAAGGAVHTRGLDFACDIYIWAPLAGTCGVLLLSLVITLYCNHRN
RRRVCKCPRPVVKSGDKPSLSARYV
>NP_741969.1 T-cell surface glycoprotein CD8 alpha chain isoform 2
precursor [Homo sapiens]
(SEQ ID NO: 296)
MALPVTALLLPLALLLHAARPSQFRVSPLDRTWNLGETVELKCQVLLSNPTSGCSWLFQPRGAAASPTFL
LYLSQNKPKAAEGLDTQRFSGKRLGDTFVLTLSDFRRENEGYYFCSALSNSIMYFSHFVPVFLPAKPTTT
PAPRPPTPAPTIASQPLSLRPEACRPAAGGAGNRRRVCKCPRPVVKSGDKPSLSARYV

By “CD8beta” or “CD8β” is meant a polypeptide having at least about 85% amino acid sequence identity to NCBI Accession Nos. XP_054200532.1, NP_757362.1, NP_742099.1, NP_742100.1, NP_004922.1, or NP_001171571.1, or a fragment thereof having co-receptor activity for ligand recognition by T cell receptors when dimerized with a CD8α. Exemplary CD8β amino acid sequences are provided below:

>XP_054200532.1 T-cell surface glycoprotein CD8 beta chain isoform X1
[Homo sapiens]
(SEQ ID NO: 297)
MRPRLWLLLAAQLTVLHGNSVLQQTPAYIKVQTNKMVMLSCEAKISLSNMRIYWLRQRQAPSSDSHHEFL
ALWDSAKGTIHGEEVEQEKIAVFRDASRFILNLTSVKPEDSGIYFCMIVGSPELTFGKGTQLSVVDFLPT
TAQPTKKSTLKKRVCRLPRPETQKGPLCSPITLGLLVAGVLVLLVSLGVAIHLCCRRRRARLRFMKQKFN
IVCLKISGFTTCCCFQILQMSREYGFGVLLQKDIGQ
>NP_757362.1 T-cell surface glycoprotein CD8 beta chain isoform 2 precursor
[Homo sapiens]
(SEQ ID NO: 298)
MRPRLWLLLAAQLTVLHGNSVLQQTPAYIKVQTNKMVMLSCEAKISLSNMRIYWLRQRQAPSSDSHHEFL
ALWDSAKGTIHGEEVEQEKIAVFRDASRFILNLTSVKPEDSGIYFCMIVGSPELTFGKGTQLSVVDFLPT
TAQPTKKSTLKKRVCRLPRPETQKGPLCSPITLGLLVAGVLVLLVSLGVAIHLCCRRRRARLRFMKQPQG
EGISGTFVPQCLHGYYSNTTTSQKLLNPWILKT
>NP_742099.1 T-cell surface glycoprotein CD8 beta chain isoform 3 precursor
[Homo sapiens]
(SEQ ID NO: 299)
MRPRLWLLLAAQLTVLHGNSVLQQTPAYIKVQTNKMVMLSCEAKISLSNMRIYWLRQRQAPSSDSHHEFL
ALWDSAKGTIHGEEVEQEKIAVFRDASRFILNLTSVKPEDSGIYFCMIVGSPELTFGKGTQLSVVDFLPT
TAQPTKKSTLKKRVCRLPRPETQKGPLCSPITLGLLVAGVLVLLVSLGVAIHLCCRRRRARLRFMKQLRL
HPLEKCSRMDY
>NP_742100.1 T-cell surface glycoprotein CD8 beta chain isoform 4 precursor
[Homo sapiens]
(SEQ ID NO: 300)
MRPRLWLLLAAQLTVLHGNSVLQQTPAYIKVQTNKMVMLSCEAKISLSNMRIYWLRQRQAPSSDSHHEFL
ALWDSAKGTIHGEEVEQEKIAVFRDASRFILNLTSVKPEDSGIYFCMIVGSPELTFGKGTQLSVVDFLPT
TAQPTKKSTLKKRVCRLPRPETQKGRRRRARLRFMKQPQGEGISGTFVPQCLHGYYSNTTTSQKLLNPWI
LKT
>NP_004922.1 T-cell surface glycoprotein CD8 beta chain isoform 5 precursor
[Homo sapiens]
(SEQ ID NO: 220)
MRPRLWLLLAAQLTVLHGNSVLQQTPAYIKVQTNKMVMLSCEAKISLSNMRIYWLRQRQAPSSDSHHEFL
ALWDSAKGTIHGEEVEQEKIAVFRDASRFILNLTSVKPEDSGIYFCMIVGSPELTFGKGTQLSVVDFLPT
TAQPTKKSTLKKRVCRLPRPETQKGPLCSPITLGLLVAGVLVLLVSLGVAIHLCCRRRRARLRFMKQFYK
>NP_001171571.1 T-cell surface glycoprotein CD8 beta chain isoform 6
precursor [Homo sapiens]
(SEQ ID NO: 301)
MRPRLWLLLAAQLTVLHGNSVLQQTPAYIKVQTNKMVMLSCEAKISLSNMRIYWLRQRQAPSSDSHHEFL
ALWDSAKGTIHGEEVEQEKIAVFRDASRFILNLTSVKPEDSGIYFCMIVGSPELTFGKGTQLSVVDFLPT
TAQPTKKSTLKKRVCRLPRPETQKGLKGKVYQEPLSPNACMDTTAILQPHRSCLTHGS

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “complementary” is meant capable of pairing to form a double-stranded nucleic acid molecule or portion thereof. In one embodiment, an antisense molecule is in large part complementary to a target sequence. The complementarity need not be perfect, but may include mismatches at 1, 2, 3, or more nucleotides.

By “corresponds” is meant comprising at least a fragment of a double-stranded gene, such that a strand of the double-stranded inhibitory nucleic acid molecule is capable of binding to a complementary strand of the gene.

The terms “complementarity determining region,” and “CDR,” generally refer to sequences of amino acids within immunoglobulin superfamily member (e.g., TCR) variable regions, which confer antigen specificity or binding affinity and are separated from one another in primary amino acid sequence by framework regions. In general, there are three CDRs in each TCR α-chain variable region (αCDR1, αCDR2, αCDR3) and three CDRs in each TCR β-chain variable region (βCDR1, βCDR2, βCDR3). In TCRs, CDR3 is thought to be the main CDR responsible for recognizing processed antigen. In general, CDR1 and CDR2 interact mainly or exclusively with the MHC.

CDR1 and CDR2 are encoded within the variable gene segment of a TCR variable region-coding sequence, whereas CDR3 is encoded by the region spanning the variable and joining segments for Vα, or the region spanning variable, diversity, and joining segments for Vβ. Thus, if the identity of the variable gene segment of a Vα or Vβ is described, the sequences of their corresponding CDR1 and CDR2 can be deduced; e.g., according to a numbering scheme as described herein. Compared with CDR1 and CDR2, CDR3 can be significantly more diverse due to the addition and loss of nucleotides during the recombination process.

TCR variable domain sequences can be aligned to a numbering scheme (e.g., Kabat, Chothia, EU, IMGT, Enhanced Chothia, and Aho), allowing equivalent residue positions to be annotated and for different molecules to be compared using, for example, ANARCI software tool (2016, Bioinformatics 15:298-300). A numbering scheme provides a standardized delineation of framework regions and CDRs in the TCR variable domains. In certain embodiments, a CDR of the present disclosure is identified according to the IMGT numbering scheme or method (Lefranc et al., Dev. Comp. Immunol. 27:55, 2003; imgt.org/IMGTindex/V-QUEST.php). In some embodiments, a CDR of the present disclosure is identified according to the Kabat numbering scheme or method. In some embodiments, a CDR of the present disclosure is identified according to the Chothia numbering scheme or method. In some embodiments, a CDR of the present disclosure is identified according to the EU numbering scheme or method. In some embodiments, a CDR of the present disclosure is identified according to the enhanced Chothia numbering scheme or method. In some embodiments, a CDR of the present disclosure is identified according to the Aho numbering scheme or method.

By “decreases” is meant reduces by at least about 5% relative to a reference level. A decrease may be by 5%, 10%, 15%, 20%, 25% or 50%, or even by as much as 75%, 85%, 95% or more and any intervening percentages

“Detect” refers to identifying the presence, absence or amount of the analyte to be detected.

By “disease” is meant any condition or disorder that damages or interferes with the normal function of a cell, tissue, or organ. In embodiments, the disease is a cancer.

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any analyte or clinical state having an alteration that can be characterized as described herein. In embodiments, the analyte is a protein or polynucleotide marker having an increase or decrease in expression that is associated with a disease state or cell state e.g., marker of differentiation.

As used herein, an “immune cell” generally refers to any cell of the immune system. In embodiments, an immune cell originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells and natural killer (NK) cells). Example immune system cells include a CD4+ T cell, a CD8+ T cell, a CD4− CD8− double negative T cell, a γδ T cell, a regulatory T cell, a natural killer cell, a natural killer T cell, and a dendritic cell. Macrophages and dendritic cells can be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

As used herein, a(n) “heterologous” or “exogenous” nucleic acid molecule, construct, or sequence refers to a nucleic acid molecule, or portion of a or nucleic acid molecule that is not native to a host cell but can be homologous to a nucleic acid molecule or portion thereof from the host cell. The source of the heterologous or exogenous nucleic acid molecule, construct or sequence can be from a different genus or species. In certain embodiments, a heterologous or exogenous nucleic acid molecule (i.e., not endogenous or native) is added to a host cell or host genome by, for example, conjugation, transformation, transfection, transduction, electroporation, or the like, wherein the added molecule can integrate into the host genome or exist as extra-chromosomal genetic material (e.g., as a plasmid or other form of self-replicating vector), and can be present in multiple copies. In addition, “heterologous” refers to a non-native enzyme, protein, polypeptide, or other activity encoded by an exogenous nucleic acid molecule introduced into the host cell, even if the host cell encodes a homologous protein or activity. Moreover, a cell comprising a “modification” or a “heterologous” polynucleotide or binding protein includes progeny of that cell, regardless of whether the progeny were themselves transduced, transfected, or otherwise manipulated or changed.

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)).

By “operably linked” refers to a functional linkage between a regulatory sequence and a coding sequence, where a first polynucleotide is positioned adjacent to a second polynucleotide that directs transcription of the first polynucleotide when appropriate molecules (e.g., transcriptional activator proteins) are bound to the second polynucleotide. The described components are therefore in a relationship permitting them to function in their intended manner. For example, placing a coding sequence under regulatory control of a promoter means positioning the coding sequence such that the expression of the coding sequence is controlled by the promoter.

The term “promoter” as used herein refers to a sequence of DNA that directs the expression (transcription) of a gene. A promoter may direct the transcription of a prokaryotic or eukaryotic gene. A promoter may be “inducible”, initiating transcription in response to an inducing agent or, in contrast, a promoter may be “constitutive”, whereby an inducing agent does not regulate the rate of transcription. A promoter may be regulated in a tissue-specific or tissue-preferred manner, such that it is only active in transcribing the operable linked coding region in a specific tissue type or types.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, at least about 100 nucleotides or at least about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In an embodiment, such a sequence is at least 60%, more preferably 80% or 85%, and 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein “T cell” or “T lymphocyte” generally refers to an immune cell that matures in the thymus and produces a T cell receptors (TCR). T cells can be naïve (“TN”; not exposed to antigen; increased expression of CD62L, CCR7, CD28, CD3, CD127, and CD45RA, and decreased or no expression of CD45RO as compared to TCM (described herein)), memory T cells (TM) (antigen experienced and long-lived), including stem cell memory T cells, and effector cells (antigen-experienced, cytotoxic). TM can be further divided into subsets of central memory T cells (TCM, expresses CD62L, CCR7, CD28, CD95, CD45RO, and CD127) and effector memory T cells (TEM, express CD45RO, decreased expression of CD62L, CCR7, CD28, and CD45RA). Effector T cells (TE) refers to antigen-experienced CD8+ cytotoxic T lymphocytes that express CD45RA, have decreased expression of CD62L, CCR7, and CD28 as compared to TCM, and are positive for granzyme and perforin. Helper T cells (TH) are CD4+ cells that influence the activity of other immune cells by releasing cytokines. CD4+ T cells can activate and suppress an adaptive immune response, and which of those two functions is induced will depend on presence of other cells and signals. T cells can be collected using suitable techniques, and the various subpopulations or combinations thereof can be enriched or depleted by suitable techniques, such as by affinity binding to antibodies, flow cytometry, or immunomagnetic selection. Other example T cells include regulatory T cells, such as CD4+ CD25+ (Foxp3+) regulatory T cells and Treg17 cells, as well as Tr1, Th3, CD8+CD28−, and Qa-1 restricted T cells.

As used herein, a “T cell receptor” (TCR) generally refers to an immunoglobulin superfamily member having a variable binding domain, a constant domain, a transmembrane region, and a short cytoplasmic tail; see, e. g., Janeway et al., Immunobiology: The Immune System in Health and Disease, 3rd Ed., Current Biology Publications, p. 433, 1997) capable of specifically binding to an antigen peptide bound to a MHC receptor. A TCR can be found on the surface of a cell or in soluble form and generally comprises heterodimer having a and β chains (also known as TCR α and TCRβ, respectively), or γ and δ chains (also known as TCRγ and TCRδ, respectively).

The term “variable region” or “variable domain” generally refers to the domain of an immunoglobulin superfamily binding protein (e.g., a TCR α-chain or β-chain (or γ chain and δ chain for γδ TCRs)) that is involved in binding of the immunoglobulin superfamily binding protein (e.g., TCR) to antigen. The variable domains of the α chain and β chain (Vα and Vβ, respectively) of a native TCR generally have similar structures, with each domain comprising four generally conserved framework regions (FRs) and three CDRs. The Vα domain is encoded by two separate DNA segments, the variable gene segment and the joining gene segment (V-J); the Vβ domain is encoded by three separate DNA segments, the variable gene segment, the diversity gene segment, and the joining gene segment (V-D-J). A single Vα or Vβ domain may be sufficient to confer antigen-binding specificity. Furthermore, TCRs that bind a particular antigen may be isolated using a Vα or Vβ domain from a TCR that binds the antigen to screen a library of complementary Vα or Vβ domains, respectively.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

Included in the current disclosure are variants of any of the enzyme described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and β chain length for one another. Such conservatively substituted variants may include variants with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identity to any of the sequences described in Table 1. In some embodiments, such conservatively substituted variants are functional variants. Such functional variants can encompass sequences with substitutions such that the activity of critical binding residues of the polypeptide or polynucleotide are not disrupted.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A and FIG. 1B show TCR activation in preliminary screening of KRAS-G12D TCRs. Shown are plots of percent GFP-positive cells as a function of peptide concentration and a chart of EC50s derived therefrom. Peptide dose-dependent responses for each TCR were assessed by analyzing GFP expression following overnight culture with A11 target cells pulsed with decreasing concentrations of peptide as indicated. Dose-response curves were fitted by non-linear regression, and EC50 values were calculated using GraphPad Prism®. TCR091 showed the highest affinity in the assay.

FIG. 2 shows a comparison of cytotoxic activity of wild type TRAC/TRBC T cells and TRAC/TRBC double knockout (dKO) T cells transduced to express a KRAS-G12D-specific TCR. Red fluorescent Hpaf-II cells, a KRAS-G12D expressing tumor cell line transduced to express HLA-A11, were cocultured at 3:1 effector:target cell ratio with TCR32-transduced CD8⁺ T cells having wild type TRAC and TRBC loci or TCR32-transduced TRAC/TRBC double knockout (dKO) CD8⁺ T cells as indicated. Red fluorescence was measured by live cell imaging using the IncuCyte S3 microscope and software package, and total red object integrated intensity is plotted over time as a measure of tumor cell volume.

FIG. 3A, FIG. 3B, FIG. 3C, FIG. 3D, and FIG. 3E show comparisons of T cell activation of wild type TRAC/TRBC T cells (upper panels) and TRAC/TRBC dKO T cells (lower panels) transduced to express KRAS-G12D-specific TCRs. Shown are plots of the percentage of CD137-positive cells assessed via FACS versus peptide concentration.

FIGS. 4A-4J show a characterization of cytotoxic activity of wild type TRAC/TRBC T cells and TRAC/TRBC dKO T cells transduced to express KRAS-G12D-specific TCRs. Plots of total red object integrated intensity are shown as a measure of tumor cell volume over time by live cell imaging using the IncuCyte S3 microscope and software package as described for FIG. 2. FIG. 4A provides data for T cells with wild type TRAC/TRBC loci incubated at a 3:1 effector to target ratio with Hpaf-II target cells. FIG. 4B provides data for T cells with TRAC/TRBC double knockout incubated at a 3:1 effector to target ratio with Hpaf-II target cells. FIG. 4C provides data for T cells with wild type TRAC/TRBC loci (upper panel) or TRAC/TRBC double knockout (lower panel) incubated at a 3:1 effector to target ratio with HuCCT1 target cells. FIG. 4D provides data for T cells with wild type TRAC/TRBC loci (upper panel) or TRAC/TRBC double knockout (lower panel) incubated at a 3:1 effector to target ratio with HuCCT1 target cells. FIG. 4E provides data for T cells with wild type TRAC/TRBC loci or TRAC/TRBC double knockout (see legend) incubated at a 6:1 effector to target ratio with HuCCT1 target cells. FIG. 4F provides data for T cells with wild type TRAC/TRBC loci or TRAC/TRBC double knockout incubated with HuCCT1 target cells. FIG. 4G provides data for T cells with wild type TRAC/TRBC loci or TRAC/TRBC double knockout (see legend) incubated at a 3:1 effector to target ratio with Hpaf-II target cells. FIG. 4H and FIG. 4I provide data for T cells from two donors with wild type TRAC/TRBC loci (upper panel) or TRAC/TRBC double knockout (lower panel) incubated at a 3:1 effector to target ratio with Hpaf-II target cells. FIG. 4J provides data for T cells with wild type TRAC/TRBC loci (upper panel) or TRAC/TRBC double knockout (lower panel) incubated at a 3:1 effector to target ratio with Panc-1 target cells.

FIG. 4K summarizes efficiency of wild type TRAC/TRBC T cells and TRAC/TRBC dKO T cells transduced to express KRAS-G12D-specific TCRs for killing Hpaf-II, HuCCT1, and Panc-1 target cells.

FIG. 5A shows a scheme for an X-scan analysis to identify potential off-target TCR interaction partners.

FIGS. 5B-5E show a charts illustrating potentially cross-reactive peptides identified from a ScanProsite search for TCR91 (FIG. 5B), TCR2 (FIG. 5C), TCR4 (FIG. 5D), and TCR5 (FIG. 5E). Figure discloses “VVVGADGVGK” as SEQ ID NO: 295 and SEQ ID NOS 306-307.

FIG. 6 demonstrates TCR91 shows superior specificity against a KRAS G12D peptide compared to a RASL11B peptide. Shown is a plot of percentage CD137-positive cells alongside a table of EC50s and Cmax derived therefrom.

FIG. 7 shows dKO boosts the sensitivity of TCR more than a single knockout (sKO). Shown is a plot of percentage CD137-positive cells alongside a table of EC50s and Cmax derived therefrom.

FIG. 8 shows a comparison of Hpaf-II tumor cell killing of sKO and dKO cells expressing KRAS G12D-recognizing TCRs. Cytotoxic activity was assessed and plots of red fluorescence measured over time by live cell imaging were generated.

FIG. 9 shows enhanced killing of Panc-1 tumor cells by T cells harboring TRAC/TRBC dKO and expressing KRAS G12D-recognizing TCRs. The data was generated using the same protocol as described for FIG. 8 but with Panc-1 tumor cells instead of HpafII tumor cells.

FIG. 10A and FIG. 10B shows a Tetramer analysis of TCR91-transduced primary CD4+/CD8⁺ T cells having wild type TRAC/TRBC loci, a TRBC single knockout (sKO), or TRAC/TRBC dKO. Cell surface expression of correctly paired G12D TCR and its functionality was assessed using fluorophore labeled tetramers of MHC-peptide complex. FIG. 10A shows a bar graph of the percentage of tetramer-binding for each condition, and FIG. 10B shows mean fluorescence intensity (MFI) values.

FIG. 11 shows stimulation of KRASG12D specific TCR-T cells with G12D 10-mer. Shown is a plot of the percentage of CD137-positive cells versus peptide concentration for all cells (top panel) or transduced cells (bottom panel). TCR91-transduced TRAC/TRBC dKO cells exhibited increased activation compared to WT and TRBC sKO cells.

FIG. 12 shows an analysis of T cell cytotoxic activity of TCR91-transduced primary CD4+/CD8⁺ T cells having wild type TRAC/TRBC loci, a TRBC single knockout (sKO), or TRAC/TRBC dKO. Cytotoxic activity was assessed using the IncuCyte assay described for FIG. 2.

FIG. 13A and FIG. 13B show results of an experiment where primary CD4+ and CD8+ T cells were transduced with a KRAS-G12D-specific T cell receptor (TCR-91) at 35% (FIG. 13A) or 70% (FIG. 13B) transduction efficiency, with or without knockout of TRAC and TRBC. Shown are plots of total red object integrated intensity (a measure of tumor cell volume) over time by live cell imaging using the IncuCyte S3 microscope and software package as described for FIG. 2. The results demonstrate that the TRAC/TRBC dKO cells are more effective at controlling HpafII tumor cells relative to the TRAC/TRBC wild type cells.

FIG. 14A and FIG. 14B show results of an experiment where primary CD4+ and CD8+ T cells were transduced with a KRAS-G12D-specific T cell receptor 91 at 35% (FIG. 14A) or 70% (FIG. 14B) transduction efficiency, with or without knockout of TRAC and TRBC. Cytotoxic activity against Panc-1 tumor cells was evaluated at a 10:1 effector:target cell ratio using the IncuCyte assay. The results demonstrate that the TRAC/TRBC dKO cells are more effective at controlling Panc-1 tumor cells relative to the TRAC/TRBC wild type cells.

FIG. 15A and FIG. 15B shows a Tetramer analysis of TCR91-transduced primary CD4⁺/CD8⁺ T cells having wild type TRAC/TRBC loci, a TRAC single knockout (sKO), or TRAC/TRBC dKO. Cell surface expression of correctly paired G12D TCR and its functionality was assessed using fluorophore labeled tetramers of MHC-peptide complex. Shown are bar graphs of the percent of cells positive for tetramer binding (FIG. 15A) and level of tetramer binding to cells as measured by mean fluorescent intensity (MFI; FIG. 15B) for each condition. TRAC/TRBC dKO cells showed greater tetramer binding as compared to WT or TRAC sKO cells.

FIG. 15C provides representative scatterplots showing cell surface expression of correctly paired G12D TCR and its binding to fluorophore labeled tetramers of MHC-peptide complex. Primary CD4⁺ & CD8⁺ T cells were TCR-transduced, with or without TRAC/TRBC dKO.

FIG. 16A illustrates tumor volume over time for NSG mice implanted subcutaneously with CL40 colon adenocarcinoma cells and treated day 9 post-implant with 10×10\hat{ }6 T cells administered intravenously (control T cells with knockout of endogenous TRAC and TRBC, or T cells expressing a KRAS G12D-specific TCR disclosed herein with co-expression of a CD8 co-receptor (CD8αβ) and knockout of endogenous TRAC and TRBC).

FIG. 16B is a survival curve of NSG mice implanted subcutaneously with CL40 colon adenocarcinoma cells and treated day 9 post-implant with 10×10\hat{ }6 T cells administered intravenously (control T cells with knockout of endogenous TRAC and TRBC, or T cells expressing a KRAS G12D-specific TCR disclosed herein with co-expression of a CD8 co-receptor (CD8αβ) and knockout of endogenous TRAC and TRBC).

FIG. 17A shows enhanced STAT5 phosphorylation in engineered cells with the addition of a chimeric fusion protein comprising an IL-7 receptor intracellular signaling domain. “IL2” indicates a control condition in which cells were treated with recombinant IL2 to induce STAT5 phosphorylation.

FIG. 17B shows proliferation of T cells engineered to comprise KRAS G12D-specific TCRs disclosed herein in response to tumor cells. Data are shown comparing engineered T cells with or without addition of a chimeric fusion protein comprising an IL-7 receptor intracellular signaling domain.

FIG. 17C shows killing of tumor cells by T cells engineered to comprise a KRAS G12D-specific TCR disclosed herein, with or without addition of a chimeric fusion protein comprising an IL-7 receptor intracellular signaling domain.

FIG. 17D illustrates tumor volume over time for mice implanted subcutaneously with HuCCT1 cells and treated day 9 post-implant with 1×10\hat{ }7 T cells administered intravenously (control T cells with knockout of endogenous TRAC and TRBC, or T cells expressing a KRAS G12D-specific TCR disclosed herein with co-expression of a CD8 co-receptor (CD8αβ) and knockout of endogenous TRAC and TRBC, with (+ILR) or without (−ILR) a chimeric fusion protein comprising an IL-7 receptor intracellular signaling domain).

FIG. 18A is a chart demonstrating efficiency of non-viral knock in of a G12D KRAS TCR disclosed herein in primary CD4+ and CD8+ T cells.

FIG. 18B shows killing of Panc1 tumor cells by T cells engineered to comprise a KRAS G12D-specific TCR disclosed herein via lentiviral transduction (LV) or a non-viral knock in technique (KI).

FIG. 18C shows killing of HuCCT1 tumor cells by T cells engineered to comprise a KRAS G12D-specific TCR disclosed herein via lentiviral transduction (LVV) or a non-viral knock in technique (KI).

FIG. 19A shows the percentage of cells CD4/CD8 T cells expressing CD3 on the cell surface after targeting TRAC and TRBC loci with nuclease MG29-1. Activity was comparable to CRISPR/Cas9 system.

FIG. 19B shows the percent indel frequency at the TRAC and TRBC loci after MG29-1-mediated gene editing. UTD=untransduced.

FIG. 20 shows indel activity evaluated at 590 predicted potential off-targets (OTs) selected across TRAC and TRBC gRNAs that had up to 6 mismatches. High on-target editing efficiency was observed but no off-target activity was detected above background beyond the quantification limit of the assay (0.05%).

FIG. 21 shows an oligo-capture analysis performed in primary T cells to further evaluate the specificity of these nuclease/gRNA combinations.

FIG. 22A shows the frequencies of translocation events between TRAC and TRBC loci in edited primary T cells as detected by dPCR. FIG. 22B shows the frequencies of translocation events between TRAC and TRBC loci as detected by karyotyping. FIG. 22C and FIG. 22D show comparisons of activation as measured by the percentage of 2A+ T cells expressing CD137 of T cells modified to express a KRAS-G12D-specific TCR and having wild type TRAC/TRBC loci (FIG. 22C) or either TRAC sKO or TRAC/TRBC dKO (FIG. 22D) and after stimulation with the cognate peptide recognized by the TCR. FIG. 22E shows comparisons of activation as measured by the percentage of 2A+ T cells expressing CD137 of T cells having either TRAC sKO or TRAC/TRBC dKO after coculture with A11 antigen presenting cells. FIGS. 22F-22M show comparisons of activation as measured by the percentage of 2A+ T cells expressing CD137 of T cells modified to express a KRAS-G12D-specific TCR and having wild type TRAC/TRBC loci or either TRAC sKO or TRAC/TRBC dKO and after coculture with A11-expressing cancer cell lines. FIG. 22F shows data for T cells modified with the pGE106, pGE116, pGE107, or pGE129 constructs after coculture with HPAF-II cells. FIG. 22G shows data for T cells modified with the pGE106, pGE116, pGE107, or pGE129 constructs after coculture with HuCCT1 cells. FIG. 22H shows data for T cells modified with the pGE106, pGE116, pGE107, or pGE129 constructs after coculture with Panc1 cells. FIG. 22I shows data for T cells modified with the pGE106, pGE116, pGE107, or pGE129 constructs after coculture with CL40 cells. FIG. 22J shows data for T cells modified with the pGE106, pGE114, or pGE129 constructs after coculture with HPAF cells. FIG. 22K shows data for T cells modified with the pGE106, pGE114, or pGE129 constructs after coculture with HuCCT1 cells. FIG. 22L shows data for T cells modified with the pGE106, pGE114, or pGE129 constructs after coculture with Panc1 cells. FIG. 22M shows data for T cells modified with the pGE106, pGE114, or pGE129 constructs after coculture with CL40 cells. FIGS. 22N-22T show comparisons of in vitro proliferation of T cells modified via electroporation-mediated knockin or LVV-mediated transduction to express a KRAS-G12D-specific TCR and having wild type TRAC/TRBC loci or either TRAC sKO or TRAC/TRBC dKO and after coculture with A11-expressing cancer cell lines. FIG. 22N shows data for cells modified to express either TCR2 and CD8 alpha/beta or TCR2, CD34-IL7R, and CD8 alpha/beta after coculture with HuCCT1 cells at a 3:1 effector to target ratio. FIG. 22O shows data for cells modified to express either TCR2 and CD8 alpha/beta or TCR2, CD34-IL7R, and CD8 alpha/beta after coculture with Panc1 cells at a 3:1 effector to target ratio. FIG. 22P shows data for cells modified to express either TCR2 and CD8 alpha/beta or TCR2, CD34-IL7R, and CD8 alpha/beta after coculture with with HPAF-II cells at a 1:1 effector to target ratio. FIG. 22Q shows data for cells modified to express either TCR2 and CD8 alpha/beta or TCR2, CD34-IL7R, and CD8 alpha/beta after coculture with CL40 cells at a 1:1 effector to target ratio. FIG. 22R shows data for T cells modified with the pGE106, pGE116, pGE107, or pGE129 constructs after coculture with HPAF cells at a 1:1 effector to target ratio. FIG. 22S shows data for T cells modified with the pGE106, pGE116, pGE107, or pGE129 constructs after coculture with HuCCT1 cells at a 6:1 effector to target ratio. FIG. 22T shows data for T cells modified with the pGE106, pGE116, pGE107, or pGE129 constructs after coculture with Panc1 cells at a 6:1 effector to target ratio. FIG. 22U shows comparisons of cytotoxic activity of T cells modified to express either TCR2, CD34-IL7R, and CD8 alpha/beta or TCR2, CD58-IL7R, and CD8 alpha/beta after coculture with HuCCT1 cells at a 10:1 effector to target cell ratio.

FIG. 23 provides graphs showing that T cells engineered with a non-viral targeted knock-in (KI) at the TCRα constant chain (TRAC) locus to express a multi-cistronic cassette that includes 1) a high-affinity TCR specific for the KRAS G12D mutation, 2) a CD8αβ coreceptor, and 3) a chimeric cytokine receptor (G12D TCR-T cells), bind KRAS G12D peptide with high functional avidity and show robust cytotoxicity in vitro. G12D TCR-T TCR T cells bind specifically to the KRAS G12D peptide even at sub-nanomolar concentrations (left) and showed robust cytotoxicity against HuCCT1 tumor cells (endogenous KRAS G12D and HLA-A*11-01) even upon rechallenge with tumor cells.

FIG. 24 provides graphs showing that Engineered T cells show robust tumor cell control in vivo. Engineered CD4/CD8 TCR T cells were intravenously administered in NSG mice after subcutaneous inoculation of HuCCT1 tumor cells (left) or CL40 tumor cells (right). Treatment with G12D TCR-T cells resulted in durable robust responses in murine xenograft models.

FIG. 25 provides a heat map and a graph showing that G12D TCR-T cells show low risk of cross-reactivity. Engineered TCR T cells were incubated with a library of X-scan peptides and activation of TCR T cells was measured to reveal the recognition motif for the KRAS G12D TCR. The recognition motif was used to scan for matches to human peptides. None of the potentially cross-reactive peptides activated TCR T cells even at supraphysiologic 500 nM concentration demonstrating high specificity of the KRAS G12D TCR. Figure discloses SEQ ID NO: 295.

FIGS. 26A-26B provide graphs showing that gene editing reagents show high specificity. To evaluate off-target activity for gene editing reagents, potential off-target sites were identified using in silico prediction (FIG. 26A) and oligo-capture analysis (FIG. 26B) performed in primary T cells. Each of these potential off-targets was assayed for insertions and deletions in G12D TCR-T cells using a targeted sequencing assay. While G12D TCR-T cells showed high on-target activity, none of the potential off-targets showed significant off-target activity confirming high specificity of the GE reagents used for manufacturing G12D TCR-T cells.

FIG. 27 provides graphs showing that non-viral KI achieves high efficiency of transgene integration. Non-viral knock-in can achieve >40% transgene integration efficiency in T cells from healthy donors. The process performed similarly at a research scale and a 10× scale up version. Engineered T cells expanded robustly achieving enough cell doses for clinical application demonstrating manufacturability.

FIG. 28 provides graphs showing that KI-engineered T cells from patient donors show robust cytotoxicity. T cells from patient donors and healthy donors were engineered using non-viral KI, and evaluated for KI efficiency, growth kinetics and functionality using in vitro cytotoxicity assay. Patient TCR T cells performed similarly to healthy donor TCR T cells.

FIG. 29 provides a schematic and graphs showing that lentiviral delivery is limited by cargo size. The schematic shows the transgene construct used in Example 14. The graphs show that virus titer decreases with increasing transgene size (left) and that transduction efficiency decreases with increasing transgene size (right).

FIGS. 30A-30B provide a schematic and graphs showing that non-viral KI can achieve high transgene integration frequency even with large transgenes. FIG. 30A shows a schematic of transgenes inserted into the endogenous TRAC gene via CRISPR/Cas driven homology-directed repair which allows for larger cargo capacity. FIG. 30B shows graphs showing that the optimized KI process can achieve ˜50% KI efficiency and yield sufficient number of engineered TCR T cells to meet clinical dose. KI process does not skew T cell populations.

FIG. 31 provides a graph showing improved tetramer binding by KI cells. KI Engineered TCR T cells show improved binding to KRAS G12D tetramer even with fewer transgene vector copies per cell (VCN), indicative of higher TCR expression driven by the EF-1a promoter.

FIG. 32 provides a graph showing increased avidity for KI cells. KI engineered TCR T cells bind the KRAS G12D peptide with higher functional avidity than LVV cells consistent with the increased TCR expression.

FIG. 33 provides a graph showing that KI cells show superior activity in vivo. KI cells show superior tumor control after a single intravenous administration of 5 million CD4/CD8 TCR-T 10 days after subcutaneous inoculation of HuCCT1 tumor cells in NSG mice.

FIG. 34 provides a schematic of EF-1α and promoter-less constructs for KI. Non-viral KI provides flexibility of utilizing endogenous promoter or an exogenous promoter like EF-Ta. Leveraging the endogenous TRAC promoter for driving transgenes allows for native TCR expression control.

FIG. 35 provides a graph showing reduced transgene expression with the TRAC promoter. EF-1a promoter drives higher levels of TCR, CD8ab and ILR as compared to the endogenous TRAC promoter.

FIG. 36 provides a graph showing lower functional avidity with TRAC promoter. KI engineered TCR T cells utilizing the EF-1a promoter bind the KRAS G12D peptide with higher functional avidity than cells utilizing the TRAC promoter.

FIG. 37 provides a graph showing less in vivo activity using TRAC promoter. KI cells utilizing the EF-1a promoter show superior tumor control after a single intravenous administration of 5 million CD4/CD8 TCR-T 10 days after subcutaneous inoculation of HuCCT1 tumor cells in NSG mice.

FIG. 38 provides a schematic for the study of Example 15. The schematic contains the following abbreviations: 2L+=second line plus; BOIN12=Bayesian optimal interval Phase I/II; CRC=colorectal cancer; DL=dose levels; DLT=dose-limiting toxicity; EOS=end of study, HLA=human leukocyte antigen; ICF=informed consent form; KRAS=Kirsten rat sarcoma virus; NSCLC=non small cell lung cancer; OBD=optimal biological dose; PD=progressive disease; PDAC=pancreatic ductal adenocarcinoma; PTFD=post treatment follow-up discontinuation; SMC=Safety Monitoring Committee; TCR=T cell receptor.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

### Host Cells or Compositions

In some aspects, the present disclosure provides for a host cell comprising an extracellular binding protein wherein the binding protein is capable of binding to a peptide:HLA complex, wherein the peptide comprises a KRAS G12 mutant peptide. In some embodiments, the peptide:HLA complex comprises an HLA-A*11 allele. In some embodiments, the peptide:HLA complex comprises an HLA-A*11:01 allele. In some embodiments, the peptide:HLA complex comprises an HLA allele that binds or is predicted to bind a KRAS mutant peptide (e.g., the G12 mutant peptide, such as G12D) with a suitable affinity for presentation and TCR activation, for example, a binding affinity or KD of at most 1000 nM, at most 750 nM, at most 500 nM, at most 250 nM, at most 100 nM, at most 50 nM, or at most 10 nM.

In some cases, the host cell comprises an immune cell or a precursor thereof. In some cases, the immune cell comprises a T cell, a NK cell, a NK-T cell, a dendritic cell, a macrophage, a monocyte, or any combination thereof. In some cases, the immune cell is a T cell, wherein the T cell comprises a CD4+ T cell, a CD8+ T cell, a CD4− CD8− double negative T cell, a CD4+ CD8+ double positive T cell, a γδ T cell, or any combination thereof. In some cases, the host cell further comprises a transgenic polynucleotide encoding a polypeptide that comprises an extracellular portion of a CD8 co-receptor α (CD8α) chain or a polynucleotide encoding a polypeptide that comprises an extracellular portion of a CD8 co-receptor β (CD8β) chain. In some cases, when the host cell is in the presence of a tumor cell that expresses a KRAS G12D mutant peptide, CD137 expression of the host cell is elevated as compared to: (i) CD137 expression by a reference human T cell not expressing the binding protein, when the reference human T cell is in the presence of the tumor cell; or (ii) CD137 expression by the human T cell expressing the binding protein when not in the presence of the tumor cell or when not in the presence of an antigen-presenting cell expressing a peptide:HLA complex.

### Host Cells

The disclosure provides host cells (e.g., engineered immune cells) and populations thereof that comprise, encode, and/or are capable of expressing an extracellular binding protein disclosed herein.

A host cell can be a peripheral blood mononuclear cell (PBMC). A host cell can be a lymphoid cell. A host cell can be a lymphocyte. A host cell can be a T cell. A host cell can be a B cell. A host cell can be a natural killer (NK) cell. A host cell can be a Natural Killer T (NKT) cell. A host cell can be a mammalian cell. A host cell can be a human cell.

A host cell can be a primary cell. A host cell can be an immortalized cell. A host cell can be of a cell line. A host cell can be differentiated from a stem cell, for example, an induced pluripotent stem cell (iPSC), embryonic stem cell, hematopoietic stem cell (HSC), or the like.

In some cases, the host cells are “off-the-shelf” cells that are engineered from an immune cell line, for instance, a T cell line or an NK cell line (e.g., NK-92, or e.g., NK-YS, KHYS-1, NKL, NKG, SNK-6, or IMC-1). Such host cells can be readily available and formulated for direct administration to a subject in need thereof.

A host cell can be an alpha beta T cell. A host cell can be a gamma delta T cell. In some embodiments, a host cell comprises a disruption or deletion of one or more endogenous TCR-encoding genes, such as TRAC, TRB (e.g., TRBC1 and/or TRBC2), TRG, and/or TRD. In some embodiments, a host cell comprises a disruption or deletion of a variable region of one or more endogenous TCR-encoding genes, such as a disruption or deletion in TRAC, TRB, TRG, and/or TRD. In some embodiments, a host cell comprises a disruption or deletion of a constant region of one or more endogenous TCR-encoding genes.

### Extracellular Binding Protein

The extracellular binding protein can comprise a TCR or a portion thereof. In some embodiments, the extracellular binding protein comprises a T cell receptor (TCR) a chain variable (Vα) region, a TCR β chain variable (Vβ) region, a T cell receptor (TCR) a chain constant (Cα) region, and/or a T cell receptor (TCR) β chain constant (Cβ) region. The extracellular binding protein can also comprise a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region, or a combination thereof.

The extracellular binding protein can comprise a component of a TCR signaling complex, for example, an extracellular domain, transmembrane domain, and/or cytoplasmic domain of a TCR signaling complex, such as a human TCR signaling complex.

The extracellular binding protein can comprise (i) an extracellular domain of TCR alpha chain constant region, TCR beta chain constant region, TCR gamma chain constant region, or TCR delta chain constant region; (ii) a transmembrane domain of TCR alpha chain, TCR beta chain, TCR gamma chain, or TCR delta chain; and/or (iii) a cytoplasmic domain of TCR alpha chain, TCR beta chain, TCR gamma chain, or TCR delta chain. The extracellular binding protein can comprise a full length or substantially full length TCR alpha chain, TCR beta chain, TCR gamma chain, and/or TCR delta chain.

In some cases, the extracellular binding protein comprises a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprising a sequence having at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to any of the TCR Vα, Vβ, FR1, CDR1, FR2, CDR2, FR3, CDR3, or FR4 sequences described in Table 1.

An extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region disclosed herein can comprise, consist essentially of, or consist of an amino acid sequence with at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to any one of the TCR Vα, Vβ, FR1, CDR1, FR2, CDR2, FR3, CDR3, or FR4 sequences disclosed in Table 1.

An extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region disclosed herein can comprise, consist essentially of, or consist of an amino acid sequence with at most about 70%, at most about 71%, at most about 72%, at most about 73%, at most about 74%, at most about 75%, at most about 76%, at most about 77%, at most about 78%, at most about 79%, at most about 80%, at most about 81%, at most about 82%, at most about 83%, at most about 84%, at most about 85%, at most about 86%, at most about 87%, at most about 88%, at most about 89%, at most about 90%, at most about 91%, at most about 92%, at most about 93%, at most about 94%, at most about 95%, at most about 95.5%, at most about 96%, at most about 96.5%, at most about 97%, at most about 97.5%, at most about 98%, at most about 98.5%, at most about 99%, or at most about 99.5% sequence identity or sequence similarity to any one of the TCR Vα, Vβ, FR1, CDR1, FR2, CDR2, FR3, CDR3, or FR4 sequences disclosed in Table 1.

In some embodiments, an extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises, consists essentially of, or consists of an amino acid sequence with about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 95.5%, about 96%, about 96.5%, about 97%, about 97.5%, about 98%, about 98.5%, about 99%, about 99.5% or about 100% sequence identity or sequence similarity to any one of the TCR Vα, Vβ, FR1, CDR1, FR2, CDR2, FR3, CDR3, or FR4 sequences disclosed in Table 1.

The degree of sequence identity between two sequences can be determined, for example, by comparing the two sequences using computer programs designed for this purpose, such as global or local alignment algorithms. Non-limiting examples include BLASTp, BLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method or algorithm. A global alignment algorithm, such as a Needleman and Wunsch algorithm, can be used to align two sequences over their entire length, maximizing the number of matches and minimizes the number of gaps. Default settings can be used.

To generate similarity scores for two amino acid sequences, scoring matrices can be used that assign positive scores for some non-identical amino acids (e.g., amino acids with similar physio-chemical properties and/or amino acids that exhibit frequent substitutions in orthologs, homologs, or paralogs), Non-limiting examples of scoring matrices include PAM30, PAM70, PAM250, BLOSUM45, BLOSUM50, BLOUM62, BLOSUM80, and BLOSUM90.

In some embodiments, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises, consists essentially of, or consists of the amino acid sequence of any one of the TCR Vα, Vβ, FR1, CDR1, FR2, CDR2, FR3, CDR3, or FR4 sequences disclosed in Table 1.

In some embodiments, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises an amino acid sequence with one or more insertions, deletions, and/or substitutions relative to any one of the sequences disclosed in Table 1.

For example, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid insertions relative to any one of the sequences disclosed in Table 1.

In some embodiments, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of the sequences disclosed in Table 1.

In some embodiments, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises 1, 2, 3, 4, 5, 6, 7, 8, 9, 10, 11, 12, 13, 14, 15, 16, 17, 18, 19, 20, 25, 30, 35, 40, 45, or 50 amino acid insertions relative to any one of the sequences disclosed in Table 1.

The one or more insertions can be at the N-terminus, the C-terminus, within the amino acid sequence, or a combination thereof. The one or more insertions can be contiguous, non-contiguous, or a combination thereof.

In some embodiments, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid deletions relative to any one of the sequences disclosed in Table 1.

In some embodiments, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of the sequences disclosed in Table 1.

In some embodiments, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises 1, 2, 3, 4, 5, 6, 7, 8, 9, 10, 11, 12, 13, 14, 15, 16, 17, 18, 19, 20, 25, 30, 35, 40, 45, or 50 amino acid deletions relative to any one of the sequences disclosed in Table 1.

The one or more deletions can be at the N-terminus, the C-terminus, within the amino acid sequence, or a combination thereof. The one or more deletions can be contiguous, non-contiguous, or a combination thereof.

In some embodiments, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 amino acid substitutions relative to any one of the sequences disclosed in Table 1.

In some embodiments, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of the sequences disclosed in Table 1.

In some embodiments, the extracellular binding protein, a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region comprises 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to any one of the sequences disclosed in Table 1.

The one or more substitutions can be at the N-terminus, the C-terminus, within the amino acid sequence, or a combination thereof. The one or more substitutions can be contiguous, non-contiguous, or a combination thereof. In some embodiments the one or more substitutions are conservative. In some embodiments the one or more substitutions are non-conservative.

In some cases, the extracellular binding protein comprises a TCR α chain; a TCR β chain; a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region that is human, humanized, or chimeric.

In some cases, the KRAS G12 mutant peptide is a KRAS G12D mutant peptide. In some cases, the KRAS G12 mutant peptide comprises the amino acid sequence VVVGADGVGK (SEQ ID NO: 295). In some cases, the extracellular binding protein is selective for the KRAS G12D mutant peptide, e.g., specifically, selectively or preferentially binds the KRAS G12D mutant peptide. In some cases, the extracellular binding protein is at least 2-, 3-, 5-, 10-, 25-, 50-, 100-, 200-, 500-, or 1000-, 2000-, 3000-, 4000-, 5000-fold, or 10,000-fold selective for the KRAS G12D mutant peptide versus other 10-mer peptides, for example, a corresponding wild type peptide, or a peptide encoded by a genome of the cell (e.g., that binds to a different KRAS G12-specific TCR, or that is predicted to exhibit off-target binding to the extracellular binding protein).

In some cases, the extracellular binding protein has a log 10EC50 for the KRAS G12 mutant peptide of about −6.0 or less, about −6.1 or less, about −6.2 or less, about −6.3 or less, about −6.4 or less, about −6.5 or less, about −6.6 or less, about −6.7 or less, about −6.8 or less, about −6.9 or less, about −7.0 or less, about −7.1 or less, about −7.2 or less, about −7.3 or less, about −7.4 or less, about −7.5 or less, about −7.6 or less, about −7.7 or less, about −7.8 or less, about −7.9 or less, about −8.0 or less, about −8.1 or less, about −8.2 or less, about −8.3 or less, about −8.4 or less, about −8.5 or less, about −8.6 or less, about −8.7 or less, about −8.8 or less, about −8.9 or less, about −9 or less, about −9.1 or less, or about −9.2 or less.

In some embodiments, a host cell disclosed herein comprises an extracellular binding protein (e.g., TCR) that binds a target antigen of the extracellular binding protein (for example, a KRAS G12 mutant peptide, such as KRAS G12D mutant peptide, e.g., present in a peptide:HLA complex) with an EC50 (e.g., peptide dose at which a half-maximal activation of a T cell population is reached) of less than about 100 mM, less than about 10 mM, less than about 1 mM, less than about 500 μM, less than about 100 μM, less than about 50 μM, less than about 10 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 500 μM, or less than about 100 μM. The host cell can comprise, for example, a modification (e.g., genomic mutation) that results in decreased expression of endogenous TRAC, TRBC1, and/or TRBC2, a combination thereof. The extracellular binding protein can be a TCR that comprises a Vα and Vβ regions and/or CDRs disclosed herein.

In some embodiments, a host cell disclosed herein comprises an extracellular binding protein (e.g., TCR) that binds a target antigen of the extracellular binding protein (for example, a KRAS G12 mutant peptide, such as KRAS G12D mutant peptide, e.g., present in a peptide:HLA complex) with an EC50 (e.g., peptide dose at which a half-maximal activation of a T cell population is reached) of at least about 100 mM, at least about 10 mM, at least about 1 mM, at least about 500 μM, at least about 100 μM, at least about 50 μM, at least about 10 μM, at least about 5 μM, at least about 4 μM, at least about 3 μM, at least about 2 μM, at least about 1 μM, at least about 900 nM, at least about 800 nM, at least about 700 nM, at least about 600 nM, at least about 500 nM, at least about 400 nM, at least about 300 nM, at least about 200 nM, at least about 100 nM, at least about 90 nM, at least about 80 nM, at least about 70 nM, at least about 60 nM, at least about 50 nM, at least about 40 nM, at least about 30 nM, at least about 20 nM, at least about 10 nM, at least about 5 nM, at least about 1 nM, at least about 500 μM, at least about 100 μM. The host cell can comprise, for example, a modification (e.g., genomic mutation) that results in decreased expression of endogenous TRAC, TRBC1, and/or TRBC2, a combination thereof. The extracellular binding protein can be a TCR that comprises a Vα and Vβ regions and/or CDRs disclosed herein.

In some embodiments, an extracellular binding protein (e.g., TCR) binds a target (for example, a KRAS G12 mutant peptide, such as KRAS G12D mutant peptide, e.g., present in a peptide:HLA complex) with a KD of less than about 100 mM, less than about 10 mM, less than about 1 mM, less than about 500 μM, less than about 100 μM, less than about 50 μM, less than about 10 μM, less than about 5 μM, less than about 4 μM, less than about 3 μM, less than about 2 μM, less than about 1 μM, less than about 900 nM, less than about 800 nM, less than about 700 nM, less than about 600 nM, less than about 500 nM, less than about 400 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 500 μM, or less than about 100 μM.

### Modifications for Reduced Expression of Endogenous TCR Genes

The host cell can further comprise one or more modifications (e.g., genomic mutation(s)) that causes or contributes to decreased expression of an endogenous T cell receptor α constant (TRAC), a T cell receptor β constant 1 (TRBC1) locus, or a T cell receptor β constant 2 (TRBC2) locus. Decreased expression of TRAC, TRBC1, and/or TRBC2 can, for example, reduce mispairing of transgenic TCR chains that are introduced into a host cell (e.g., anti-KRAS G12D TCRs disclosed herein) with endogenous TCR chains, improve functional expression of the transgenic TCRs, and improve TCR complex signaling and functionality by freeing the available pool of CD3 proteins to bind to the transgenic TCR rather than endogenous TCR chains.

In some embodiments, the modification can facilitate enhanced in vitro, ex vivo, or in vivo tumor cell killing by engineered immune cells than comparable control cells lacking the modification. In some embodiments, the modification can facilitate enhanced sensitivity to a given (e.g., low) density of a target antigen (e.g., KRAS G12 mutant peptide) compared to a corresponding control cell lacking the modification.

In some embodiments, the genomic mutation that causes or contributes to decreased expression of an endogenous T cell receptor α constant (TRAC), a T cell receptor β constant 1 (TRBC1) locus, or a T cell receptor β constant 2 (TRBC2) locus comprises an indel in the TRAC, TRBC1, or TRBC2 locus. In some embodiments, the genomic mutation that causes or contributes to decreased expression of an endogenous T cell receptor α constant (TRAC), a T cell receptor β constant 1 (TRBC1) locus, or a T cell receptor β constant 2 (TRBC2) locus is a missense mutation and may result in reduced function or stability of a T cell receptor α or T cell receptor β polypeptide encoded in the genome of the host cell. In some embodiments, the genomic mutation that causes or contributes to decreased expression of an endogenous T cell receptor α constant (TRAC), a T cell receptor β constant 1 (TRBC1) locus, or a T cell receptor β constant 2 (TRBC2) locus also results in premature termination of a T cell receptor α or T cell receptor β polypeptide translated from a genomic mRNA of the cell. In some cases, the host cell comprises genomic mutations that cause or contribute to decreased expression of both (i) TRAC; and (ii) TRBC1 or TRBC2. In some cases, the host cell comprises genomic mutations that causes or contributes to decreased expression of TRAC, TRBC1, and TRBC2. The genomic mutation can be or can comprise an insertion, e.g., of an expression cassette. The genomic mutation can be or can comprise a deletion. The genomic mutation can be or can comprise a substitution.

In some embodiments, the modification comprises deletion of, for example, at least 10%, at least 20%, at least 30%, at least 40%, at least 50%, at least 60%, at least 70%, at least 80%, or at least 90% of the open reading frame of endogenous TRAC, TRBC1, or TRBC2.

In some embodiments, the modification comprises knockdown of expression of the TRAC, TRBC1, or TRBC2, for example, using a shRNA or siRNA. In some embodiments, the modification comprises a genomic disruption. In some embodiments, the modification comprises insertion of, for example, a transposon, or a premature stop codon.

In some embodiments, expression of endogenous TRAC, TRBC1, and/or TRBC2 is reduced by at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, or at least 5000 fold. In some embodiments, expression of TRAC, TRBC1, and/or TRBC2 is eliminated or substantially eliminated. In some embodiments, expression of TRAC, TRBC1, and/or TRBC2 is reduced to below a limit of detection. The reduced expression of TRAC, TRBC1, and/or TRBC2 can be determined, for example, by a flow cytometric assay (e.g., for proportion of positive cells or mean fluorescence intensity, in a population of interest).

In some embodiments, the reduction in expression of endogenous TRAC, TRBC1, and/or TRBC2 is found in at least 5%, at least 10%, at least 20%, at least 30%, at least 40%, at least 50%, at least 60%, at least 70%, at least 80%, at least 90%, at least 95%, at least 97%, or at least 99% of host cells in a population. In some cases, the reduction of expression is found by a genomic sequencing method.

In some embodiments, a population of host cells disclosed herein comprising one or more modifications (e.g., genomic mutation(s)) that result in decreased expression of endogenous TRAC, TRBC1, and/or TRBC2 exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, or at least 5000 fold increased killing of target cells as compared to a population of control cells (for example, cells without reduced expression of TRAC, TRBC1, and/or TRBC2). The killing of target cells can be, for example, as determined by an in vitro cytotoxicity assay. The host cells can comprise an extracellular binding protein (e.g., a TCR comprising Vα and Vβ regions and/or CDRs disclosed herein) that binds a target antigen (for example, a KRAS G12 mutant peptide, such as KRAS G12D mutant peptide, e.g., present in a peptide:HLA complex).

In some embodiments, a population of host cells disclosed herein comprising one or more modifications (e.g., genomic mutation(s)) that result in decreased expression of endogenous TRAC, TRBC1, and/or TRBC2 exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, or at least 5000 fold increased activation in response to target cells as compared to a population of control cells (for example, cells without reduced expression of TRAC, TRBC1, and/or TRBC2). The host cells can comprise an extracellular binding protein (e.g., a TCR comprising Vα and Vβ regions and/or CDRs disclosed herein) that binds a target antigen (for example, a KRAS G12 mutant peptide, such as KRAS G12D mutant peptide, e.g., present in a peptide:HLA complex). The activation can be, for example, as determined by an assay for determining expression an activation marker (e.g., CD137, CD69, Granzyme B, CD107a, IFN-gamma, TNF-α, IL-12, a cytokine, an interleukin, an interferon) upon exposure to target cells that express or present the target antigen.

In some embodiments, a population of host cells disclosed herein comprising one or more modifications (e.g., genomic mutation(s)) that result in decreased expression of endogenous TRAC, TRBC1, and/or TRBC2 exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 20 fold, at least 30 fold, at least 40 fold, at least 50 fold, at least 60 fold, at least 70 fold, at least 80 fold, at least 90 fold, at least 100 fold, at least 150 fold, at least 200 fold, at least 250 fold, at least 300 fold, at least 350 fold, at least 400 fold, at least 500 fold, at least 600 fold, at least 700 fold, at least 800 fold, at least 900 fold, at least 1000 fold, or at least 5000 fold increased avidity for a target antigen of an extracellular binding protein as compared to a population of control cells (for example, cells without reduced expression of TRAC, TRBC1, and/or TRBC2). The host cells can comprise an extracellular binding protein (e.g., a TCR comprising Vα and Vβ regions and/or CDRs disclosed herein) that binds a target antigen (for example, a KRAS G12 mutant peptide, such as KRAS G12D mutant peptide, e.g., present in a peptide:HLA complex). The increase in avidity can be, for example, as determined by an assay for determining expression an activation marker (e.g., CD137, CD69, Granzyme B, CD107a, IFN-gamma, TNF-α, IL-12, a cytokine, an interleukin, an interferon) upon exposure to target cells that express or present the target antigen, or and/or an assay to determine EC50 (e.g., peptide dose at which a half-maximal activation of a T cell population is reached).

In some embodiments, a population of host cells disclosed herein comprising one or more modifications (e.g., genomic mutation(s)) that result in decreased expression of endogenous TRAC, TRBC1, and/or TRBC2 exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 10 fold, at least 20 fold, or at least 50 fold increased binding to a target antigen of an extracellular binding protein as compared to a population of control cells (for example, cells without reduced expression of TRAC, TRBC1, and/or TRBC2). The host cells can comprise an extracellular binding protein (e.g., a TCR comprising Vα and Vβ regions and/or CDRs disclosed herein) that binds the target antigen (for example, a KRAS G12 mutant peptide, such as KRAS G12D mutant peptide, e.g., present in a peptide:HLA complex). The increase in binding can be, for example, as determined by an assay comprising staining with peptide-HLA multimers (e.g., tetramers or pentamers).

In some embodiments, a population of host cells disclosed herein comprising one or more modifications (e.g., genomic mutation(s)) that result in decreased expression of endogenous TRAC, TRBC1, and/or TRBC2 exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 10 fold, at least 20 fold, at least 50 fold, at least 100-fold, or at least 500-fold increased expression (e.g., cell surface expression) of an extracellular binding protein as compared to a population of control cells (for example, cells without reduced expression of TRAC, TRBC1, and/or TRBC2). The extracellular binding protein can be a TCR comprising an alpha chain and beta chain (e.g., with Vα and Vβ regions and/or CDRs disclosed herein) that binds the target antigen (for example, a KRAS G12 mutant peptide, such as KRAS G12D mutant peptide, e.g., present in a peptide:HLA complex). The increase in expression can be, for example, as determined by an assay comprising staining with peptide-HLA multimers (e.g., tetramers or pentamers) specific for the extracellular binding protein.

In some embodiments, the population of host cells comprises decreased expression of endogenous TRAC relative to the control cells. In some embodiments, the population of host cells comprises decreased expression of endogenous TRBC1 relative to the control cells. In some embodiments, the population of host cells comprises decreased expression of endogenous TRBC2 relative to the control cells. In some embodiments, the population of host cells comprises decreased expression of endogenous TRAC and TRBC1 relative to the control cells. In some embodiments, the population of host cells comprises decreased expression of endogenous TRAC and TRBC2 relative to the control cells. In some embodiments, the population of host cells comprises decreased expression of endogenous TRBC1 and TRBC2 relative to the control cells. In some embodiments, the population of host cells comprises decreased expression of endogenous TRAC, TRBC1, and TRBC2 relative to the control cells.

### Additional Polypeptides

A host cell can be engineered to comprise further modifications in addition to an extracellular binding protein and a modification that causes or contributes to decreased expression of TRAC, TRBC1, and/or TRBC2.

A host cell can comprise a transgenic polynucleotide encoding a polypeptide that comprises a CD8 co-receptor α (CD8α) chain or an extracellular portion thereof, and/or a transgenic polynucleotide encoding a CD8 co-receptor β (CD8β) chain polypeptide or an extracellular portion thereof. A host cell can be engineered to express a CD8 co-receptor disclosed herein, e.g., a CD8α chain and/or a CD8β chain. Illustrative, non-limiting examples of CD8α and CD8β amino acid sequences that can be used include those provided SEQ ID NO: 219, SEQ ID NO: 220, and variants thereof.

A host cell can comprise a transgenic polynucleotide encoding a Fas-41BB fusion protein. The Fas-41BB fusion protein can comprise, for example, an extracellular domain of Fas or a FasL-binding fragment thereof, and an intracellular signaling domain of 41BB or a signaling domain thereof. A Fas-41BB fusion protein can be useful for, for example, converting a signal initiated by the binding of Fas to its target (e.g., FasL) into a positive (e.g., costimulatory) signal generated by the 4-1BB intracellular signaling domain, thereby improving anti-cancer immune functionality of a host cell disclosed herein (e.g., increased proliferation, survival in the tumor microenvironment, and metabolism to support T cell activation and memory development). The extracellular component can comprise all or a portion of the extracellular domain of Fas, or can be truncated to maintain a short spatial distance between the host cell and an interaction partner (e.g., ˜9aas) upon receptor-ligand interaction. The Fas-41BB fusion protein can comprise a transmembrane domain, for example, a Fas, 4-1BB, or CD28 transmembrane domain. An illustrative, non-limiting example of a Fas-41BB fusion protein is provided in SEQ ID NO: 221.

A host cell can comprise a transgenic polynucleotide encoding a chimeric fusion protein that comprises an IL7R intracellular signaling domain. The chimeric fusion protein can comprise, for example, an intracellular portion of an Interleukin 7 Receptor A (IL7RA) polypeptide, or a portion or variant thereof that is capable of contributing to an IL-7 signal in a host cell. A chimeric IL7R fusion protein can, for example, provide a “signal 3” to increase STAT5 phosphorylation and host cell functionality, enhance proliferation of a host cell, increase host cell survival (e.g., in the tumor microenvironment), and/or enhance chemokine receptor expression.

Interleukin-7 receptor subunit alpha can also be referred to as IL7R-α, as IL7RA, as IL-7R-alpha, as ILRA, as Interleukin-7 receptor-α, as interleukin 7 receptor, as Cluster of Differentiation 127 as CD127, or as CDW127.

An IL7R intracellular signaling domain can comprise an amino acid sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 95.5%, at least about 96%, at least about 96.5%, at least about 97%, at least about 97.5%, at least about 98%, at least about 98.5%, at least about 99%, at least about 99.5%, or about 100% sequence identity or sequence similarity to SEQ ID NO: 224.

In some embodiments, the IL7R intracellular signaling domain comprises (a) one or more residues of a BOX1 motif corresponding to residues 8-15 (VWPSLPDH (SEQ ID NO: 302)) relative to SEQ ID NO: 224 when optimally aligned, or (b) Y185 relative to SEQ ID NO: 224 when optimally aligned. In some embodiments, the IL7R intracellular signaling domain comprises one or more residues of a FERM domain corresponding to residues 1-6 (KKRIKPI (SEQ ID NO: 303)) or residues 16-28 (KKTLEHLCKKPRK (SEQ ID NO: 304)) relative to SEQ ID NO:224 when optimally aligned.

In some embodiments, the chimeric fusion protein comprises an IL7R transmembrane domain. The IL7R transmembrane domain can comprise an amino acid sequence with at least 80%, at least 85%, at least 90%, at least 95%, at least 99%, or 100% sequence identity to SEQ ID NO: 225. In some embodiments, the IL7R transmembrane domain comprises a mutation relative to SEQ ID NO: 225. In some embodiments, the mutation is, or comprises, the insertion of one or more cysteines, and/or one or more prolines, into the amino acid sequence of SEQ ID NO: 225. In some embodiments, the mutation enables or facilitates homodimerization of the receptor. In some embodiments, the mutation comprises an insertion of a trimer peptide of cysteine, proline, threonine (CPT) into the transmembrane domain. In some embodiments, the threonine of the CPT insertion is not threonine but another amino acid, and in at least specific cases that other amino acid is or is not cysteine or proline.

In some embodiments, the chimeric fusion protein comprises a transmembrane domain of IL7R, IL2RA, IL2RB, IL2RG, IL14R, IL15R, IL9R, IL21R, CD2, CD40L, CD58, CD80, or SIRPα.

In some embodiments, the chimeric fusion protein comprises an extracellular component comprising: (i) an extracellular domain of a Cluster of Differentiation 80 (CD80) polypeptide, or a portion or variant thereof that is capable of binding a CD28 or CTLA-4 polypeptide; (ii) an extracellular domain of a Cluster of Differentiation 58 (CD58) polypeptide, or a portion or variant thereof that is capable of binding a Cluster of Differentiation 2 (CD2) polypeptide; (iii) an extracellular domain of a Signal Regulatory Protein Alpha (SIRPα) polypeptide, or a portion or variant thereof that is capable of binding a Cluster of Differentiation 47 (CD47) polypeptide; (iv) an extracellular domain of a Cluster of Differentiation 40L (CD40L) polypeptide, or a portion or variant thereof that is capable of binding a CD40 polypeptide; (v) an extracellular domain of a Cluster of Differentiation 2 (CD2) receptor, or a portion or variant thereof that is capable of binding a CD58 polypeptide; or (vi) an extracellular domain of a Cluster of Differentiation 34 (CD34) polypeptide.

In some embodiments, the chimeric fusion protein comprises an extracellular component comprising an extracellular domain of a Cluster of Differentiation 80 (CD80) polypeptide, or a portion or variant thereof that is capable of binding a CD28 or CTLA-4 polypeptide. In some embodiments, the extracellular domain of CD80 comprises an amino acid sequence with at least 80%, at least 85%, at least 90%, at least 95%, at least 98%, at least 99%, or 100% sequence identity to SEQ ID NO: 226.

In some embodiments, the chimeric fusion protein comprises an extracellular component comprising an extracellular domain of a Cluster of Differentiation 58 (CD58) polypeptide, or a portion or variant thereof that is capable of binding a CD28 or CTLA-4 polypeptide. In some embodiments, the extracellular domain of CD80 comprises an amino acid sequence with at least 80%, at least 85%, at least 90%, at least 95%, at least 98%, at least 99%, or 100% sequence identity to SEQ ID NO: 227.

In some embodiments, the chimeric fusion protein comprises an extracellular component comprising an extracellular domain of CD34. In some embodiments, the extracellular domain of CD34 comprises an amino acid sequence with at least 80%, at least 85%, at least 90%, at least 95%, at least 98%, at least 99%, or 100% sequence identity to SEQ ID NO: 228.

In some embodiments, a population of host cells comprising one or more modifications disclosed herein (e.g., expression of a Fas-41BB fusion protein or chimeric IL7R polypeptide disclosed herein) exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 10 fold, at least 50 fold, or at least 100 fold, at least 500 fold, or at least 1000 fold increased proliferation in response to target cells (e.g., that present a KRAS G12D peptide) as compared to a population of control cells (for example, corresponding cells lacking the Fas-41BB fusion protein or chimeric IL7R polypeptide). The proliferation can be, for example, as determined by an in vitro lymphoproliferation assay or measurement of host cell numbers after co-incubation. The host cells can comprise an extracellular binding protein (e.g., a TCR comprising Vα and Vβ regions and/or CDRs disclosed herein), and/or a modification that results in decreased expression of endogenous TRAC, TRBC1, and/or TRBC2.

In some embodiments, a population of host cells comprising one or more modifications disclosed herein (e.g., expression of a Fas-41BB fusion protein or chimeric IL7R polypeptide disclosed herein) exhibits at least 5%, at least 10%, at least 20%, at least 30%, at least 40%, at least 50%, at least 60%, at least 70%, at least 80%, at least 90%, at least 2-fold, at least 3 fold, at least 4 fold, at least 5 fold, at least 10 fold, at least 50 fold, or at least 100 fold, at least 500 fold, or at least 1000 fold increased killing of target cells as compared to a population of control cells (for example, corresponding cells lacking the Fas-41BB fusion protein or chimeric IL7R polypeptide). The killing of target cells can be, for example, as determined by an in vitro cytotoxicity assay. The host cells can comprise an extracellular binding protein (e.g., a TCR comprising Vα and Vβ regions and/or CDRs disclosed herein), and/or a modification that results in decreased expression of endogenous TRAC, TRBC1, and/or TRBC2.

A nucleic acid encoding a polypeptide disclosed herein (e.g., extracellular binding protein, CD8 co-receptor chain or an extracellular portion thereof, Fas-41BB fusion protein, or chimeric IL7R fusion protein) can encode a signal peptide. In some cases, a polypeptide of the disclosure comprises a signal peptide. A signal peptide can be cleaved off during processing of the polypeptide, thus in some cases a mature polypeptide disclosed herein does not contain a signal peptide.

A signal peptide at the N-terminus of a protein can be involved in transport of the protein to or through a membrane, transport to different a membranous cellular compartment, or secretion of the protein from the cell. A nucleic acid encoding a protein of the disclosure can encode a signal peptide to facilitate membrane insertion and surface localization of the protein. A signal peptide can be selected for its ability to facilitate ER processing and cell surface localization of the protein. Any suitable signal peptide can be used. In some cases, the signal peptide can comprise a G-CSF signal peptide or a CD8α signal peptide. A signal peptide can be about 10 to about 40 amino acids in length. In some cases, a signal peptide is at least about 10, 15, 16, 20, 21, 22, 25, or 30 amino acids in length, or more. In some cases, a signal peptide is at most about 15, 16, 20, 21, 22, 25, or 30 amino acids in length, or less. In some cases, a signal peptide is about 16-30 amino acids in length.

### Compositions

In some cases, the present disclosure provides for a pharmaceutically acceptable composition comprising a plurality of host cells described herein and a pharmaceutically acceptable carrier, excipient, or diluent. In some cases, the composition comprises a CD4+ T cell population and/or a CD8+ T cell population bearing: (i) the extracellular binding protein; and (ii) one or more genomic mutations that causes or contributes to decreased expression of an endogenous T cell receptor α constant (TRAC), a T cell receptor β constant 1 (TRBC1) locus, a T cell receptor β constant 2 (TRBC2) locus, or a combination thereof. In some embodiments, the composition comprises a CD4+ cell population comprising (i) at least about 20%, at least about 30%, at least about 40%, at least about 50%, at least about 60%, at least about 70%, at least about 80%, at least about 85%, at least about 90%, or at least about 95% modified CD4+ T cells. In some embodiments, the composition comprises a CD8+ cell population comprising (ii) at least about 20%, at least about 30%, at least about 40%, at least about 50%, at least about 60%, at least about 70%, at least about 80%, at least about 85%, at least about 90%, or at least about 95% modified CD8+ T cells.

In some embodiments, the CD4+ and/or CD8+ cells further comprise a transgenic polynucleotide encoding a polypeptide that comprises an extracellular portion of a CD8 co-receptor α (CD8α) chain or a transgenic polynucleotide encoding a polypeptide that comprises an extracellular portion of a CD8 co-receptor β (CD8β) chain. In some embodiments, the CD4+ and/or CD8+ cells express a CD8 co-receptor disclosed herein, e.g., a CD8α chain and/or a CD8β chain.

In some cases, the composition comprises a CD4+ T cell population and/or a CD8+ T cell population bearing: (i) the extracellular binding protein; and (ii) one or more genomic mutations that causes or contributes to decreased expression of an endogenous T cell receptor α constant (TRAC), a T cell receptor β constant 1 (TRBC1) locus, a T cell receptor β constant 2 (TRBC2) locus, or a combination thereof. In some embodiments, the composition comprises a CD4+ cell population comprising (i) at least about 30%, at least about 40%, at least about 50%, at least about 60%, at least about 70%, at least about 80%, at least about 85%, at least about 90%, or at least about 95% modified CD4+ T cells. In some embodiments, the composition comprises a CD8+ cell population comprising (ii) at least about 30%, at least about 40%, at least about 50%, at least about 60%, at least about 70%, at least about 80%, at least about 85%, at least about 90%, or at least about 95% modified CD8+ T cells. In some embodiments, the CD4+ or CD8+ cells further comprise a transgenic polynucleotide encoding a polypeptide that comprises an extracellular portion of a CD8 co-receptor α (CD8α) chain or a resulting polynucleotide encoding a polypeptide that comprises an extracellular portion of a CD8 co-receptor β (CD8β) chain. In some cases, the composition comprises both CD4+ and CD8+ cells bearing: (i) the extracellular binding protein and (ii) a genomic mutation that causes or contributes to decreased expression of an endogenous T cell receptor α constant (TRAC), a T cell receptor β constant 1 (TRBC1) locus, or a T cell receptor β constant 2 (TRBC2) locus. In some cases, the composition comprises both of (ii) a transgenic polynucleotide encoding a polypeptide that comprises an extracellular portion of a CD8 co-receptor α (CD8α) chain or a polynucleotide encoding a polypeptide that comprises an extracellular portion of a CD8 co-receptor β (CD8β) chain.

In some cases, the composition comprises both CD4+ and CD8+ cells bearing: (i) the extracellular binding protein; and either or both of (ii) a genomic mutation that causes or contributes to decreased expression of an endogenous T cell receptor α constant (TRAC), a T cell receptor β constant 1 (TRBC1) locus, or a T cell receptor β constant 2 (TRBC2) locus. In some embodiments, the CD4+ or CD8+ cells further comprise a transgenic polynucleotide encoding a polypeptide that comprises an extracellular portion of a CD8 co-receptor α (CD8α) chain or a resulting polynucleotide encoding a polypeptide that comprises an extracellular portion of a CD8 co-receptor β (CD8β) chain. In some embodiments, the CD4+ or CD8+ cells further comprise a transgenic polynucleotide encoding a polypeptide that comprises an extracellular portion of a CD8 co-receptor α (CD8α) chain or a resulting polynucleotide encoding a polypeptide that comprises an extracellular portion of a CD8 co-receptor β (CD8β) chain. In some embodiments, the composition comprises a CD4+ cell population comprising (i) at least about 30%, at least about 40%, at least about 50%, at least about 60%, at least about 70%, at least about 80%, at least about 85%, at least about 90%, or at least about 95% modified CD4+ T cells. In some embodiments, the composition further comprises a CD8+ cell population comprising (ii) at least about 30%, at least about 40%, at least about 50%, at least about 60%, at least about 70%, at least about 80%, at least about 85%, at least about 90%, or at least about 95% modified CD8+ T cells. In some cases, the composition comprises a reduced amount or substantially no naïve T cells. In some embodiments, the composition comprises about a 1:1 ratio of CD4+ to CD8+ T cells. In some cases, the composition comprises both CD4+ and CD8+ cells bearing: (i) the extracellular binding protein; and either or both of (ii) a genomic mutation that causes or contributes to decreased expression of an endogenous T cell receptor α constant (TRAC), a T cell receptor β constant 1 (TRBC1) locus, or a T cell receptor β constant 2 (TRBC2) locus. In some embodiments, the CD4+ or CD8+ cells further comprise a transgenic polynucleotide encoding a polypeptide that comprises an extracellular portion of a CD8 co-receptor α (CD8α) chain or a resulting polynucleotide encoding a polypeptide that comprises an extracellular portion of a CD8 co-receptor β (CD8β) chain. In some embodiments, the composition comprises a CD4+ cell population comprising (i) at least about 30%, at least about 40%, at least about 50%, at least about 60%, at least about 70%, at least about 80%, at least about 85%, at least about 90%, or at least about 95% modified CD4+ T cells. In some embodiments, the composition further comprises a CD8+ cell population comprising at least about 30%, at least about 40%, at least about 50%, at least about 60%, at least about 70%, at least about 80%, at least about 85%, at least about 90%, or at least about 95% modified CD8+ T cells. In some cases, the composition comprises a reduced amount or substantially no naïve T cells. In some embodiments, the composition comprises about a 1:1 ratio, about a 1:2 ratio, about a 1:3 ratio, about a 1:4 ratio, about a 1:5 ratio, about a 1:6 ratio, about a 1:7 ratio, about a 1:8 ratio, about a 1:9 ratio, about a 1:10 ratio, about a 2:1 ratio, about a 3:1 ratio, about a 4:1 ratio, about a 5:1 ratio, about a 6:1 ratio, about a 7:1 ratio, about an 8:1 ratio, about a 9:1 ratio, or about a 10:1 ratio of CD4+ to CD8+ T cells.

In some cases, the carrier or excipient comprises albumin. In some cases, the diluent comprises physiologically normal saline. Suitable excipients can also include water, saline, dextrose, glycerol, or the like, and combinations thereof. In some embodiments, a composition comprises a suitable infusion media. Suitable infusion media can be any isotonic medium formulation, normal saline, Normosol R (Abbott) or Plasma-Lyte A (Baxter), 5% dextrose in water, or Ringer's lactate can be utilized. An infusion medium can be supplemented with human serum albumin or other human serum components.

### Polynucleotides or Vectors

In some aspects, the present disclosure provides for a polynucleotide comprising a open reading frame encoding an extracellular binding protein (e.g., an extracellular binding protein capable of binding a KRAS mutant peptide). The open reading frame can be operatively linked to a promoter (e.g., heterogenous promoter). The extracellular binding protein can comprise a TCR α chain variable (Vα) domain; a TCR β chain variable (Vβ) domain; a TCRα FR1, CDR1, FR2, CDR2, FR3, CDR3, or FR4 region; or a TCRβ FR1, CDR1, FR2, CDR2, FR3, CDR3, or FR4 region disclosed herein, for example, comprising a sequence having at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% identity to any of the sequences described herein (e.g., in Table 1). The polynucleotide can be codon optimized. The extracellular binding protein can comprise an amino acid sequence of any one of SEQ ID NOs: 1-112, 118-218, and 229-278.

In some cases, the heterogenous promoter is not a mammalian promoter. In some cases, the heterogenous promoter is a constitutive promoter that is not a TCR promoter. In some cases, the heterogenous promoter is a viral promoter. In some cases, the heterogenous promoter is a mammalian promoter. In some cases, the heterogenous promoter is a human promoter. In some cases, the heterogenous promoter is a synthetic promoter. In some cases, the heterogenous promoter is an inducible promoter. In some cases, the heterogenous promoter is a tissue-specific promoter. In some cases, the heterogenous promoter is an immune cell-specific promoter.

In some embodiments, the promoter is a murine stem cell virus (MSCV) promoter. In some embodiments, the polynucleotide is promoterless. In some embodiments, the promoterless polynucleotide is designed to be operatively linked to an endogenous promoter at the site of insertion. In some embodiments, the promoter is an elongation factor-1 alpha (EF-1α) promoter. In some embodiments, the promoter has at least about 85% sequence identity to the following exemplary sequence:

### EF-1α Promoter

(SEQ ID NO: 279)
GGCTCCGGTGCCCGTCAGTGGGCAGAGCGCACATCGCCCACAGTCCCCG
AGAAGTTGGGGGGAGGGGTCGGCAATTGAACCGGTGCCTAGAGAAGGTG
GCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACG
TTCTTTTTCGCAACGGGTTTGCCGCCAGAACACAGGTAAGTGCCGTGTG
TGGTTCCCGCGGGCCTGGCCTCTTTACGGGTTATGGCCCTTGCGTGCCT
TGAATTACTTCCACTGGCTGCAGTACGTGATTCTTGATCCCGAGCTTCG
GGTTGGAAGTGGGTGGGAGAGTTCGAGGCCTTGCGCTTAAGGAGCCCCT
TCGCCTCGTGCTTGAGTTGAGGCCTGGCCTGGGCGCTGGGGCCGCCGCG
TGCGAATCTGGTGGCACCTTCGCGCCTGTCTCGCTGCTTTCGATAAGTC
TCTAGCCATTTAAAATTTTTGATGACCTGCTGCGACGCTTTTTTTCTGG
CAAGATAGTCTTGTAAATGCGGGCCAAGATCTGCACACTGGTATTTCGG
TTTTTGGGGCCGCGGGCGGCGACGGGGCCCGTGCGTCCCAGCGCACATG
TTCGGCGAGGCGGGGCCTGCGAGCGCGGCCACCGAGAATCGGACGGGGG
TAGTCTCAAGCTGGCCGGCCTGCTCTGGTGCCTGGCCTCGCGCCGCCGT
GTATCGCCCCGCCCTGGGCGGCAAGGCTGGCCCGGTCGGCACCAGTTGC
GTGAGCGGAAAGATGGCCGCTTCCCGGCCCTGCTGCAGGGAGCTCAAAA
TGGAGGACGCGGCGCTCGGGAGAGCGGGCGGGTGAGTCACCCACACAAA
GGAAAAGGGCCTTTCCGTCCTCAGCCGTCGCTTCATGTGACTCCACGGA
GTACCGGGCGCCGTCCAGGCACCTCGATTAGTTCTCGAGCTTTTGGAGT
ACGTCGTCTTTAGGTTGGGGGGAGGGGTTTTATGCGATGGAGTTTCCCC
ACACTGAGTGGGTGGAGACTGAAGTTAGGCCAGCTTGGCACTTGATGTA
ATTCTCCTTGGAATTTGCCCTTTTTGAGTTTGGATCTTGGTTCATTCTC
AAGCCTCAGACAGTGGTTCAAAGTTTTTTTCTTCCATTTCAGGTGTCGT
GA

In some embodiments, the binding protein is capable of binding to a peptide:HLA complex, wherein the peptide comprises a KRAS G12 mutant peptide. In some embodiments, the KRAS peptide is a KRAS G12D mutant peptide. In some embodiments, the KRAS G12D mutant peptide comprises an amino acid sequence of VVVGADGVGK (SEQ ID NO: 295). In some embodiments, the extracellular binding protein is human, humanized, or chimeric. In some embodiments, the extracellular binding protein is selective for the KRAS G12D mutant peptide. In some embodiments, the extracellular binding protein has a log 10EC50 for the KRAS G12 mutant peptide of about −6.0 or less, about −6.1 or less, about −6.2 or less, about −6.3 or less, about −6.4 or less, about −6.5 or less, about −6.6 or less, about −6.7 or less, about −6.8 or less, about −6.9 or less, about −7.0 or less, about −7.1 or less, about −7.2 or less, about −7.3 or less, about −7.4 or less, about −7.5 or less, about −7.6 or less, about −7.7 or less, about −7.8 or less, about −7.9 or less, about −8.0 or less, about −8.1 or less, about −8.2 or less, about −8.3 or less, about −8.4 or less, about −8.5 or less, about −8.6 or less, about −8.7 or less, about −8.8 or less, about −8.9 or less, about −9 or less, about −9.1 or less, or about −9.2 or less.

In some aspects, the present disclosure provides for a vector comprising any of the polynucleotides described herein. In some cases, the vector is a viral vector, such as a lentiviral vector, a γ-retroviral vector, or an adeno-associated virus (AAV) vector. In some embodiments, the vector is a non-viral vector, for example, a plasmid, nanoplasmid, minicircle, a midge, a MIP, or a doggybone, a lipid-based nanoparticle, a liposome, a circular polynucleotide (e.g., DNA or RNA), a linear polynucleotide (e.g., a DNA or RNA), or a combination thereof.

In some embodiments, the polynucleotide comprises a nucleic acid sequence encoding a self-cleaving peptide (e.g., P2A) between the nucleic acid sequence encoding the TCR receptor variable a (Vα) region and the nucleic acid sequence encoding the TCR receptor variable R (Vβ) region. In some embodiments, the polynucleotide further comprises a nucleic acid sequence encoding a self-cleaving peptide (e.g., T2A) disposed between the TCR receptor and an IL7R fusion protein (e.g., CD58-IL7R or CD34-IL7R). In some embodiments, the IL7R fusion protein comprises an intracellular domain and/or a transmembrane domain of a constitutively active IL7R subunit alpha. In some embodiments, the polynucleotide further comprises a nucleic acid sequence encoding a self-cleaving peptide (e.g., P2A) disposed between the sequence encoding the IL7R fusion protein and a sequence encoding a CD8 co-receptor (e.g., a sequence encoding CD8 co-receptor α chain and a sequence encoding CD8 co-receptor β chain). In some embodiments, the polynucleotide further comprises a nucleic acid sequence encoding a self-cleaving peptide (e.g., T2A, PTA, E2A, a furin peptide or other self-cleaving peptide) between the sequence encoding the CD8 co-receptor α chain and the sequence encoding the CD8 co-receptor β chain. In some embodiments, the polynucleotide further comprises a nucleic acid sequence that encodes a self-cleaving peptide that is disposed between the nucleic acid sequence encoding a binding protein and the nucleic acid sequence encoding a polypeptide comprising an extracellular portion of a CD8 co-receptor α chain; and/or the nucleic acid sequence encoding a binding protein and the nucleic acid sequence encoding a polypeptide comprising an extracellular portion of a CD8 co-receptor β chain. In some embodiments, the polynucleotide further comprises, operably linked in-frame:
(i) (pnBP)-(pnSCP₁)-(pnCD8α)-(pnSCP₂)-(pnCD8β)-(pnFP);
(ii) (pnBP)-(pnSCP₁)-(pnCD8β)-(pnSCP₂)-(pnCD8α)-(pnFP);
(iii) (pnBP)-(pnSCP₁)-(pnFP)-(pnSCP₁)-(pnCD8α)-(pnSCP₂)-(pnCD8β); or
(iv) (pnBP)-(pnSCP₁)-(pnFP)-(pnSCP₁)-(pnCD8β)-(pnSCP₂)-(pnCD8α); wherein pnCD8α is the nucleic acid sequence encoding a polypeptide that comprises an extracellular portion of a CD8 co-receptor α chain, wherein pnCD8β is the nucleic acid sequence encoding a polypeptide that comprises an extracellular portion of a CD8 co-receptor α chain, wherein pnBP is the nucleic acid sequence encoding a binding protein, wherein pnFP is the nucleic acid sequence encoding a fusion protein, and wherein pnSCP₁ and pnSCP₂ are each independently a polynucleotide encoding a self-cleaving peptide, wherein the polynucleotides and/or the encoded self-cleaving peptides are optionally the same or different. In some embodiments, the self-cleaving peptide is a P2A, T2A, E2A, or a furin peptide. In some embodiments, the furin peptide comprises the amino acid sequence RAKR (SEQ ID NO: 305). In some embodiments, the binding protein and fusion protein are encoded in a single construct or continuous genomic segment. In some embodiments, the binding protein, fusion protein, and CD8α or CD8β or both are encoded in a single construct or continuous genomic segment. In some embodiments, the binding protein and fusion protein are encoded in a single open reading frame. In some embodiments, binding protein and fusion protein are operably linked to a single promoter. In some embodiments, binding protein and fusion protein are operably linked to different promoters.

### Methods of Treatment

In some aspects, the present disclosure provides for a method of treating a disease or disorder associated with a KRAS G12 mutation in a subject, comprising administering to the subject an effective amount of any of the host cells or compositions described herein. In some embodiments, the host cell is autologous to the subject. In some embodiments, the host cell is allogenic to the subject. In some embodiments, the host cell is HLA-matched to the subject, for example, HLA matched at all typed HLA alleles. In some embodiments, a host cell and a subject can be HLA-typed HLA-A, HLA-B, HLA-C, and/or HLA-DR alleles. In some embodiments, the host cell and subject are matched for at least 1, at least 2, at least 3, at least 4, at least 5, at least 6, at least 7, or at least 8 HLA alleles. In some embodiments, the host cell is haploidentical to the subject.

In some embodiments, the subject is positive for an HLA-A*11 allele. In some embodiments, the subject is positive for an HLA-A*11:01 allele. In some embodiments, the mutation is a KRAS G12D mutation.

In some embodiments, the disease or disorder comprises a cancer. In some embodiments, the cancer is a solid cancer. In some embodiments, the cancer is a hematological malignancy. In some embodiments, the disease or disorder is selected from a pancreas cancer or carcinoma, optionally a pancreatic ductal adenocarcinoma (PDAC); a colorectal cancer or carcinoma; a lung cancer, optionally a non-small-cell lung carcinoma; a biliary cancer; an endometrial cancer or carcinoma; a cervical cancer; an ovarian cancer; a bladder cancer; a liver cancer; a myeloid leukemia, optionally myeloid leukemia such as acute myeloid leukemia; a myelodysplastic syndrome; a lymphoma such as Non-Hodgkin lymphoma; Chronic Myelomonocytic Leukemia; Acute Lymphoblastic Leukemia (ALL); a cancer of the urinary tract; a cancer of the small intestine; a breast cancer or carcinoma; a melanoma (optionally a cutaneous melanoma, an anal melanoma, or a mucosal melanoma); a glioma; a poorly differentiated thyroid gland carcinoma; a neuroblastoma; a histiocytic and dendritic cell neoplasm; neurofibromatosis Type 1; rhabdomyosarcoma; a soft tissue sarcoma; a bladder carcinoma; a sarcoma; a glioblastoma; a squamous cell lung carcinoma; an anaplastic astrocytoma; chronic myeloid leukemia; diffuse large B-cell lymphoma; double-hit lymphoma; head and neck carcinoma; head and neck squamous cell carcinoma; hepatocellular carcinoma; malignant peripheral nerve sheath tumor; mantle cell lymphoma; myelodysplastic/myeloproliferative neoplasm, unclassifiable; peripheral T cell lymphoma; prostate carcinoma; refractory anemia with excess blasts-2; renal cell carcinoma; rhabdoid tumor; schwannoma; secondary AML; small cell lung carcinoma; therapy-related AML; thymic carcinoma; thyroid gland follicular carcinoma; malignant thyroid gland neoplasm; thyroid gland carcinoma; thyroid gland adenocarcinoma; urothelial carcinoma; or thyroid gland papillary carcinoma.

In some embodiments, the method further comprises genotyping a tumor of the subject for a KRAS G12D allele prior to the administering. In some embodiments, the method further comprises genotyping the subject for an HLA-A allele prior to the administering. In some cases, the subject is determined to carry a KRAS G12D allele prior to the administering. In some cases, the subject has been genotyped for an HLA-A allele prior to the administering.

An effective amount of a pharmaceutical composition can describe an amount sufficient, at dosages and for periods of time needed, to achieve the predetermined clinical results or beneficial treatment. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

Administration may be affected continuously or intermittently, and parenterally. A composition can be administered locally (e.g., intratumoral) or systemically (e.g., intravenously). Administration may be for treating a subject already confirmed as having a recognized condition, disease or disease state, or for treating a subject susceptible to or at risk of developing such a condition, disease or disease state. Co-administration with an adjunctive therapy may include simultaneous or sequential delivery of multiple agents in any order and on any dosing schedule.

Methods disclosed herein may further include administering one or more additional agents to treat the disease or disorder in a combination therapy. For example, in certain embodiments, a combination therapy comprises administering an engineered host cell with (concurrently, simultaneously, or sequentially) an immune checkpoint inhibitor. In some embodiments, a combination therapy comprises administering a host cell with an agonist of a stimulatory immune checkpoint agent. In some embodiments, a combination therapy comprises administering a host cell with a secondary therapy, such as chemotherapeutic agent, a radiation therapy, a surgery, an antibody, or any combination thereof.

### Methods of Manufacturing

In some aspects, the present disclosure provides for a method of manufacturing a host cell, comprising contacting to the host cell: (a) any of the polynucleotides or vectors described herein. In some cases, the method further comprises contacting to the host cell: (b) an endonuclease (e.g., Cas endonuclease, such as a class II, type V Cas endonuclease); and (c) a guide RNA compatible with the endonuclease (e.g., compatible with the class II, type V Cas endonuclease), wherein the guide RNA is configured to hybridize to an endogenous T cell receptor constant region locus of the host cell prior to the polynucleotide or vector or after the polynucleotide or vector. In some embodiments, the contacting comprises transfection or transduction. In some embodiments, the transfection comprises electroporation. In some embodiments, the T cell receptor constant region locus is a TRAC, TRBC1, or TRBC2 locus. In some embodiments, the guide RNA comprises a sequence having at least 80% sequence identity to any of the guide RNA sequences recited in Table 1. In some embodiments, the guide RNA comprises a pattern of modifications according to any of the guide RNA sequences recited in Table 1. In some embodiments, the class II, type V Cas endonuclease is a type V-A Cas endonuclease. In some embodiments, the class II, type V Cas endonuclease comprises a sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or 100% sequence identity to SEQ ID NO: 117. In some embodiments, the host cell comprises an immune cell or a precursor thereof. In some embodiments, the immune cell comprises a T cell, a NK cell, a NK-T cell, a dendritic cell, a macrophage, a monocyte, or any combination thereof. In some embodiments, the immune cell comprises a T cell, wherein the T cell comprises a CD4+ T cell, a CD8+ T cell, a CD4− CD8− double negative T cell, a γδ T cell, or any combination thereof.

Cells can be engineered to comprise or be capable of expressing an extracellular binding protein, an additional polypeptide disclosed herein, and/or to reduce expression of an endogenous TCR gene. For example, cell engineering techniques disclosed herein and/or known to a skilled person can be used to modify cells to comprise a recombinant nucleic acid that encodes an extracellular binding protein of the disclosure, and/or to introduce a modification to reduce expression of endogenous TRAC, TRBC1 and/or TRBC2, thereby generating host cells (such as engineered T cells).

The methods can comprise contacting a cell with a recombinant nucleic acid, or with a vector that comprises the recombinant nucleic acid, under conditions that permit uptake of the recombinant nucleic acid by the cell. A recombinant nucleic acid can comprise a nucleotide sequence that encodes an extracellular binding protein disclosed herein or a component thereof. In some cases, a recombinant nucleic acid is utilized to alter a genome of a cell.

A recombinant nucleic can be a substance whose molecules comprise or consist essentially of nucleotides linked in a chain. Non-limiting examples of the recombinant nucleic include a circular nucleic acid, a DNA, a single stranded DNA, a double stranded DNA, a genomic DNA, a plasmid, a nanoplasmid, a plasmid DNA, a viral DNA, a minicircle (e.g., lacking a bacterial origin of replication), and an RNA.

A recombinant nucleic acid can include one or more homology arms, for example, comprising sequences that are complementary to a genomic DNA sequence to be targeted for insertion (e.g., via homologous recombination). A recombinant nucleic acid can comprise one or more promoter regions, barcodes, restriction sites, cleavage sites, endonuclease recognition sites, primer binding sites, selectable markers, unique identification sequences, resistance genes, linker sequences, or any combination thereof. In some aspects, these sites may be useful for enzymatic digestion, amplification, sequencing, targeted binding, purification, providing resistance properties (e.g., antibiotic resistance for selection), or any combination thereof. A recombinant nucleic acid may also include transcriptional or translational regulatory sequences, for example, one or more promoters, enhancers, insulators, internal ribosome entry sites, sequences encoding 2A linkers and/or polyadenylation signals.

Recombinant nucleic acids can be assembled by a variety of methods, e.g., by automated solid-phase synthesis. A recombinant nucleic acid can be constructed using standard solid-phase DNA/RNA synthesis. A recombinant nucleic acid can also be constructed using a synthetic procedure. A recombinant nucleic acid can be synthesized manually or in a fully automated fashion. In some cases, a synthetic procedure may comprise 5′-hydroxyl oligonucleotides that can be initially transformed into corresponding 5′-H-phosphonate mono esters, subsequently oxidized in the presence of imidazole to activated 5′-phosphorimidazolidates, and finally reacted with pyrophosphate on a solid support. This procedure may include a purification step after the synthesis such as PAGE, HPLC, MS, or any combination thereof. Recombinant nucleic acids can be purchased commercially.

Recombinant nucleic acid described herein can be modified. In some cases, a recombinant nucleic acid can be modified to make it less immunogenic and more stable for transfection into a cell.

For targeted integration, a recombinant nucleic acid sequence to be inserted can be flanked by homology arms comprising sequences that are complementary to a genomic DNA sequence to be targeted for insertion (e.g., via homologous recombination and/or homology-directed repair, HDR). A double stranded break can be introduced at a target site in the genome, and the homology arms can promote insertion of the recombinant nucleic acid. In some cases, a recombinant nucleic acid can be excised from a vector, such as a nanoplasmid (e.g., via a nuclease), and inserted into the genome of the cell.

A recombinant nucleic acid can be inserted in a safe harbor locus. A safe harbor can comprise a genomic location where a recombinant nucleic acid can integrate and function without substantially perturbing endogenous activity, for example, with a relatively low impact on local or global gene expression. For example, one or more recombinant nucleic acids can be inserted into any one of HPRT, an AAVS site (E.G., AAVS1, AAVS2, etc.), CCR5, hROSA26, and/or any combination thereof. A recombinant nucleic acid can be inserted in an intergenic region. A recombinant nucleic acid can be inserted in a non-coding region. A recombinant nucleic acid can be inserted within a gene. In some cases, a recombinant nucleic acid can disrupt a gene it is inserted into (e.g., reduce or eliminate expression of the disrupted gene). A disrupted gene can be for example, an endogenous TCR gene (e.g., TRAC, TCRB, TCRBC1, TRBC2, TRG, TRD), or an immune checkpoint gene (e.g., PD-1, CTLA-4). A recombinant nucleic acid can be inserted adjacent to or near to a promoter.

A variety of enzymes can catalyze generation of a double-stranded break in the genome and/or insertion of foreign DNA into a host genome. Non-limiting examples of gene editing tools and techniques include CRISPR systems, CRISPR-associated polypeptide (Cas), TALEN, zinc finger nuclease (ZFN), zinc finger associate gene regulation polypeptide, meganuclease, Mega-TAL, transposon-based systems, natural master transcription factors, epigenetic modifying enzymes, recombinase, flippase, transposase, RNA-binding proteins (RBP), an Argonaute protein, any derivative thereof, any variant thereof, or any fragment thereof.

A CRISPR system can be utilized to facilitate insertion of a recombinant nucleic acid encoding an extracellular binding protein or a component thereof into a cell genome. For example, a CRISPR system can introduce a double stranded break at a target site in a genome or a random site of a genome.

In some cases, a CRISPR system comprises CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, or type VI CRISPR-associated (Cas) polypeptides a derivative, variant, or functional fragment thereof.

In some embodiments, a CRISPR system comprises a Class I system or endonuclease (e.g., Type I, Type III or Type IV Cas proteins). A class I system can be of the I-A, I-B, I-C, I-U, I-D, I-E, I-F, IV-A, IV-B, III-A, III-D, III-C, or III-B subtype.

In some embodiments, a CRISPR system comprises a Class II system or endonuclease (e.g., Type II, Type V, or Type VI). A class II, Type II system can be of the II-A, II-B, II-C1, or II-C2 subtype. A class II, Type V systems can of the V-A, V-B1, V-B2, V-C, V-D, V-E, V-F1, V-F1(V-U3), V-F2, V-F3, V-G, V-H, V-I, V-K (V-U5), V-U1, V-U2, or V-U4 subtype. A Class II, Type IV systems can be of the: VI-A, VI-B1, VI-B2, VI-C, or VI-D subtype.

In some embodiments, a Cas protein used in a method disclosed herein is a class II endonuclease. In some embodiments, a Cas protein used in a method disclosed herein is a class II, type V Cas endonuclease. In some embodiments, a Cas protein used in a method disclosed herein is a class II, type V-A Cas endonuclease.

Non-limiting examples of Cas proteins that can be used in the CRISPR systems include Cas1, Cas1B, Cas2, Cas3, Cas4, Cas5, Cas6, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, and modified versions thereof. An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within or within about 1, 2, 3, 4, 5, 6, 7, 8, 9, 10, 15, 20, 25, 50, 100, 200, 500, or more base pairs from the first or last nucleotide of a target sequence. A Cas protein can be a high-fidelity Cas protein. Alternatives to S. pyogenes Cas9 may include RNA-guided endonucleases from the Cpf1 family that display cleavage activity in mammalian cells.

In some embodiments, a gene editing system comprises a Cas protein, and the system further comprises a guide RNA (gRNA) which complexes with the Cas protein. In some embodiments, the gene editing moiety comprises an RBP complexed with a gRNA which is able to form a complex with a Cas protein. In some embodiments, the gRNA comprises a targeting segment which exhibits at least 80%, at least 85%, at least 90%, at least 91%, at least 92%, at least 93%, at least 94%, at least 95%, at least 96%, at least 97%, at least 98%, or at least 99%, or 100% sequence identity to a target polynucleotide. Multiple gRNAs can be used, e.g., to simultaneously or sequentially target TRAC, TRBC1, and/or TRBC2.

In some cases, a dual nickase approach may be used to introduce a double stranded break. Cas proteins can be mutated at certain amino acids within either nuclease domains, thereby deleting activity of one nuclease domain and generating a nickase Cas protein capable of generating a single strand break. A nickase along with two distinct guide RNAs targeting opposite strands may be utilized to generate a DSB within a target site (often referred to as a “double nick” or “dual nickase” CRISPR system).

A transposon-based system can be utilized for insertion of a recombinant nucleic acid encoding an extracellular binding protein of the disclosure or a component thereof into a genome, or for disruption of a TCR encoding gene. A transposon can comprise a recombinant nucleic acid that can be inserted into a DNA sequence. A class I transposon can be transcribed into an RNA intermediate, then reverse transcribed and inserted into a DNA sequence. A class II transposon can comprise a DNA sequence that is excised from one DNA sequence and/or inserted into another DNA sequence. A class II transposon system can comprise (i) a transposon vector that contains a sequence (e.g., comprising a transgene) flanked by inverted terminal repeats, and (ii) a source for the transposase enzyme. A transposon system (e.g., class II transposon system) can direct the integration of a recombinant nucleic acid sequence encoding an extracellular binding protein or a component thereof, while leaving behind the rest of the vector. A transposon and a transposase can be introduced into a cell. In some cases, a vector that encodes a transposase and comprises a recombinant nucleic acid is introduced into a cell, and the transposase is expressed and mediates insertion of the transposon into the genome.

Examples of transposon-based systems that can be used include, but are not limited to, sleeping beauty (e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol2 (e.g., derived from medaka fish); and spinON.

In some embodiments an extracellular binding protein or other polypeptide can be expressed in an host cell without genomic integration of a recombinant nucleic acid that encodes the extracellular binding protein or other polypeptide. For example, an extracellular binding protein or other polypeptide can be expressed from an episomal vector, such as a DNA, RNA, circular DNA, circular RNA, minicircle, and the like. An extracellular binding protein or other polypeptide can be transiently expressed. For example, expression of an extracellular binding protein or other polypeptide can be reduced as a nucleic acid that encodes it is degraded. One method of generating host cells is through the use of a ribonucleic acid (RNA) system, e.g., a system that involves delivering one or more recombinant nucleic acids as an RNA. In some cases, the use of RNA can minimize DNA-induced toxicity and immunogenicity sometimes observed with the use of DNA.

In some cases, one or more recombinant nucleic acids of the disclosure can be inserted randomly into the genome of a cell. For instance, a recombinant nucleic acid can encode its own promoter or can be inserted into a position where it is under the control of an endogenous promoter. Alternatively or additionally, a recombinant nucleic acid can be inserted into a gene, such as an intron of a gene, an exon of a gene, a promoter, or a non-coding region.

One or more recombinant nucleic acids and/or gene editing components can be delivered to a cell by any suitable method, for example, using any suitable vector. A vector can be or can comprise a viral vector, a gamma-retroviral vector, a lentiviral vector, an adeno-associated viral vector, a transposon, and the like. Any vector systems can be used including, but not limited to, DNA vectors, RNA vectors, ribonucleoprotein vectors, hybrid DNA-RNA vectors, plasmid vectors, nanoplasmid vectors, minicircle vectors, retroviral vectors, lentiviral vectors, adenovirus vectors, poxvirus vectors; herpesvirus vectors and adeno-associated virus vectors, etc. Non-viral vector delivery systems can include DNA plasmids, naked nucleic acid, and nucleic acid complexed with a delivery vehicle such as a liposome, lipid nanoparticle, or poloxamer. Viral vector delivery systems can include DNA and RNA viruses, which have either episomal or integrated genomes after delivery to the cell. In some cases, one vector is used. In some cases, two vectors are used. In some cases, three or more vectors are used.

In some cases, recombinant nucleic acids and/or gene editing components of the disclosure can be delivered to cells without the use of vectors. In some cases, one or more recombinant nucleic acids and/or gene editing components of the disclosure can be delivered to cells via vectors, and one or more recombinant nucleic acids and/or gene editing components can be delivered without the use of vectors.

Cells can be genetically engineered to comprise a recombinant nucleic acid that encodes an extracellular binding protein and/or modification to reduce expression of TRAC, TRB, TRBC1, and/or TRBC2 ex vivo. For example, cells can be taken from a subject in one or more blood draws and/or apheresis procedures, modified ex vivo, optionally selected and/or expanded before and/or after genetic modification, and optionally re-introduced into the subject or a different subject by infusion or injection.

In some cases, cells are genetically engineered to comprise an extracellular binding protein of the disclosure and/or modification to reduce expression of endogenous TRAC, TRB, TRBC1, and/or TRBC2 in vivo. For example, a vector can be used to deliver gene editing components to cells in a subject without removing the cells from the subject. Vectors can be delivered in vivo by administration to an individual subject, for example, by parenteral administration (e.g., intravenous, intraperitoneal, intramuscular, subdermal, or intracranial infusion) or topical application.

Methods to introduce gene editing components into a cell include, but are not limited to, electroporation, sonoporation, use of a gene gun, lipofection, calcium phosphate transfection, use of dendrimers, microinjection, and use of viral vectors including adenoviral, AAV, and retroviral vectors.

Electroporation using, for example, the Neon® Transfection System (ThermoFisher Scientific), the Xenon Electroporation System (ThermoFisher Scientific), or the AMAXA® Nucleofector (AMAXA® Biosystems) can also be used for delivery of nucleic acids into a cell. Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some cases, the electroporation pulse voltage, the electroporation pulse width, number of pulses, cell density, and tip type may be adjusted to optimize transfection efficiency and/or cell viability.

Cells can be selected or enriched for having or not having one or more given factors (e.g., cells may be separated based on the presence or absence of one or more factors). Selection techniques include positive selection and negative selection techniques, e.g., fluorescent activated cell sorting (FACS) or magnetic activated cell sorting (MACS). In some cases, cells can be selected before gene editing, for example, to enrich for a population of cells disclosed herein (e.g., immune cells, such as T cells or a T cell subset disclosed herein, such as gamma delta T cells or alpha beta T cells). Cells can be selected after gene editing, for example, to enrich for a population of cells disclosed herein (e.g., host cells that express an extracellular binding protein or additional polypeptide, and/or comprise a modification to reduce expression of endogenous TRAC, TRB, TRBC1, and/or TRBC2). Host cells can be selected or enriched based on a tag or marker, such as an epitope tag. The tag or marker can be appended to the extracellular binding protein. In some embodiments, the tag or marker is not appended to the extracellular binding protein. The tag or marker can be co-expressed with the extracellular binding protein as disclosed herein. The tag or marker can comprise a reporter gene, such as a fluorescent protein.

Cells can be selected, enriched, or expanded on the basis of being positive or negative for a given factor. In some embodiments, cells are selected, enriched, or expanded on the basis of being positive for two or more factors. In some embodiments, cells can be selected, enriched, or expanded on the basis of being positive for one or more factors, and negative for one or more factors.

In some cases, a selectable marker is introduced to a cell, e.g., together with or as part of a recombinant nucleic acid encoding an extracellular binding protein, so that cells that comprise the extracellular binding protein or modification express the selectable marker and can be selected, enriched, or expanded. In some cases, a selectable marker is an antibiotic resistance gene, and cells that do not express the antibiotic resistance gene can be killed by treatment with the antibiotic (e.g., to select or enrich for cells that comprise an extracellular binding protein). In some embodiments, the selectable marker is an epitope tag.

Expression of an extracellular binding protein, TRAC, TRB, TRBC1, and/or TRBC2 of the disclosure can be quantified, for example, by qPCR, RNA sequencing, western blot, or flow cytometry.

In some embodiments, selected cells can be expanded ex vivo and/or in vitro before gene editing or delivery of a recombinant nucleic acid, after gene editing or delivery of a recombinant nucleic acid, before selection, after selection, before expansion, after expansion, or a combination thereof. In some embodiments, selected cells can be expanded ex vivo and/or in vitro before gene editing or delivery of a recombinant nucleic acid. In some embodiments, selected cells can be expanded ex vivo and/or in vitro after gene editing or delivery of a recombinant nucleic acid. In some embodiments, selected cells can be expanded ex vivo and/or in vitro before selection and/or enrichment. In some embodiments, selected cells can be expanded ex vivo and/or in vitro after selection and/or enrichment. In some embodiments, selected cells can be expanded ex vivo and/or in vitro before expansion. In some embodiments, selected cells can be expanded ex vivo and/or in vitro after expansion.

### Illustrative Sequences

TABLE 1
Sequences of Example Nucleotide and Polypeptide Components Described
Herein.

| SEQ ID | | |
|---|---|---|
| NO: | DESC | SEQUENCE |
| 1 | TCR91b | MGPGLLHWMALCLLGTGHGDAMVIQNPRYQVTQFGK |
| | | PVTLSCSQTLNHNVMYWYQQKSSQAPKLLFHYYDKD |
| | | FNNEADTPDNFQSRRPNTSFCFLDIRSPGLGDAAMYLC |
| | | ATSTGRVYQPQHFGDGTRLSILEDLNKVFPPEVAVFEP |
| | | SEAEISHTQKATLVCLATGFFPDHVELSWWVNGKEVH |
| | | SGVCTDPQPLKEQPALNDSRYCLSSRLRVSATFWQNPR |
| | | NHFRCQVQFYGLSENDEWTQDRAKPVTQIVSAEAWG |
| | | RADCGFTSVSYQQGVLSATILYEILLGKATLYAVLVSA |
| | | LVLMAMVKRKDFGGSG |
| 2 | TCR91b | DAMVIQNPRYQVTQFGKPVTLSCSQT |
| | FR1 | |
| 3 | TCR91b | LNHNV |
| | CDR1 | |
| 4 | TCR91b | MYWYQQKSSQAPKLLFH |
| | FR2 | |
| 5 | TCR91b | YYDKDF |
| | CDR2 | |
| 6 | TCR91b | NNEADTPDNFQSRRPNTSFCFLDIRSPGLGDAAMYLC |
| | FR3 | |
| 7 | TCR91b | ATSTGRVYQPQH |
| | CDR3 | |
| 8 | TCR91b | FGDGTRLSIL |
| | FR4 | |
| 9 | TCR91a | MLTASLLRAVIASICVVSSMAQKVTQAQTEISVVEKED |
| | | VTLDCVYETRDTTYYLFWYKQPPSGELVFLIRRNSFDE |
| | | QNEISGRYSWNFQKSTSSFNFTITASQVVDSAVYFCAL |
| | | NEGGGSTLGRLYFGRGTQLTVWPDIQNPDPAVYQLRD |
| | | SKSSDKSVCLFTDFDSQTNVSQSKDSDVYITDKCVLDM |
| | | RSMDFKSNSAVAWSNKSDFACANAFNNSIIPEDTFFPSP |
| | | ESSCDVKLVEKSFETDTNLNFQNLSVIGFRILLLKVAGF |
| | | NLLMTLRLWSS |
| 10 | TCR91a | AQKVTQAQTEISVVEKEDVTLDCVYE |
| | FR1 | |
| 11 | TCR91a | TRDTTYY |
| | CDR1 | |
| 12 | TCR91a | LFWYKQPPSGELVFLIR |
| | FR2 | |
| 13 | TCR91a | RNSFDEQN |
| | CDR2 | |
| 14 | TCR91a | EISGRYSWNFQKSTSSFNFTITASQVVDSAVYFC |
| | FR3 | |
| 15 | TCR91a | ALNEGGGSTLGRLY |
| | CDR3 | |
| 16 | TCR91a | FGRGTQLTVWP |
| | FR4 | |
| 17 | TCR80b | MDTWLVCWAIFSLLKAGLTEPEVTQTPSHQVTQMGQE |
| | | VILRCVPISNHLYFYWYRQILGQKVEFLVSFYNNEISEK |
| | | SEIFDDQFSVERPDGSNFTLKIRSTKLEDSAMYFCASSG |
| | | VDRAYGYTFGSGTRLTVVEDLNKVFPPEVAVFEPSEAE |
| | | ISHTQKATLVCLATGFFPDHVELSWWVNGKEVHSGVC |
| | | TDPQPLKEQPALNDSRYCLSSRLRVSATFWQNPRNHFR |
| | | CQVQFYGLSENDEWTQDRAKPVTQIVSAEAWGRADC |
| | | GFTSVSYQQGVLSATILYEILLGKATLYAVLVSALVLM |
| | | AMVKRKDFGGSG |
| 18 | TCR80b | EPEVTQTPSHQVTQMGQEVILRCVPI |
| | FR1 | |
| 19 | TCR80b | SNHLY |
| | CDR1 | |
| 20 | TCR80b | FYWYRQILGQKVEFLVS |
| | FR2 | |
| 21 | TCR80b | FYNNEI |
| | CDR2 | |
| 22 | TCR80b | SEKSEIFDDQFSVERPDGSNFTLKIRSTKLEDSAMYFC |
| | FR3 | |
| 23 | TCR80b | ASSGVDRAYGYT |
| | CDR3 | |
| 24 | TCR80b | FGSGTRLTVV |
| | FR4 | |
| 25 | TCR80a | MACPGFLWALVISTCLEFSMAQTVTQSQPEMSVQEAE |
| | | TVTLSCTYDTSESDYYLFWYKQPPSRQMILVIRQEAYK |
| | | QQNATENRFSVNFQKAAKSFSLKISDSQLGDAAMYFC |
| | | ASTLSSGDKLTFGTGTRLAVRPNIQKPDPAVYQLRDSK |
| | | SSDKSVCLFTDFDSQTNVSQSKDSDVYITDKCVLDMRS |
| | | MDFKSNSAVAWSNKSDFACANAFNNSIIPEDTFFPSPES |
| | | SCDVKLVEKSFETDTNLNFQNLSVIGFRILLLKVAGFNL |
| | | LMTLRLWSS |
| 26 | TCR80a | AQTVTQSQPEMSVQEAETVTLSCTYD |
| | FR1 | |
| 27 | TCR80a | TSESDYY |
| | CDR1 | |
| 28 | TCR80a | LFWYKQPPSRQMILVIR |
| | FR2 | |
| 29 | TCR80a | QEAYKQQN |
| | CDR2 | |
| 30 | TCR80a | ATENRFSVNFQKAAKSFSLKISDSQLGDAAMYFC |
| | FR3 | |
| 31 | TCR80a | ASTLSSGDKLT |
| | CDR3 | |
| 32 | TCR80a | FGTGTRLAVRP |
| | FR4 | |
| 33 | TCR32b | MGPGLLCWVLLCLLGAGSVETGVTQSPTHLIKTRGQQ |
| | | VTLRCSSQSGHNTVSWYQQALGQGPQFIFQYYREEEN |
| | | GRGNFPPRFSGLQFPNYSSELNVNALELDDSALYLCAS |
| | | SDRGWNEQFFGPGTRLTVLEDLKNVFPPEVAVFEPSEA |
| | | EISHTQKATLVCLATGFYPDHVELSWWVNGKEVHSGV |
| | | CTDPQPLKEQPALNDSRYCLSSRLRVSATFWQNPRNHF |
| | | RCQVQFYGLSENDEWTQDRAKPVTQIVSAEAWGRAD |
| | | CGFTSESYQQGVLSATILYEILLGKATLYAVLVSALVL |
| | | MAMVKRKDSRGGSG |
| 34 | TCR32b | ETGVTQSPTHLIKTRGQQVTLRCSSQ |
| | FR1 | |
| 35 | TCR32b | SGHNT |
| | CDR1 | |
| 36 | TCR32b | VSWYQQALGQGPQFIFQ |
| | FR2 | |
| 37 | TCR32b | YYREEE |
| | CDR2 | |
| 38 | TCR32b | NGRGNFPPRFSGLQFPNYSSELNVNALELDDSALYLC |
| | FR3 | |
| 39 | TCR32b | ASSDRGWNEQF |
| | CDR3 | |
| 40 | TCR32b | FGPGTRLTVL |
| | FR4 | |
| 41 | TCR32a | MTRVSLLWAVVVSTCLESGMAQTVTQSQPEMSVQEA |
| | | ETVTLSCTYDTSENNYYLFWYKQPPSRQMIL VIRQEAY |
| | | KQQNATENRFSVNFQKAAKSFSLKISDSQLGDTAMYF |
| | | CAFMTPITQGGSEKLVFGKGTKLTVNPYIQNPDPAVYQ |
| | | LRDSKSSDKSVCLFTDFDSQTNVSQSKDSDVYITDKCV |
| | | LDMRSMDFKSNSAVAWSNKSDFACANAFNNSIIPEDTF |
| | | FPSPESSCDVKLVEKSFETDTNLNFQNLSVIGFRILLLKV |
| | | AGFNLLMTLRLWSS |
| 42 | TCR32a | AQTVTQSQPEMSVQEAETVTLSCTYD |
| | FR1 | |
| 43 | TCR32a | TSENNYY |
| | CDR1 | |
| 44 | TCR32a | LFWYKQPPSRQMILVIR |
| | FR2 | |
| 45 | TCR32a | QEAYKQQN |
| | CDR2 | |
| 46 | TCR32a | ATENRFSVNFQKAAKSFSLKISDSQLGDTAMYFC |
| | FR3 | |
| 47 | TCR32a | AFMTPITQGGSEKLV |
| | CDR3 | |
| 48 | TCR32a | FGKGTKLTVNP |
| | FR4 | |
| 49 | TCR69b | MDTWLVCWAIFSLLKAGLTEPEVTQTPSHQVTQMGQE |
| | | VILRCVPISNHLYFYWYRQILGQKVEFLVSFYNNEISEK |
| | | SEIFDDQFSVERPDGSNFTLKIRSTKLEDSAMYFCASSE |
| | | WAGTEAFFGQGTRLTVVEDLNKVFPPEVAVFEPSEAEI |
| | | SHTQKATLVCLATGFFPDHVELSWWVNGKEVHSGVC |
| | | TDPQPLKEQPALNDSRYCLSSRLRVSATFWQNPRNHFR |
| | | CQVQFYGLSENDEWTQDRAKPVTQIVSAEAWGRADC |
| | | GFTSVSYQQGVLSATILYEILLGKATLYAVLVSALVLM |
| | | AMVKRKDF |
| 50 | TCR69b | EPEVTQTPSHQVTQMGQEVILRCVPI |
| | FR1 | |
| 51 | TCR69b | SNHLY |
| | CDR1 | |
| 52 | TCR69b | FYWYRQILGQKVEFLVS |
| | FR2 | |
| 53 | TCR69b | FYNNEI |
| | CDR2 | |
| 54 | TCR69b | SEKSEIFDDQFSVERPDGSNFTLKIRSTKLEDSAMYFC |
| | FR3 | |
| 55 | TCR69b | ASSEWAGTEAF |
| | CDR3 | |
| 56 | TCR69b | FGQGTRLTVV |
| | FR4 | |
| 57 | TCR69a | MVLKFSVSILWIQLAWVSTQLLEQSPQFLSIQEGENLTV |
| | | YCNSSSVFSSLQWYRQEPGEGPVLLVTVVTGGEVKKL |
| | | KRLTFQFGDARKDSSLHITAAQPGDTGLYLCAGKTTDS |
| | | WGKLQFGAGTQVVVTPDIQNPDPAVYQLRDSKSSDKS |
| | | VCLFTDFDSQTNVSQSKDSDVYITDKCVLDMRSMDFK |
| | | SNSAVAWSNKSDFACANAFNNSIIPEDTFFPSPESSCDV |
| | | KLVEKSFETDTNLNFQNLSVIGFRILLLKVAGFNLLMTL |
| | | RLWSS |
| 58 | TCR69a | TQLLEQSPQFLSIQEGENLTVYCNSS |
| | FR1 | |
| 59 | TCR69a | SVFSS |
| | CDR1 | |
| 60 | TCR69a | LQWYRQEPGEGPVLLVT |
| | FR2 | |
| 61 | TCR69a | VVTGGEV |
| | CDR2 | |
| 62 | TCR69a | KKLKRLTFQFGDARKDSSLHITAAQPGDTGLYLC |
| | FR3 | |
| 63 | TCR69a | AGKTTDSWGKLQ |
| | CDR3 | |
| 64 | TCR69a | FGAGTQVVVTP |
| | FR4 | |
| 65 | TCR79b | MGTSLLCWMALCLLGADHADTGVSQDPRHKITKRGQ |
| | | NVTFRCDPISEHNRLYWYRQTLGQGPEFLTYFQNEAQL |
| | | EKSRLLSDRFSAERPKGSFSTLEIQRTEQGDSAMYLCAT |
| | | PLGRTNEQFFGPGTRLTVLEDLKNVFPPEVAVFEPSEAE |
| | | ISHTQKATLVCLATGFYPDHVELSWWVNGKEVHSGVC |
| | | TDPQPLKEQPALNDSRYCLSSRLRVSATFWQNPRNHFR |
| | | CQVQFYGLSENDEWTQDRAKPVTQIVSAEAWGRADC |
| | | GFTSESYQQGVLSATILYEILLGKATLYAVLVSALVLM |
| | | AMVKRKDSRGGGSG |
| 66 | TCR79b | DTGVSQDPRHKITKRGQNVTFRCDPI |
| | FR1 | |
| 67 | TCR79b | SEHNR |
| | CDR1 | |
| 68 | TCR79b | LYWYRQTLGQGPEFLTY |
| | FR2 | |
| 69 | TCR79b | FQNEAQ |
| | CDR2 | |
| 70 | TCR79b | LEKSRLLSDRFSAERPKGSFSTLEIQRTEQGDSAMYLC |
| | FR3 | |
| 71 | TCR79b | ATPLGRTNEQF |
| | CDR3 | |
| 72 | TCR79b | FGPGTRLTVL |
| | FR4 | |
| 73 | TCR79a | MKSLRVLLVILWLQLSWVWSQQKEVEQNSGPLSVPEG |
| | | AIASLNCTYSDRVSQSFFWYRQYSGKSPELIMSIYSNGD |
| | | KEDGRFTAQLNKASQYVSLLIRDSQPSDSATYLCAVR |
| | | WAGGTSYGKLTFGQGTILTVHPNIQNPDPAVYQLRDS |
| | | KSSDKSVCLFTDFDSQTNVSQSKDSDVYITDKCVLDM |
| | | RSMDFKSNSAVAWSNKSDFACANAFNNSIIPEDTFFPSP |
| | | ESSCDVKLVEKSFETDTNLNFQNLSVIGFRILLLKVAGF |
| | | NLLMTLRLWSS |
| 74 | TCR79a | QKEVEQNSGPLSVPEGAIASLNCTYS |
| | FR1 | |
| 75 | TCR79a | DRVSQS |
| | CDR1 | |
| 76 | TCR79a | FFWYRQYSGKSPELIMS |
| | FR2 | |
| 77 | TCR79a | IYSNGD |
| | CDR2 | |
| 78 | TCR79a | KEDGRFTAQLNKASQYVSLLIRDSQPSDSATYLC |
| | FR3 | |
| 79 | TCR79a | AVRWAGGTSYGKLT |
| | CDR3 | |
| 80 | TCR79a | FGQGTILTVHP |
| | FR4 | |
| 81 | TCR81b | MGIRLLCRVAFCFLAVGLVDVKVTQSSRYLVKRTGEK |
| | | VFLECVQDMDHENMFWYRQDPGLGLRLIYFSYDVKM |
| | | KEKGDIPEGYSVSREKKERFSLILESASTNQTSMYLCAS |
| | | SFRLREGLGEQFFGPGTRLTVLEDLKNVFPPEVAVFEPS |
| | | EAEISHTQKATLVCLATGFYPDHVELSWWVNGKEVHS |
| | | GVCTDPQPLKEQPALNDSRYCLSSRLRVSATFWQNPR |
| | | NHFRCQVQFYGLSENDEWTQDRAKPVTQIVSAEAWG |
| | | RADCGFTSESYQQGVLSATILYEILLGKATLYAVLVSA |
| | | LVLMAMVKRKDSRGGGSG |
| 82 | TCR81b | DVKVTQSSRYLVKRTGEKVFLECVQD |
| | FR1 | |
| 83 | TCR81b | MDHEN |
| | CDR1 | |
| 84 | TCR81b | MFWYRQDP GLGLRLIYF |
| | FR2 | |
| 85 | TCR81b | SYDVKM |
| | CDR2 | |
| 86 | TCR81b | KEKGDIPEGYSVSREKKERFSLILESASTNQTSMYLC |
| | FR3 | |
| 87 | TCR81b | ASSFRLREGLGEQF |
| | CDR3 | |
| 88 | TCR81b | FGPGTRLTVL |
| | FR4 | |
| 89 | TCR81a | MACPGFLWALVISTCLEFSMAQTVTQSQPEMSVQEAE |
| | | TVTLSCTYDTSESDYYLFWYKQPPSRQMILVIRQEAYK |
| | | QQNATENRFSVNFQKAAKSFSLKISDSQLGDAAMYFC |
| | | AYCNPNAGKSTFGDGTTLTVKPNIQNPDPAVYQLRDS |
| | | KSSDKSVCLFTDFDSQTNVSQSKDSDVYITDKCVLDM |
| | | RSMDFKSNSAVAWSNKSDFACANAFNNSIIPEDTFFPSP |
| | | ESSCDVKLVEKSFETDTNLNFQNLSVIGFRILLLKVAGF |
| | | NLLMTLRLWSS |
| 90 | TCR81a | AQTVTQSQPEMSVQEAETVTLSCTYD |
| | FR1 | |
| 91 | TCR81a | TSESDYY |
| | CDR1 | |
| 92 | TCR81a | LFWYKQPPSRQMILVIR |
| | FR2 | |
| 93 | TCR81a | QEAYKQQN |
| | CDR2 | |
| 94 | TCR81a | ATENRFSVNFQKAAKSFSLKISDSQLGDAAMYFC |
| | FR3 | |
| 95 | TCR81a | AYCNPNAGKST |
| | CDR3 | |
| 96 | TCR81a | FGDGTTLTVKP |
| | FR4 | |
| 97 | TCR91-b- | MGPGLLHWMALCLLGTGHGDAMVIQNPRYQVTQFGK |
| | ST | PVTLSCSQTLNHNVMYWYQQKSSQAPKLLFHYYDKD |
| | | FNNEADTPDNFQSRRPNTSFCFLDIRSPGLGDAAMYLC |
| | | ATSTGRVYQPQHFGDGTRLSILEDLNKVFPPEVAVFEP |
| | | SEAEISHTQKATLVCLATGFFPDHVELSWWVNGKEVH |
| | | SGVSTDPQPLKEQPALNDSRYCLSSRLRVSATFWQNPR |
| | | NHFRCQVQFYGLSENDEWTQDRAKPVTQIVSAEAWG |
| | | RADCGFTSVSYQQGVLSATILYEILLGKATLYAVLVSA |
| | | LVLMAMVKRKDFGGSG |
| 98 | TCR91-b- | DAMVIQNPRYQVTQFGKPVTLSCSQT |
| | ST FR1 | |
| 99 | TCR91-b- | LNHNV |
| | ST CDR1 | |
| 100 | TCR91-b- | MYWYQQKSSQAPKLLFH |
| | ST FR2 | |
| 101 | TCR91-b- | YYDKDF |
| | ST CDR2 | |
| 102 | TCR91-b- | NNEADTPDNFQSRRPNTSFCFLDIRSPGLGDAAMYLC |
| | ST FR3 | |
| 103 | TCR91-b- | ATSTGRVYQPQH |
| | ST CDR3 | |
| 104 | TCR91-b- | FGDGTRLSIL |
| | ST FR4 | |
| 105 | TCR91-a- | MLTASLLRAVIASICVVSSMAQKVTQAQTEISVVEKED |
| | ST | VTLDCVYETRDTTYYLFWYKQPPSGELVFLIRRNSFDE |
| | | QNEISGRYSWNFQKSTSSFNFTITASQVVDSAVYFCAL |
| | | NEGGGSTLGRLYFGRGTQLTVWPDIQNPDPAVYQLRD |
| | | SKSSDKSVCLFTDFDSQTNVSQSKDSDVYITDKTVLDM |
| | | RSMDFKSNSAVAWSNKSDFACANAFNNSIIPEDTFFPSP |
| | | ESSCDVKLVEKSFETDTNLNFQNLSVIGFRILLLKVAGF |
| | | NLLMTLRLWSS |
| 106 | TCR91-a- | AQKVTQAQTEISVVEKEDVTLDCVYE |
| | ST FR1 | |
| 107 | TCR91-a- | TRDTTYY |
| | ST CDR1 | |
| 108 | TCR91-a- | LFWYKQPPSGELVFLIR |
| | ST FR2 | |
| 109 | TCR91-a- | RNSFDEQN |
| | ST CDR2 | |
| 110 | TCR91-a- | EISGRYSWNFQKSTSSFNFTITASQVVDSAVYFC |
| | ST | |
| | FR3 | |
| 111 | TCR91-a- | ALNEGGGSTLGRLY |
| | ST CDR3 | |
| 112 | TCR91-a- | FGRGTQLTVWP |
| | ST FR4 | |
| 117 | MG29-1 | MFNNFIKKYSLQKTLRFELKPVGETADYIEDFKSEYLK |
| | Cas | DTVLKDEQRAKDYQEIKTLIDDYHREYIEECLREPVDK |
| | endonuclea | KTGEILDFTQDLEDAFSYYQKLKENPTENRVGWEKEQ |
| | se | ESLRKKLVTSFVGNDGLFKKEFITRDLPEWLQKKGLW |
| | | GEYKDTVENFKKFTTYFSGFHENRKNMYTAEAQSTAI |
| | | ANRLMNDNLPKFFNNYLAYQTIKEKHPDLVFRLDDAL |
| | | LQAAGVEHLDEAFQPRYFSRLFAQSGITAFNELIGGRTT |
| | | ENGEKIQGLNEQINLYRQQNPEKAKGFPRFMPLFKQIL |
| | | SDRETHSFLPDAFENDKELLQALRDYVDAATSEEGMIS |
| | | QLNKAMNQFVTADLKRVYIKSAALTSLSQELFHFFGVI |
| | | SDAIAWY AEKRLSPKKAQESFLKQEVYAIEELNQAVV |
| | | GYIDQLEDQSELQQLLVDLPDPQKPVSSFILTHWQKSQ |
| | | EPLQAVIAKVEPLFELEELSKNKRAPKHDKDQGGEGFQ |
| | | QVDAIKNMLDAFMEVSHAIKPLYLVKGRKAIDMPDVD |
| | | TGFY ADFAEAYSAYEQVTVSLYNKTRNHLSKKPFSKD |
| | | KIKINFDAPTLLNGWDLNKESDNKSIILRKDGNFYLAIM |
| | | HPKHTKVFDCYSASEAAGKCYEKMNYKLLSGANKML |
| | | PKVFFSKKGIETFSPPQEILDLYKNNEHKKGATFKLESC |
| | | HKLIDFFKRNIPKYKVHPTDNFGWDVFGFHFSPTSSYG |
| | | DLSGFYREVEAQGYKLWFSDVSEAYINKCVEEGKLFL |
| | | FQIYNKDFSPNSTGKPNLHTLYWKGLFEPENLKDVVLK |
| | | LNGEAEIFYRKHSIKHEDKTIHRAKDPIANKNADNPKK |
| | | QSVFDYDIIKDKRYTQDKFFFHVPISLNFKSQGVVRFN |
| | | DKINGLLAAQDDVHVIGIDRGERHLLYYTVVNGKGEV |
| | | VEQGSLNQVATDQGYVVDYQQKLHAKEKERDQARK |
| | | NWSTIENIKELKAGYLSQVVHKLAQLIVKHNAIVCLED |
| | | LNFGFKRGRFKVEKQVYQKFEKALIDKLNYLVFKERG |
| | | ATQAGGYLNAYQLAAPFESFEKLGKQTGILYYVRSDY |
| | | TSKIDPATGFVDFLKPKYESMAKSKVFFESFERIQWNQ |
| | | AKGYFEFEFDYKKMCPSRKFGDYRTRWVVCTFGDTR |
| | | YQNRRNKSSGQWETETIDVTAQLKALFAAYGITYNQE |
| | | DNIKDAIAAVKYTKFYKQLYWLLRLTLSLRHSVTGTD |
| | | EDFILSPVADENGVFFDSRKATDKQPKDADANGAYHI |
| | | ALKGLWNLQQIRQHDWNVEKPKKLNLAMKNEEWFG |
| | | FAQKKKFRA |
| 118 | TCR alpha | IQNPDPAVYQLRDSKSSDKSVCLFTDFDSQTNVSQSKD |
| | chain | SDVYITDKTVLDMRSMDFKSNSAVAWSNKSDFACAN |
| | constant | AFNNSIIPEDTFFPSPESSCDVKLVEKSFETDTNLNFQNL |
| | (human) | SVIGFRILLLKVAGFNLLMTLRLWSS |
| 119 | TCR beta | DLNKVFPPEVAVFEPSEAEISHTQKATLVCLATGFFPDH |
| | constant | VELSWWVNGKEVHSGVSTDPQPLKEQPALNDSRYCLS |
| | (human) | SRLRVSATFWQNPRNHFRCQVQFYGLSENDEWTQDR |
| | | AKPVTQIVSAEAWGRADCGFTSVSYQQGVLSATILYEI |
| | | LLGKATLYAVLVSALVLMAMVKRKDF |
| 120 | TCR | DKQLDADVSPKPTIFLPSIAETKLQKAGTYLCLLEKFFP |
| | gamma | DVIKIHWQEKKSNTILGSQEGNTMKTNDTYMKFSWLT |
| | constant 1 | VPEKSLDKEHRCIVRHENNKNGVDQEIIFPPIKTDVITM |
| | (human) | DPKDNCSKDANDTLLLQLTNTSAYYMYLLLLLKSVVY |
| | | FAIITCCLLRRTAFCCNGEKS |
| 121 | TCR | DKQLDADVSPKPTIFLPSIAETKLQKAGTYLCLLEKFFP |
| | gamma | DIIKIHWQEKKSNTILGSQEGNTMKTNDTYMKFSWLT |
| | constant 2 | VPEESLDKEHRCIVRHENNKNGIDQEIIFPPIKTDVTTVD |
| | (human) | PKYNYSKDANDVITMDPKDNWSKDANDTLLLQLTNT |
| | | SAYYTYLLLLLKSVVYFAIITCCLLRRTAFCCNGEKS |
| 122 | TCR delta | SQPHTKPSVFVMKNGTNVACLVKEFYPKDIRINLVSSK |
| | constant | KITEFDPAIVISPSGKYNAVKLGKYEDSNSVTCSVQHD |
| | (human) | NKTVHSTDFEVKTDSTDHVKPKETENTKQPSKSCHKP |
| | | KAIVHTEKVNMMSLTVLGLRMLFAKTVAVNFLLTAKL |
| | | FFL |
| 123 | TCRla | MNYSPGLVSLILLLLGRTRGDSVTQMEGPVTLSEEAFL |
| | | TINCTYTATGYPSLFWYVQYPGEGLQLLLKATKADDK |
| | | GSNKGFEATYRKETTSFHLEKGSVQVSDSAVYFCALSD |
| | | RVGGARLMFGDGTQLVVKPNIQNPDPAVYQLRDSKSS |
| | | DKSVCLFTDFDSQTNVSQSKDSDVYITDKCVLDMRSM |
| | | DFKSNSAVAWSNKSDFACANAFNNSIIPEDTFFPSPESS |
| | | CDVKLVEKSFETDTNLNFQNLSVIGFRILLLKVAGENLL |
| | | MTLRLWSS |
| 124 | TCRla FR1 | GDSVTQMEGPVTLSEEAFLTINCTYT |
| 125 | TCRla | ATGYPS |
| | CDR1 | |
| 126 | TCRla FR2 | LFWYVQYPGEGLQLLLK |
| 127 | TCRla | ATKADDK |
| | CDR2 | |
| 128 | TCRla FR3 | GSNKGFEATYRKETTSFHLEKGSVQVSDSAVYFC |
| 129 | TCRla | ALSDRVGGARLM |
| | CDR3 | |
| 130 | TCRla FR4 | FGDGTQLVVKP |
| 131 | TCR1b | MTIRLLCYMGFYFLGAGLMEADIYQTPRYLVIGTGKKI |
| | | TLECSQTMGHDKMYWYQQDPGMELHLIHYSYGVNST |
| | | EKGDLSSESTVSRIRTEHFPLTLESARPSHTSQYLCASSE |
| | | GLYNEQFFGPGTRLTVLEDLKNVFPPEVAVFEPSEAEIS |
| | | HTQKATLVCLATGFYPDHVELSWWVNGKEVHSGVCT |
| | | DPQPLKEQPALNDSRYCLSSRLRVSATFWQNPRNHFRC |
| | | QVQFYGLSENDEWTQDRAKPVTQIVSAEAWGRADCG |
| | | FTSESYQQGVLSATILYEILLGKATLYAVLVSALVLMA |
| | | MVKRKDSRG |
| 132 | TCR1b | EADIYQTPRYLVIGTGKKITLECSQT |
| | FR1 | |
| 133 | TCR1b | MGHDK |
| | CDR1 | |
| 134 | TCR1b | MYWYQQDPGMELHLIHY |
| | FR2 | |
| 135 | TCR1b | SYGVNS |
| | CDR2 | |
| 136 | TCR1b | TEKGDLSSESTVSRIRTEHFPLTLESARPSHTSQYLC |
| | FR3 | |
| 137 | TCR1b | ASSEGLYNEQF |
| | CDR3 | |
| 138 | TCR1b | FGPGTRLTVL |
| | FR4 | |
| 139 | TCR2a | MTRVSLLWAVVVSTCLESGMAQTVTQSQPEMSVQEA |
| | | ETVTLSCTYDTSENNYYLFWYKQPPSRQMILVIRQEAY |
| | | KQQNATENRFSVNFQKAAKSFSLKISDSQLGDTAMYF |
| | | CAFMYPSQGGSEKLVFGKGMKLTVNPNIQNPDPAVYQ |
| | | LRDSKSSDKSVCLFTDFDSQTNVSQSKDSDVYITDKCV |
| | | LDMRSMDFKSNSAVAWSNKSDFACANAFNNSIIPEDTF |
| | | FPSPESSCDVKLVEKSFETDTNLNFQNLSVIGFRILLLKV |
| | | AGFNLLMTLRLWSS |
| 140 | TCR2a FR1 | AQTVTQSQPEMSVQEAETVTLSCTYD |
| 141 | TCR2a | TSENNYY |
| | CDR1 | |
| 142 | TCR2a FR2 | LFWYKQPPSRQMILVIR |
| 143 | TCR2a | QEAYKQQN |
| | CDR2 | |
| 144 | TCR2a FR3 | ATENRFSVNFQKAAKSFSLKISDSQLGDTAMYFC |
| 145 | TCR2a | AFMYPSQGGSEKLV |
| | CDR3 | |
| 146 | TCR2a FR4 | FGKGMKLTVNP |
| 147 | TCR2b | MGPGLLCWVLLCLLGAGSVETGVTQSPTHLIKTRGQQ |
| | | VTLRCSSQSGHNTVSWYQQALGQGPQFIFQYYREEEN |
| | | GRGNFPPRFSGLQFPNYSSELNVNALELDDSALYLCAS |
| | | SSPGFRSYGYTFGSGTRLTVVEDLKNVFPPEVAVFEPSE |
| | | AEISHTQKATLVCLATGFYPDHVELSWWVNGKEVHSG |
| | | VCTDPQPLKEQPALNDSRYCLSSRLRVSATFWQNPRN |
| | | HFRCQVQFYGLSENDEWTQDRAKPVTQIVSAEAWGR |
| | | ADCGFTSESYQQGVLSATILYEILLGKATLYAVLVSAL |
| | | VLMAMVKRKDSRG |
| 148 | TCR2b | ETGVTQSPTHLIKTRGQQVTLRCSSQ |
| | FR1 | |
| 149 | TCR2b | SGHNT |
| | CDR1 | |
| 150 | TCR2b | VSWYQQALGQGPQFIFQ |
| | FR2 | |
| 151 | TCR2b | YYREEE |
| | CDR2 | |
| 152 | TCR2b | NGRGNFPPRFSGLQFPNYSSELNVNALELDDSALYLC |
| | FR3 | |
| 153 | TCR2b | ASSSPGFRSYGYT |
| | CDR3 | |
| 154 | TCR2b | FGSGTRLTVV |
| | FR4 | |
| 155 | TCR3a | MACPGFLWALVISTCLEFSMAQTVTQSQPEMSVQEAE |
| | | TVTLSCTYDTSESDYYLFWYKQPPSRQMIL VIRQEAYK |
| | | QQNATENRFSVNFQKAAKSFSLKISDSQLGDAAMYFC |
| | | AYRSDGGATNKLIFGTGTLLAVQPNIQNPDPAVYQLRD |
| | | SKSSDKSVCLFTDFDSQTNVSQSKDSDVYITDKCVLDM |
| | | RSMDFKSNSAVAWSNKSDFACANAFNNSIIPEDTFFPSP |
| | | ESSCDVKLVEKSFETDTNLNFQNLSVIGFRILLLKVAGF |
| | | NLLMTLRLWSS |
| 156 | TCR3a FR1 | AQTVTQSQPEMSVQEAETVTLSCTYD |
| 157 | TCR3a | TSESDYY |
| | CDR1 | |
| 158 | TCR3a FR2 | LFWYKQPPSRQMILVIR |
| 159 | TCR3a | QEAYKQQN |
| | CDR2 | |
| 160 | TCR3a FR3 | ATENRFSVNFQKAAKSFSLKISDSQLGDAAMYFC |
| 161 | TCR3a | AYRSDGGATNKLI |
| | CDR3 | |
| 162 | TCR3a FR4 | FGTGTLLAVQP |
| 163 | TCR3b | MGPQLLGYVVLCLLGAGPLEAQVTQNPRYLITVTGKK |
| | | LTVTCSQNMNHEYMSWYRQDPGLGLRQIYYSMNVEV |
| | | TDKGDVPEGYKVSRKEKRNFPLILESPSPNQTSLYFCAS |
| | | SLGAGGYNSPLHFGNGTRLEDLKNVFPPEVAVFEPSEA |
| | | EISHTQKATLVCLATGFYPDHVELSWWVNGKEVHSGV |
| | | CTDPQPLKEQPALNDSRYCLSSRLRVSATFWQNPRNHF |
| | | RCQVQFYGLSENDEWTQDRAKPVTQIVSAEAWGRAD |
| | | CGFTSESYQQGVLSATILYEILLGKATLYAVLVSALVL |
| | | MAMVKRKDSRG |
| 164 | TCR3b | EAQVTQNPRYLITVTGKKLTVTCSQN |
| | FR1 | |
| 165 | TCR3b | MNHEY |
| | CDR1 | |
| 166 | TCR3b | MSWYRQDPGLGLRQIYY |
| | FR2 | |
| 167 | TCR3b | SMNVEV |
| | CDR2 | |
| 168 | TCR3b | TDKGDVPEGYKVSRKEKRNFPLILESPSPNQTSLYFC |
| | FR3 | |
| 169 | TCR3b | ASSLGAGGYNSPLH |
| | CDR3 | |
| 170 | TCR3b | FGNGTRL |
| | FR4 | |
| 171 | TCR4a | MMKSLRVLLVILWLQLSWVWSQQKEVEQDPGPLSVP |
| | | EGAIVSLNCTYSNSAFQYFMWYRQYSRKGPELLMYTY |
| | | SSGNKEDGRFTAQVDKSSKYISLFIRDSQPSDSATYLCA |
| | | MGALNSGAGSYQLTFGKGTKLSVIPNIQNPDPAVYQLR |
| | | DSKSSDKSVCLFTDFDSQTNVSQSKDSDVYITDKCVLD |
| | | MRSMDFKSNSAVAWSNKSDFACANAFNNSIIPEDTFFP |
| | | SPESSCDVKLVEKSFETDTNLNFQNLSVIGFRILLLKVA |
| | | GFNLLMTLRLWSS |
| 172 | TCR4a FR1 | QKEVEQDPGPLSVPEGAIVSLNCTYS |
| 173 | TCR4a | NSAFQY |
| | CDR1 | |
| 174 | TCR4a FR2 | FMWYRQYSRKGPELLMY |
| 175 | TCR4a | TYSSGN |
| | CDR2 | |
| 176 | TCR4a FR3 | KEDGRFTAQVDKSSKYISLFIRDSQPSDSATYLC |
| 177 | TCR4a | AMGALNSGAGSYQLT |
| | CDR3 | |
| 178 | TCR4a FR4 | FGKGTKLSVIP |
| 179 | TCR4b | MGSRLLCWVLLCLLGAGPVKAGVTQTPRYLIKTRGQQ |
| | | VTLSCSPISGHRSVSWYQQTPGQGLQFLFEYFSETQRN |
| | | KGNFPGRFSGRQFSNSRSEMNVSTLELGDSALYLCASS |
| | | LSSGTGTEAFFGQGTRLTVEDLKNVFPPEVAVFEPSEA |
| | | EISHTQKATLVCLATGFYPDHVELSWWVNGKEVHSGV |
| | | CTDPQPLKEQPALNDSRYCLSSRLRVSATFWQNPRNHF |
| | | RCQVQFYGLSENDEWTQDRAKPVTQIVSAEAWGRAD |
| | | CGFTSESYQQGVLSATILYEILLGKATLYAVLVSALVL |
| | | MAMVKRKDSRG |
| 180 | TCR4b | KAGVTQTPRYLIKTRGQQVTLSCSPI |
| | FR1 | |
| 181 | TCR4b | SGHRS |
| | CDR1 | |
| 182 | TCR4b | VSWYQQTPGQGLQFLFE |
| | FR2 | |
| 183 | TCR4b | YFSETQ |
| | CDR2 | |
| 184 | TCR4b | RNKGNFPGRFSGRQFSNSRSEMNVSTLELGDSALYLC |
| | FR3 | |
| 185 | TCR4b | ASSLSSGTGTEAF |
| | CDR3 | |
| 186 | TCR4b | FGQGTRLTV |
| | FR4 | |
| 187 | TCR5a | MKTFAGFSFLFLWLQLDCMSRGEDVEQSLFLSVREGD |
| | | SSVINCTYTDSSSTYLYWYKQEPGAGLQLLTYIFSNMD |
| | | MKQDQRLTVLLNKKDKHLSLRIADTQTGDSAIYFCAE |
| | | RDAGNNRKLIWGLGTSLAVNPNIQNPDPAVYQLRDSK |
| | | SSDKSVCLFTDFDSQTNVSQSKDSDVYITDKCVLDMRS |
| | | MDFKSNSAVAWSNKSDFACANAFNNSIIPEDTFFPSPES |
| | | SCDVKLVEKSFETDTNLNFQNLSVIGFRILLLKVAGFNL |
| | | LMTLRLWSS |
| 188 | TCR5a FR1 | GEDVEQSLFLSVREGDSSVINCTYT |
| 189 | TCR5a | DSSSTY |
| | CDR1 | |
| 190 | TCR5a FR2 | LYWYKQEPGAGLQLLTY |
| 191 | TCR5a | IFSNMDM |
| | CDR2 | |
| 192 | TCR5a FR3 | KQDQRLTVLLNKKDKHLSLRIADTQTGDSAIYFC |
| 193 | TCR5a | AERDAGNNRKLIWG |
| | CDR3 | |
| 194 | TCR5a FR4 | LGTSLAVNP |
| 195 | TCR5b | MGTRLLCWVVLGFLGTDHTGAGVSQSPRYKVAKRGQ |
| | | DVALRCDPISGHVSLFWYQQALGQGPEFLTYFQNEAQ |
| | | LDKSGLPSDRFFAERPEGSVSTLKIQRTQQEDSAVYLC |
| | | ASSLEGGDTQYFGPGTRLTVLEDLKNVFPPEVAVFEPS |
| | | EAEISHTQKATLVCLATGFYPDHVELSWWVNGKEVHS |
| | | GVCTDPQPLKEQPALNDSRYCLSSRLRVSATFWQNPR |
| | | NHFRCQVQFYGLSENDEWTQDRAKPVTQIVSAEAWG |
| | | RADCGFTSESYQQGVLSATILYEILLGKATLYAVLVSA |
| | | LVLMAMVKRKDSRG |
| 196 | TCR5b | GAGVSQSPRYKVAKRGQDVALRCDPI |
| | FR1 | |
| 197 | TCR5b | SGHVS |
| | CDR1 | |
| 198 | TCR5b | LFWYQQALGQGPEFLTY |
| | FR2 | |
| 199 | TCR5b | FQNEAQ |
| | CDR2 | |
| 200 | TCR5b | LDKSGLPSDRFFAERPEGSVSTLKIQRTQQEDSAVYLC |
| | FR3 | |
| 201 | TCR5b | ASSLEGGDTQY |
| | CDR3 | |
| 202 | TCR5b | FGPGTRLTVL |
| | FR4 | |
| 203 | TCR6a | MLLEHLLIILWMQLTWVSGQQLNQSPQSMFIQEGEDV |
| | | SMNCTSSSIFNTWLWYKQDPGEGPVLLIALYKAGELTS |
| | | NGRLTAQFGITRKDSFLNISASIPSDVGIYFCAGRREGA |
| | | QKLVFGQGTRLTINPNIQNPDPAVYQLRDSKSSDKSVC |
| | | LFTDFDSQTNVSQSKDSDVYITDKCVLDMRSMDFKSN |
| | | SAVAWSNKSDFACANAFNNSIIPEDTFFPSPESSCDVKL |
| | | VEKSFETDTNLNFQNLSVIGFRILLLKVAGFNLLMTLRL |
| | | WSS |
| 204 | TCR6a FR1 | GQQLNQSPQSMFIQEGEDVSMNCTSS |
| 205 | TCR6a | SIFNT |
| | CDR1 | |
| 206 | TCR6a FR2 | WLWYKQDPGEGPVLLIA |
| 207 | TCR6a | LYKAGEL |
| | CDR2 | |
| 208 | TCR6a FR3 | TSNGRLTAQFGITRKDSFLNISASIPSDVGIYFC |
| 209 | TCR6a | AGRREGAQKLV |
| | CDR3 | |
| 210 | TCR6a FR4 | FGQGTRLTINP |
| 211 | TCR6b | MGTRLLCWVVLGFLGTDHTGAGVSQSPRYKVAKRGQ |
| | | DVALRCDPISGHVSLFWYQQALGQGPEFLTYFQNEAQ |
| | | LDKSGLPSDRFFAERPEGSVSTLKIQRTQQEDSAVYLC |
| | | ASSSTLMGVNIQYFGAGTRLSVLEDLKNVFPPEVAVFE |
| | | PSEAEISHTQKATLVCLATGFYPDHVELSWWVNGKEV |
| | | HSGVCTDPQPLKEQPALNDSRYCLSSRLRVSATFWQNP |
| | | RNHFRCQVQFYGLSENDEWTQDRAKPVTQIVSAEAW |
| | | GRADCGFTSESYQQGVLSATILYEILLGKATLYAVLVS |
| | | ALVLMAMVKRKDSRG |
| 212 | TCR6b | GAGVSQSPRYKVAKRGQDVALRCDPI |
| | FR1 | |
| 213 | TCR6b | SGHVS |
| | CDR1 | |
| 214 | TCR6b | LFWYQQALGQGPEFLTY |
| | FR2 | |
| 215 | TCR6b | FQNEAQ |
| | CDR2 | |
| 216 | TCR6b | LDKSGLPSDRFFAERPEGSVSTLKIQRTQQEDSAVYLC |
| | FR3 | |
| 217 | TCR6b | ASSSTLMGVNIQY |
| | CDR3 | |
| 218 | TCR6b | FGAGTRLSVL |
| | FR4 | |
| 219 | CD8a | MALPVTALLLPLALLLHAARPSQFRVSPLDRTWNLGET |
| | | VELKCQVLLSNPTSGCSWLFQPRGAAASPTFLLYLSQN |
| | | KPKAAEGLDTQRFSGKRLGDTFVLTLSDFRRENEGYYF |
| | | CSALSNSIMYFSHFVPVFLPAKPTTTPAPRPPTPAPTIAS |
| | | QPLSLRPEACRPAAGGAVHTRGLDFACDIYIWAPLAGT |
| | | CGVLLLSLVITLYCNHRNRRRVCKCPRPVVKSGDKPSL |
| | | SARYV |
| 220 | CD8b | MRPRLWLLLAAQLTVLHGNSVLQQTPAYIKVQTNKM |
| | | VMLSCEAKISLSNMRIYWLRQRQAPSSDSHHEFLALW |
| | | DSAKGTIHGEEVEQEKIAVFRDASRFILNLTSVKPEDSG |
| | | IYFCMIVGSPELTFGKGTQLSVVDFLPTTAQPTKKSTLK |
| | | KRVCRLPRPETQKGPLCSPITLGLLVAGVLVLLVSLGV |
| | | AIHLCCRRRRARLRFMKQFYK |
| 221 | Fas-41BB | MLGIWTLLPLVLTSVARLSSKSVNAQVTDINSKGLELR |
| | | KTVTTVETQNLEGLHHDGQFCHKPCPPGERKARDCTV |
| | | NGDEPDCVPCQEGKEYTDKAHFSSKCRRCRLCDEGHG |
| | | LEVEINCTRTQNTKCRCKPNFFCNSTVCEHCDPCTKCE |
| | | HGIIKECTLTSNTKCKEEGSRSNLGWLCLLLLPIPLIVW |
| | | VKRGRKKLLYIFKQPFMRPVQTTQEEDGCSCRFPEEEE |
| | | GGCEL |
| 222 | CD34IL7R | MLVRRGARAGPRMPRGWTALCLLSLLPSGFMSLDNN |
| | A | GTATPELPTQGTFSNVSTNVSYQETTTPSTLGSTSLHPV |
| | | SQHGNEATTNITETTVKFTSTSVITSVYGNTNSSVQSQT |
| | | SVISTVFTTPANVSTPETTLKPSLSPGNVSDLSTTSTSLA |
| | | TSPTKPYTSSSPILSDIKAEIKCSGIREVKLTQGICLEQNK |
| | | TSSCAEFKKDRGEGLARVLCGEEQADADAGAQVCSLL |
| | | LAQSEVRPQCLLLVLANRTEISSKLQLMKKHQSDLKKL |
| | | GILDFTEQDVASHQSYSQKTPILLTCPTISILSFFSVALLV |
| | | ILACVLWKKRIKPIVWPSLPDHKKTLEHLCKKPRKNLN |
| | | VSFNPESFLDCQIHRVDDIQARDEVEGFLQDTFPQQLEE |
| | | SEKQRLGGDVQSPNCPSEDVVITPESFGRDSSLTCLAG |
| | | NVSACDAPILSSSRSLDCRESGKNGPHVYQDLLLSLGT |
| | | TNSTLPPPFSLQSGILTLNPVAQGQPILTSLGSNQEEAYV |
| | | TMSSFYQNQ |
| 223 | CD58IL7RA | MVAGSDAGRALGVLSVVCLLHCFGFISCFSQQIYGVV |
| | | YGNVTFHVPSNVPLKEVLWKKQKDKVAELENSEFRAF |
| | | SSFKNRVYLDTVSGSLTIYNLTSSDEDEYEMESPNITDT |
| | | MKFFLYVLESLPSPTLTCALTNGSIEVQCMIPEHYNSHR |
| | | GLIMYSWDCPMEQCKRNSTSIYFKMENDLPQKIQCTLS |
| | | NPLFNTTSSIILTTCIPSSGHSRHRPILLTCPTISILSFFSVA |
| | | LLVILACVLWKKRIKPIVWPSLPDHKKTLEHLCKKPRK |
| | | NLNVSFNPESFLDCQIHRVDDIQARDEVEGFLQDTFPQ |
| | | QLEESEKQRLGGDVQSPNCPSEDVVITPESFGRDSSLTC |
| | | LAGNVSACDAPILSSSRSLDCRESGKNGPHVYQDLLLS |
| | | LGTTNSTLPPPFSLQSGILTLNPVAQGQPILTSLGSNQEE |
| | | AYVTMSSFYQNQGSG |
| 224 | IL7RA | KKRIKPIVWPSLPDHKKTLEHLCKKPRKNLNVSFNPES |
| | intracellular | FLDCQIHRVDDIQARDEVEGFLQDTFPQQLEESEKQRL |
| | domain | GGDVQSPNCPSEDVVITPESFGRDSSLTCLAGNVSACD |
| | | APILSSSRSLDCRESGKNGPHVYQDLLLSLGTTNSTLPP |
| | | PFSLQSGILTLNPVAQGQPILTSLGSNQEEAYVTMSSFY |
| | | QNQ |
| 225 | IL7RA | PILLTISILSFFSVALLVILACVLW |
| | transmembrane | |
| | domain | |
| 226 | CD80 | MGHTRRQGTSPSKCPYLNFFQLLVLAGLSHFCSGVIHV |
| | extracellular | TKEVKEVATLSCGHNVSVEELAQTRIYWQKEKKMVLT |
| | domain | MMSGDMNIWPEYKNRTIFDITNNLSIVILALRPSDEGTY |
| | | ECVVLKYEKDAFKREHLAEVTLSVKADFPTPSISDFEIP |
| | | TSNIRRIICSTSGGFPEPHLSWLENGEELNAINTTVSQDP |
| | | ETELYAVSSKLDFNMTTNHSFMCLIKYGHLRVNQTFN |
| | | WNTTKQEHFPDN |
| 227 | CD58 | MVAGSDAGRALGVLSVVCLLHCFGFISCFSQQIYGVV |
| | extracellular | YGNVTFHVPSNVPLKEVLWKKQKDKVAELENSEFRAF |
| | domain | SSFKNRVYLDTVSGSLTIYNLTSSDEDEYEMESPNITDT |
| | | MKFFLYVLESLPSPTLTCALTNGSIEVQCMIPEHYNSHR |
| | | GLIMYSWDCPMEQCKRNSTSIYFKMENDLPQKIQCTLS |
| | | NPLFNTTSSIILTTCIPSSGHSRHR |
| 228 | CD34 | MLVRRGARAGPRMPRGWTALCLLSLLPSGFMSLDNN |
| | extracellular | GTATPELPTQGTFSNVSTNVSYQETTTPSTLGSTSLHPV |
| | domain | SQHGNEATTNITETTVKFTSTSVITSVYGNTNSSVQSQT |
| | | SVISTVFTTPANVSTPETTLKPSLSPGNVSDLSTTSTSLA |
| | | TSPTKPYTSSSPILSDIKAEIKCSGIREVKLTQGICLEQNK |
| | | TSSCAEFKKDRGEGLARVLCGEEQADADAGAQVCSLL |
| | | LAQSEVRPQCLLLVLANRTEISSKLQLMKKHQSDLKKL |
| | | GILDFTEQDVASHQSYSQKT |
| 229 | TCRl′a | MNYSPGLVSLILLLLGRTRGDSVTQMEGPVTLSEEAFL |
| | | TINCTYTATGYPSLFWYVQYPGEGLQLLLKATKADDK |
| | | GSNKGFEATYRKETTSFHLEKGSVQVSDSAVYFCALSD |
| | | RVGGARLMFGDGTQLVVKPNIQNPDPAVYQLRDSKSS |
| | | DKSVCLFTDFDSQTNVSQSKDSDVYITDKTVLDMRSM |
| | | DFKSNSAVAWSNKSDFACANAFNNSIIPEDTFFPSPESS |
| | | CDVKLVEKSFETDTNLNFQNLSVIGFRILLLKVAGENLL |
| | | MTLRLWSS |
| 230 | TCRl′a var | MNYSPGLVSLILLLLGRTRGDSVTQMEGPVTLSEEAFL |
| | | TINCTYTATGYPSLFWYVQYPGEGLQLLLKATKADDK |
| | | GSNKGFEATYRKETTSFHLEKGSVQVSDSAVYFCALSD |
| | | RVGGARLMFGDGTQLVVKP |
| 231 | TCRl′a | TATGYPS |
| | CDR1 | |
| 232 | TCRl′a | ATKADDK |
| | CDR2 | |
| 233 | TCRl′a | CALSDRVGGARLMF |
| | CDR3 | |
| 234 | TCR1′b | MTIRLLCYMGFYFLGAGLMEADIYQTPRYLVIGTGKKI |
| | | TLECSQTMGHDKMYWYQQDPGMELHLIHYSYGVNST |
| | | EKGDLSSESTVSRIRTEHFPLTLESARPSHTSQYLCASSE |
| | | GLYNEQFFGPGTRLTVLEDLKNVFPPEVAVFEPSEAEIS |
| | | HTQKATLVCLATGFYPDHVELSWWVNGKEVHSGVST |
| | | DPQPLKEQPALNDSRYCLSSRLRVSATFWQNPRNHFRC |
| | | QVQFYGLSENDEWTQDRAKPVTQIVSAEAWGRADCG |
| | | FTSESYQQGVLSATILYEILLGKATLYAVLVSALVLMA |
| | | MVKRKDSRG |
| 235 | TCR1′b var | MTIRLLCYMGFYFLGAGLMEADIYQTPRYLVIGTGKKI |
| | | TLECSQTMGHDKMYWYQQDPGMELHLIHYSYGVNST |
| | | EKGDLSSESTVSRIRTEHFPLTLESARPSHTSQYLCASSE |
| | | GLYNEQFFGPGTRLTVL |
| 236 | TCR1′b | MGHDK |
| | CDR1 | |
| 237 | TCR1′b | SYGVNS |
| | CDR2 | |
| 238 | TCR1′b | CASSEGLYNEQFF |
| | CDR3 | |
| 239 | TCR2′a | MTRVSLLWAVVVSTCLESGMAQTVTQSQPEMSVQEA |
| | | ETVTLSCTYDTSENNYYLFWYKQPPSRQMILVIRQEAY |
| | | KQQNATENRFSVNFQKAAKSFSLKISDSQLGDTAMYF |
| | | CAFMYPSQGGSEKLVFGKGMKLTVNPNIQNPDPAVYQ |
| | | LRDSKSSDKSVCLFTDFDSQTNVSQSKDSDVYITDKTV |
| | | LDMRSMDFKSNSAVAWSNKSDFACANAFNNSIIPEDTF |
| | | FPSPESSCDVKLVEKSFETDTNLNFQNLSVIGFRILLLKV |
| | | AGFNLLMTLRLWSS |
| 240 | TCR2′a var | MTRVSLLWAVVVSTCLESGMAQTVTQSQPEMSVQEA |
| | | ETVTLSCTYDTSENNYYLFWYKQPPSRQMIL VIRQEAY |
| | | KQQNATENRFSVNFQKAAKSFSLKISDSQLGDTAMYF |
| | | CAFMYPSQGGSEKLVFGKGMKLTVNP |
| 241 | TCR2′a | TSENNYY |
| | CDR1 | |
| 242 | TCR2′a | QEAYKQQN |
| | CDR2 | |
| 243 | TCR2′a | CAFMYPSQGGSEKLVF |
| | CDR3 | |
| 244 | TCR2′b | MGPGLLCWVLLCLLGAGSVETGVTQSPTHLIKTRGQQ |
| | | VTLRCSSQSGHNTVSWYQQALGQGPQFIFQYYREEEN |
| | | GRGNFPPRFSGLQFPNYSSELNVNALELDDSALYLCAS |
| | | SSPGFRSYGYTFGSGTRLTVVEDLNKVFPPEVAVFEPSE |
| | | AEISHTQKATLVCLATGFFPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRA |
| | | DCGFTSVSYQQGVLSATILYEILLGKATLYAVLVSALV |
| | | LMAMVKRKDF |
| 245 | TCR2′b var | MGPGLLCWVLLCLLGAGSVETGVTQSPTHLIKTRGQQ |
| | | VTLRCSSQSGHNTVSWYQQALGQGPQFIFQYYREEEN |
| | | GRGNFPPRFSGLQFPNYSSELNVNALELDDSALYLCAS |
| | | SSPGFRSYGYTFGSGTRLTVV |
| 246 | TCR2′b | SGHNT |
| | CDR1 | |
| 247 | TCR2′b | YYREEE |
| | CDR2 | |
| 248 | TCR2′b | CASSSPGFRSYGYTF |
| | CDR3 | |
| 249 | TCR3′a | MACPGFLWALVISTCLEFSMAQTVTQSQPEMSVQEAE |
| | | TVTLSCTYDTSESDYYLFWYKQPPSRQMILVIRQEAYK |
| | | QQNATENRFSVNFQKAAKSFSLKISDSQLGDAAMYFC |
| | | AYRSDGGATNKLIFGTGTLLAVQPNIQNPDPAVYQLRD |
| | | SKSSDKSVCLFTDFDSQTNVSQSKDSDVYITDKTVLDM |
| | | RSMDFKSNSAVAWSNKSDFACANAFNNSIIPEDTFFPSP |
| | | ESSCDVKLVEKSFETDTNLNFQNLSVIGFRILLLKVAGF |
| | | NLLMTLRLWSS |
| 250 | TCR3′a var | MACPGFLWALVISTCLEFSMAQTVTQSQPEMSVQEAE |
| | | TVTLSCTYDTSESDYYLFWYKQPPSRQMILVIRQEAYK |
| | | QQNATENRFSVNFQKAAKSFSLKISDSQLGDAAMYFC |
| | | AYRSDGGATNKLIFGTGTLLAVQP |
| 251 | TCR3′a | TSESDYY |
| | CDR1 | |
| 252 | TCR3′a | QEAYKQQN |
| | CDR2 | |
| 253 | TCR3′a | CAYRSDGGATNKLIF |
| | CDR3 | |
| 254 | TCR3′b | MGPQLLGYVVLCLLGAGPLEAQVTQNPRYLITVTGKK |
| | | LTVTCSQNMNHEYMSWYRQDPGLGLRQIYYSMNVEV |
| | | TDKGDVPEGYKVSRKEKRNFPLILESPSPNQTSLYFCAS |
| | | SLGAGGYNSPLHFGNGTRLTVTEDLNKVFPPEVAVFEP |
| | | SEAEISHTQKATLVCLATGFFPDHVELSWWVNGKEVH |
| | | SGVSTDPQPLKEQPALNDSRYCLSSRLRVSATFWQNPR |
| | | NHFRCQVQFYGLSENDEWTQDRAKPVTQIVSAEAWG |
| | | RADCGFTSVSYQQGVLSATILYEILLGKATLYAVLVSA |
| | | LVLMAMVKRKDF |
| 255 | TCR3′b var | MGPQLLGYVVLCLLGAGPLEAQVTQNPRYLITVTGKK |
| | | LTVTCSQNMNHEYMSWYRQDPGLGLRQIYYSMNVEV |
| | | TDKGDVPEGYKVSRKEKRNFPLILESPSPNQTSLYFCAS |
| | | SLGAGGYNSPLHFGNGTRLTVT |
| 256 | TCR3′b | MNHEY |
| | CDR1 | |
| 257 | TCR3′b | SMNVEV |
| | CDR2 | |
| 258 | TCR3′b | CASSLGAGGYNSPLHF |
| | CDR3 | |
| 259 | TCR4′a | MMKSLRVLLVILWLQLSWVWSQQKEVEQDPGPLSVP |
| | | EGAIVSLNCTYSNSAFQYFMWYRQYSRKGPELLMYTY |
| | | SSGNKEDGRFTAQVDKSSKYISLFIRDSQPSDSATYLCA |
| | | MGALNSGAGSYQLTFGKGTKLSVIPNIQNPDPAVYQLR |
| | | DSKSSDKSVCLFTDFDSQTNVSQSKDSDVYITDKTVLD |
| | | MRSMDFKSNSAVAWSNKSDFACANAFNNSIIPEDTFFP |
| | | SPESSCDVKLVEKSFETDTNLNFQNLSVIGFRILLLKVA |
| | | GFNLLMTLRLWSS |
| 260 | TCR4′a var | MMKSLRVLLVILWLQLSWVWSQQKEVEQDPGPLSVP |
| | | EGAIVSLNCTYSNSAFQYFMWYRQYSRKGPELLMYTY |
| | | SSGNKEDGRFTAQVDKSSKYISLFIRDSQPSDSATYLCA |
| | | MGALNSGAGSYQLTFGKGTKLSVIP |
| 261 | TCR4′a | NSAFQY |
| | CDR1 | |
| 262 | TCR4′a | TYSSGN |
| | CDR2 | |
| 263 | TCR4′a | CAMGALNSGAGSYQLTF |
| | CDR3 | |
| 264 | TCR4′b | MGSRLLCWVLLCLLGAGPVKAGVTQTPRYLIKTRGQQ |
| | | VTLSCSPISGHRSVSWYQQTPGQGLQFLFEYFSETQRN |
| | | KGNFPGRFSGRQFSNSRSEMNVSTLELGDSALYLCASS |
| | | LSSGTGTEAFFGQGTRLTVVEDLNKVFPPEVAVFEPSE |
| | | AEISHTQKATLVCLATGFFPDHVELSWWVNGKEVHSG |
| | | VSTDPQPLKEQPALNDSRYCLSSRLRVSATFWQNPRNH |
| | | FRCQVQFYGLSENDEWTQDRAKPVTQIVSAEAWGRA |
| | | DCGFTSVSYQQGVLSATILYEILLGKATLYAVLVSALV |
| | | LMAMVKRKDF |
| 265 | TCR4′b var | MGSRLLCWVLLCLLGAGPVKAGVTQTPRYLIKTRGQQ |
| | | VTLSCSPISGHRSVSWYQQTPGQGLQFLFEYFSETQRN |
| | | KGNFPGRFSGRQFSNSRSEMNVSTLELGDSALYLCASS |
| | | LSSGTGTEAFFGQGTRLTVV |
| 266 | TCR4′b | SGHRS |
| | CDR1 | |
| 267 | TCR4′b | YFSETQ |
| | CDR2 | |
| 268 | TCR4′b | CASSLSSGTGTEAFF |
| | CDR3 | |
| 269 | TCR5′a | MKTFAGFSFLFLWLQLDCMSRGEDVEQSLFLSVREGD |
| | | SSVINCTYTDSSSTYLYWYKQEPGAGLQLLTYIFSNMD |
| | | MKQDQRLTVLLNKKDKHLSLRIADTQTGDSAIYFCAE |
| | | RDAGNNRKLIWGLGTSLAVNPNIQNPDPAVYQLRDSK |
| | | SSDKSVCLFTDFDSQTNVSQSKDSDVYITDKTVLDMRS |
| | | MDFKSNSAVAWSNKSDFACANAFNNSIIPEDTFFPSPES |
| | | SCDVKLVEKSFETDTNLNFQNLSVIGFRILLLKVAGFNL |
| | | LMTLRLWSS |
| 270 | TCR5′a var | MKTFAGFSFLFLWLQLDCMSRGEDVEQSLFLSVREGD |
| | | SSVINCTYTDSSSTYLYWYKQEPGAGLQLLTYIFSNMD |
| | | MKQDQRLTVLLNKKDKHLSLRIADTQTGDSAIYFCAE |
| | | RDAGNNRKLIWGLGTSLAVNP |
| 271 | TCR5′a | DSSSTY |
| | CDR1 | |
| 272 | TCR5′a | IFSNMDM |
| | CDR2 | |
| 273 | TCR5′a | CAERDAGNNRKLIW |
| | CDR3 | |
| 274 | TCR5′b | MGTRLLCWVVLGFLGTDHTGAGVSQSPRYKVAKRGQ |
| | | DVALRCDPISGHVSLFWYQQALGQGPEFLTYFQNEAQ |
| | | LDKSGLPSDRFFAERPEGSVSTLKIQRTQQEDSAVYLC |
| | | ASSLEGGDTQYFGPGTRLTVLEDLKNVFPPEVAVFEPS |
| | | EAEISHTQKATLVCLATGFYPDHVELSWWVNGKEVHS |
| | | GVSTDPQPLKEQPALNDSRYCLSSRLRVSATFWQNPRN |
| | | HFRCQVQFYGLSENDEWTQDRAKPVTQIVSAEAWGR |
| | | ADCGFTSESYQQGVLSATILYEILLGKATLYAVLVSAL |
| | | VLMAMVKRKDSRG |
| 275 | TCR5′b var | MGTRLLCWVVLGFLGTDHTGAGVSQSPRYKVAKRGQ |
| | | DVALRCDPISGHVSLFWYQQALGQGPEFLTYFQNEAQ |
| | | LDKSGLPSDRFFAERPEGSVSTLKIQRTQQEDSAVYLC |
| | | ASSLEGGDTQYFGPGTRLTVL |
| 276 | TCR5′b | SGHVS |
| | CDR1 | |
| 277 | TCR5′b | FQNEAQ |
| | CDR2 | |
| 278 | TCR5′b | CASSLEGGDTQYF |
| | CDR3 | |
| 279 | elongation | GGCTCCGGTGCCCGTCAGTGGGCAGAGCGCACATCG |
| | factor-1 | CCCACAGTCCCCGAGAAGTTGGGGGGAGGGGTCGG |
| | alpha (EF- | CAATTGAACCGGTGCCTAGAGAAGGTGGCGCGGGGT |
| | 1α) | AAACTGGGAAAGTGATGTCGTGTACTGGCTCCGCCT |
| | promoter | TTTTCCCGAGGGTGGGGGAGAACCGTATATAAGTGC |
| | | AGTAGTCGCCGTGAACGTTCTTTTTCGCAACGGGTTT |
| | | GCCGCCAGAACACAGGTAAGTGCCGTGTGTGGTTCC |
| | | CGCGGGCCTGGCCTCTTTACGGGTTATGGCCCTTGCG |
| | | TGCCTTGAATTACTTCCACTGGCTGCAGTACGTGATT |
| | | CTTGATCCCGAGCTTCGGGTTGGAAGTGGGTGGGAG |
| | | AGTTCGAGGCCTTGCGCTTAAGGAGCCCCTTCGCCT |
| | | CGTGCTTGAGTTGAGGCCTGGCCTGGGCGCTGGGGC |
| | | CGCCGCGTGCGAATCTGGTGGCACCTTCGCGCCTGT |
| | | CTCGCTGCTTTCGATAAGTCTCTAGCCATTTAAAATT |
| | | TTTGATGACCTGCTGCGACGCTTTTTTTCTGGCAAGA |
| | | TAGTCTTGTAAATGCGGGCCAAGATCTGCACACTGG |
| | | TATTTCGGTTTTTGGGGCCGCGGGCGGCGACGGGGC |
| | | CCGTGCGTCCCAGCGCACATGTTCGGCGAGGCGGGG |
| | | CCTGCGAGCGCGGCCACCGAGAATCGGACGGGGGT |
| | | AGTCTCAAGCTGGCCGGCCTGCTCTGGTGCCTGGCC |
| | | TCGCGCCGCCGTGTATCGCCCCGCCCTGGGCGGCAA |
| | | GGCTGGCCCGGTCGGCACCAGTTGCGTGAGCGGAAA |
| | | GATGGCCGCTTCCCGGCCCTGCTGCAGGGAGCTCAA |
| | | AATGGAGGACGCGGCGCTCGGGAGAGCGGGCGGGT |
| | | GAGTCACCCACACAAAGGAAAAGGGCCTTTCCGTCC |
| | | TCAGCCGTCGCTTCATGTGACTCCACGGAGTACCGG |
| | | GCGCCGTCCAGGCACCTCGATTAGTTCTCGAGCTTTT |
| | | GGAGTACGTCGTCTTTAGGTTGGGGGGAGGGGTTTT |
| | | ATGCGATGGAGTTTCCCCACACTGAGTGGGTGGAGA |
| | | CTGAAGTTAGGCCAGCTTGGCACTTGATGTAATTCTC |
| | | CTTGGAATTTGCCCTTTTTGAGTTTGGATCTTGGTTC |
| | | ATTCTCAAGCCTCAGACAGTGGTTCAAAGTTTTTTTC |
| | | TTCCATTTCAGGTGTCGTGA |
| 280 | SV80 | GGTGTGGAAAGTCCCCAGGCTCCCCAGCAGGCAGAA |
| | enhancer | GTATGCAAAGCATGCATCTCAATTAGTCAGCAACCA |
| 281 | P2A/2A | GSGATNFSLLKQAGDVEENPGP |
| | peptide | |
| | sequence | |
| 282 | P2A | GGAAGCGGCGCCACAAACTTCTCACTGCTGAAACAG |
| | nucleotide | GCCGGCGACGTGGAAGAGAATCCCGGACCT |
| | sequence 1 | |
| 283 | P2A | GGTTCCGGAGCCACGAACTTCTCTCTGTTAAAGCAA |
| | nucleotide | GCAGGAGACGTGGAAGAAAACCCCGGTCCC |
| | sequence 2 | |
| 284 | T2A/2A | RAKRGSGEGRGSLLTCGDVEENPGP |
| | peptide | |
| | sequence | |
| 285 | T2A | AGAGCCAAAAGAGGATCTGGCGAAGGCAGAGGCTC |
| | nucleotide | TCTGCTGACATGTGGCGACGTCGAAGAAAACCCTGG |
| | sequence | GCCA |
| 286 | Self- | GSGEGRGSLLTCGDVEENPGP |
| | cleaving | |
| | peptide 3 | |
| | peptide | |
| | sequence | |
| 287 | Self- | GGCAGCGGAGAAGGCAGAGGCTCCCTGCTTACATGC |
| | cleaving | GGCGACGTGGAAGAGAACCCCGGACCT |
| | peptide 3 | |
| | nucleotide | |
| | sequence | |
| 288 | TCR2a | ATGACCAGAGTGTCCCTGCTGTGGGCCGTCGTGGTG |
| | | TCTACATGTCTGGAATCTGGCATGGCCCAGACAGTG |
| | | ACCCAGAGCCAGCCTGAGATGTCTGTGCAAGAGGCC |
| | | GAAACCGTGACACTGAGCTGCACCTACGACACCAGC |
| | | GAGAACAACTACTACCTGTTCTGGTACAAGCAGCCT |
| | | CCTAGCCGGCAGATGATCCTGGTCATCAGACAAGAG |
| 289 | TCR2b | GCCTATAAGCAGCAGAACGCCACCGAGAACAGGTTC |
| | | AGCGTGAACTTCCAGAAGGCCGCCAAGAGCTTCAGC |
| | | CTGAAGATCAGCGATAGCCAGCTGGGCGACACCGCC |
| | | ATGTACTTTTGCGCCTTCATGTACCCCAGCCAAGGCG |
| | | GCTCTGAGAAACTGGTGTTTGGCAAGGGCATGAAGC |
| | | TGACCGTGAATCCCAACATTCAGAACCCCGATCCTG |
| | | CCGTGTAtCAGCTGcGgGAtAGCAAGAGCAGCGACAA |
| | | GAGCGTGTGCCTGTTCACCGACTTCGACAGCCAGAC |
| | | CAACGTGTCCCAGAGCAAGGACAGCGACGTGTACAT |
| | | CACCGATAAGTGCGTGCTGGACATGCGGAGCATGGA |
| | | CTTCAAGAGCAACAGCGCCGTGGCCTGGTCCAACAA |
| | | GAGCGATTTCGCCTGCGCCAACGCCTTCAACAACAG |
| | | CATTATCCCCGAGGACACATTCTTCCCAAGTCCTGA |
| | | GTCCAGCTGCGACGTGAAGCTGGTGGAAAAGAGCTT |
| | | CGAGACAGACACCAACCTGAATTTCCAGAACCTGAG |
| | | CGTGATCGGCTTCCGCATCCTGCTGCTGAAAGTGGC |
| | | CGGATTCAACCTGCTGATGACCCTGCGACTGTGGTC |
| | | TAGC |
| | | ATGGGACCTGGATTGCTTTGTTGGGTGCTGCTGTGTC |
| | | TGCTCGGAGCCGGATCTGTGGAAACAGGCGTGACAC |
| | | AGAGCCCCACACACCTGATCAAGACCAGAGGCCAG |
| | | CAAGTGACCCTGAGATGCAGCTCTCAGAGCGGCCAC |
| | | AATACCGTGTCCTGGTATCAGCAGGCCCTCGGACAG |
| | | GGCCCTCAGTTCATCTTCCAGTACTACAGAGAGGAA |
| | | GAGAACGGCAGAGGCAACTTCCCACCTAGATTCAGC |
| | | GGCCTGCAGTTCCCCAACTACAGCAGCGAGCTGAAC |
| | | GTGAACGCCCTGGAACTGGATGACAGCGCCCTGTAC |
| | | CTGTGTGCCTCTAGCAGCCCTGGCTTCAGAAGCTAC |
| | | GGCTACACATTTGGCAGCGGCACCAGACTGACCGTG |
| | | GTGGAAGATCTGAAGAACGTGTTCCCACCTGAGGTG |
| | | GCCGTGTTCGAACCTTCTGAGGCCGAGATCAGCCAC |
| | | ACACAGAAAGCCACACTCGTGTGTCTGGCCACCGGC |
| | | TTCTATCCCGATCACGTGGAACTGTCTTGGTGGGTCA |
| | | ACGGCAAAGAGGTGCACAGCGGCGTCTGTACCGATC |
| | | CTCAGCCTCTGAAAGAGCAGCCCGCTCTGAACGACA |
| | | GCAGATACTGCCTGAGCAGCAGACTGAGAGTGTCCG |
| | | CCACCTTCTGGCAGAACCCCAGAAACCACTTCAGAT |
| | | GCCAGGTGCAGTTCTACGGCCTGAGCGAGAACGATG |
| | | AGTGGACCCAGGATAGAGCCAAGCCTGTGACTCAGA |
| | | TCGTGTCTGCCGAAGCCTGGGGCAGAGCCGATTGTG |
| | | GCTTTACCAGCGAGTCTTACCAGCAGGGCGTGCTGT |
| | | CTGCCACCATCCTGTATGAGATCCTGCTGGGCAAAG |
| | | CCACTCTGTACGCCGTGCTGGTTTCTGCCCTGGTGCT |
| | | GATGGCCATGGTCAAGCGGAAGGATAGCAGAGGC |
| 290 | CD34-IL7R | ATGCTCGTCAGAAGAGGCGCTAGAGCCGGACCTAGA |
| | | ATGCCCAGAGGATGGACAGCCCTCTGCCTCCTGTCT |
| | | CTGCTGCCTAGCGGCTTCATGAGCCTGGACAACAAC |
| | | GGCACAGCCACACCTGAGCTGCCTACACAGGGCACC |
| | | TTCAGCAATGTGTCCACAAACGTGTCCTACCAAGAG |
| | | ACAACCACACCTAGCACACTGGGCAGCACATCTCTG |
| | | CACCCTGTGTCTCAGCACGGCAATGAGGCCACCACC |
| | | AATATCACCGAGACAACCGTGAAGTTCACCAGCACC |
| | | AGCGTGATCACCTCCGTGTACGGCAACACCAACAGC |
| | | AGCGTGCAGAGCCAGACCTCCGTGATCAGCACCGTG |
| | | TTTACAACCCCTGCCAATGTGTCTACCCCTGAGACA |
| | | ACTCTGAAGCCCAGCCTGTCTCCTGGGAACGTGTCC |
| | | GATCTGAGCACCACCTCTACCAGCCTGGCCACCTCT |
| | | CCTACAAAGCCCTACACAAGCAGCAGCCCCATCCTG |
| | | AGCGATATCAAGGCCGAAATCAAGTGCAGCGGCATC |
| | | CGGGAAGTGAAACTGACCCAGGGCATCTGCCTGGAA |
| | | CAGAACAAGACCAGCAGCTGCGCCGAGTTCAAGAA |
| | | GGACAGAGGCGAAGGACTGGCCAGAGTGCTGTGTG |
| | | GCGAAGAACAGGCCGATGCTGATGCTGGCGCTCAAG |
| | | TCTGTTCACTGCTGCTGGCCCAGTCTGAAGTGCGGCC |
| | | TCAATGTCTGCTTCTGGTCCTGGCCAACCGGACCGA |
| | | GATCTCTAGCAAACTGCAGCTGATGAAGAAGCACCA |
| | | GAGCGACCTGAAGAAGCTGGGCATCCTGGACTTCAC |
| | | CGAGCAGGATGTGGCCAGCCACCAGAGCTACAGCC |
| | | AGAAAACACCTATCCTGCTGACCTGTCCGACAATCA |
| | | GCATCCTGTCCTTTTTCAGCGTGGCCCTGCTCGTGAT |
| | | CCTGGCCTGTGTGCTGTGGAAGAAGCGGATCAAGCC |
| | | CATCGTGTGGCCTAGCCTGCCTGACCACAAAAAGAC |
| | | CCTGGAACACCTGTGCAAAAAGCCCCGGAAGAACCT |
| | | GAATGTGTCTTTCAACCCCGAGAGCTTCCTGGACTG |
| | | CCAGATCCACAGAGTGGACGACATCCAGGCCAGAG |
| | | ATGAGGTGGAAGGCTTTCTGCAGGACACCTTTCCGC |
| | | AGCAGCTGGAAGAGTCCGAGAAGCAGAGACTCGGC |
| | | GGAGATGTGCAGTCCCCTAATTGCCCTAGCGAGGAC |
| | | GTGGTCATCACCCCTGAGTCCTTCGGAAGAGACTCC |
| | | AGCCTGACTTGTCTGGCCGGAAATGTGTCTGCCTGC |
| | | GACGCCCCTATCCTGTCCAGCTCTAGAAGCCTGGAC |
| | | TGTAGAGAGAGCGGCAAGAACGGCCCTCATGTGTAC |
| | | CAGGATCTGCTGCTGTCCCTGGGCACCACCAACTCT |
| | | ACACTGCCTCCACCATTCAGCCTGCAGTCCGGCATC |
| | | CTGACACTGAATCCTGTGGCTCAGGGCCAGCCAATC |
| | | CTGACAAGCCTGGGCTCCAATCAAGAAGAGGCTTAC |
| | | GTCACCATGAGCAGCTTCTACCAGAATCAA |
| 291 | CD8a | ATGGCTCTGCCTGTGACAGCTCTGCTGCTGCCTCTGG |
| | | CTCTGCTTCTGCATGCCGCTAGACCCAGCCAGTTCAG |
| | | AGTGTCCCCTCTGGACAGAACCTGGAACCTGGGCGA |
| | | GACAGTGGAACTGAAGTGCCAGGTGCTGCTGAGCAA |
| | | TCCTACCAGCGGCTGCAGCTGGCTGTTTCAGCCTAG |
| | | AGGTGCTGCCGCCTCTCCTACCTTTCTGCTGTACCTG |
| | | AGCCAGAACAAGCCCAAGGCCGCCGAAGGACTGGA |
| | | CACCCAGAGATTCAGCGGCAAGAGACTGGGCGACA |
| | | CCTTCGTGCTGACCCTGAGCGACTTCAGAAGAGAGA |
| | | ACGAGGGCTACTACTTCTGCAGCGCCCTGAGCAACA |
| | | GCATCATGTACTTCAGCCACTTCGTGCCCGTGTTTCT |
| | | GCCCGCCAAGCCTACAACAACCCCTGCTCCTAGACC |
| | | TCCTACACCAGCTCCTACAATCGCCAGCCAGCCTCT |
| | | GTCTCTGAGGCCAGAAGCTTGTAGACCTGCTGCTGG |
| | | CGGAGCCGTGCATACAAGAGGACTGGATTTCGCCTG |
| | | CGACATCTACATCTGGGCCCCTCTGGCTGGAACATG |
| | | TGGCGTGCTGCTGCTGTCCCTGGTCATCACCCTGTAC |
| | | TGCAACCACCGGAACAGGCGGAGAGTGTGCAAGTG |
| | | CCCTAGACCTGTGGTCAAGAGCGGCGACAAGCCTAG |
| | | CCTGAGCGCCAGATATGTT |
| 292 | CD8b | ATGAGGCCTAGACTGTGGCTGCTTCTGGCTGCCCAG |
| | | CTGACAGTGCTGCACGGCAATTCTGTCCTGCAGCAG |
| | | ACCCCTGCCTACATCAAGGTGCAGACCAACAAGATG |
| | | GTCATGCTGAGCTGCGAGGCCAAGATCAGCCTGTCC |
| | | AACATGCGGATCTACTGGCTGCGGCAGAGACAGGCC |
| | | CCTAGCTCTGATAGCCACCACGAGTTTCTGGCCCTGT |
| | | GGGATTCTGCCAAGGGCACCATTCACGGCGAGGAAG |
| | | TGGAACAAGAGAAGATCGCCGTGTTCCGGGACGCCA |
| | | GCAGATTCATCCTGAACCTGACCAGCGTGAAGCCCG |
| | | AGGACAGCGGCATCTATTTCTGCATGATCGTGGGCA |
| | | GCCCCGAGCTGACATTTGGCAAGGGAACACAGCTGA |
| | | GCGTGGTGGACTTCCTGCCTACTACAGCCCAGCCTA |
| | | CCAAGAAGTCTACCCTGAAGAAACGCGTGTGCAGAC |
| | | TGCCCAGGCCTGAGACACAAAAGGGCCCTCTGTGCA |
| | | GCCCTATCACACTGGGATTGCTGGTGGCTGGCGTTCT |
| | | GGTCCTGCTGGTGTCTCTGGGAGTTGCCATCCACCTG |
| | | TGCTGTAGAAGAAGGCGGGCCAGACTGCGGTTCATG |
| | | AAGCAGTTCTACAAA |
| 293 | TCR4a | ATGATGAAGTCCCTGAGAGTGCTGCTGGTCATCCTG |
| | | TGGCTGCAGCTGTCTTGGGTCTGGTCCCAGCAGAAA |
| | | GAGGTGGAACAGGACCCTGGACCTCTGTCTGTTCCT |
| | | GAGGGCGCCATCGTGTCCCTGAATTGCACCTACAGC |
| | | AACTCCGCCTTCCAGTACTTCATGTGGTACAGACAG |
| | | TACAGCCGGAAGGGCCCCGAGCTGCTGATGTACACA |
| | | TACAGCAGCGGCAACAAAGAGGACGGCCGGTTTAC |
| | | AGCCCAGGTGGACAAGAGCAGCAAGTACATCTCCCT |
| | | GTTCATCCGGGACAGCCAGCCTAGCGATAGCGCCAC |
| | | ATACCTGTGTGCTATGGGCGCCCTGAATTCTGGCGC |
| | | CGGAAGCTATCAGCTGACCTTCGGCAAGGGCACCAA |
| | | GCTGAGCGTGATCCCCAACATTCAGAACCCCGATCC |
| | | TGCCGTGTAtCAGCTGcGgGAtAGCAAGTCCAGCGAC |
| | | AAGAGCGTGTGCCTGTTCACCGACTTCGACAGCCAG |
| | | ACCAACGTGTCCCAGAGCAAGGACAGCGACGTGTAC |
| | | ATTACCGATAAGTGCGTGCTGGACATGCGGAGCATG |
| | | GACTTCAAGAGCAACAGCGCCGTGGCCTGGTCCAAC |
| | | AAGAGCGATTTCGCCTGCGCCAACGCCTTCAACAAC |
| | | AGCATTATCCCCGAGGACACATTCTTCCCAAGTCCT |
| | | GAGTCCAGCTGCGACGTGAAGCTGGTGGAAAAGAG |
| | | CTTCGAGACAGACACCAACCTGAACTTCCAGAACCT |
| | | GTCCGTGATCGGCTTCAGAATCCTGCTGCTGAAGGT |
| | | GGCCGGATTCAACCTGCTGATGACCCTCAGACTGTG |
| | | GTCTAGC |
| 294 | TCR4b | ATGGGATCTAGATTGCTTTGTTGGGTGCTGCTGTGCC |
| | | TGCTCGGAGCCGGACCTGTGAAAGCTGGCGTTACCC |
| | | AGACACCTAGATACCTGATCAAGACCAGAGGCCAGC |
| | | AAGTGACCCTGAGCTGCTCTCCTATCAGCGGCCACA |
| | | GAAGCGTGTCCTGGTATCAGCAGACACCTGGACAGG |
| | | GCCTGCAGTTCCTGTTCGAGTACTTCAGCGAGACAC |
| | | AGCGGAACAAGGGCAACTTCCCCGGCAGATTTTCCG |
| | | GCAGACAGTTCAGCAACAGCCGCAGCGAGATGAAC |
| | | GTGTCCACACTGGAACTGGGCGACAGCGCCCTGTAT |
| | | CTGTGTGCCTCTTCTCTGTCCTCCGGAACCGGCACCG |
| | | AGGCCTTTTTCGGACAAGGCACCAGACTGACCGTGG |
| | | AAGATCTGAAGAACGTGTTCCCACCTGAGGTGGCCG |
| | | TGTTCGAGCCTTCTGAGGCCGAGATCAGCCACACAC |
| | | AGAAAGCCACACTCGTGTGTCTGGCCACCGGCTTCT |
| | | ATCCCGATCACGTGGAACTGTCTTGGTGGGTCAACG |
| | | GCAAAGAGGTGCACAGCGGCGTCTGTACCGATCCTC |
| | | AGCCTCTGAAAGAGCAGCCCGCTCTGAACGACAGCA |
| | | GATACTGCCTGAGCAGCAGACTGAGAGTGTCCGCCA |
| | | CCTTCTGGCAGAACCCCAGAAACCACTTCAGATGCC |
| | | AGGTGCAGTTCTACGGCCTGAGCGAGAACGATGAGT |
| | | GGACCCAGGATAGAGCCAAGCCTGTGACACAGATC |
| | | GTGTCTGCCGAAGCCTGGGGCAGAGCCGATTGTGGC |
| | | TTTACCAGCGAGAGCTACCAGCAGGGCGTGCTGTCT |
| | | GCCACAATCCTGTACGAGATCCTGCTGGGCAAAGCC |
| | | ACTCTGTACGCCGTGCTGGTTTCTGCCCTGGTGCTGA |
| | | TGGCCATGGTCAAGCGGAAGGATAGCAGAGGC |

### EXAMPLES

### Example 1: CRISPR-Mediated Disruption of Trac/Trbc in Immune Cells with or Without Concurrent Modification with KRAS Peptide-Specific Binding Proteins (General Procedure)

CRISPR-mediated disruption of TRAC and TRBC was carried out using the following protocol. On day 0, 100×10\hat{ }6 CD4+ or CD8+ T cells were thawed and transactivated (with 1:100 Transact). Cells were cultured in a 6-well G-rex plate (˜25×10\hat{ }6 cells/well). Approximately 45-55% of cells die by day 2; the number of cells was therefore calculated according to the conditions needed for knockout (KO) on day 2. T cells were cultured in complete T cell media comprising XVivo™ 15 Serum-free Hematopoietic Cell Medium (Lonza, Basel, Switzerland), 2% Immune Cell Serum Replacement (ICSR), 100 IU/mL IL-2, 5 ng/mL IL-7, and +5 ng/mL IL-15.

On day 1, T cells were transduced with virus encoding an extracellular binding protein as described herein (e.g., a KRAS G12mutant-specific TCR or any of the TCR sequences recited in TABLE 1) or were left as-is to transactivate until day 2. If transducing with lentivirus (LV), cells were counted and transduced at 5-10×10\hat{ }6 cells per condition.

On day 2, cells were electroporated with ribonucleoprotein complexes (RNPs) to knock out TRAC and/or TRBC1/2 genes (e.g., using the guide RNAs in SEQ ID NO: 113 or 115). For each electroporation, 250 pmol guide RNA and enough nuclease for a 2.5:1 guide RNA to nuclease ratio (MG-29) was assembled in 7.5 μL total volume at room temperature for 30 minutes to form RNP complexes. While RNP complexes were forming, cells were counted and 5-10×10\hat{ }6 cells were used per electroporation for the 100 μL electroporate volume using Neon (ThermoFisher). If cells were transduced on day 1, cells were generally not counted again on day 2.

To set up the Neon electroporator, CTS Xenon electroporation buffer (Thermo Cat #A4997901) and E2 buffers were first thawed to RT in BSE. E2 Electrolytic buffer is a high osmolarity buffer that is commercially available from ThermoFisher Scientific. The required volume of electroporation buffer was then transferred in a microfuge or 15 mL tube. 7 mL of complete T cell media (X-VIVO15 (Lonza, (Lexington, MA) 04-418Q)+2% PLTGold (Mill Creek Life Sciences (Rochester, MN) PLTGOLD100 GMP) was then added to each well of a 24 well plate used for non-adherent cell propagation, which is commercially available as Grex plate, and transferred to 37° C. (for culturing cells after they have been electroporated).

Subsequently, 5-10×10\hat{ }6 cells per electroporation condition were spun down in 15 mL tubes. The media was carefully aspirated and the cell pellet resuspended with 100 μL electroporation buffer (if performing single KO (sKO)) or 95 μL GE electroporation buffer (if performing double KO (dKO)). The total volume desired for electroporation is 120 μL. If performing dKO, the TRAC RNP was combined with TRBC RNP (7.5 μL each for required number of conditions). 7.5 μL of RNP (after the 30 min incubation) was added if doing sKO or 15 μL of RNP if doing dKO to the cells in GE buffer. The cells and RNP were mixed by gently flicking the tube.

Approximately 100 μL of the mix containing RNP and electroporation buffer was carefully pipetted using the Neon 100 μL pipettor and tip. Care was taken to have no bubbles in the pipet tip. If bubbles form despite 2-3 attempts, the tip was changed or 10-15 μL of electroporation buffer was added to the tube and the step was repeated. The cell media can be aspirated, and the cell pellet left as-is in the BSE for 15-20 mins until ready for electroporation.

3 mL of E2 buffer was then transferred to a Neon electroporation tube and placed in a tube holder.

The electroporation pipet with tip containing cells and RNP was transferred to the tube containing E2 buffer in the tube holder, and the electroporation protocol on the Neon was carried out using suitable settings on the electroporator. An example setting for the Neon electroporator is 2300V, 4 pulses, and 3 ms pulse width.

After electroporation, the cells were then transferred to a well of a 24 well plate (standard tissue culture (TC) plate can be adherent or non-adherent)—ElectroPoration (“EP plate”) and incubated in BSE at room temperature for about 10 minutes. About 1 mL of T cell media from G-rex plate is used to wash cells from the 24 well EP plate and transfer to wells in the G-rex plate. The plate was returned to 37° C. with 5% CO2.

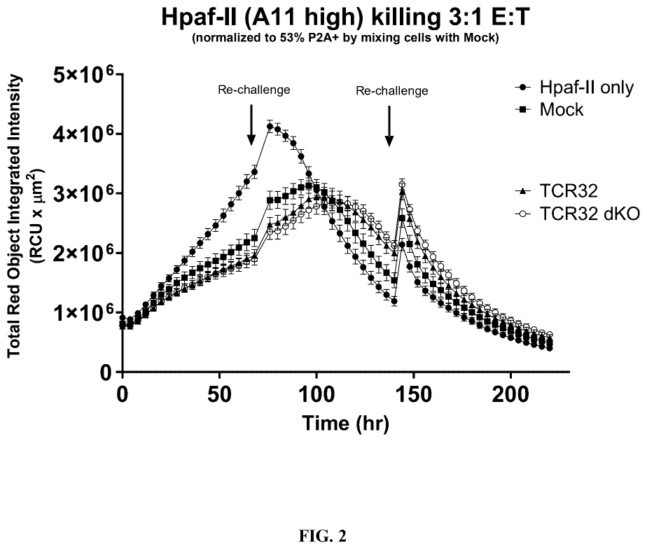

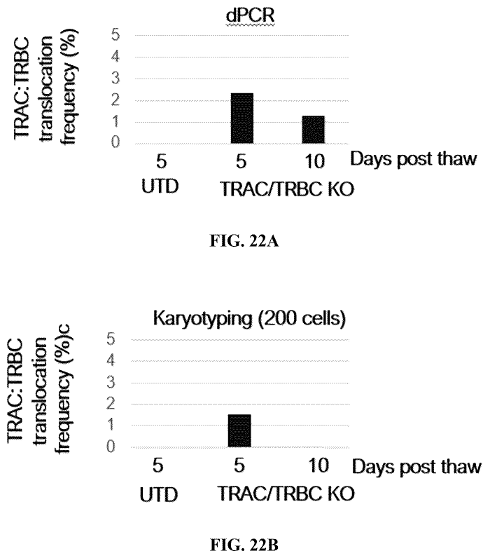

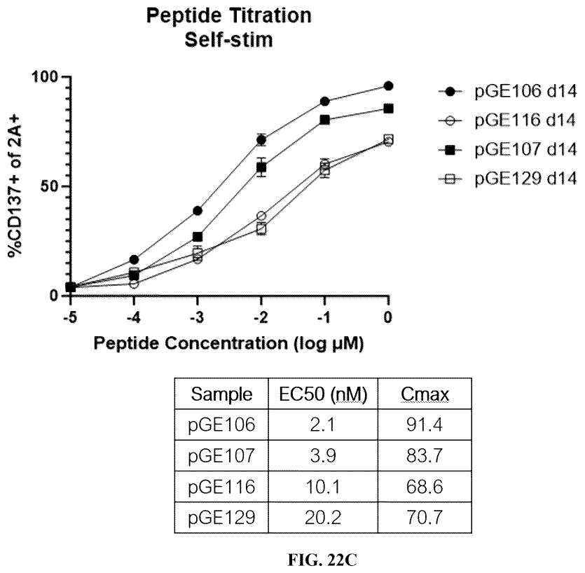

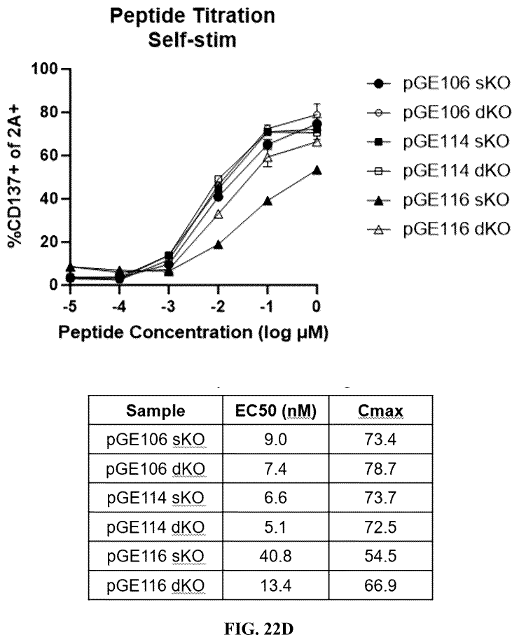

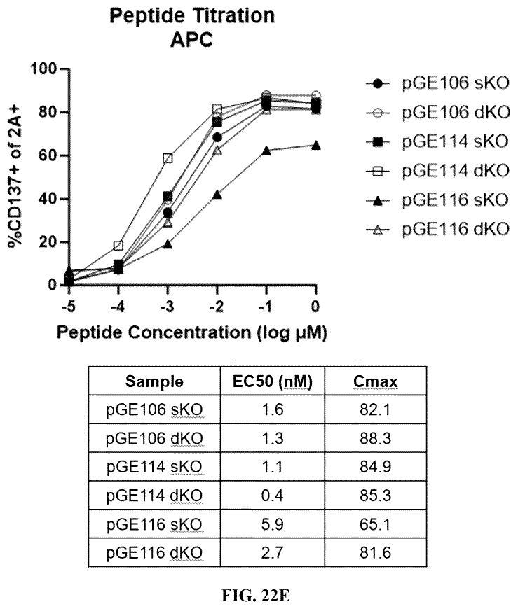

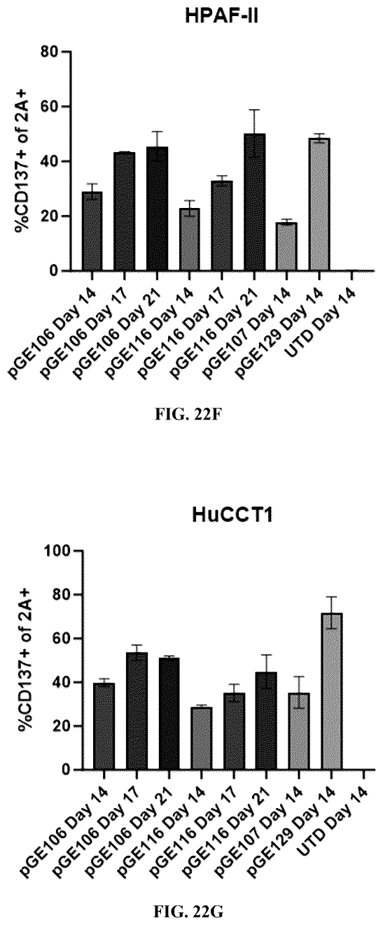

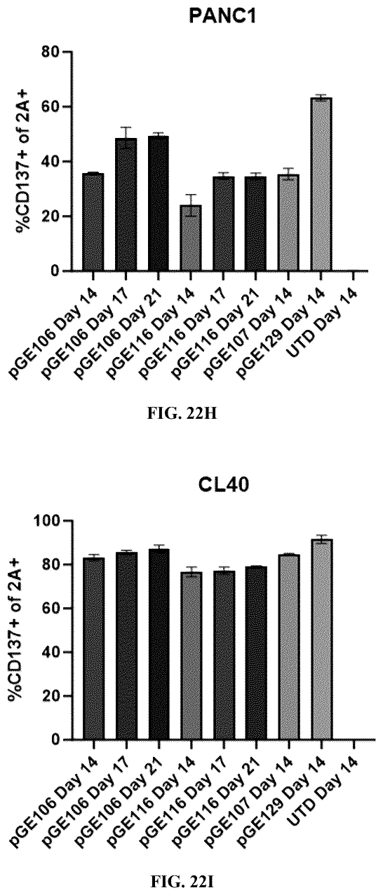

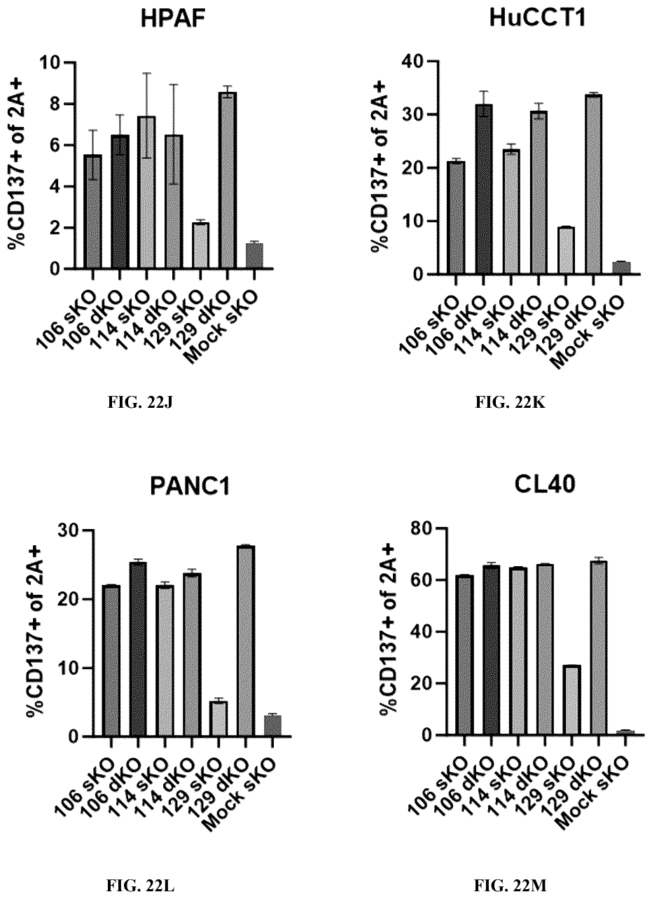

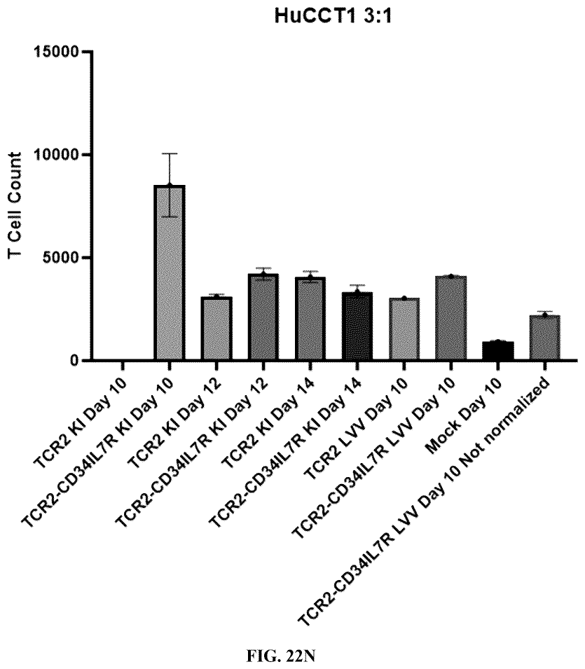

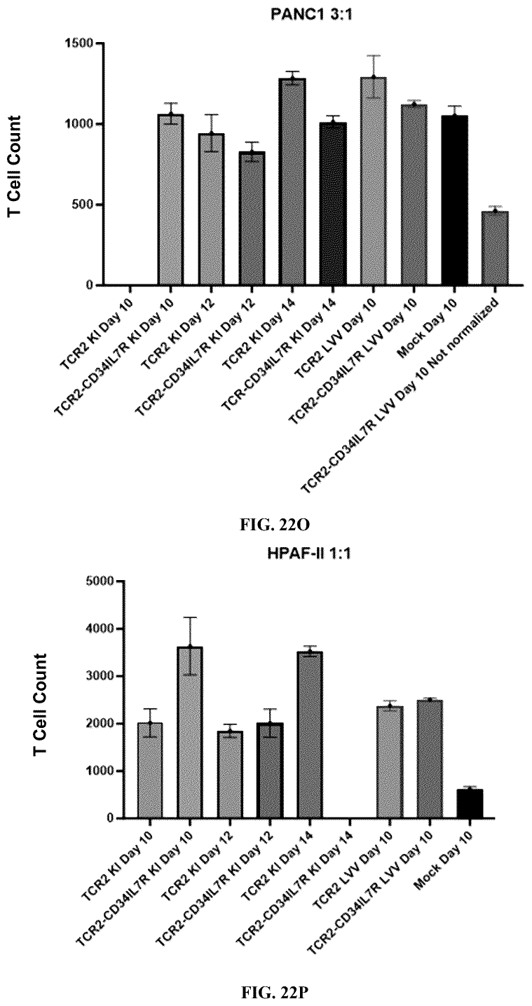

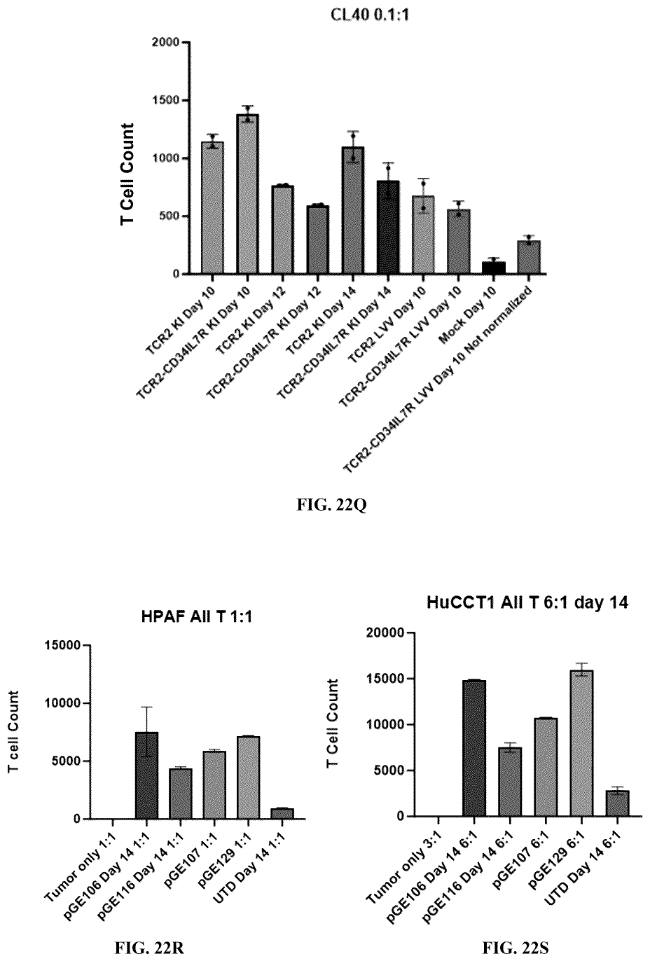

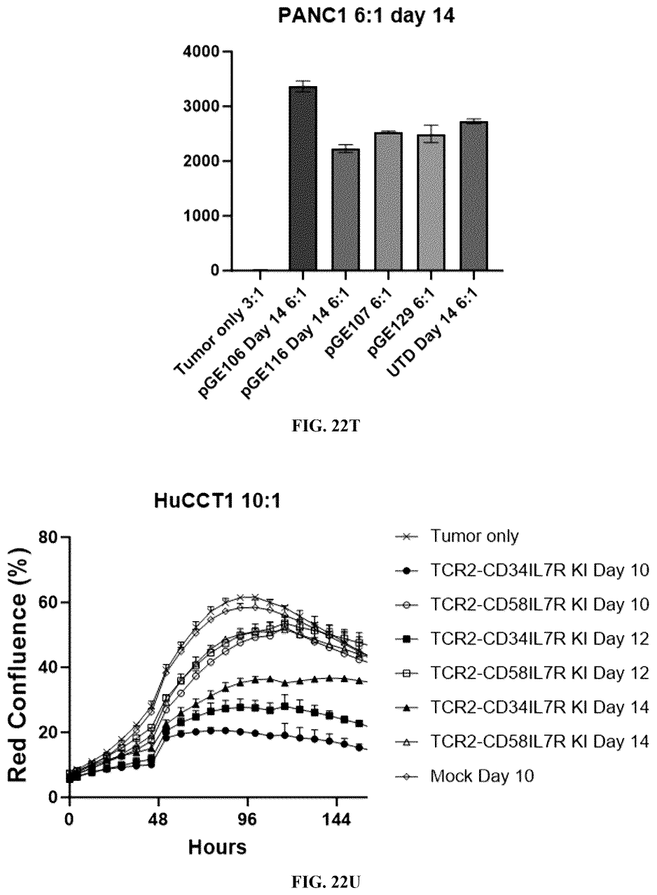

Next, peptide dose-dependent responses were studied for cells modified with the following constructs: pGE106 (encoding TCR2, CD34-IL7R, and CD8 alpha/beta), pGE116 (encoding TCR2, CD34-IL7R, and CD8 alpha/beta, which is designed to use the endogenous TRAC promoter), pGE107 (same as pGE106 but with shorter homology arms), and pGE129 (encoding TCR4, CD34-IL7R, and CD8 alpha beta). The responses were assessed by analyzing the percentage of 2A-positive cells expressing CD137 following stimulation with KRAS-G12D peptide. Dose-response curves were fitted by non-linear regression, and EC50 values were calculated using GraphPad Prism®. (FIG. 22C). The experiment was repeated using cells modified with pGE106, pGE114 (encoding TCR2, CD34IL7R, CD8 alpha/beta, and an SV40 enhancer), and pGE116 and further modified to have single knockout of the TRAC locus or a double knockout of the TRAC and TRBC loci. (FIG. 22D). Similar results were observed for cells cocultured with antigen presenting cells (APCs) instead of cognate peptide. (FIG. 22E), as well as when cocultured with certain cancer cell lines (FIGS. 2F-2M).

T cells modified via either electroporation or LVV-mediated transduction with constructs encoding either TCR2 and CD8 alpha/beta or TCR2, CD34-IL7R, and CD8 alpha/beta exhibited in vitro proliferation when co-cultured with different KRAS-G12D-positive cell lines (FIGS. 22N-22T).

Cytotoxic activity was assessed for cells modified to express TCR2, CD34IL7R, and CD8 alpha/beta or TCR2, CD58IL7R, and CD8 alpha/beta, and plots of red fluorescence measured over time by live cell imaging were generated. The results demonstrate that the cells are more effective at controlling HuCCT1 tumor cells relative to controls (FIG. 22U).

### Example 2: Editing Efficiency and Specificity of CRISPR-Mediated Disruption of Trac/Trbc in Immune Cells

CD4+ and CD8+ T cells were subjected to CRISPR-mediated disruption of TRAC and TRBC as described in EXAMPLE 1.

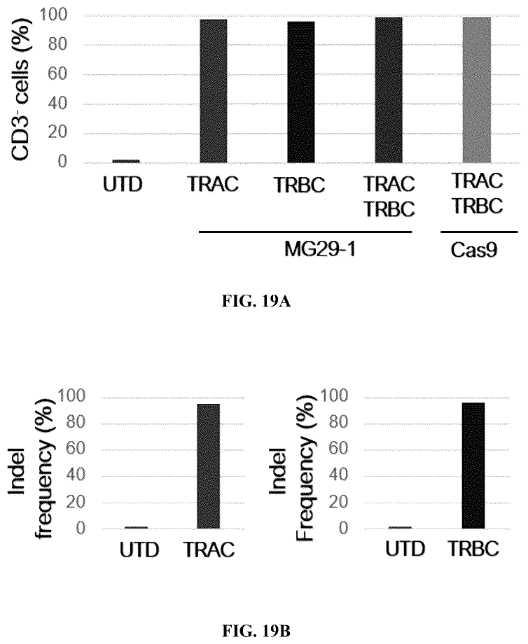

CD4/CD8 T cells electroporated with gRNAs targeting TRAC and TRBC genes were assessed for expression of CD3 on the cell surface and indels at on-target genomic loci. High editing efficiency was achieved using a CRISPR-associated nuclease type V, termed MG29-1 at both TRAC and TRBC loci. The editing activity was comparable to a CRISPR/Cas9 system. (FIGS. 19A and 19B).

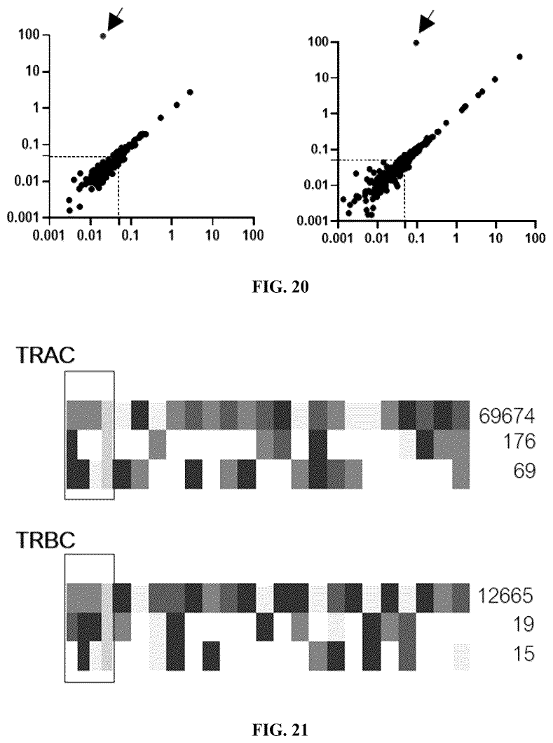

Five hundred ninety (590) computationally predicted potential off-target sites (OTs) were selected across TRAC and TRBC gRNAs that had up to 6 mismatches with the target sequence. Indel activity was evaluated at each of these potential Ots in primary T cells treated with the TRAC and TRBC gRNAs. High on-target editing efficiency was observed, but no off-target activity was detected above background beyond the quantification limit of the assay (0.05%). (FIG. 20).

Oligo-capture analysis was performed in primary T cells to further evaluate the specificity of these nuclease/gRNA combinations. A few potential off-target sites (OTs) were identified, each of which had at least 100-fold fewer barcodes than the target sites and had multiple mismatches (>9) to the target site suggesting the low likelihood of these Ots being true positives. (FIG. 21).

Frequencies of potential translocation outcomes between TRAC and TRBC loci were evaluated in edited primary T cells using digital PCR (dPCR) and karyotyping. Editing T cells with TRAC/TRBC gRNAs resulted in a low frequency of translocation events. No enrichment of translocation events was observed over culture time. (FIGS. 22A and 22B).

Next, peptide dose-dependent responses were studied in cells modified with the following constructs: pGE106 (encoding TCR2, CD34-IL7R, and CD8 alpha/beta), pGE116 (encoding TCR2, CD34-IL7R, and CD8 alpha/beta, which is designed to use the endogenous TRAC promoter), pGE107 (same as pGE106 but with shorter homology arms), and pGE129 (encoding TCR4, CD34-IL7R, and CD8 alpha beta). The responses were assessed by analyzing the percentage of 2A-positive cells expressing CD137 following stimulation with KRAS-G12D peptide. Dose-response curves were fitted by non-linear regression, and EC50 values were calculated using GraphPad Prism®. (FIG. 22C). The experiment was repeated using cells modified with pGE106, pGE114 (encoding TCR2, CD34IL7R, CD8 alpha/beta, and an SV40 enhancer), and pGE116 and further modified to have single knockout of the TRAC locus or a double knockout of the TRAC and TRBC loci. (FIG. 22D). Similar results were observed for cells cocultured with antigen presenting cells (APCs) instead of cognate peptide. (FIG. 22E), as well as when cocultured with certain cancer cell lines (FIGS. 22F-22M).

T cells modified via either electroporation or LVV-mediated transduction with constructs encoding either TCR2 and CD8 alpha/beta or TCR2, CD34-IL7R, and CD8 alpha/beta exhibited in vitro proliferation when co-cultured with different KRAS-G12D-positive cell lines (FIGS. 22N-22T).

Cytotoxic activity was assessed for cells modified to express TCR2, CD34IL7R, and CD8 alpha/beta or TCR2, CD58IL7R, and CD8 alpha/beta, and plots of red fluorescence measured over time by live cell imaging were generated. The results demonstrate that the cells are more effective at controlling HuCCT1 tumor cells relative to controls (FIG. 22U).

### Example 3: Activation of T Cells Expressing Candidate KRAS-G12D-Binding TCRs

This example demonstrates activation of T cells expressing TCRs disclosed herein by target cells pulsed with a KRAS-G12D peptide.

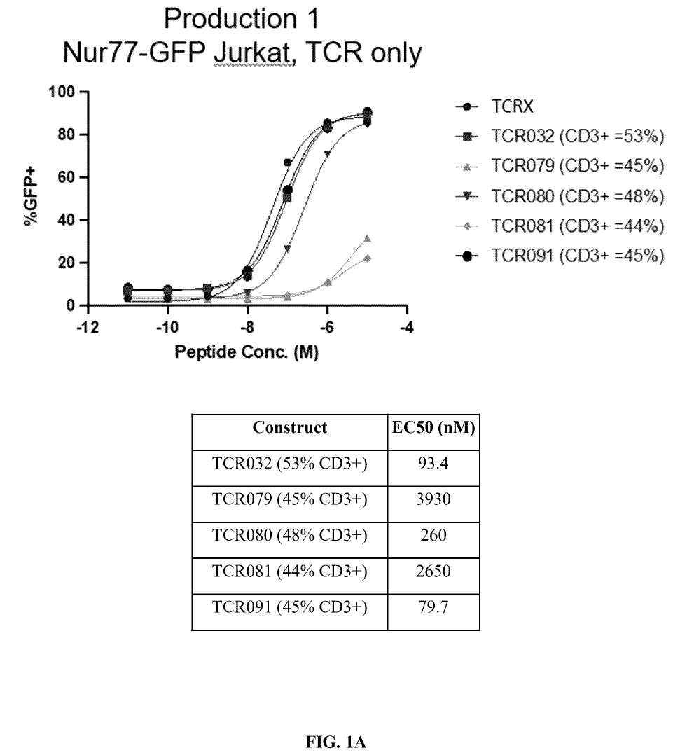

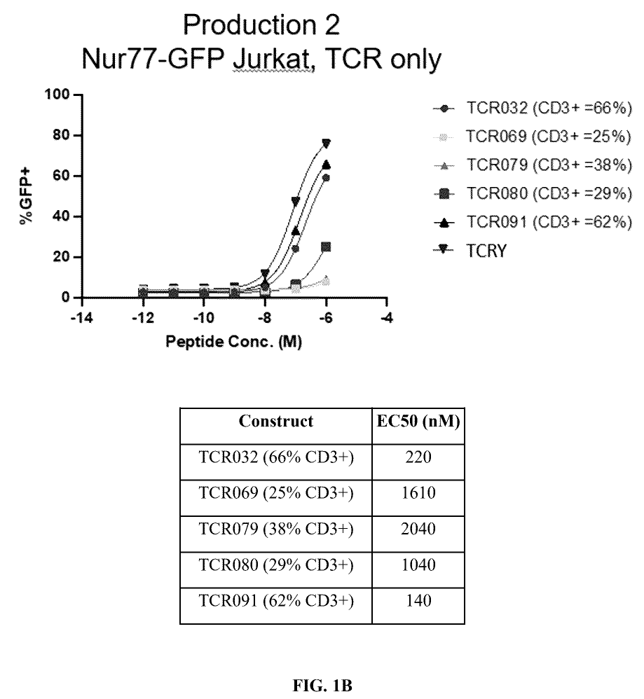

Paired TCR alpha/beta sequences from identified clonotypes were assembled and synthesized as P2A-linked expression cassettes as described, for example, by Hilgarth and Lanigan, MethodsX 7 (2020) 100759, and lentivirally transduced into reporter Jurkat cells that express GFP under the control of the Nur77 locus to indicate TCR activation (Nur77-GFP-Jurkats). Peptide dose-dependent responses for each TCR were assessed by analyzing GFP expression following overnight culture with A11 target cells pulsed with decreasing concentrations of peptide. Dose-response curves were fitted by non-linear regression, and EC50 values were calculated using GraphPad Prism®. TCR091 showed the highest affinity in two repeats of the assay (FIG. 1A and FIG. 1B).

### Example 4: Target Cell Killing by T Cells Expressing Candidate KRAS-G12D Specific TCRs

This example demonstrates killing of KRAS-G12D-presenting target cells by T cells expressing a TCR disclosed herein, with or without knockout of TRAC and TRBC.

Red fluorescent Hpaf-II cells, a KRAS-G12D expressing tumor cell line transduced to express HLA-A11, were cocultured at 3:1 effector:target cell ratio with TCR32-transduced CD8⁺ T cells having wild type TRAC and TRBC loci or TCR32-transduced TRAC/TRBC double knockout (dKO) CD8⁺ T cells. Total red object integrated intensity (a measure of tumor cell volume) was evaluated over time. Red fluorescence was measured by live cell imaging using a life cell analysis system, the IncuCyte S3 microscope and software package. CD8⁺ T cell cytotoxicity is indicated by a decrease in the total red target cell area per well as compared to no treatment wells. Additional tumor cells were added at 72 and 140 hours to assess TCR-mediated tumor cell lysis by transduced T cells in the presence of persistent antigen. Reductions in tumor cells were observed when co-cultured with TCR-transduced cells (FIG. 2).

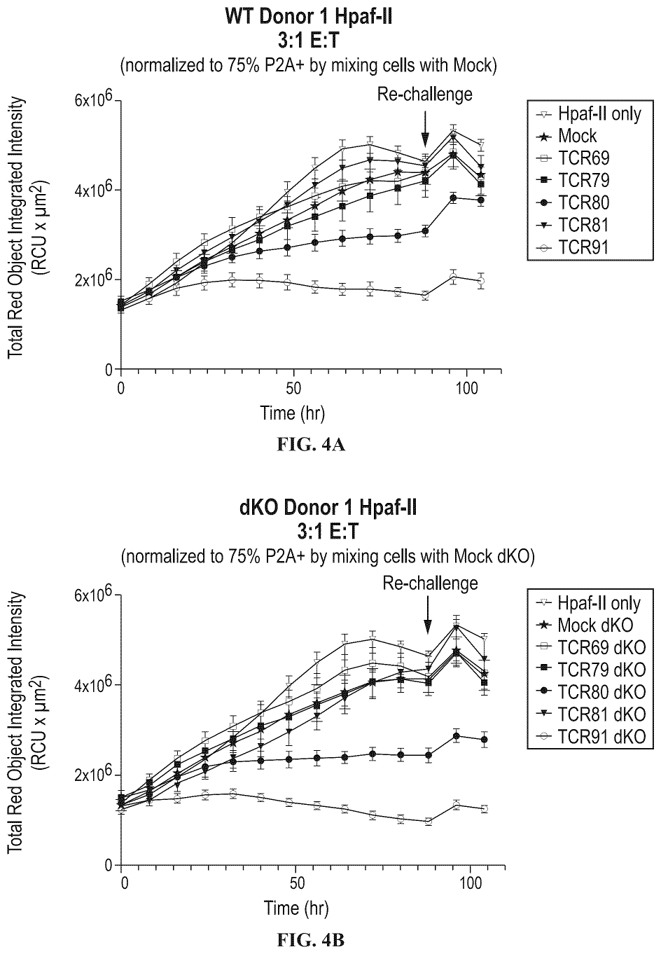

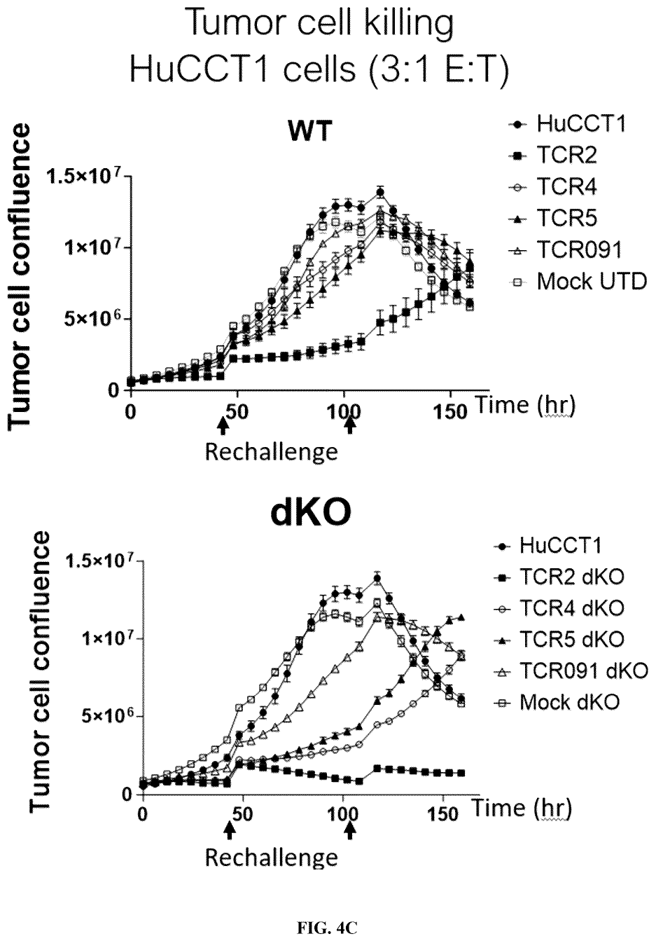

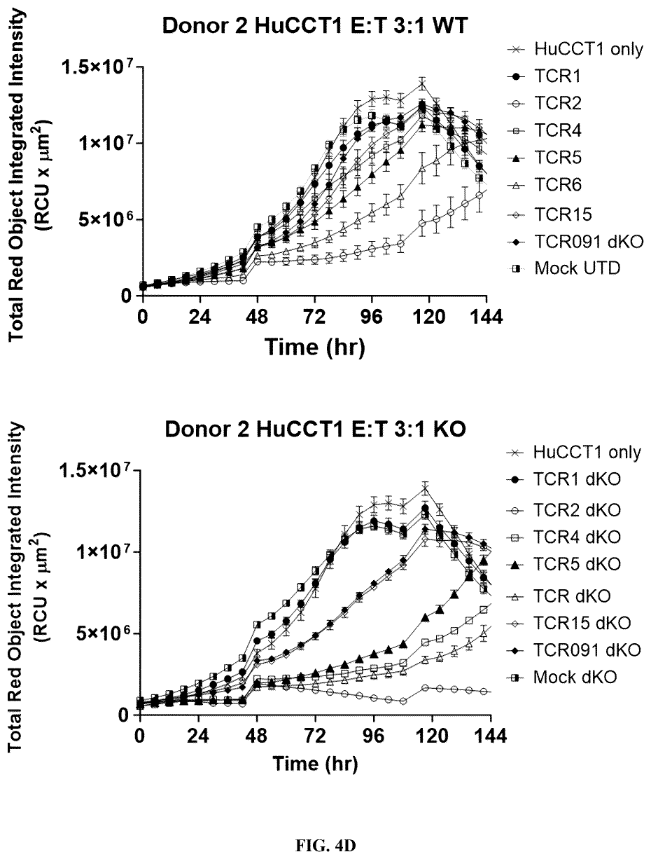

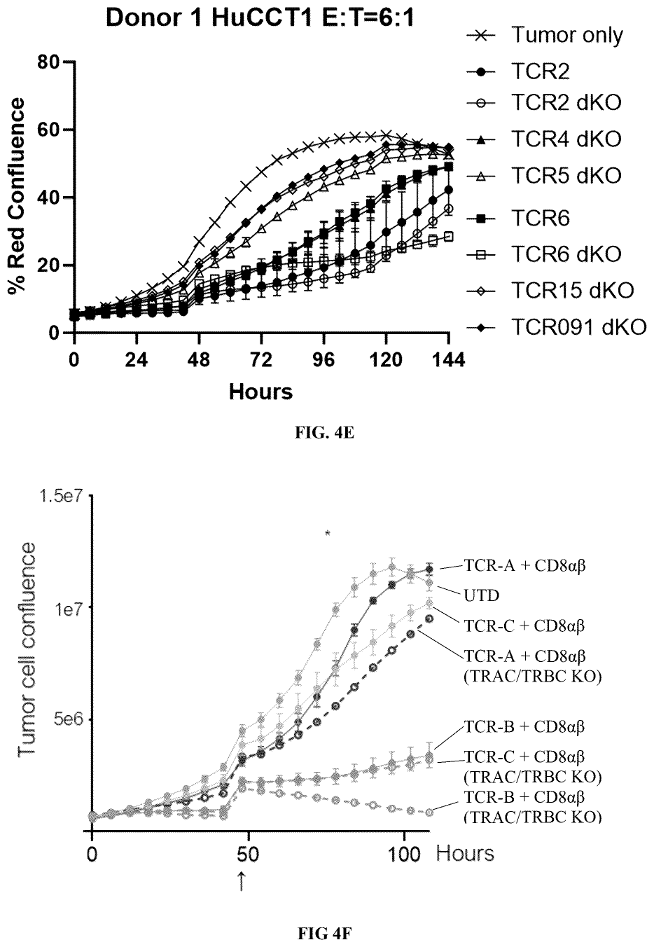

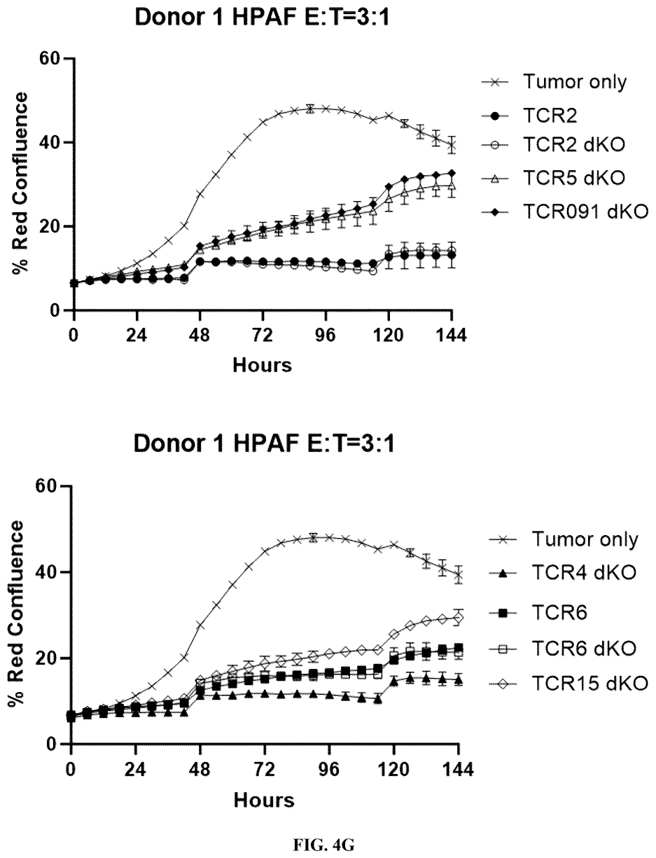

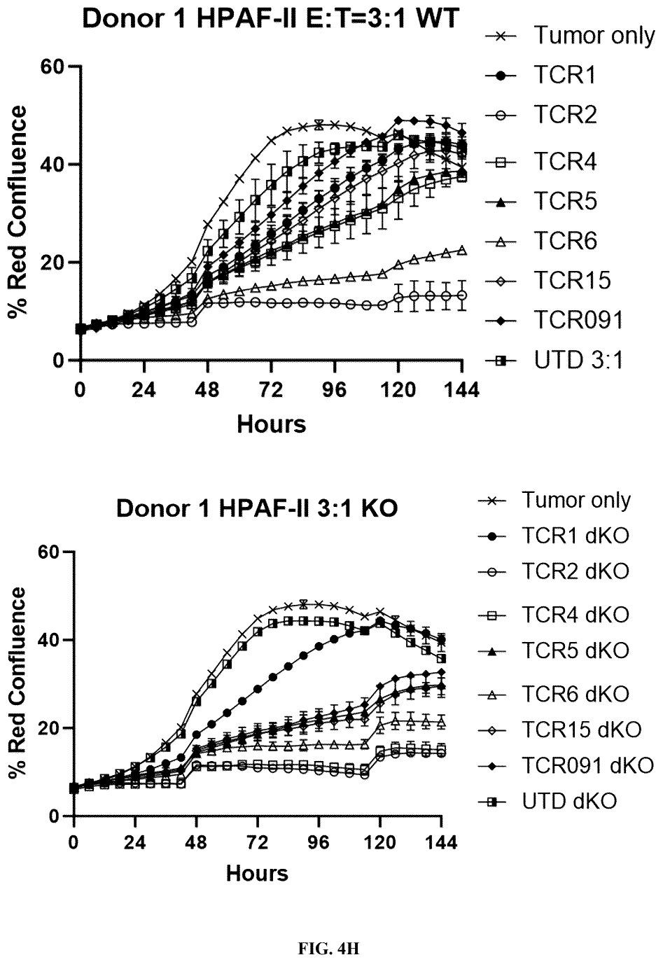

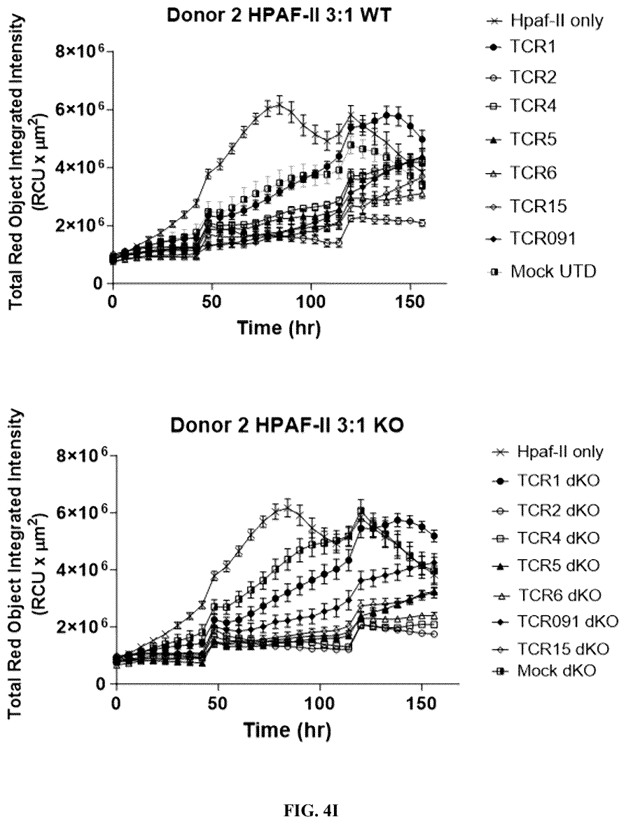

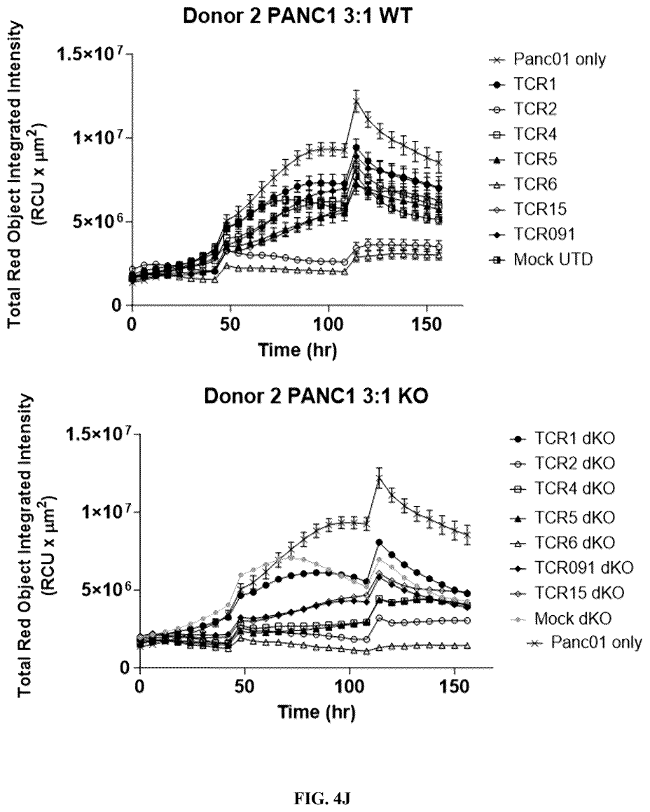

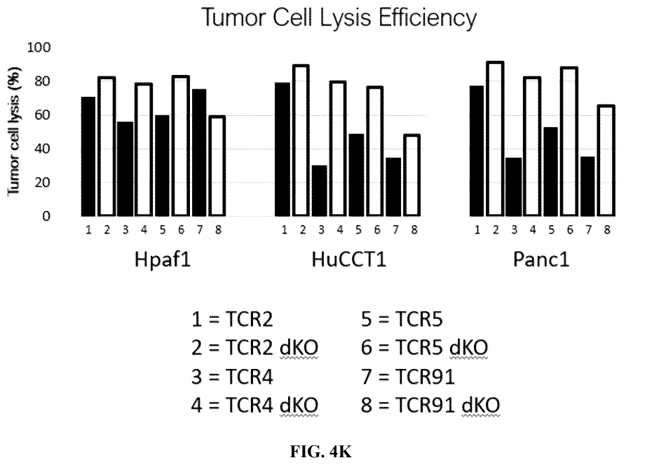

In a similar experiment, primary CD4+ and CD8+ T cells transduced with further KRAS G12D-specific T cell receptors, with or without knockout of TRAC and TRBC, were assessed for cytotoxicity of Hpaf-II cells. In this case a single rechallenge with tumor cells at 72 hours was included. Improved T cell killing activity was generally observed in the TRAC/TRBC dKO T cells (FIG. 4B) compared to cells with intact wild type TRAC/TRBC (FIG. 4A).

The impact of TRAC and TRBC knockout on cancer cell killing was also evaluated for additional KRAS-G12D-specific T cell receptors using KRAS G12D-presenting HuCCT1 (cholangiocarcinoma; FIGS. 4C-4F), Hpaf-II (pancreatic adenocarcinoma; FIGS. 4G-4I), and Panc1 (Pancreatic ductal adenocarcinoma; FIG. 4J) target cells. In some cases cell death was observed at late time points for tumor cell only or mock treated conditions due to the tumor cells reaching confluence. KRAS-G12D-specific engineered T cells with knockout of endogenous TRAC/TRBC genes showed enhanced killing of multiple cell lines as summarized in FIG. 4K.

### Example 5: Activation of T Cells Expressing Candidate of KRAS-G12D TCRs is Enhanced by Knockout of TRAC and TRBC

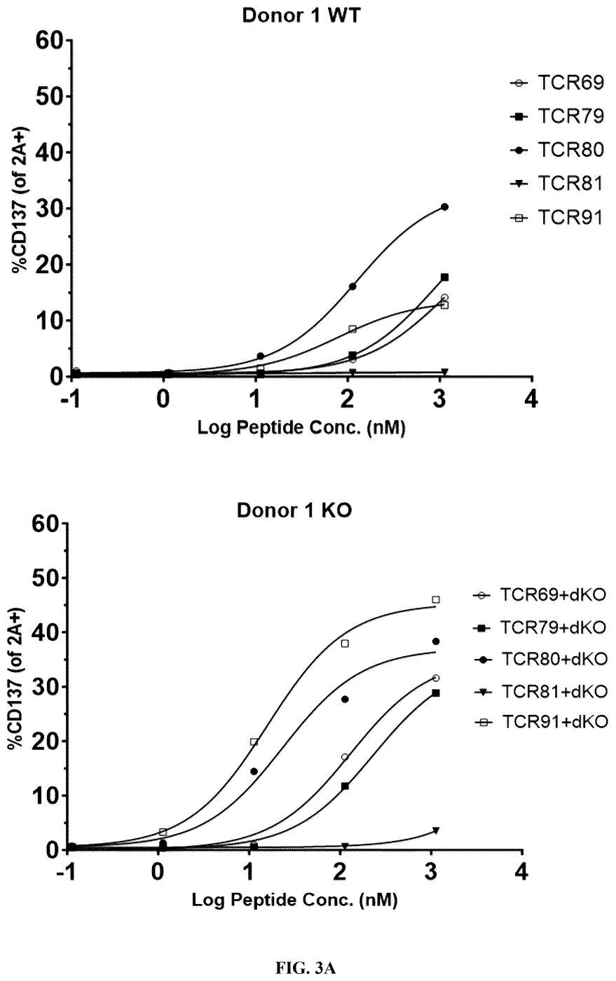

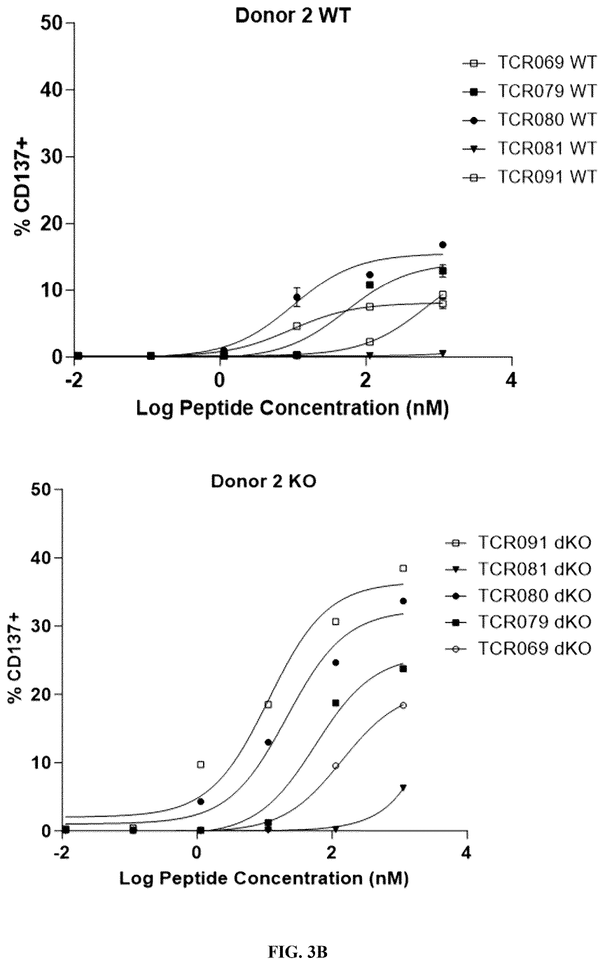

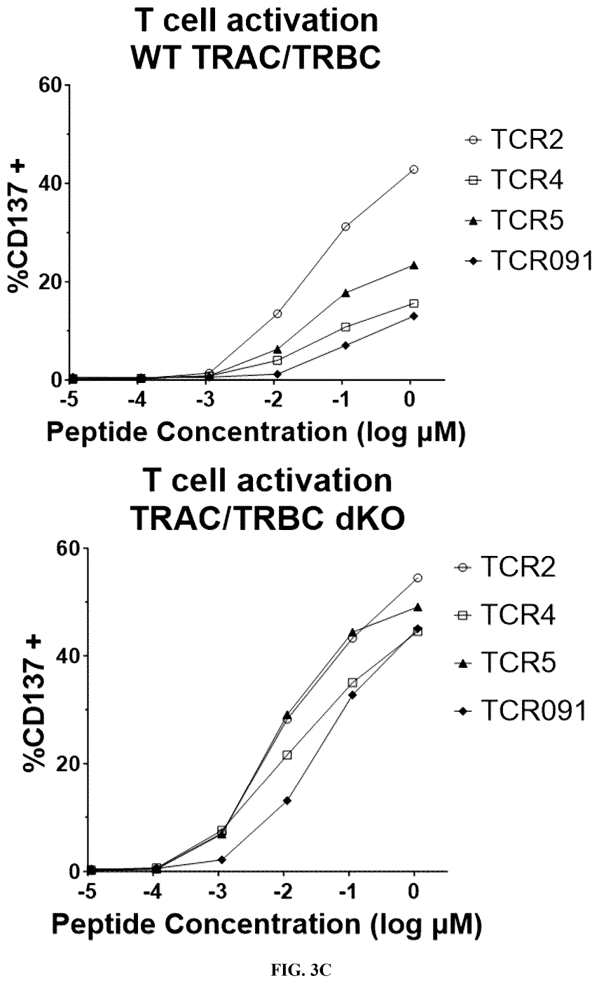

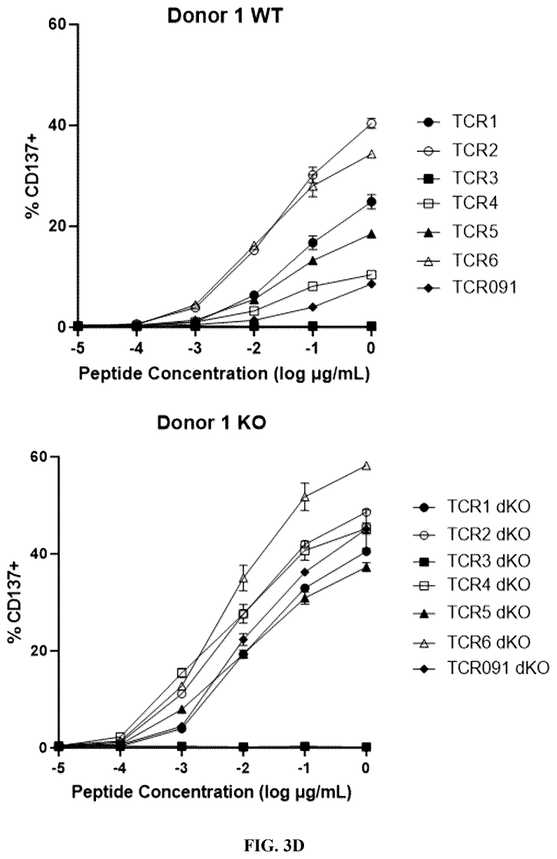

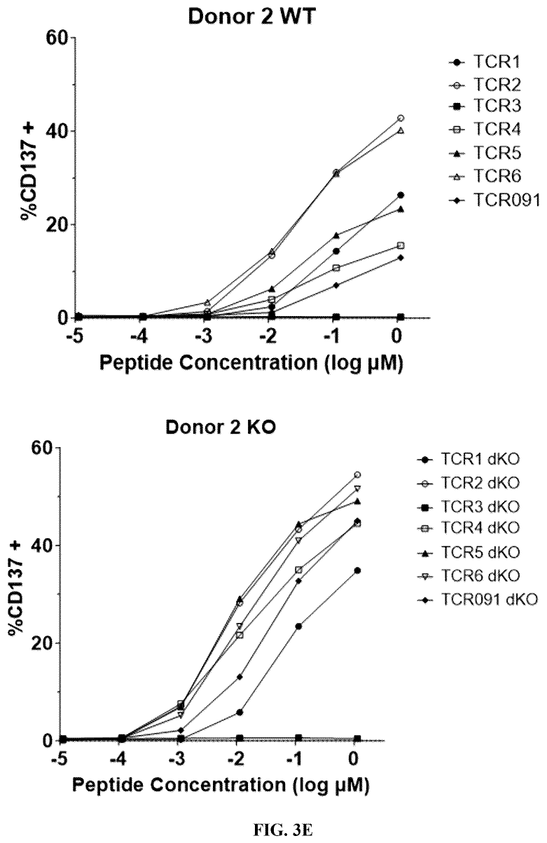

This example demonstrates that knockout of TRAC and TRBC can enhance activation of T cells expressing candidate KRAS G12D-specific TCRs disclosed herein. Primary CD4+ and CD8+ T cells were transduced with a KRAS-G12D-specific T cell receptor (TCR69, TCR79, TCR80, TCR81, or TCR91), and were either edited to knockout TRAC and TRBC or left with unmodified TRAC and TRBC genes, and expanded for several days. Engineered T cells were incubated with G12D peptide at decreasing concentrations and T cell responses were assessed by measuring CD137 expression. The data demonstrate that knocking out the TRAC/TRBC loci improved TCR avidity in cells sourced from two donors (FIG. 3A and FIG. 3B).

In additional experiments, the effect of TRAC and TRBC knockout was evaluated for primary CD4+ and CD8+ T cells transduced with further KRAS-G12D-specific TCRs. The double knockout improved TCR avidity for multiple TCRs as shown in FIG. 3C (corresponding EC50 values in TABLE 2 and Cmax values in TABLE 3), FIG. 3D (corresponding EC50 values in TABLE 4), and FIG. 3E (corresponding EC50 values in TABLE 5). TCR2, TCR4, TCR5, and TCR6 exhibited the lowest EC50 values in this screen. In in many cases substantially lower EC50 values were observed with knockout of TRAC and TRBC (e.g., 3-5 fold improvements).

TABLE 2
TABLE 2: T cell activation EC50 (nM) values for
T cells expressing candidate KRAS G12D-specific
TCRs with and without knockout of TRAC and TRBC.

| Construct | WT | Double knockout |
|---|---|---|
| TCR2 | 31.6 | 10.1 |
| TCR4 | 49.8 | 11.8 |
| TCR5 | 36.3 | 7.6 |
| TCR91 | 131.3 | 36.5 |

TABLE 3
TABLE 3: Cmax (%) values for T cells expressing candidate KRAS
G12D-specific TCRs (without knockout of TRAC and TRBC).

| Construct | Cmax |
|---|---|
| TCR2 | 42.68 |
| TCR4 | 15.97 |
| TCR5 | 23.91 |
| TCR91 | 14.43 |

TABLE 4
TABLE 4: T cell activation EC50 (nM) values for
T cells expressing candidate KRAS G12D-specific
TCRs with and without knockout of TRAC and TRBC.

| Construct | WT | Double knockout |
|---|---|---|
| TCR1 | 51.1 | 12.2 |
| TCR2 | 22.2 | 7.3 |
| TCR4 | 31.1 | 4.4 |
| TCR5 | 37.8 | 9.4 |
| TCR6 | 12.2 | 6.4 |
| TCR15 | 114.4 | 27.8 |
| TCR91 | 178.9 | 14.4 |

TABLE 5
TABLE 5: T cell activation EC50 values in nM for
T cells expressing candidate KRAS G12D-specific
TCRs with and without knockout of TRAC and TRBC.

| Construct | WT | Double knockout |
|---|---|---|
| TCR1 | 119.1 | 64.1 |
| TCR2 | 31.6 | 10.1 |
| TCR4 | 49.8 | 11.8 |
| TCR5 | 36.3 | 7.6 |
| TCR6 | 24.0 | 14.3 |
| TCR15 | 170.3 | 52.2 |
| TCR91 | 131.3 | 36.5 |

### Example 6: TCR Selectivity

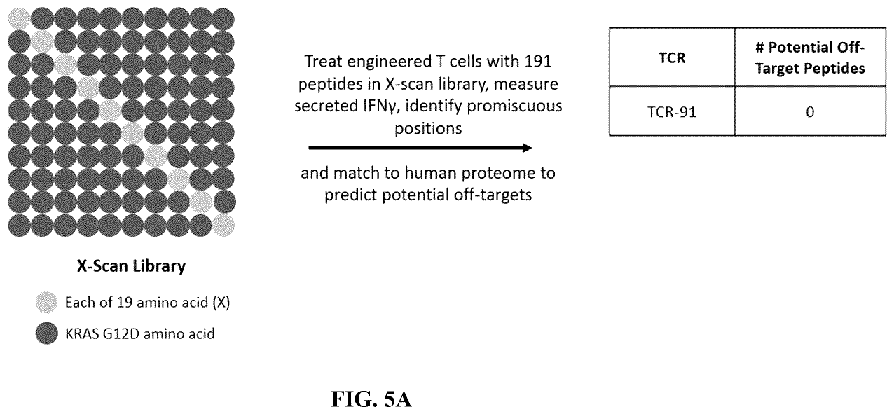

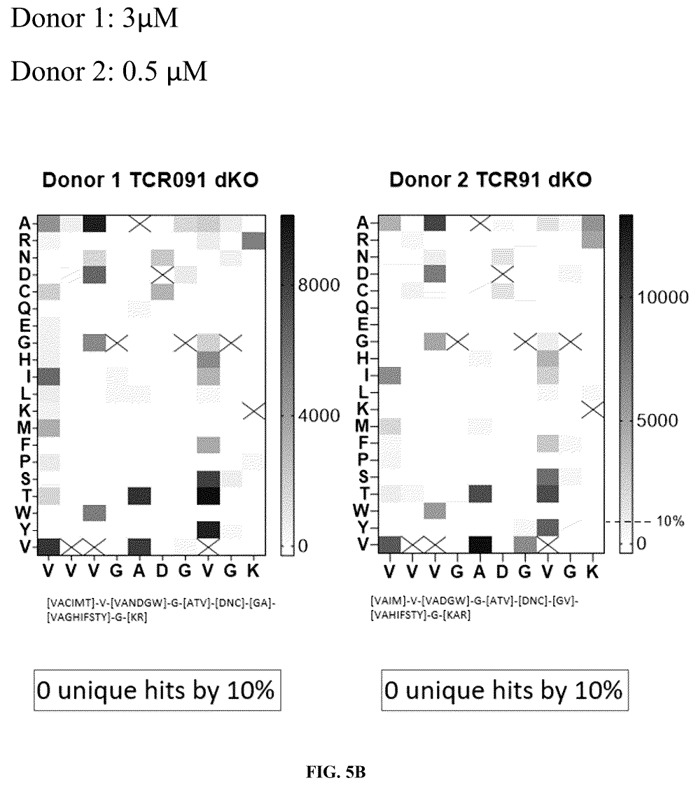

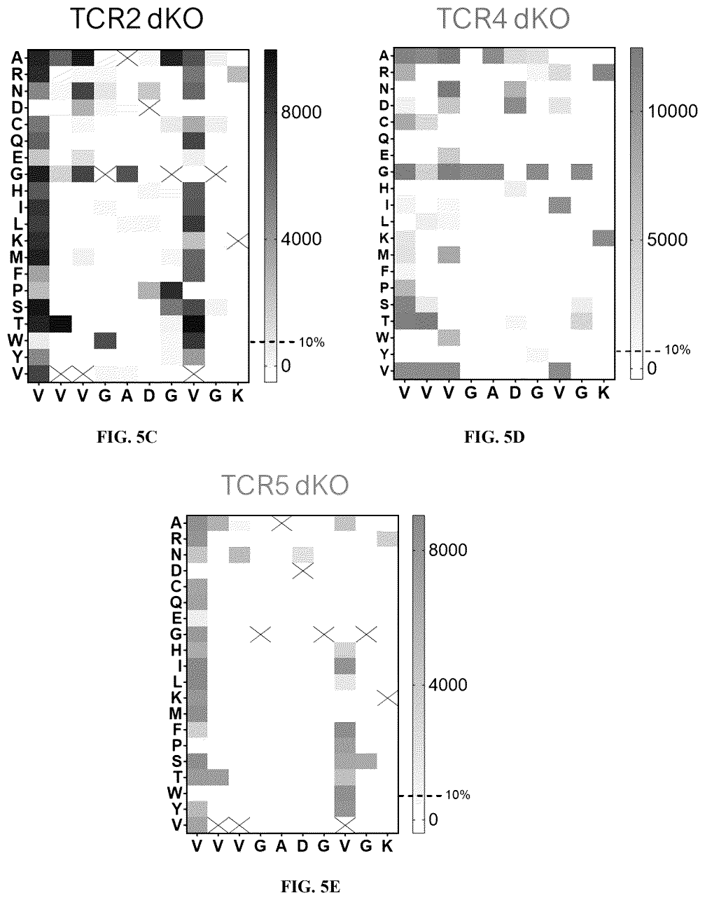

To assess potential TCR off-target activity, CD4+/CD8+ T cells expressing TCRs disclosed herein were cultured overnight with a panel of 191 positional scanning peptides containing a substitution of every possible amino acid at each position of the cognate KRAS G12D peptide (FIG. 5A). Secreted IFNγ levels were used to identify TCR cross-reactivity to promiscuous positions. Potentially antigenic peptides were then matched against the human proteome to predict potential in vivo off-targets using ScanProsite (prosite.expasy.org/scanprosite/). Peptides that elicited a response of greater than 10% of the maximum signal were considered positive in this assay. Few or no potential off-targets were identified for G12D-recognizing TCR91 (FIG. 5B), TCR2 (FIG. 5C), TCR4 (FIG. 5D), and TCR5 (FIG. 5E), as summarized in TABLE 6.

TABLE 6

| | # Potential Off- |
|---|---|
| TCR | Target Peptides |
| TCR2 | 21 |
| TCR4 | 9 |
| TCR5 | 1 |
| TCR91 | 0 |

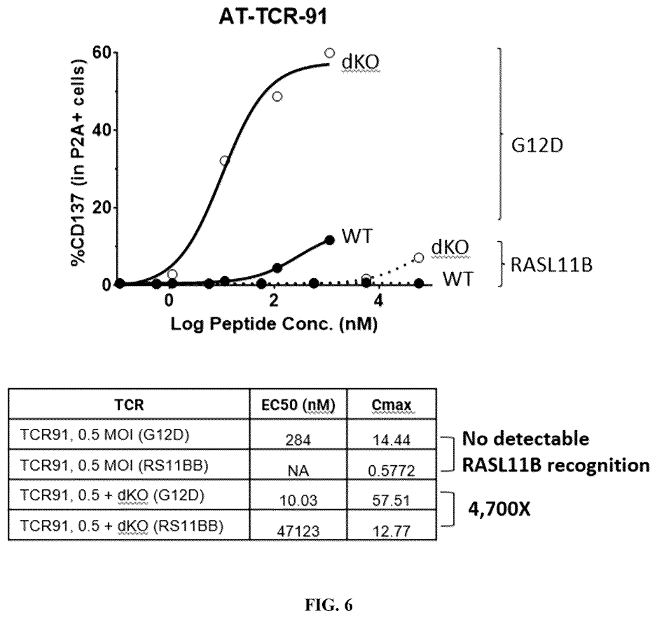

An in vitro assay was conducted to evaluate specificity of TCR091 for KRAS-G12D peptide and potential off-target reactivity to RASL11B, an off-target interaction partner of a non-related KRAS-G12D TCR. TCR91-transduced primary CD4+/CD8⁺ T cells having wild type TRAC/TRBC loci or TRAC/TRBC dKO were cultured overnight with decreasing concentrations of KRAS-G12D peptide or RASL11B peptide and CD137 expression was assessed by flow cytometry. Dose-response curves were fitted by non-linear regression, and EC50 values were calculated using GraphPad Prism®. T cell activation in response to KRAS-G12D peptide was greater in dKO T cells than in TRAC/TRBC wild type cells (FIG. 6). Additionally, only a very low-level response (approximately 10%) was detected for RASL11B.

### Example 7: Activation and Target Cell Killing by T Cells Expressing Candidate of KRAS-G12D TCRs is Enhanced by Knockout of TRAC and TRBC

This example further demonstrates that knockout of TRAC and TRBC can enhance activation of and target cell killing by T cells expressing candidate KRAS G12D-specific TCRs disclosed herein.

TCR91-transduced primary CD4⁺/CD8⁺ T cells having wild type TRAC/TRBC loci, a TRAC single knockout (sKO), or TRAC/TRBC dKO were expanded for 10 days, and treated with G12D peptide at decreasing concentrations. T cell activation was measured using CD137 expression as assessed by flow cytometry. Dose-response curves were fitted by non-linear regression, and EC50 values were calculated using GraphPad Prism®. TCR91-transduced TRAC/TRBC dKO cells exhibited enhanced activation compared to WT and TRAC single KO cells (FIG. 7).

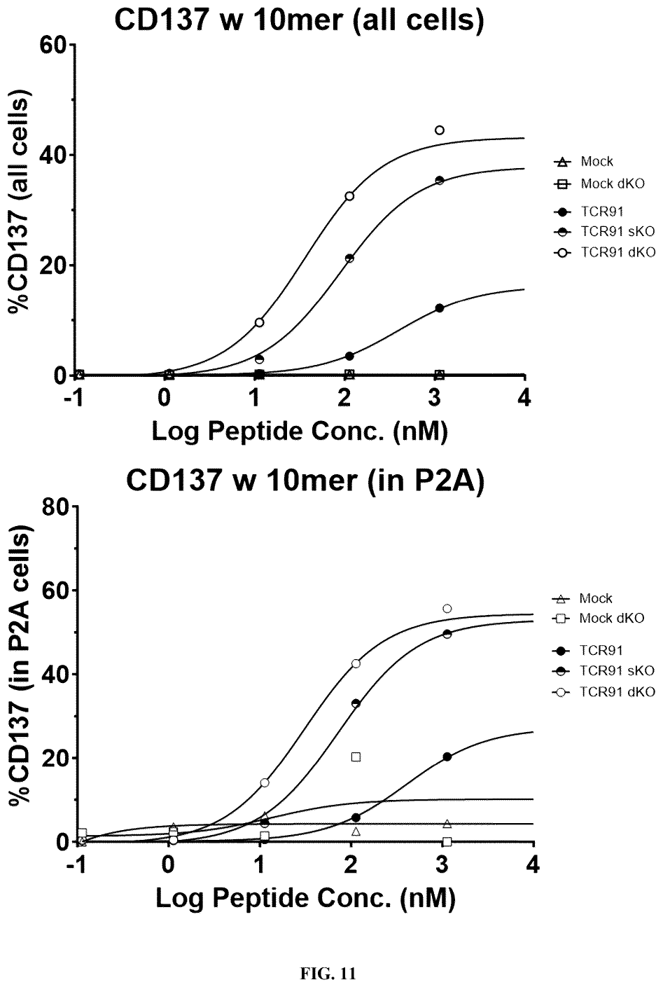

In a further experiment, TCR91-transduced TRAC/TRBC dKO cells exhibited increased activation compared to WT and TRBC sKO cells (FIG. 11).

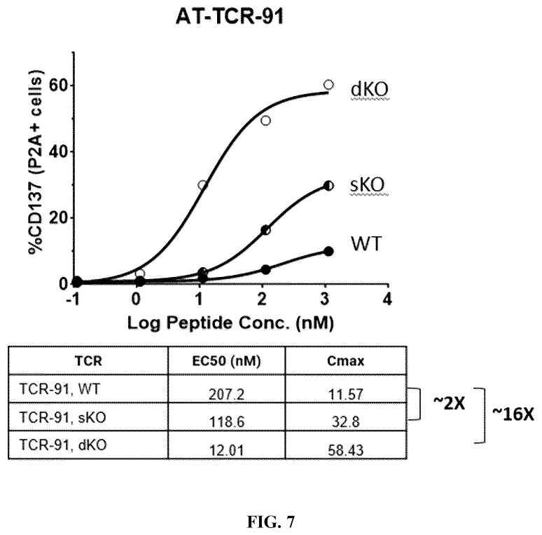

To evaluate the impact of single or double TRAC/TRBC knockout on target cell killing, TCR91-transduced primary CD4+/CD8⁺ T cells having wild type TRAC/TRBC loci, a TRAC single knockout (sKO), or TRAC/TRBC dKO were expanded for 10 days, and co-cultured with HpafII cells at a 3:1 effector:target cell ratio. Cytotoxic activity was assessed and plots of red fluorescence measured over time by live cell imaging were generated. The results demonstrate that the TRAC/TRBC dKO cells are more effective at controlling HpafII tumor cells relative to either the TRAC sKO or TRAC/TRBC wild type cells (FIG. 8). Enhanced cytotoxicity was also observed in an assay using Panc-1 tumor cells as target cells (FIG. 9).

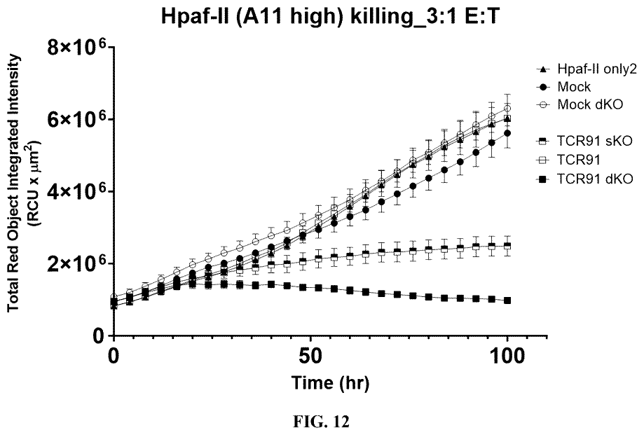

In a further experiment, cytotoxicity of the dKO was compared to single knockout (sKO) of TRBC. TCR91-transduced primary CD4+/CD8⁺ T cells having wild type TRAC/TRBC loci, a TRBC sKO, or TRAC/TRBC dKO were expanded for 10 days, and co-cultured with fluorescent HpafII cells at a 3:1 effector:target cell ratio. The TRAC/TRBC dKO TCR91-transduced cells were more effective at controlling HpafII tumor cells relative to either the TRBC sKO or TRAC/TRBC wild type cells (FIG. 12).

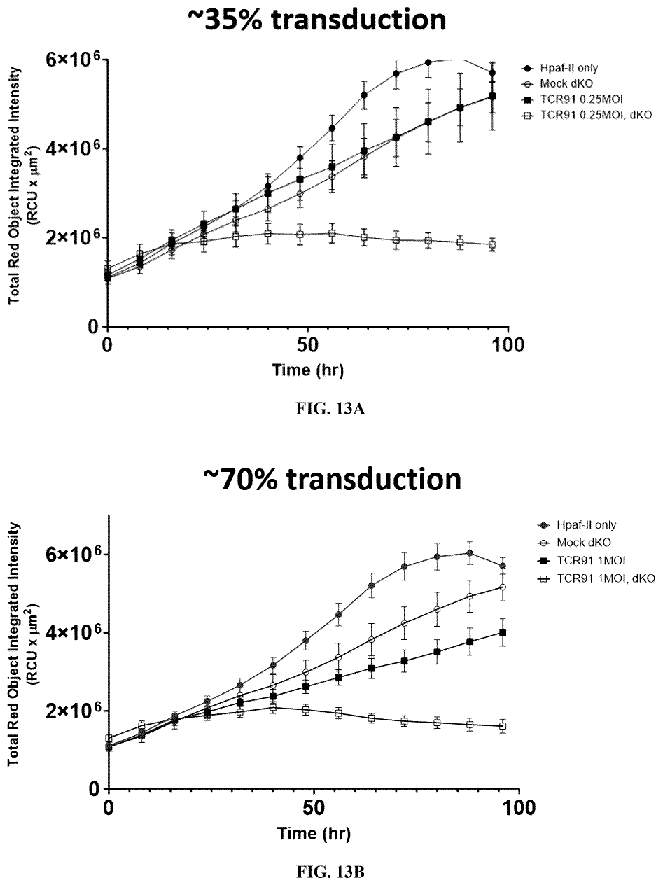

In another assay, primary CD4+ and CD8+ T cells were transduced with a KRAS-G12D-specific T cell receptor (TCR-91) at 35% (FIG. 13A) or 70% (FIG. 13B) transduction efficiency, with or without knockout or TRAC and TRBC. After expansion for several days, engineered cells were co-cultured with HpafII cells at a 3:1 effector:target cell ratio. The results demonstrated that the TRAC/TRBC dKO cells are more effective at controlling HpafII tumor cells relative to the TRAC/TRBC wild type cells.

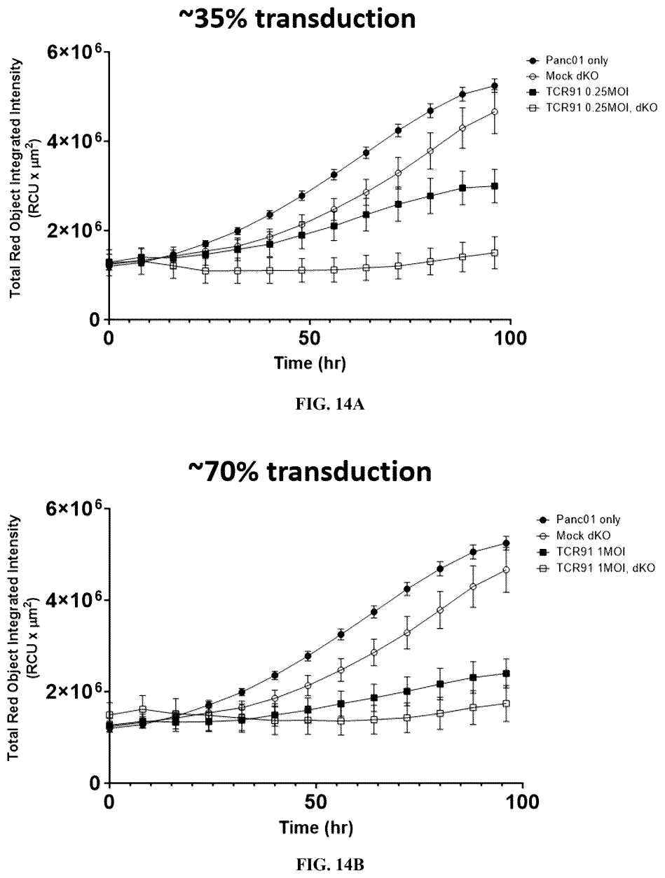

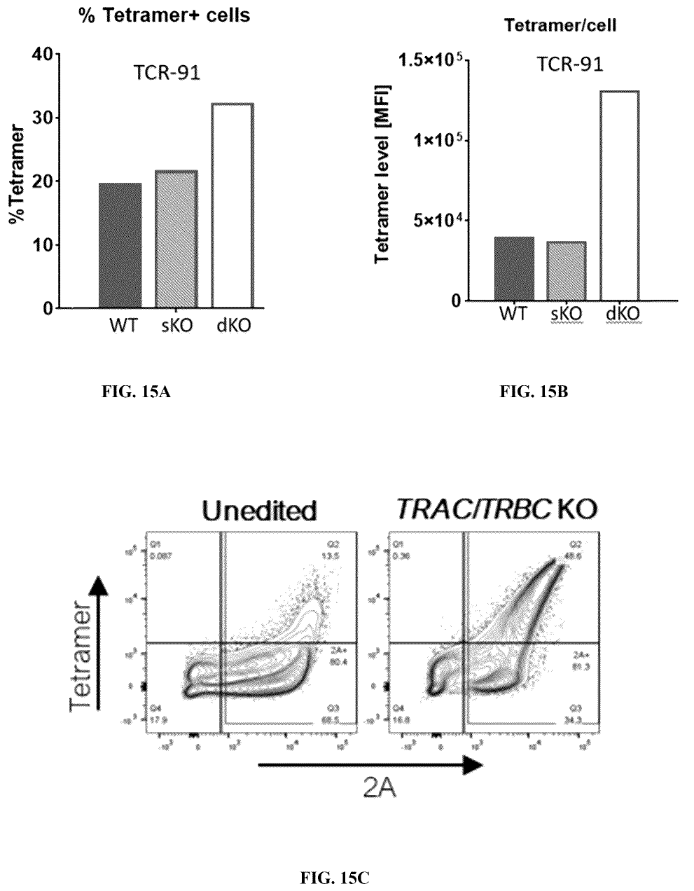

A similar study was performed with Panc-1 tumor cells as target cells. Primary CD4+ and CD8+ T cells were transduced with a KRAS-G12D-specific TCR91 at 35% (FIG. 14A) or 70% (FIG. 14B) transduction efficiency, with or without knockout of TRAC and TRBC. After expansion for several days, engineered cells were co-cultured with fluorescent Panc-1 cells at a 10:1 effector:target cell ratio. Cytotoxic activity was assessed using the IncuCyte assay. The results demonstrate that the TRAC/TRBC dKO cells are more effective at controlling Panc-1 tumor cells relative to the TRAC/TRBC wild type cells.

### Example 8: Tetramer Analysis of KRAS-G12D TCRs with Knockout of TRAC and/or TRBC

This example demonstrates enhanced surface expression and peptide-MHC binding of KRAS-G12D TCRs disclosed herein with knockout of TRAC and TRBC.

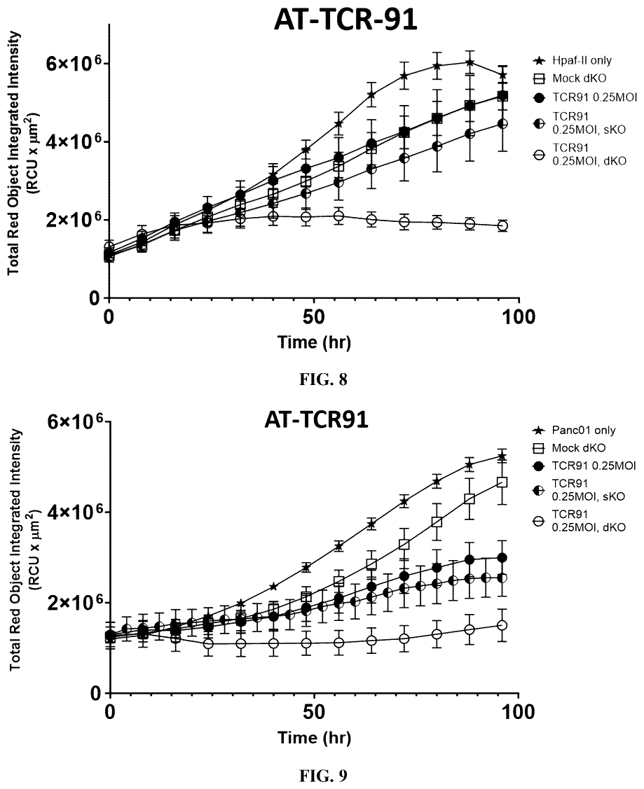

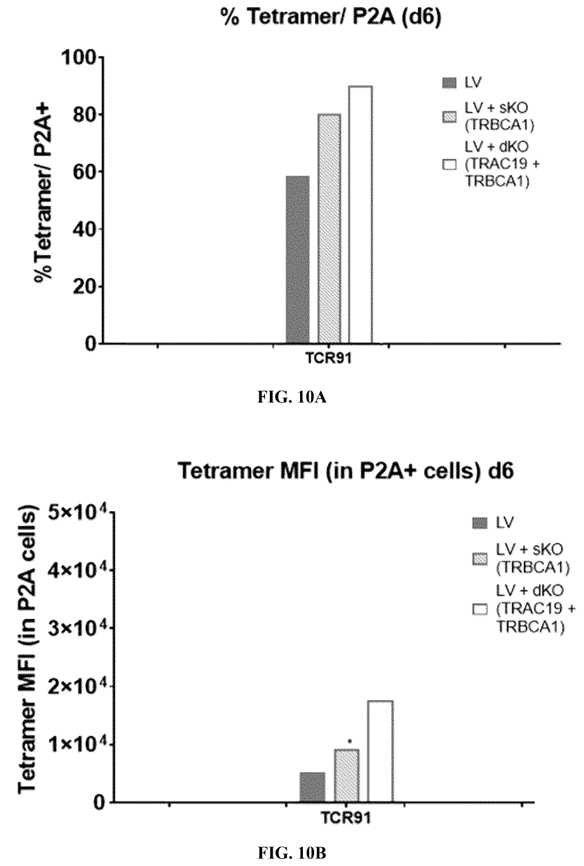

TCR91-transduced primary CD4+/CD8⁺ T cells having wild type TRAC/TRBC loci, a TRBC single knockout (sKO), or TRAC/TRBC dKO were expanded for 10 days. Cell surface expression of correctly paired G12D TCR and its functionality was assessed using fluorophore labeled tetramers of MHC-peptide complex. Increases in the percentage of cells staining positive for peptide-MHC binding (FIG. 10A) and in the mean fluorescence intensity (MFI, reflecting average signal per cell; FIG. 10B) were observed in cells with knockout of endogenous TCR encoding genes, with greater increases upon TRAC/TRBC dKO.

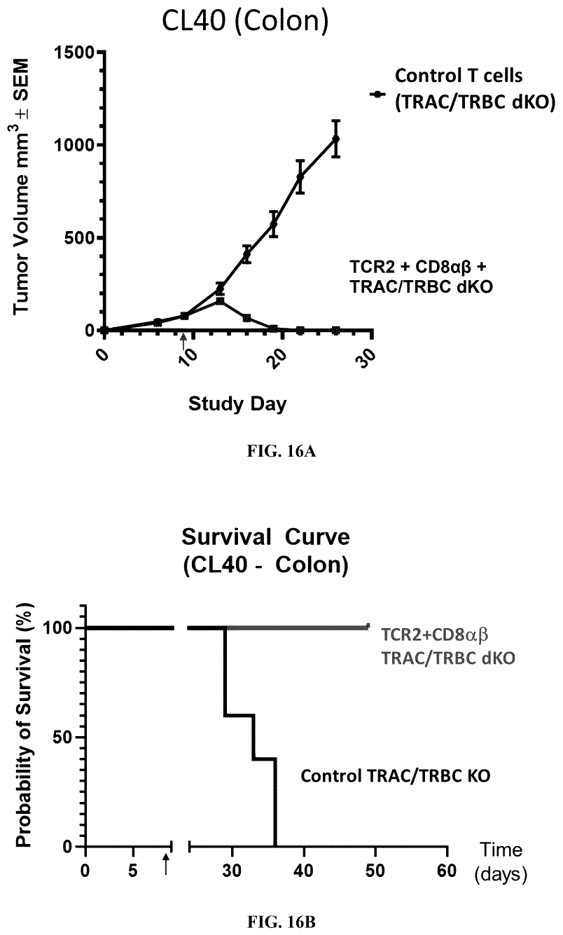

In a similar study, a comparison was done to TRAC single knockout. TCR91-transduced primary CD4+/CD8⁺ T cells having wild type TRAC/TRBC loci, a TRAC single knockout (sKO), or TRAC/TRBC dKO were expanded for 10 days and evaluated for cell surface expression of correctly paired G12D TCR via binding of fluorophore labeled MHC-peptide tetramers. Increases in the percentage of cells staining positive for peptide-MHC binding (FIG. 15A) and in the mean fluorescence intensity (MFI, reflecting average signal per cell; FIG. 15B) were observed in cells with knockout of endogenous TRAC/TRBC (dKO) compared to WT or TRAC sKO cells.

Additional experiments using primary CD4⁺/CD8⁺ T cells also demonstrated enhanced surface expression and peptide-MHC binding of three KRAS G12D-specific TCRs (TABLE 7; see also representative flow plot in FIG. 15C, in which the percent of tetramer positive, 2A positive cells was 48.6% with dKO versus 13.5% without knockout).

TABLE 7

| | |
|---|---|
| KRAS | |
| A11.G12D | Tetramer MFI |

| TCR | Unedited | dKO |
|---|---|---|
| TCR-A | 1197 | 3214 |
| TCR-B | 7629 | 14250 |
| TCR-C | 369 | 2505 |

### Example 9: In Vivo Efficacy of T Cells Expressing Candidate of KRAS-G12D TCRs with Knockout of TRAC and TRBC

This example demonstrates in vivo efficacy of T cells expressing a KRAS G12D-specific TCR disclosed herein with knockout of TRAC and TRBC.

Nod scid gamma (NSG) mice were implanted subcutaneously with CL40 colon adenocarcinoma cells, randomized (n=5 per group), and treated day 9 post-implant with 10×10\hat{ }6 T cells administered intravenously (control T cells with knockout of endogenous TRAC and TRBC, or T cells expressing a KRAS G12D-specific TCR disclosed herein with co-expression of a CD8 co-receptor (CD8αβ) and knockout of endogenous TRAC and TRBC). A 1:1 ratio of CD4+ and CD8+ T cells was administered. The engineered cells demonstrated robust anti-tumor efficacy as demonstrated by control of tumor volume (FIG. 16A). In the study period, 100% complete responses and 100% survival was observed for the group administered the T cells expressing the KRAS G12D-specific TCR with dKO, while no animals that were administered control T cells survived (FIG. 16B).

### Example 10: KRAS G12D TCR T Cells Armored with IL7R Signaling Show Enhanced Proliferation and Tumor Cell Killing

This example demonstrates enhanced proliferation and tumor cell killing via engineered cells disclosed herein.

CD4+ and CD8+ T cells were engineered to comprise: (i) a KRAS G12D-specific TCR disclosed herein; (ii) a CD8 co-receptor (CD8αβ); (iii) knockout of endogenous TRAC and TRBC expression; and/or (iv) a chimeric fusion protein comprising an IL-7 receptor intracellular signaling domain, a transmembrane domain, and an extracellular domain (e.g., an extracellular domain from CD34, CD58, or CD80).

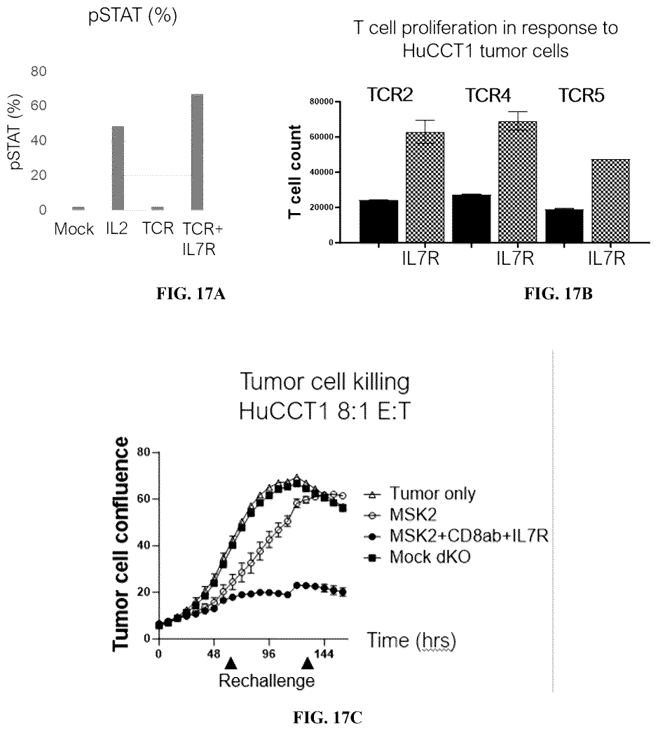

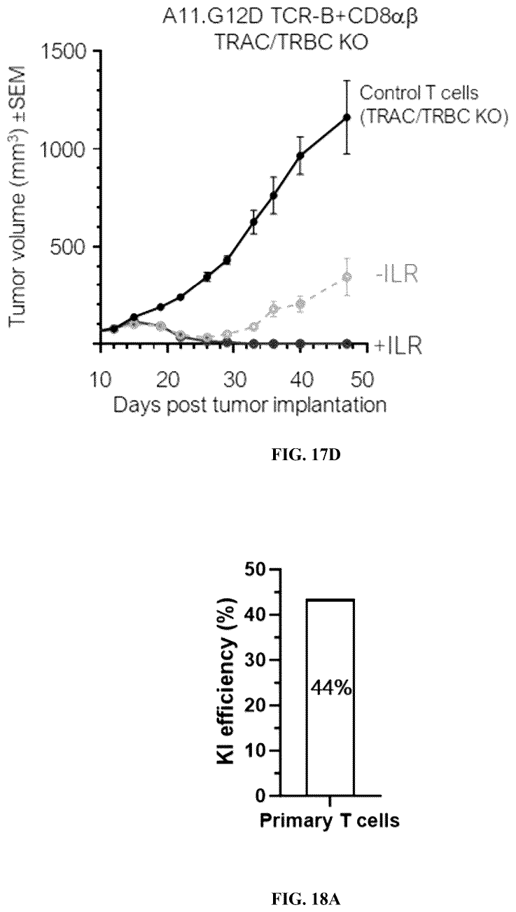

The engineered cells were co-incubated with HuCCT1 tumor cells and assessed for STAT5 phosphorylation, proliferation, and tumor cell killing. Cells harboring the chimeric IL7R fusion protein exhibited increased STAT5 phosphorylation (FIG. 17A; indicating activity of IL7R), proliferation (FIG. 17B), and tumor cell killing (including upon multiple rechallenges; FIG. 17C) relative to cells lacking the chimeric IL7R fusion protein.

1×10\hat{ }7 of the engineered cells were intravenously administered to mice 9 days after subcutaneous inoculation of HuCCT1 tumor cells. Inclusion of the chimeric IL-7R fusion protein further improved the anti-tumor response, resulting in complete responses (FIG. 17D)

### Example 11: Non-Viral Transgene Integration can Achieve High Efficiency of Transgenesis and Improve Engineered T Cell Functionality

This example provides a comparison of lentiviral versus non-viral targeted transgene integration, and functionality of resulting engineered T cells.

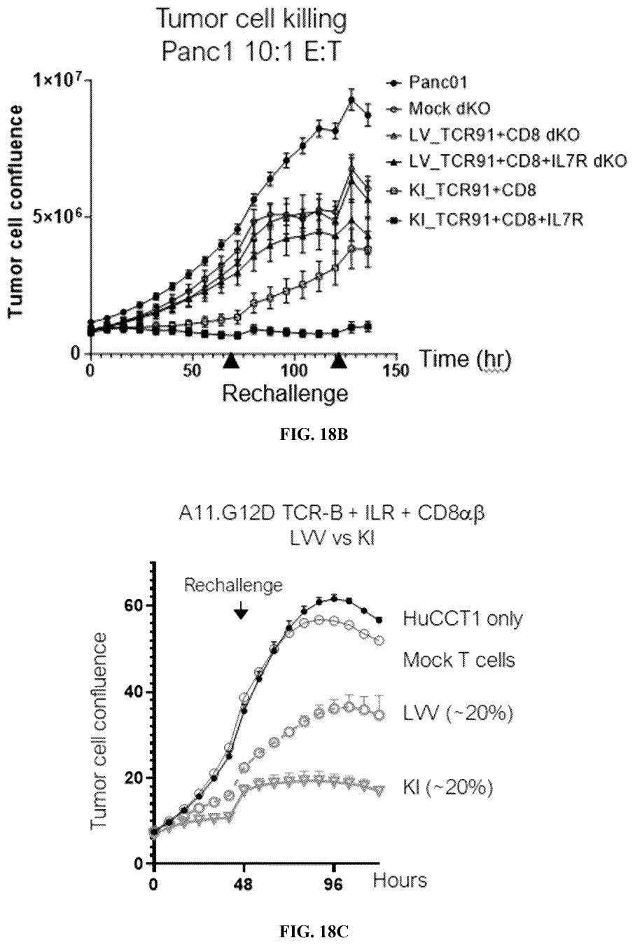

For non-viral knock in and concurrent knockout of TRAC and TRBC, a CRISPR system was used, with electroporation of Cas-gRNA RNPs to generate double stranded breaks in the TRAC and TRBC loci. A nanoplasmid was used to provide a template for insertion of an expression cassette into the TRAC locus via homology directed repair (HDR). RNP and nanoplasmid DNA were electroporated 48 hrs after activation of T cells. Electroporation was performed using Xenon. Non-viral knock-in yielded up to 44% integration efficiency in primary CD4+ and CD8+ T cells (FIG. 18A).

After non-viral knock-in or lentiviral transduction, killing of Panc-1 tumor cells was evaluated using the IncuCyte assay described for FIG. 2, with cells incubated at a 10:1 effector to target ratio. Enhanced killing was observed for engineered T cells generated by non-viral knock-in (FIG. 18B). Enhanced killing was also observed with introduction of the chimeric fusion protein comprising an IL-7 receptor intracellular signaling domain as described in EXAMPLE 10.

In an additional experiment, following non-viral knock-in or lentiviral transduction, killing of HuCCT1 tumor cells was evaluated using the IncuCyte assay. T cells engineered via non-viral knock-in exhibited improved tumor cell killing, including after rechallenge with fresh tumor cells after 48 hours (FIG. 18C).

### Example 12: Illustrative Non-Viral Transgene Integration Protocol

This example provides illustrative methods for non-viral knock-in of a transgene/expression cassette combined with knockout of TRAC, TRBC1, and/or TRBC2. Materials: (i) T-cell donor of interest; (ii) Nanoplasmid (5 mg/mL stock); (iii) X-Vivo-15, cytokine; (iv) 6 and 24 Well GREX Plate; (v) CTS Xenon Electroporation Buffer (Gibco, Cat #A4997901) or CTS Xenon Genome Editing Buffer (Gibco, Cat #A4998001); (vi) 96 well flat bottom plate (assay plate); (vii) 96 well U bottom plate (staining plate); (viii) Miltenyi T-cell activating Transact (Miltenyi, Cat #130-111-160); (ix) Antibodies for: CD4, CD8, TCRab, P2A, Live/Dead, tetramer peptide conjugate; (x) BD CytoFix; and (xi) 10× BD Perm/Wash.

### Day 0: Thawing Cells.

Fresh medium for thawing T-cells comprises either X-vivo 15+2% Immune Cell Serum Replacement (ICSR)+IL2 (100 Units), IL7 (5 ng/mL), and IL15 (5 ng/mL); or X-Vivo 15+IL2 (100 Units), IL7 (500 Units), and IL15 (500 Units).

The cells are subjected to a rapid thaw and then resuspended in complete medium (e.g., a final volume of 10 mL). T cells are counted (Celleca, AOPI staining) to determine number of cells and viability. The T cells are spun at 300×g for 5 minutes, and the medium is then removed and the cell pellet is responded in 1 mL of fresh media.

The resuspended cells are transferred to a well on a 6-well GREX plate (about 24 mL of medium per well). The well is supplemented with 1:100 Miltenyi Transact (e.g., 250 μL for 25 mL medium) to activate the T cells and incubated overnight.

### Day 2: Knock in Electroporation.

RNP is complexed at a 2:1 nuclease to guide MOLAR ratio for 20-30 minutes at room temperature: (a) sgRNA (300 μM stock): 2.1 μL; (b) MG-21 nuclease (6.93 mg/mL stock): 5.4 μL; (c) Total RNP per single reaction: ˜7.5 μL. While the RNP complexes, donor T-cells from the bottom of the GREX plate are resuspended, pooled, and counted. T cells are collected and spun down 300×g for 5 minutes. Neon electroporation reactions use 5×10\hat{ }6 cells per reaction, and Xenon electroporation reactions use 50×10\hat{ }6 cells per reaction. Post-EP medium is X-vivo medium+IL 2, 7, 15, with 5% ICSR. If double knock outs are performed, the respective RNPs are mixed 1:1 for easier pipetting (15 μL per EP reaction in Neon, 150 μL for Xenon). Cells are pelleted and only resuspended in EP buffer when ready to electroporate, as buffer can be toxic to the cells.

Neon electroporation option: reaction conditions include: (a) 100 μL electroporation buffer to resuspend cells; (b) 7.5 μL per RNP; (c) KI nanoplasmid if used to concentration of interest (20 μg): 4 μL plasmid; and (d) Electroporation: 2300 V, 4 pulses, 3 ms pulse width.

Place an electroporation tube in the electroporation unit, and fill the tube with 3-5 mL of E2 buffer to connect the conductors. Attach the electroporation tip to the syringe (press down hard to attach, gold plated conductor rod should not be more than 30% of the way down on a non-depressed syringe). Use the syringe to gently pipette the sample and draw it into the tip. No bubbles should be present in the tip at the top as it interferes with electroporation. Once loaded into the Neon Tip, transfer to the casing and click the syringe into place in the tube. Run the Neon electroporator, which will beep once to acknowledge selection and again upon completion. Depress tip into a 24 well flat bottom plate to allow cells to rest for 10 minutes. After 10 minutes, take 1 mL of medium from the final destination well and resuspend the rested cells. Incubate cells at 37° C. to allow growth and count cells on day 4.

Xenon electroporation option: reaction conditions include: (a) 800 μL electroporation buffer to resuspend cells; (b) 200 μL Master Editing Mix (see below); and (c) EP Protocol: 2300 V, 4 pulses, 3 ms pulse width, 500 ms pulse interval.

Make a 200 μL Master mix of RNP, buffer (the same as for Neon electroporation), and nanoplasmid (or other nucleic acid molecule): 75 μL per RNP, 40 μL nanoplasmid (200 μg, if using), Q.S. using electroporation buffer to 200 μL. Pellet cells at 300×g for 5 minutes. Resuspend pellet in 800 μL of buffer. Add the 200 μL Editing Master Mix to the 800 μL. Transfer entire volume (1 mL) into a Xenon One Shot cartridge by removing the top and filling the unit. Cell solution should be slightly convex to ensure contact with the conductor at the top of the cartridge. Insert into Xenon One Shot cartridge into the instrument and run the electroporator. The Xenon electroporator will initiate pulses and inform you when the process is complete. Remove and allow cells to rest in the cartridge for 10 minutes before transferring into 40 mL Xvivo15+5% ICSR+cytokines and incubate at 37° C. On the following day, add 60 mL of fresh medium to the well.

### Example 13: a KRAS G12D-Specific TCR Expressing a CD8Ab Co-Receptor and a Chimeric Cytokine Receptor Enhanced Activity In Vitro and In Vivo

T cells engineered with T cell receptors (TCRs) recognizing epitopes derived from intracellular oncogenic drivers, such as mutant KRAS, the most frequently altered gene in human cancers, are likely to induce durable responses in patients with solid tumors. The T cells were engineered with a non-viral targeted knock-in (KI) at the TCRα constant chain (TRAC) locus to express a multi-cistronic cassette that includes 1) a high-affinity TCR specific for the KRAS G12D mutation, 2) a CD8αβ coreceptor, and 3) a chimeric cytokine receptor. The engineered T cells demonstrated cytotoxicity against endogenously expressing HLA-A*11:01+/KRAS G12D+ cell lines in vitro, and mediated robust anti-tumor activity in vivo. Engineered cells also demonstrated a favorable safety profile for the KRAS G12D specific TCR and gene editing reagents. This data supports the planned clinical development of these engineered T cells as a novel non-viral Knock In (KI) TCR-engineered T cell therapy for KRAS-mutant solid tumors.

Autologous CD4+ and CD8+ T cells were engineered using a type V CRISPR-Cas system (Goltsman et al., Novel Type V-A CRISPR effectors are active nucleases with expanded targeting capabilities, CRISPR J., 2020; Lamothe et al., Novel CRISPR-associated gene-editing systems discovered in metagenomic samples enable efficient & specific genome engineering. CRISPR J. 2023) to knock-out the endogenous TRAC locus and simultaneously knock-in (KI) (Schober et al., Orthotopic replacement of T-cell receptor α- and β-chains with preservation of near-physiological T-cell function, Nature Biomed. Engg., 2019) non-virally delivered transgenes within the TRAC locus. These cells (G12D TCR-T cells) were engineered to express: i) a high avidity HLA-A*11:01-restricted TCR specific for the KRAS G12D mutant peptide; ii) CD8 α/β co-receptor which drives a coordinated CD8/CD4 T cell response by allowing for CD4 stimulation that promotes CD8+ T cell functional persistence; and iii) Interleukin Receptor, ILR, a fusion protein that promotes anti-tumor activity through increased T cell proliferation and survival.

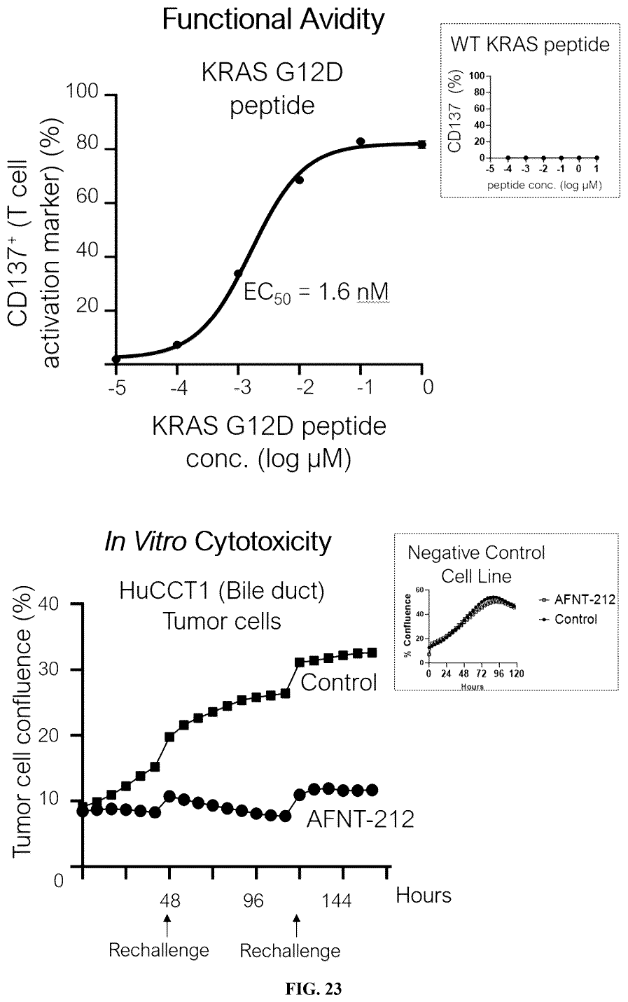

The engineered cells were tested for functional avidity (FIG. 23, left) through binding to the KRAS G12D peptide. The engineered cells specifically bound the KRAS G12D peptide even at sub-nanomolar concentrations, as indicated by T-cell activation using CD137 as an activation marker. The engineered cells were next tested against HuCCT1 (bile duct tumor) cells (FIG. 23, right). The engineered cells showed robust cytotoxicity, even after rechallenge with tumor cells at 48 hours and 120 hours after administration.

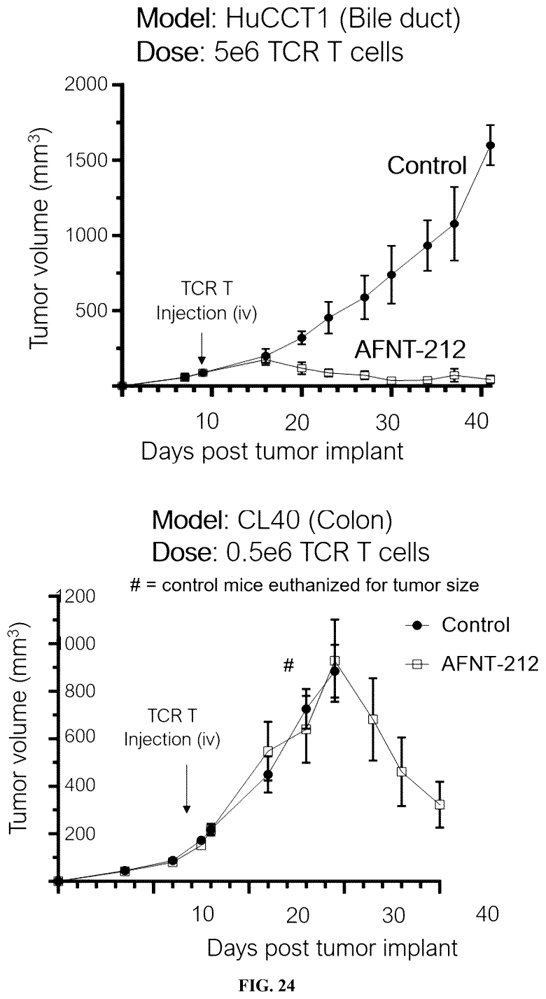

The engineered cells were next intravenously (IV) administered to NOD SCID gamma (NSG) mice that were first subcutaneously inoculated with HuCCT1 tumor cells, a human cholangiocellular carcinoma cell line (FIG. 24, left) or CL40 colorectal cancer tumor cells (FIG. 24, right). The engineered cells demonstrated robust tumor cell control in vivo in response to either tumor cell line.

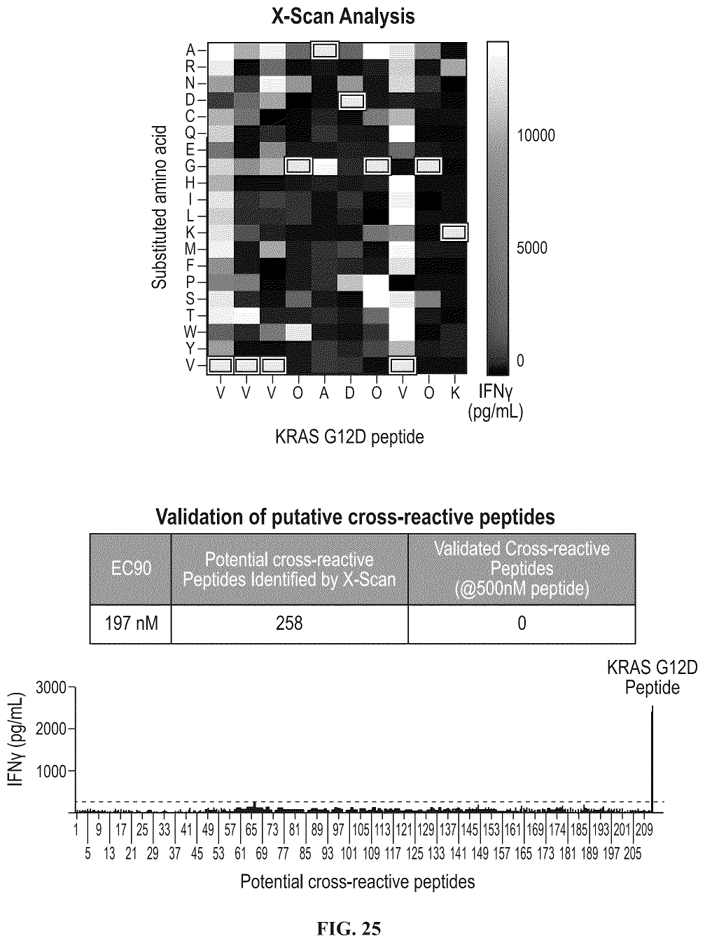

The engineered cells were also incubated with a library of X-scan peptides. Activation of the cells was measured to screen for the recognition motif for the KRAS G12D TCR (FIG. 25). The recognition motif was then referenced against human peptides to identify any potential cross-activity. The engineered cells showed undetectable levels of cross-reactivity, even at 500 nM concentration of peptide, demonstrating that the engineered cells were highly specific for KRAS G12D, with low risk of cross-reactivity to other human peptides in vivo.

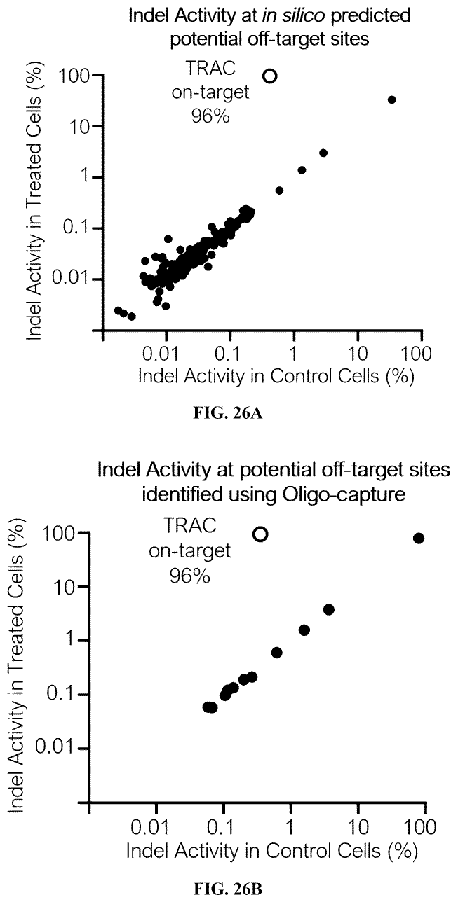

The gene editing reagents used to engineer the cell were further tested for any potential off-target activity. First, potential off-target sites were identified in silico (FIG. 26A) and using oligo-capture analysis in primary T cells (FIG. 26B). For each potential off-target, a targeted sequencing array was used to evaluate the presence of insertions and/or deletions in the engineered cells. The results indicated that the engineered cells showed high on-target activity, with insignificant amounts of off-target activity. This demonstrated that the gene editing reagents were highly specific in producing the engineered cells.

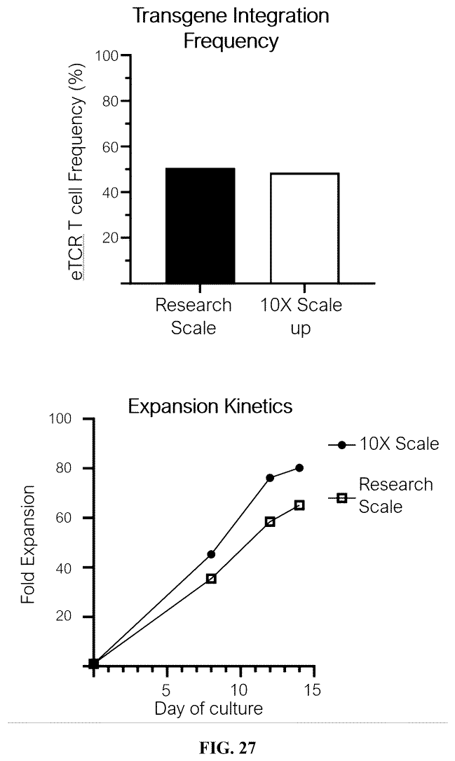

The efficiency of the non-viral knock-in (KI) process was also tested (FIG. 27). The results indicated that greater than 40% of the T cells demonstrated integration of the transgene. This was also tested using small (research) scale and scaled up (10×) sized batches, each of which showed robust expansion kinetics.

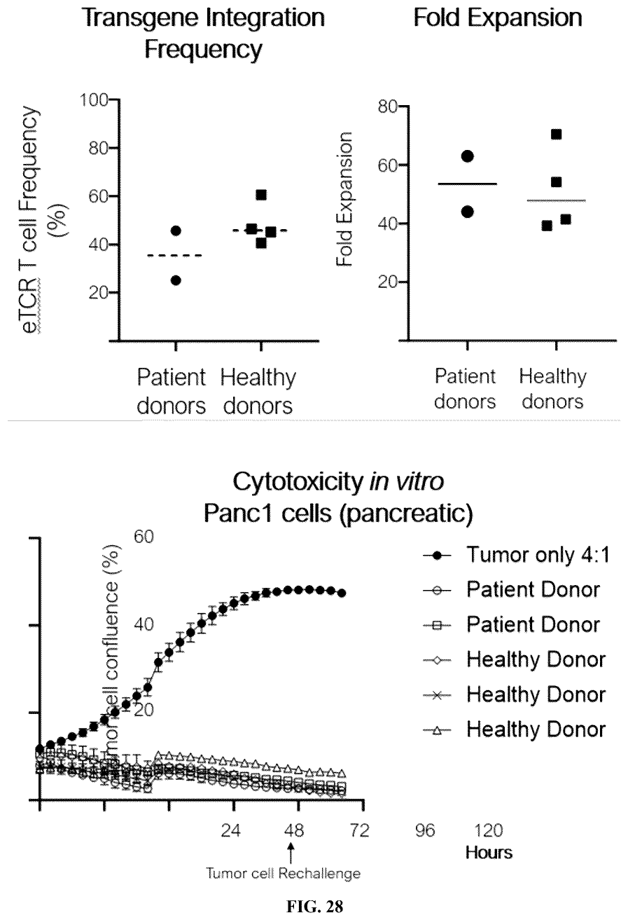

T cells from patient donors were next engineered using the KI process used above. The subsequent engineered patient cells were then tested for KI efficiency, growth kinetics, and functionality (FIG. 28). The results showed that the engineered patient cells performed very similarly to healthy donor T cells.

### Example 14: Non-Viral Targeted Knock-In of a KRAS G12D Specific TCR, CD8αβ, and Chimeric Cytokine Receptor in the TRAC Locus Outperforms Lentiviral-Based Engineering of T Cells

T cells engineered with T cell receptors (TCRs) provide for targeting of stably-expressed oncogenic driver mutations and are likely to induce durable responses in patients with solid tumors. Viral vectors, including lentivirus (LVV), have been a standard modality to deliver transgenes for T cell therapies, but are severely limiting in their manufacturing time, cost, and cargo size. In contrast, non-viral targeted gene knock-in (KI) overcomes these limitations, substantially reducing manufacturing complexity. Here, primary human T cells engineered using LVV or KI processes were compared. The primary human T cells were engineered to express a TCR recognizing a KRAS G12D mutant peptide presented on HLA*A11:01, the CD8α/β coreceptor, and a chimeric interleukin receptor (ILR) using either process. A non-viral manufacturing process was developed and optimized that uses a novel CRISPR-Cas12a system to knock-in the transgene cassette within the TRAC locus and to simultaneously knock out the endogenous TCR1. This non-viral KI platform edits cells with high efficiency and it was shown that KI engineered TCR T cells perform better in functional assays relative to LVV-engineered cells. Together, these data support the utility of a non-viral gene KI approach and its planned incorporation into clinical development.

The engineered T cells for the KI process were engineered using MG29-1 nuclease from Metagenomi, a Type V CRISPR-Cas system (Goltsman, D. S. A. et al. Novel Type V-A CRISPR Effectors Are Active Nucleases with Expanded Targeting Capabilities, CRISPR J., 2020) to knockout endogenous TCRs and drive transgene integration via homologous recombination.

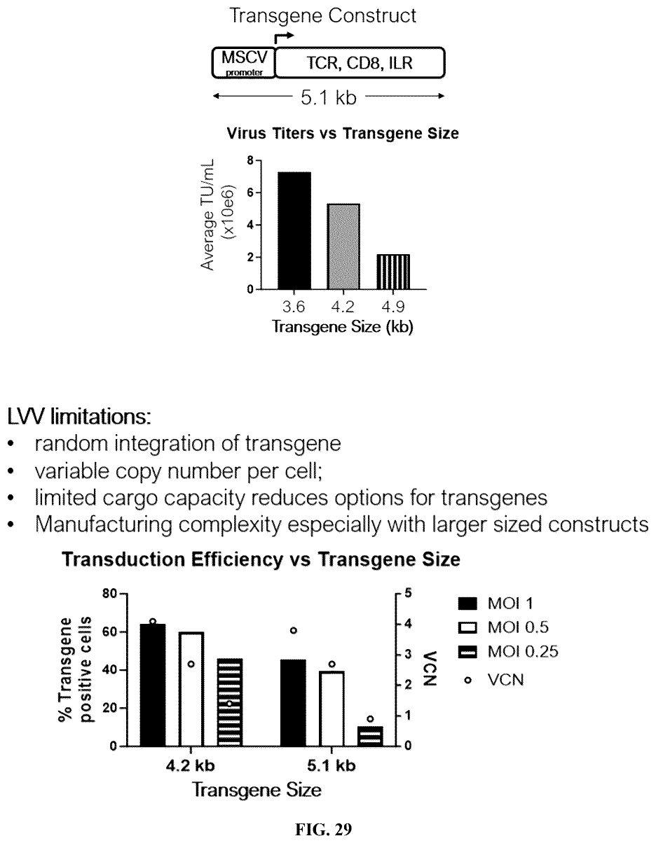

The transgene construct used for the LVV process included a Murine Stem Cell Virus (MSCV) promoter driving expression of a transcript including a TCR recognizing a KRAS G12D mutant peptide presented on HLA*A11:01, a CD8α/β coreceptor, and a chimeric interleukin receptor (ILR) (FIG. 29). First, the impact of size of the transgene construct on transduction efficiency and virus titer was tested for the LVV process. The results (FIG. 29) showed that virus titer decreases with increasing transgene size (left) and that transduction efficiency decreases with increasing transgene size (right). This demonstrated that LVV process efficiency was limited by transgene size.

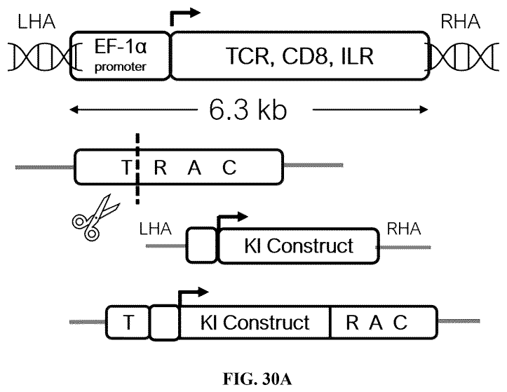

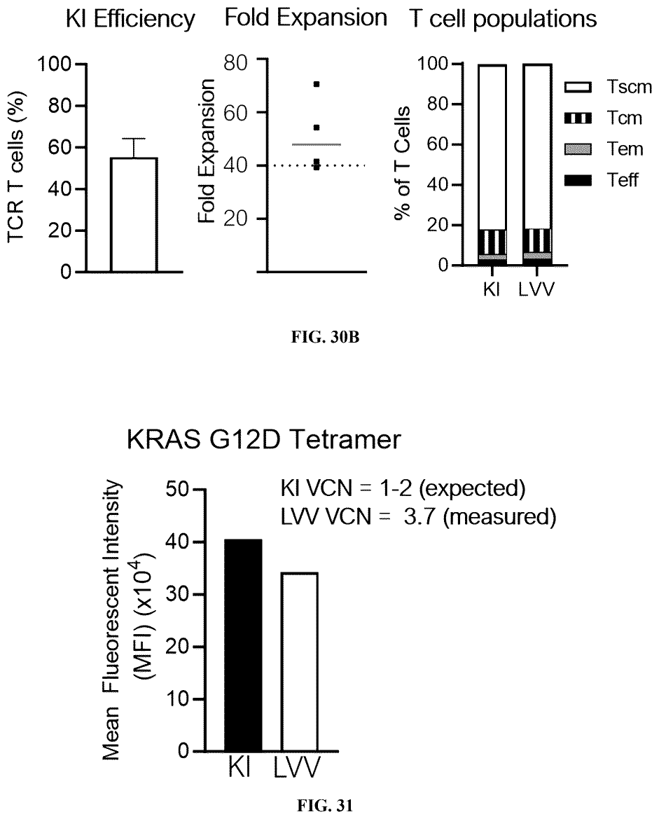

The transgene construct used for the KI process included a elongation factor-1 alpha (EF-1α) promoter driving expression of a transcript including a TCR recognizing a KRAS G12D mutant peptide presented on HLA*A11:01, a CD8α/β coreceptor, and a chimeric interleukin receptor (ILR) (FIG. 30A). The knock-in process was designed to insert the transgene construct into the endogenous TRAC gene via CRISPR/Cas driven homology directed repair. The efficiency of the KI process was then tested (FIG. 30B). The results indicated that this optimized KI process reached over 50% efficiency, while producing a similar T cell population to the LVV process.

Next, the T cells engineered using the KI process were tested for binding to the KRAS G12D tetramer (FIG. 31). The results demonstrated that the KI process showed improved binding to KRAS G12D tetramer as compared to the LVV process. This is despite fewer expected copies of the transgene construct per cell, indicating that the EF-1α promoter was able to drive higher TCR expression.

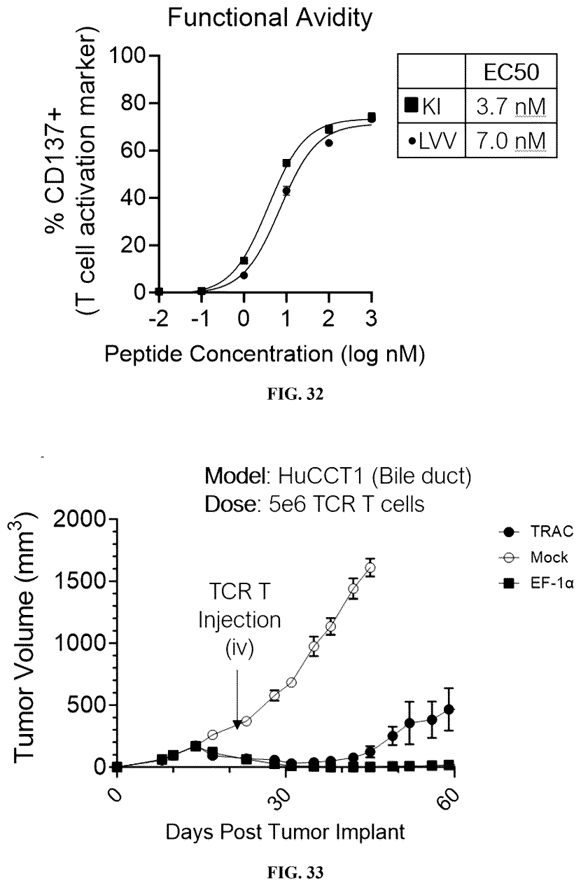

Next, the T cells engineered using the KI process were tested for functional avidity (FIG. 32). The KI process T cells again showed superior results compared to the LVV process T cells, with a higher functional avidity resulting in higher T cell activation, as measured by the T cell activation marker CD137.

The engineered T cells using the KI process were next tested against engineered T cells using the LVV process in vivo in NSG mice. 10 days after the NSG mice were subcutaneously inoculated with HuCCT1 tumor cells, 5 million CD4/CD8 engineered T cells from either the KI process or the LVV process were intravenously administered to the mice (FIG. 33). The results demonstrated that the T cells engineered using the KI process also demonstrated superior tumor control when compared to the T cells engineered using the LVV process.

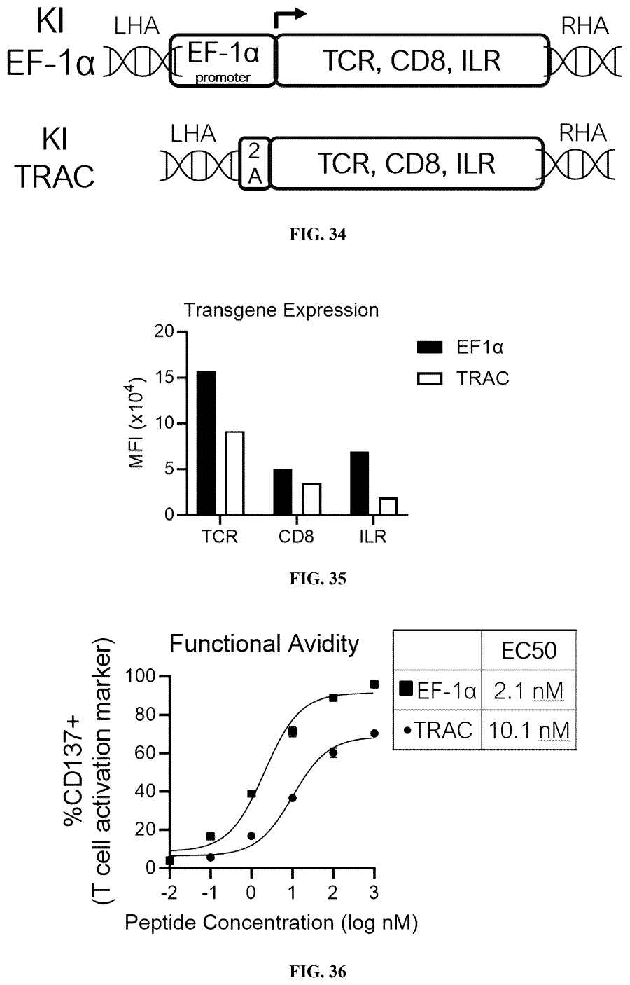

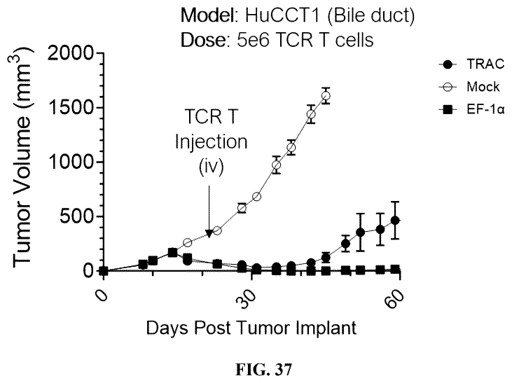

Promoterless transgene constructs for the KI process were also developed, alongside the original construct (FIG. 34). This allows for driving expression of the transgene construct transcript using the endogenous TRAC promoter instead of the EF-1α promoter. This promoterless transgene construct was then tested against the EF-1α transgene construct (FIGS. 35-37). The results demonstrated that using the endogenous TRAC promoter resulted in less transgene expression (FIG. 35), lower functional avidity (FIG. 36), and less in vivo activity (FIG. 37), as compared to the EF-1α promoter.

### Example 15: Phase I Study of Autologous CD8+ and CD4+ Engineered T Cell Receptor T Cells in Subjects with Advanced or Metastatic Solid Tumors

A Phase 1, First-in-Human (FIH), multicenter, open-label study of a KRAS G12D TCR T cell will be conducted. This study will consist of a dose escalation part and a dose expansion part. The study will enroll adult male and female subjects who are HLA-A*11:01-positive with KRAS G12D-positive advanced or metastatic cancers and have progressed on or are intolerant to at least 1 prior line of systemic therapy for the current malignancy.

Approximately 10 study sites for dose escalation and approximately 40 sites for dose expansion are planned; approximately 100 subjects will be enrolled.

Subjects will be followed in the main study for a maximum of 24 months, or until documented disease progression. After post-treatment discontinuation, subjects will be followed for up to fifteen years following the last KRAS G12D TCR T cell infusion. The study will consist of 3 periods (Pre-Treatment, Treatment, and Post Treatment):

The Pre-treatment Period will consist of:
KRAS G12D-HLA Early Screening: Subjects may consent to start early screening to determine KRAS G12D and HLA status. This screening can occur at any time prior to eligibility screening.
Eligibility Screening: Up to 30 days to determine full eligibility for enrollment starting with signing the main consent. Leukapheresis will only proceed upon confirmation of eligibility.
Leukapheresis: Leukapheresis will be performed in eligible subjects followed by a manufacturing period of approximately 28 days. Bridging therapy may be administered during the product manufacturing period.
Baseline evaluation: Assessment prior to lymphodepleting chemotherapy (LDC).

The Treatment Period will consist of:
Lymphodepleting chemotherapy with cyclophosphamide 500 mg/m\hat{ }2 on Day −6 to Day −3 and fludarabine 30 mg/m\hat{ }2 on Day −6 to Day −3. Alternative regimens are permitted.
KRAS G12D TCR T cell Infusion on Day 1
   Subjects may be re-treated if they meet re-treatment criteria.

The Post-treatment Period will consist of:
Post-Treatment Follow-up: To monitor safety and antitumor activity for a maximum of 24 months post KRAS G12D TCR T cell infusion or until disease progression (PD), whichever occurs first. After completion of the Post-treatment Follow-up, at the post treatment follow-up discontinuation, end of study visit, subjects will be consented for the 15-year long-term follow-up study.

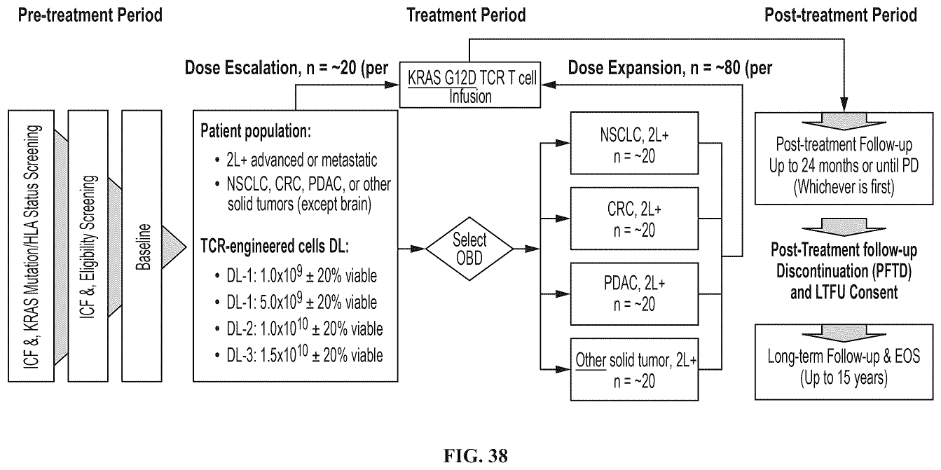

This study will continue until all subjects have discontinued from study participation (including complete withdrawal of consent), died, are lost to follow-up, or have rolled over to a separate Long-Term Follow-up protocol if a separate follow-up protocol becomes available. A schema of the study is shown in FIG. 38.

### Study Duration

It is anticipated that this study will take approximately 18 months to enroll the dose escalation part and approximately 12 months to enroll the dose expansion part. Subjects will have Posttreatment Follow-up (PTFU) for a maximum of 24 months or until documented disease progression. After completing the PTFU Period or documented PD, subjects will transition to LTFU for up to 15 years from the last KRAS G12D TCR T cell infusion. The study objectives and endpoints are shown in TABLE 8.

TABLE 8
Study Objectives and Endpoints

| | | |
|---|---|---|
| Objectives | Endpoint | Description |

Primary

| | | |
|---|---|---|
| Safety and tolerability | Safety | Incidence and severity of treatment- |
| of KRAS G12D TCR T | | emergent adverse events (TEAEs), serious |
| cell | | adverse events (SAEs), and dose-limiting |
| OBD and RP2D of | | toxicities (DLTs) |
| KRAS G12D TCR T | | OBD: Quantify the desirability of a dose in |
| cell | | terms of toxicity-efficacy tradeoff. |
| | | RP2D: Selected based on BOIN12 design |
| | | recommendation and the totality of |
| | | benefit-risk evidence |

Secondary

| | | |
|---|---|---|
| Preliminary antitumor | Overall Response | Percentage of subjects who achieved |
| activity of KRAS G12D | Rate (ORR) | partial response (PR), or complete |
| TCR T cell | | response (CR) as determined by Response |
| | | Evaluation Criteria in Solid Tumors |
| | | (RECIST) v1.1 |
| | Duration of | Time from first documentation of response |
| | Response (DOR) | of PR or better to first documentation of |
| | | disease progression or death from any |
| | | cause, whichever occurs first. |
| | Progression-free | From enrollment to documentation of first |
| | survival (PFS) | disease progression or death from any |
| | | cause, whichever occurs first. |
| | Time to | Time from first KRAS G12D TCR T cell |
| | Response (TTR) | infusion to first documentation of PR or |
| | | better. |
| | Clinical Benefit | Percentage of subjects who have achieved |
| | Rate (CBR) | PR, CR, or had stable disease (SD) for 6 |
| | | months or more. |
| | Overall Survival | From time of enrollment to death from any |
| | (OS) | cause |
| Assess the feasibility of | Manufacturability | Ability to reproducibly generate and infuse |
| reproducibly generating | | T cells at the planned dose level for eligible |
| KRAS G12D TCR T | | participants. |
| cells from autologous | | |
| participant cells. | | |

Exploratory

| | | |
|---|---|---|
| Pharmacokinetics (PK) | Pharmacology | Expansion and persistence of KRAS G12D |
| of KRAS G12D TCR T | | TCR T cells in the peripheral blood over |
| cells | | time |
| Cytokine kinetics | Pharmacology | Concentration of cytokines in serum over |
| | | time |
| Concordance study of | Selection assay | Concordance between local laboratory |
| local laboratory tests | concordance | tests with preferred assays for KRAS |
| with preferred selection | | G12D and HLA-A*11:01 status from |
| assay | | tumor and/or blood |
| Tumor | Biomarker | Presence and change in KRAS G12D |
| microenvironment | | mutation frequency in pre- and post- |
| changes and other | | treatment biopsies and/or circulating tumor |
| resistance mechanisms | | DNA (ctDNA) samples |
| | | Major histocompatibility complex (MHC)- |
| | | I expression in tumor biopsies |
| | | Genetic alterations of the MHC-I, MHC-I |
| | | related genes, and other relevant pathways |
| | | T cell clonality and epitope spreading |
| TCR-T cell migration | Biomarker | Infiltration of KRAS G12D TCR T cells |
| into tumor and anti- | | into tumor and changes in the tumor |
| tumor activity | | microenvironment during treatment |
| | | Phenotype of KRAS G12D TCR T cells |
| | | and other immune cells in the final |
| | | product, peripheral blood, and tumor over |
| | | time |
| | | Tumor burden over time by ctDNA |
| Pharmacokinetics and | Biomarker | Assessment of fludarabine and |
| pharmacodynamics of | | cyclophosphamide in serum before, during |
| lymphodepletion | | and after lymphodepletion period |
| | | Assessment of B, T and NK cell |
| | | frequencies in peripheral blood before, |
| | | during and after lymphodepletion period |
| Health Related Quality | Subject Reported | Subject-reported outcomes as measured by |
| of Life (HRQoL) | Outcomes | European Organization for Research and |
| | | Treatment of Cancer Core Quality of Life |
| | | Questionnaire (EORTC-QLQ-C30) and |
| | | Patient Reported Outcome Common |
| | | Terminology Criteria for Adverse Events |
| | | (PRO-CTCAE) |

KRAS G12D TCR T cells Construct:

An autologous KRAS G12D TCR T cell product that targets the KRAS G12D mutation will be used herein. The KRAS G12D TCR T cell product used is an autologous CD4+ and CD8+ TCR T cell therapy that expresses a multi-cistronic cassette consisting of 1) a high-avidity TCR specific for the KRAS G12D mutation, 2) a constitutively active IL-7Rα fused to the CD34 ectodomain, and 3) a CD8α/β coreceptor. The T cells are engineered using a CRISPR-Cas12a system to knock-in (KI) the transgenes within the TRAC locus and simultaneously knock-out (KO) the endogenous TRAC locus. The non-viral delivery to T cells uses a nanoplasmid that encodes the five transgenes (KRAS G12D TCR α and β chains, the constitutively active IL-7Rα, and the CD8 α and β chains).

### Overall Study Design:

This is a Phase 1, first-in-human (FIH), multicenter, open-label study of KRAS G12D TCR T cells consisting of a dose escalation part and a dose expansion part.

The study will enroll adult male and female subjects who are HLA-A*11:01-positive with KRAS-positive advanced or metastatic cancers and have progressed on or are intolerant of at least 1 prior line of systemic therapy for the current malignancy. Approximately 10 study sites for dose escalation and approximately 40 sites for dose expansion are planned; approximately 100 subjects will be enrolled.

### Sample Size Determination

The total number of subjects planned to be enrolled in both arms of the study is approximately 100 and includes the following:
Dose escalation: up to 20 subjects
Dose expansion: up to 20 subjects per cohort across a maximum of 4 cohorts for a total of up to approximately 80 subjects

The sample size for dose escalation is chosen to be consistent with typical Phase 1 studies and calibrated based on simulation to obtain reasonable operating characteristics. The sample size of dose expansion is chosen based on the Bayesian optimal Phase 2 (BOP2) design (Zhou et al., 2017, Stat Med. 36(21):3302-14) with prespecified type I error and power, as described below.

### Dose Finding/Escalation Part

A total sample size of up to 20 subjects will be enrolled in the dose finding/escalation part of the study. Each dose cohort will consist of approximately 2 to 4 subjects, with staggering of at least 28 days between the first and second subjects' KRAS G12D TCR T cell infusion in each cohort where there is a new dose level (DL).

All subjects in a cohort must complete the full dose-limiting toxicity (DLT) Period before the next cohort can enter the Treatment Period.

The BOIN12 study design (Lin et al., 2020, JCO Precis Oncol. 2020; 4) will be employed to find the optimal biological dose (OBD). The BOIN12 design uses utility to quantify the desirability of a dose in terms of toxicity-efficacy tradeoff, and adaptively allocates subjects to the dose that has the highest estimated desirability.

The desirability (ie, the risk-benefit tradeoff) of a dose is quantified using utility. The utility ascribed to each possible efficacy-toxicity outcome is described in TABLE 9, where a higher value indicates a more desirable outcome (0 and 100 present the least and most desirable outcome, respectively). Let u₁, . . . , u₄ denote these utilities. Given a dose j, let p₁, . . . , p₄ denote the corresponding probabilities of observing each of the possible toxicity-efficacy outcomes. Then, the mean utility of dose j is

$${u}_{j} = {p}_{1} {u}_{1} + {p}_{2} {u}_{2} + {p}_{3} {u}_{3} + {p}_{4} {u}_{4} .$$

A higher value of uⱼ indicates a higher desirability of dose j in terms of risk-benefit tradeoff.

TABLE 9
Utility Table for Binary Efficacy and Toxicity Endpoints

| | Efficacy | No Efficacy |
|---|---|---|
| No toxicity | 100 | 40 |
| Toxicity | 60 | 0 |

To safeguard subjects from toxic and/or futile doses, 2 dose acceptability criteria are used by BOIN12 to decide which doses may be used to treat subjects. A dose is deemed admissible and eligible for treating subjects if it satisfies the following safety and efficacy criteria:
Safety: Pr(πT>0.3|data)<0.95
Efficacy: Pr(πE<0.25|data)<0.9
   where πT and πE are the true DLT rate and efficacy rate, respectively. These 2 conditions ensure that the admissible dose cannot be overly toxic or futile. Doses that are not admissible will be eliminated from the study. If a dose is eliminated due to toxicity, all doses higher than that dose will also be eliminated. The objective is to identify the OBD, defined as the dose that is admissible and has the highest desirability. A maximum of 20 subjects will be enrolled with a cohort size of 2 to 4 subjects.

After dose finding is complete, the OBD at the dose that is admissible and that has the highest estimated utility based on the isotonic estimation method described in Lin et al. (2020) will be selected. The recommended phase II dose (RP2D) will be selected based on the design recommendation and the totality of benefit-risk evidence.

### Dose Expansion Part

After the determination of RP2D, additional subjects will be enrolled in dose expansion cohorts to further assess the safety and efficacy of KRAS G12D TCR T cells. The dose expansion part may consist of up to 4 cohorts with up to approximately 20 subjects per cohort. Subjects entering the dose expansion part will be treated at the RP2D determined at the end of the dose escalation part. Cohorts in the dose expansion part will be indication-specific and may include advanced or metastatic non-small cell lung cancer (NSCLC), colorectal cancer (CRC), pancreatic ductal adenocarcinoma (PDAC), and any other tumor type harboring the KRAS G12D mutation in which potential antitumor activity of KRAS G12D TCR T cells is plausible. The BOP2 design (Zhou et al., 2017) will be used to monitor both toxicity and futility.

Futility monitoring will be performed for each of the 4 indication-specific cohorts separately. The Sponsor will monitor the 6-month overall response rate (ORR) using the BOP2 design when approximately 10 subjects are treated. In addition, for subject safety, toxicity will be monitored for every 10 subjects in the combined cohorts, up to 80 subjects.

### Analysis Populations

Data will be presented and analyzed in the following populations:
Screened: All subjects who sign the Eligibility Screening Informed Consent
Enrolled: All subjects who undergo leukapheresis
KRAS G12D TCR T cell Treated: All enrolled subjects who receive KRAS G12D TCR T cells at the target DL
Intent-to-Treat (ITT): All enrolled subjects
Efficacy Evaluable: All subjects in the KRAS G12D TCR T cell Treated population who have at least 1 post-baseline disease response assessment, or discontinuation due to PD, or death prior to the first post-treatment disease response assessment
Safety Analysis: All subjects who received any low-dose chemotherapy (LDC) and/or KRAS G12D TCR T cells
   a. The ITT population will be used for the summary of demographics and baseline characteristics, biomarker and efficacy analyses, and subgroup analyses, including subgroup efficacy and safety analyses based on receipt of bridging chemotherapy will be completed. The primary safety analysis will be based on the Safety Analysis population.
   b. Details for analysis will be prospectively specified in statistical analysis plan (SAP).

### Statistical Analyses

### General Considerations

Where appropriate, summary statistics will be provided (number of non-missing values, mean, median, standard deviation, minimum, and maximum for continuous variables and number and percentage for categorical variables), including demographic and baseline values and safety measures. No formal statistical inferences will be made for safety parameters. No imputation will be used for missing data.

Safety summaries will display results for each part separately and for all subjects combined.

### Primary Endpoints

### Safety

The primary safety endpoint will be assessed via the occurrence of adverse events (AEs), serious adverse events (SAEs), and DLTs. AEs will be coded using the current version of the Medical Dictionary for Regulatory Activities (MedDRA). AE data will be summarized using descriptive statistics.

### Secondary Endpoints:

The secondary objective is the preliminary antitumor response as measured by ORR (partial response (PR)+complete response (CR)) per RECIST v1.1, duration of response (DOR), progression-free survival (PFS), time to response (TTR), clinical benefit rate (CBR), and overall survival (OS). The ORR and its 95% confidence interval (CI) will be estimated.

### Exploratory Endpoints:

Analysis details for exploratory endpoints will be described in the SAP. Contingent on sample availability and ability to derive parameters, PK parameters of KRAS G12D TCR T cells including maximum concentration, time-to-max concentration, AUC, and half-life may be calculated. Exposure-safety and exposure-efficacy analyses may be explored.

Given the small size of the study, correlative biomarker studies are underpowered for formal statistical analyses, and therefore are not anticipated to result in direct conclusions, but instead inform hypotheses for future studies. Where applicable, standard statistical methods will be used to describe the exploratory analyses, considering the sample size at each stage of the study.

### DLT Definitions and Stopping Rules:

### Dose-Limiting Toxicity Monitoring

During the Dose Escalation part of the study, subjects will be monitored for DLTs. DLTs are defined as AEs that occur in the first 28 days after the first KRAS G12D TCR T cell infusion, that are assessed as possibly related or related to KRAS G12D TCR T cells, and that meet the following criteria:
1. Any Grade 5 death that is not related to underlying disease and is at least possibly related to KRAS G12D TCR T cells.
2. Any treatment-emergent Grade 4 or 5 CRS
3. Any treatment-emergent Grade 3 CRS that does not resolve to Grade 2 within 7 days
4. Grade 3 or higher neurotoxicity that does not resolve to Grade 2 within 72 hours
5. Grade 3 or higher Immune Effector Cell-Associated Hemophagocytic Lymphohistiocytosis-Like Syndrome (IEC-HS)
6. Grade 3 or greater allergic reaction related to KRAS G12D TCR T cell infusion
7. Any treatment-emergent autoimmune toxicity Grade 3 or higher
8. Grade 3 or higher organ toxicity (cardiac, dermatologic, gastrointestinal, hepatic, pulmonary, renal/genitourinary), not pre-existing or not due to the underlying malignancy occurring within 30 days of cell infusion.
   a. Any Grade 3 or higher KRAS G12D TCR T cell-related non-hematologic toxicity that does not resolve to Grade 2 or less within 7 days
9. Grade 3 thrombocytopenia with bleeding
10. Grade 3 or higher hematologic toxicities that fail to recover to Grade 2 or lower within 7 days and are not due to administration of LDC
11. Any other clinically significant toxicity related to KRAS G12D TCR T cells not meeting the above criteria that is deemed by the Investigator to represent a DLT.
   The following AEs are not considered DLTs:
12. Grade 3 fatigue
13. Grade 3 endocrine disorder (thyroid, pituitary, and/or adrenal insufficiency) that is managed with or without systemic corticosteroids and/or hormonal replacement therapy with resolution of symptoms
14. Grade 3 hypertension that can be controlled with medical therapy
15. Grade 3 laboratory value abnormalities that are transient, asymptomatic, and clinically insignificant
16. Vitiligo or alopecia of any AE grade

Grading of DLTs will be performed in accordance with NCI-CTCAE version 5.0. For ICANS and CRS, the American Society for Transplantation and Cellular Therapy Consensus Grading will be used (Lee et al., 2019). If applicable, the DLT assessment period will be extended to follow ongoing TEAEs until resolution of the event or confirmation that the event is a DLT.

### Stopping Rules:

A Safety Monitoring Committee (SMC) will monitor cumulative adverse events (AEs) of all subjects and suspend the study if there are recurrent, clinically significant, or delayed-onset, treatment-related toxicities. The SMC will assess whether this study should be discontinued based upon excessive toxicity as described in the BOIN12 clinical study design under dose acceptability criteria. The study will be terminated if no dose level meets the safety acceptability. During dose expansion, indication specific stopping rules concerning futility will be applied.

### Study Stopping Criteria:

The SMC will assess whether this study should be discontinued based upon excessive toxicity. The study will be terminated if no dose level meets the safety acceptability criteria. The SMC will meet regularly as required.

The study may be paused or stopped if any subject experiences any of the following SAEs after KRAS G12D TCR T cell infusion:
A death occurring within 30 days from administration of the investigational product unless clearly due to disease progression.
Death within 30 days of infusion that is not related to underlying disease and is at least possibly related to KRAS G12D TCR T cells.
Occurrence of 2 Grade 4 or greater SAE in 2 study subjects
Over 33% of study subjects experience a Grade 3 regardless of the phase of the study.
Any Grade 4 hypersensitivity reaction/anaphylaxis; or Infection related to a positive sterility test result.
Life-threatening (Grade 4) toxicity attributable to KRAS G12D TCR T cells that is unexpected, unmanageable (ie, does not resolve to Grade 3 or lower within 10 days), and unrelated to bridging therapy between leukapheresis and LDC.

If paused, the study will be halted until an appropriate evaluation of the cause of the toxicity is determined and a plan of correction, if necessary, is established. In addition, the study may be terminated for the following reasons:
Any subject develops uncontrolled KRAS G12D TCR T cell proliferation leading to malignancy
Observation of an unexpected, significant risk to subjects
Failure to enroll subjects at an acceptable rate
Plans to modify, suspend, or discontinue the development of KRAS G12D TCR T cells
Sponsor, the IRB/IEC, or SMC decides that subject safety may be compromised by continuing the study

### Inclusion Criteria/Exclusion Criteria

### KRAS-HLA Screening Inclusion Criteria

Subjects must meet all of the following inclusion criteria to be eligible to participate in the KRAS-HLA Screening:
1. At least 18 years of age
2. Capable of understanding and willing to provide a written informed consent
3. Able and willing to comply with the protocol requirements
4. Histologically confirmed advanced or metastatic, unresectable solid tumor
5. Previously determined KRAS G12D mutational status and HLA-A*11:01 allele or willingness to provide samples for KRAS and HLA-A*11:01 analyses

### Study Eligibility Screening Inclusion Criteria

All of the following inclusion criteria must be met prior to leukapheresis:
1. At least 18 years of age
2. Capable of understanding and willing to provide a written informed consent
3. Able and willing to comply with the protocol requirements, including the availability of a dedicated caregiver.
4. Histologically confirmed advanced or metastatic, unresectable solid tumor
5. Positive for KRAS G12D mutation
6. Subject has HLA-A*11:01 allele
7. Progressed on or intolerant of at least 1 prior line of standard systemic therapy for the current malignancy. Subjects with tumors that have known actionable molecular alterations must have progressed on directed molecular therapy:
   For CRC: Subjects harboring genomic aberrations including but not limited to BRAFV600E mutations and HER2 amplifications for which FDA-approved targeted therapies are available must have received prior treatment with applicable FDA approved targeted therapies. Subjects whose tumors have deficient mismatch repair (dMMR)/high microsatellite instability (MSI-H) must have received an immune checkpoint inhibitor prior to enrolling in this study.
   For NSCLC: Subjects harboring genomic aberrations for which FDA-approved targeted therapies are available must have received prior treatment with the applicable FDA-approved targeted therapies. Subjects for whom such treatment is appropriate must have received treatment with an FDA-approved checkpoint inhibitor with or without chemotherapy consistent with the FDA-approved label.
   Any other solid tumors, including PDAC: Subjects harboring genomic aberrations for which FDA-approved targeted therapies are available must have received prior treatment with the applicable FDA-approved targeted therapies. Subjects whose tumors have dMMR/MSI-H must have received an immune checkpoint inhibitor prior to enrolling in this study.
8. Measurable disease per RECISTv1.1. Note: a previously irradiated or locoregionally treated lesion can be considered a target lesion if it progressed post treatment.
9. Eastern Cooperative Oncology Group (ECOG) performance status of 0 or 1
10. Adequate organ and bone marrow function based on the following laboratory values:
Absolute neutrophil count (ANC)≥1000/mm3 without granulocyte colony stimulating factor support (filgrastim within 7 days or peg-filgrastim within 14 days of screening lab used for subject eligibility)
Absolute lymphocyte count (ALC)≥200/mm³
Platelets≥75,000/mm³ without transfusion within the preceding 7 days
Hemoglobin≥8.0 g/dL (≥80 g/L); blood transfusion permitted without transfusion within the preceding 7 days
Alanine aminotransferase (ALT) and aspartate aminotransferase (AST)≤3×the upper limit of normal (ULN)
ALT and AST≤5×ULN if liver metastasis present
Total bilirubin≤1.5×ULN or ≤3×ULN in the presence of documented Gilbert's Syndrome
Albumin≥2.5 g/dL (≥25 g/L)
Prothrombin time/international normalized ratio (INR) or partial thromboplastin time (PTT) test<1.5×ULN; abnormal values are permitted if subject is on low dose low molecular weight heparin
Creatinine clearance≥50 mL/min by Chronic Kidney Disease Epidemiology Collaboration (CKD-EPI) or 24-hour urine clearance

Note: Eligibility laboratory test results, including test results from standard of care testing may be used for study enrollment if within 7 days. Eligibility labs may be used to proceed to leukapheresis if tests were performed within 7 days of leukapheresis.

Able and willing to abide by the contraception guidelines and local regulations regarding contraceptive use.

Agree to abstain from breastfeeding during study participation.

Refrain from tissue donation, including ova/sperm donation or any other tissue/blood/organ donations, for at least 1 year following the last KRAS G12D TCR T cell infusion.

### LDC Inclusion Criteria

All of the following criteria must be met at the study Baseline visit within 72 hours prior to LDC:
ECOG performance status of 0 or 1
Adequate organ and marrow function, based on the following criteria within 72 hours prior to planned LDC:
   ANC≥1000/mm³
   ALT and AST≤3×ULN
      ALT and AST≤5×ULN if liver metastasis present
   Total bilirubin≤1.5×ULN or ≤3×ULN in the presence of documented Gilbert's Syndrome
   Creatinine clearance≥50 mL/min by CKD-EPI or 24-hour urine clearance
Subjects of childbearing potential must have a negative serum or urine pregnancy test within 72 hours prior to LDC.
If applicable, toxicities due to bridging must have resolved to grade 1 or baseline prior to LDC.
If subjects receive immunotherapy during bridging therapy, prior progression on a PD-1 or PD-L1 checkpoint inhibitor should be unequivocal; progression that occurs within the first 8 weeks of treatment on these agents should be confirmed with a second CT at least 4 weeks apart.

### KRAS G12D TCR T Cell Initial and Retreatment Infusion Eligibility

Prior to infusion of KRAS G12D TCR T cells, the subject will undergo clinical evaluation and determination of suitability to proceed with the administration of AFNT. All of the following inclusion criteria must be met prior to KRAS G12D TCR T cell infusion:
ECOG performance status 0 or 1
Adequate organ and marrow function, based on the following criteria:
   ALT, AST≤3×ULN
      ALT and AST≤5×ULN if liver metastasis present
   Total bilirubin≤1.5×ULN or ≤3×ULN in the presence of documented Gilbert's Syndrome
Creatinine clearance≥50 mL/min by CKD-EPI or 24-hour urine clearance
   Note: For re-treatment, subjects must have tolerated the first infusion without DLTs

### Eligibility Exclusion Criteria

### Study Eligibility Exclusion Criteria

Any subject who meets any of the following criteria will be excluded from study participation:
Any systemic cytotoxic chemotherapy, investigational agents, or any antitumor drug from a previous treatment regimen or clinical study (including small molecules and I/O compounds) within 5 half-lives or 14 days of screening, whichever is shorter.
Any prior gene therapy utilizing an integrating vector
Previous allogeneic stem cell transplantation or prior organ transplantation
History of treated primary immunodeficiency, autoimmune, or inflammatory disease including inflammatory bowel disease, systemic lupus erythematosus, rheumatoid arthritis, myasthenia gravis, or Graves' disease
   The following autoimmune conditions are permitted: vitiligo, alopecia, hypothyroidism on stable hormone replacement therapy, stable adrenal insufficiency with or without low dose prednisone, psoriasis/eczema not requiring systemic treatment, or any other condition deemed to be clinically insignificant
Use of prohibited steroids or immunosuppressive drugs within 14 days prior to screening
Primary brain tumor
Untreated central nervous system (CNS) metastatic disease, leptomeningeal disease, or cord compression. Subjects previously treated for CNS metastases that are radiographically and neurologically stable and off steroids for at least 4 weeks prior to enrollment are eligible.
Surgery or catheter-based interventions such as transarterial chemoembolization or percutaneous coronary intervention within 4 weeks prior to screening
Uncontrolled significant intercurrent or recent illness including, but not limited to the following conditions:
   Subjects with clinically significant pulmonary dysfunction, as determined by medical history and physical exam should undergo pulmonary function testing. Subjects with a forced expiratory volume in 1 second (FEV1)<55% or diffusing capacity of the lungs for carbon monoxide (DLco)<40% will be excluded.
   Significant cardiovascular abnormalities as defined by any one of the following: uncontrolled congestive heart failure or hypertension, clinically significant hypotension, symptomatic coronary artery disease, or a documented ejection fraction (EF) of <45% as assessed by echocardiogram or multigated acquisition scan (MUGA). Any subject with an EF of 45-49% must receive clearance by a cardiologist to be eligible for the study.
Uncontrolled active bacterial, viral, fungal, or mycobacterial infection not responding to antibiotics, antimycotics, or antifungal agents, as well as long-term oral treatment with any of these agents
Pregnant or lactating subjects
Previously identified allergy, hypersensitivity, or known contraindication to cyclophosphamide, fludarabine, or any other agent associated with LDC or AFNT-212
Diagnosis of another malignancy within 2 years prior to screening
   The following diagnoses are permitted: superficial non-melanoma skin cancers, clinically localized, low-risk prostate cancer defined as T1 to T2a, a tumor in histologic grade group 1 (Gleason score≤6) on biopsy and a serum PSA<10 ng/ml, cervical carcinoma in-situ, resected breast lobular or ductal carcinoma in-situ deemed cured and not treated with systemic therapy; subjects with other early-stage cancers that have been resected with low risk of recurrence may be eligible after documented discussion with the Medical Monitor
Seropositive for hepatitis B surface antigen (HBsAg) and/or hepatitis B core antibody (HBcAb)
Seropositive for hepatitis C antibody. Subjects with a positive hepatitis C antibody test may be eligible if hepatitis C virus (HCV) RNA is undetectable on a quantitative HCV RNA assay, following discussion with the Medical Monitor.
Known human immunodeficiency virus (HIV) infection
Psychiatric illness or other comorbidities that will affect compliance with study procedures, as determined by the Investigator

### Lymphodepleting Chemotherapy Exclusion Criteria

Any subject who meets any of the following criteria will be excluded from LDC. Where applicable, LDC may be delayed, and the Investigator is to contact the medical monitor:
Radiotherapy with a wide field of radiation or chemoradiation within 4 weeks or radiotherapy with a limited field of radiation for palliation within 2 weeks of LDC
Systemic antitumor therapy prior to LDC
   Any cytotoxic chemotherapy, investigational agents, or any antitumor drug from a previous treatment regimen or clinical study (including small molecules and I/O agents) within 14 days
   Nitrosourea or mitomycin C within 6 weeks of LDC
Use of steroids or immunosuppressive drugs within the last 14 days prior to LDC
   The following treatments are permitted: intranasal, inhaled, topical, or local steroid applications; systemic corticosteroids at doses equivalent to no more than 10 mg/day prednisone; steroids as premedication for contrast dye allergy
Uncontrolled significant intercurrent or recent illness or active infection not responding to treatment with antibiotics, antimycotics, or antiviral agents
Live attenuated vaccine within 30 days prior to LDC

### Leukapheresis and Post LDC and Infusion Procedures

### Leukapheresis

Following the eligibility screening assessments, a leukapheresis collection will be performed for eligible subjects. Should a technical issue arise during the procedure or processing of the leukapheresis product, the subject may have a second collection performed. Subjects must continue to meet eligibility requirements for repeat leukapheresis.

Subjects that are ineligible for vein-to-vein apheresis may elect to have a percutaneous central venous access catheter inserted to support this collection.

If necessary, anticancer bridging therapy is allowed for disease control after leukapheresis and while KRAS G12D TCR T cells are being manufactured.

Post-LDC and KRAS G12D TCR T cell Infusion Requirements

Hospitalization of study subjects after LDC or KRAS G12D TCR T cell infusion is not mandatory under this protocol. The decision to refer study subjects to inpatient service for observation after infusion not related to the management of acute adverse events (AEs) is left to the Investigator's discretion and shall be made in accordance with institutional guidelines and processes.

Study subjects should remain within a 2-hour transportation ride to the study center for 28 days following KRAS G12D TCR T cell infusion so they can be seen at the study center in the event of toxicity. Subjects may alternatively utilize accommodations per institutional practice.

### Dedicated Caregiver

All subjects must have a dedicated caregiver for 28 days following KRAS G12D TCR T cell infusion. The caregiver will support the subject in performing at-home ICE assessments and temperature measurements.

### Lifestyle Considerations

Subjects will be given a Study Wallet Card with emergency contact information for the Investigator and/or study staff and will be instructed to keep this with them at all times.

### Study Treatments

TABLE 10
Lymphodepleting Chemotherapy

| | Lymphodepleting Chemotherapy | |
|---|---|---|
| Name | Cyclophosphamide | Fludarabine |
| Dose levels | 500 mg/m²/day | 30 mg/m²/day |
| Route of | IV infusion | IV infusion |
| administration | | |
| IMP or NIMP | NIMP | NIMP |
| Labeling | Cyclophosphamide will be | Fludarabine will be provided by |
| | provided by the study site; | the study site; labeling will be |
| | labeling will be per site policy. | per site policy. |

IMP = investigational medicinal product;
LDC = lymphodepleting chemotherapy;
IV = intravenous;
NIMP = non investigational medicinal product

Upon notification from the Sponsor that KRAS G12D TCR T cells will be available, LDC should be planned for initiation 6 days prior to planned infusion date. KRAS G12D TCR T cells must be on site before LDC begins. LDC treatment details are shown in TABLE 10. KRAS G12D TCR T cell infusion details are shown in TABLE 11. Both LDC and KRAS G12D TCR T cell infusions will be performed either in an outpatient or inpatient setting at the investigational site by qualified personnel, in accordance with institutional procedures. If the Investigator believes it is in the best interest of the subject to begin LDC prior to KRAS GD2D TCR T cell arrival on-site and has been notified that KRAS G12D TCR T cells will be available in a timely manner, the Investigator should contact the Sponsor to discuss rationale and obtain approval.

If subjects either received bridging therapy or were imaged>28 days prior to the planned Day 1 KRAS G12D TCR T cell infusion, baseline imaging assessments must be repeated (following completion of bridging therapy, if applicable) and before the start of LDC.

Subjects will receive LDC from Day −6 to Day −3 (4 days). However, if a subject is deemed by the investigator to be adequately lymphodepleted (absolute lymphocyte count 0 to 15 uL) on the third day of LDC, the fourth day may be omitted.

TABLE 11
KRAS G12D TCR T cell Infusion

| Name | KRAS G12D TCR T cell Dose Levels |
|---|---|
| Dose levels | DL-1: 1.0 × 10⁹ ± 20% viable Dextramer positive (transgenic) |
| | KRAS G12D TCR T cells |
| | DL1: 5.0 × 10⁹ ± 20% viable transgenic KRAS G12D TCR T |
| | cells |
| | DL2: 1.0 × 10¹⁰ ± 20% viable transgenic KRAS G12D TCR T |
| | cells |
| | DL3: 1.5 × 10¹⁰ ± 20% viable transgenic KRAS G12D TCR T |
| | cells |
| Route of | IV infusion |
| administration | |
| IMP or NIMP | IMP |
| Labeling | KRAS G12D TCR T cells will be provided in cryopreserved, |
| | single-use, ready-to-infuse bags with overwrap as secondary |
| | containment within an aluminum cassette for additional |
| | protection. Each external cassette and drug product bag will be |
| | affixed with a label containing the clinical study protocol number, |
| | the subject's study number, and other identifying information, if |
| | required to meet site- and/or region-specific requirements (eg, |
| | initials, birthdate). |

IMP = investigational medicinal product

Subjects must complete LDC before KRAS G12D TCR T cell infusion. Enrolled subjects will not start study treatment until the KRAS G12D TCR T cell product is manufactured, tested, authorized for release, and received at the clinical site.

If the assigned target dose range cannot be met, subjects may receive the manufactured KRAS G12D TCR T cell IMP at the investigator's discretion and after documented benefit-risk discussion with the Medical Monitor. Safety and toxicity data will be collected. However, subjects will not be DLT-evaluable, not count towards the assigned dose cohort, and need to be replaced.

At the investigator's discretion, KRAS G12D TCR T cells may be given more than 2 days after completion of LDC. However, if infusion delay will be greater than 7 days following LDC, this should be discussed with the Medical Monitor. LDC may be repeated if there is significant delay of the KRAS G12D TCR T cell infusion (e.g., beyond 2 months).

KRAS G12D TCR T cell infusion should be delayed if the subject presents with any of the following on the planned infusion day:
KRAS G12D TCR T cell infusion eligibility is not met
Suspected or active systemic infection (subjects with suspected/active infection must have negative culture for at least 24 hours on appropriate antibiotics, or negative rapid viral panel)
Hypotension requiring vasopressor support
Requirement for supplemental oxygen to keep saturation greater than 91%

### Retreatment Criteria

Subjects who received KRAS G12D TCR T cells and had a PR may receive a second infusion of KRAS G12D TCR T cells, at the Investigator's discretion. Subjects who achieved a transient CR and later progressed within the PTFU Period may also be considered for retreatment. Retreatment must be supported by benefit-risk assessment and justified by efficacy and toxicity data as well as biological activity identified with the prior administration. Subjects may be retreated following patient status review and discussion with the Medical Monitor.

To qualify for re-treatment, subjects must meet all the criteria defined below:
At least 8 weeks since first infusion
Confirmed disease progression (Expansion cohorts only)
ECOG performance status of 0 or 1
Have not received any subsequent anticancer drug since AFNT-212 infusion
Adequate organ and bone marrow function:

$$ANC \geq 1000 / {mm}^{3}$$

$$ALT , AST \leq 3 \times ULN$$

ALT and AST≤5×ULN if liver metastasis present
Total bilirubin≤1.5×ULN or ≤3×ULN in the presence of documented Gilbert's Syndrome
Creatinine clearance≥50 mL/min by CKD-EPI or 24-hour urine clearance
Subjects of childbearing potential must have a negative serum or urine pregnancy test within 72 days prior to LDC

In addition, subjects must have tolerated the initial infusion of KRAS G12D TCR T cells without occurrence of any DLTs. Retreatment must be administered at the same dose level as the first infusion of KRAS G12D TCR T cells. In cases of limited cell product availability for retreatment, a lower dose can be considered after discussion with the Medical Monitor. No LDC shall be administered if re-treatment occurs within 2 months of the first infusion of KRAS G12D TCR T cells. If retreatment occurs more than 2 months after the first infusion of KRAS G12D TCR T cells, the decision to administer LDC is at the investigator's discretion; however, subjects must meet the LDC eligibility criteria. The same LDC regimen used prior to the first KRAS G12D TCR T cell infusion should be used prior to retreatment.

AEs following re-treatment with KRAS G12D TCR T cells will be collected and reported but will not be used in the DLT analysis.

KRAS G12D TCR T cell Infusion Timing for Dose Escalation Cohorts

During the dose escalation part of the study, up to 4 dose levels of KRAS G12D TCR T cells may be evaluated.

For each dose escalation cohort, KRAS G12D TCR T cells will be administered with a 28-day stagger between the first and second subjects' dosing for each new dose level to allow monitoring for events.

### Anticancer Bridging Therapy

Experimental treatments must not be used for bridging therapy. If bridging therapy is used, it must be completed at least 14 days before LDC. Subjects may not receive bridging therapy for retreatment with KRAS G12D TCR T cells.

### Required Concomitant Medications and Procedures

At the time of LDC initiation or following LDC, anti-infectious prophylaxis may be started at the Investigator's discretion and in accordance with the recommendation of the respective professional societies and local institutional guidelines.

In some cases, tocilizumab, or another anti-interleukin (IL)-6 therapy, may be required to treat toxicities such as CRS. Anti-IL-6 therapy must be available at site prior to subject infusion.

Please refer to the currently approved Prescribing Information. Additionally, for ICANS and CRS, glucocorticoids such as dexamethasone or methylprednisolone may be utilized.

Cytopenias may be managed with growth colony stimulating factors, packed red blood cells (PRBC), and platelet transfusions per institutional guidelines and/or per PI's discretion. Leukocyte filters are encouraged for PRBC transfusions.

Fevers in the presence of neutropenia should be managed according to local institutional guidelines.

A schedule of study activities and procedures in shown in TABLE 12. A schedule of biomarker and pharmacokinetic activities is shown in TABLE 13.

TABLE 12
Schedule of Study Activities and Procedures

| Study Periods | | | | | | | | | | | | |
|---|---|---|---|---|---|---|---|---|---|---|---|---|
| Pretreatment | | | | Treatment | | | | | Post-Treatment | | | |
| Study Day/Month | | | | | | | | | | | | |
| | | | | | | | | | Safety | | | |
| KRAS- | | | | Lymphodepleting | | | | KRAS G12D | Monitoring/DLT^{s} | | | |
| HLA | Eligibility | Enrolment/ | Base- | Chemotherapy | | | | TCR T cells | Month 1 | | | |
| Screening | Screening^{a} | Leukapheresis | line^{b} | D-6 | D-5 | D-4 | D-3 | D 1 | D 3 | D 8 | D 10 | D 15 |

Visit Window (Days)

±3

Assessments
GENERAL STUDY ASSESSMENTS

| | | | | | | | | | | | | | |
|---|---|---|---|---|---|---|---|---|---|---|---|---|---|
| Informed | X | X | — | — | — | — | — | — | — | — | — | — | — |
| consent^{a} | | | | | | | | | | | | | |
| KRAS and HLA | X | — | — | — | — | — | — | — | — | — | — | — | — |
| status | | | | | | | | | | | | | |
| testing^{c} | | | | | | | | | | | | | |
| I/E | X | X | — | — | — | — | — | — | — | — | — | — | — |
| criteria^{d} | | | | | | | | | | | | | |
| Demographics | — | X | — | — | — | — | — | — | — | — | — | — | — |
| Medical | X | X | — | — | — | — | — | — | — | — | — | — | — |
| history | | | | | | | | | | | | | |
| Prior | — | X | — | — | — | — | — | — | — | — | — | — | — |
| disease | | | | | | | | | | | | | |
| history/ | | | | | | | | | | | | | |
| therapies | | | | | | | | | | | | | |
| Concomitant | X | X | X | X | X | X | X | X | X | X | X | X | X |
| medications/ | | | | | | | | | | | | | |
| procedures | | | | | | | | | | | | | |
| HRQoL | — | — | — | X | — | — | — | — | X | | X | — | X |
| Mutagenic | — | — | — | — | — | — | — | — | — | — | — | — | — |
| agent | | | | | | | | | | | | | |
| exposure | | | | | | | | | | | | | |
| assessment | | | | | | | | | | | | | |

SAFETY ASSESSMENTS

| | | | | | | | | | | | | | |
|---|---|---|---|---|---|---|---|---|---|---|---|---|---|
| AE assessments | X | X | X | X | X | X | X | X | X | X | X | X | X |
| Temperature | — | — | — | — | — | — | — | — | X | X | X | X | X |
| monitoring^{e} | | | | | | | | | | | | | |
| ICE^{f} | | — | — | X | — | — | — | — | X | X | X | X | X |
| Vital | — | X | — | X | — | — | — | — | X^{d} | X | X | X | X |
| signs^{g} | | | | | | | | | | | | | |
| O₂ saturation | — | X | X | — | — | — | — | — | X^{d} | X | X | X | X |
| test^{g} | | | | | | | | | | | | | |
| Targeted | — | — | X | — | — | — | — | — | X | X | X | X | X |
| physical | | | | | | | | | | | | | |
| examination | | | | | | | | | | | | | |
| Complete | — | X | — | X | — | — | — | — | — | — | — | — | — |
| physical | | | | | | | | | | | | | |
| exam, | | | | | | | | | | | | | |
| height | | | | | | | | | | | | | |
| Weight | — | X | X | X | — | — | — | — | X | — | — | — | — |
| Pulmonary | — | X | — | X | — | — | — | — | — | — | — | — | — |
| function^{h} | | | | | | | | | | | | | |
| 12-lead ECG | — | X | — | X | — | — | — | — | — | — | — | — | — |
| MUGA or | — | X | — | X^{h} | — | — | — | — | — | — | — | — | — |
| ECHO^{h} | | | | | | | | | | | | | |

SAFETY LABORATORY ASSESSMENTS

| | | | | | | | | | | | | | |
|---|---|---|---|---|---|---|---|---|---|---|---|---|---|
| Serum/urine | — | X | — | X | — | — | — | — | — | — | — | — | — |
| pregnancy | | | | | | | | | | | | | |
| testⁱ | | | | | | | | | | | | | |
| Chemistry | — | X | — | X | — | — | — | — | X | X | X | X | X |
| panel^{j} | | | | | | | | | | | | | |
| Hematology | — | X | — | X | — | — | — | — | X | X | X | X | X |
| panel^{j} | | | | | | | | | | | | | |
| Coagulation | — | X | — | X | — | — | — | — | X | X | X | X | X |
| panel | | | | | | | | | | | | | |
| TLS/CRS | — | — | — | — | — | — | — | — | X | X | X | X | X |
| panel | | | | | | | | | | | | | |
| Tumor | — | — | — | X | — | — | — | — | — | — | — | — | — |
| Markers^{k} | | | | | | | | | | | | | |
| Inflammatory | — | — | — | X | — | — | — | — | X | X | X | X | X |
| markers^{l} | | | | | | | | | | | | | |
| Urinalysis | — | X | — | — | — | — | — | — | X | — | — | — | — |
| G6PD | — | — | — | X | — | — | — | — | — | — | — | — | — |
| screening | | | | | | | | | | | | | |
| Viral | — | X | — | — | — | — | — | — | — | — | — | — | — |
| serology | | | | | | | | | | | | | |
| Leukapheresis^{m} | — | — | X | — | — | — | — | — | — | — | — | — | — |

STUDY TREATMENT

| | | | | | | | | | | | | | |
|---|---|---|---|---|---|---|---|---|---|---|---|---|---|
| LDC | — | — | — | — | X | X | X | X | — | — | — | — | — |
| AFNT-212 | — | — | — | — | — | — | — | — | X | — | — | — | — |
| infusion | | | | | | | | | | | | | |

EFFICACY ASSESSMENTS

| | | | | | | | | | | | | | |
|---|---|---|---|---|---|---|---|---|---|---|---|---|---|
| Disease/ | — | X^{o} | — | X^{p} | — | — | — | — | — | — | — | — | — |
| response | | | | | | | | | | | | | |
| assessment | | | | | | | | | | | | | |
| by Imagingⁿ | | | | | | | | | | | | | |
| ECOG | — | X | — | X | — | — | — | — | X | — | — | — | — |

OTHER ASSESSMENTS

| | | | | | | | | | | | | | |
|---|---|---|---|---|---|---|---|---|---|---|---|---|---|
| Subsequent | — | — | — | — | — | — | — | — | — | — | — | — | — |
| cancer | | | | | | | | | | | | | |
| treatment | | | | | | | | | | | | | |
| Survival | — | — | — | — | — | — | — | — | — | — | — | — | — |
| Follow-up^{r} | | | | | | | | | | | | | |

| | Study Periods | | | | | | | | | | | | |
|---|---|---|---|---|---|---|---|---|---|---|---|---|---|
| | Post-Treatment | | | | | | | | | | | | |
| | Study Day/Month | | | | | | | | | | | | |
| | Safety | | Post-treatment | | | | | | | | | | |
| | Monitoring/DLT^{s} | | Follow-up^{s} | | | | | | | | | | |
| | Month 1 | | Months 3-24 | | | | | | | | | | |
| | D 22 | D 29 | M 3 | M 5 | M 6 | M 7 | M 9 | M 12 | M 15 | M 18 | M 21 | M 24/PTFD | PD^{t} |
| | Visit Window (Days) | | | | | | | | | | | | |
| | ±3 | ±3 | ±7 | ±7 | ±7 | ±7 | ±7 | ±14 | ±14 | ±14 | ±14 | ±3 | |
| Assessments | | | | | | | | | | | | | |
| GENERAL STUDY ASSESSMENTS | | | | | | | | | | | | | |
| Informed | — | — | — | — | — | — | — | — | — | — | — | X | — |
| consent^{a} | | | | | | | | | | | | | |
| KRAS and HLA | — | — | — | — | — | — | — | — | — | — | — | — | — |
| status | | | | | | | | | | | | | |
| testing^{c} | | | | | | | | | | | | | |
| I/E | — | — | — | — | — | — | — | — | — | — | — | — | — |
| criteria^{d} | | | | | | | | | | | | | |
| Demographics | — | — | — | — | — | — | — | — | — | — | — | — | — |
| Medical | — | — | — | — | — | — | — | — | — | — | — | — | — |
| history | | | | | | | | | | | | | |
| Prior | — | — | — | — | — | — | — | — | — | — | — | — | — |
| disease | | | | | | | | | | | | | |
| history/ | | | | | | | | | | | | | |
| therapies | | | | | | | | | | | | | |
| Concomitant | X | X | X | X | X | X | X | X | X | X | X | X | X |
| medications/ | | | | | | | | | | | | | |
| procedures | | | | | | | | | | | | | |
| HRQoL | — | X | X | X | — | X | X | X | — | — | — | X | — |
| Mutagenic | — | — | X | X | X | X | X | X | X | X | X | X | X |
| agent | | | | | | | | | | | | | |
| exposure | | | | | | | | | | | | | |
| assessment | | | | | | | | | | | | | |
| SAFETY ASSESSMENTS | | | | | | | | | | | | | |
| AE assessments | X | X | X | X | X | X | X | X | X | X | X | X | X |
| Temperature | X | X | — | — | — | — | — | — | — | — | — | — | — |
| monitoring^{e} | | | | | | | | | | | | | |
| ICE^{f} | X | X | X | — | — | — | — | — | — | — | — | X | — |
| Vital | X | X | — | — | — | — | — | — | — | — | — | — | — |
| signs^{g} | | | | | | | | | | | | | |
| O₂ saturation | X | X | X | X | — | X | X | X | X | X | X | X | — |
| test^{g} | | | | | | | | | | | | | |
| Targeted | X | X | X | X | — | X | X | X | X | X | X | X | — |
| physical | | | | | | | | | | | | | |
| examination | | | | | | | | | | | | | |
| Complete | — | — | — | — | — | X | — | — | — | — | — | — | — |
| physical | | | | | | | | | | | | | |
| exam, | | | | | | | | | | | | | |
| height | | | | | | | | | | | | | |
| Weight | — | X | X | X | — | X | X | X | X | X | — | X | — |
| Pulmonary | — | — | — | — | — | — | — | — | — | — | — | — | — |
| function^{h} | | | | | | | | | | | | | |
| 12-lead ECG | — | — | — | — | — | — | — | — | — | — | — | — | — |
| MUGA or | — | — | — | — | — | — | — | — | — | — | — | — | — |
| ECHO^{h} | | | | | | | | | | | | | |
| SAFETY LABORATORY ASSESSMENTS | | | | | | | | | | | | | |
| Serum/urine | — | X | X | X | — | X | X | X | X | X | X | X | — |
| pregnancy | | | | | | | | | | | | | |
| testⁱ | | | | | | | | | | | | | |
| Chemistry | X | X | X | X | — | X | X | X | X | X | X | X | — |
| panel^{j} | | | | | | | | | | | | | |
| Hematology | X | X | X | X | — | X | X | X | X | X | X | X | — |
| panel^{j} | | | | | | | | | | | | | |
| Coagulation | X | X | — | — | — | — | — | — | — | — | — | — | — |
| panel | | | | | | | | | | | | | |
| TLS/CRS | X | X | — | — | — | — | — | — | — | — | — | — | — |
| panel | | | | | | | | | | | | | |
| Tumor | — | X | X | X | — | X | X | X | X | X | X | X | X |
| Markers^{k} | | | | | | | | | | | | | |
| Inflammatory | X | X | — | — | — | — | — | — | — | — | — | — | — |
| markers^{l} | | | | | | | | | | | | | |
| Urinalysis | — | — | — | — | — | — | — | — | — | — | — | — | — |
| G6PD | — | — | — | — | — | — | — | — | — | — | — | — | — |
| screening | | | | | | | | | | | | | |
| Viral | — | — | — | — | — | — | — | — | — | — | — | — | — |
| serology | | | | | | | | | | | | | |
| Leukapheresis^{m} | — | — | — | — | — | — | — | — | — | — | — | — | — |
| STUDY TREATMENT | | | | | | | | | | | | | |
| LDC | — | — | — | — | — | — | — | — | — | — | — | — | — |
| AFNT-212 | — | — | — | — | — | — | — | — | — | — | — | — | — |
| infusion | | | | | | | | | | | | | |
| EFFICACY ASSESSMENTS | | | | | | | | | | | | | |
| Disease/ | — | X | X | X | — | X | X | X | X | X | X | X | X |
| response | | | | | | | | | | | | | |
| assessment | | | | | | | | | | | | | |
| by Imagingⁿ | | | | | | | | | | | | | |
| ECOG | — | X | X | X | — | X | X | X | X | X | X | X | X |
| OTHER ASSESSMENTS | | | | | | | | | | | | | |
| Subsequent | — | — | — | — | — | — | — | — | — | — | — | — | X |
| cancer | | | | | | | | | | | | | |
| treatment | | | | | | | | | | | | | |
| Survival | — | — | — | — | — | — | — | — | — | — | — | X | X |
| Follow-up^{r} | | | | | | | | | | | | | |

Abbreviations: AE=adverse event; CRS=cytokine release syndrome; CT=computed tomography; D=day; DLT=dose-limiting toxicity; ECG=electrocardiogram; ECHO=echocardiogram; ECOG=Eastern Cooperative Oncology Group; EoT=end of treatment; G6PD=Glucose-6-phosphate dehydrogenase; HLA-A*11:01=human leukocyte antigen-A; HRQoL=health-related quality of life; ICANS=Immune effector cell-associated neurotoxicity syndrome; ICE=immune effector cell-associated encephalopathy; ICF=informed consent form; KRAS=Kirsten rat sarcoma viral oncogene homolog; LDC=lymphodepleting chemotherapy; LTFU=long-term follow-up; M=month; MRI=magnetic resonance imaging; MUGA=multigated acquisition scan; 02=oxygen; PD=progressive disease; PET=position emission tomography; PK=pharmacokinetics; PTFD=post-treatment follow-up discontinuation; RECIST=Response Evaluation Criteria in Solid Tumors; SOCBP=subjects of childbearing potential; TLS=tumor lysis syndrome; yrs=years
All assessments and sample collections will be performed prior to LDC and AFNT-212 infusion unless otherwise noted.
   Additional assessments to evaluate subject safety and efficacy may be performed at unscheduled visits and will be recorded in the eCRFs. Refer to the Study Laboratory Manual for further details on sample collection and shipment to a central laboratory.
a. The ICFs must be signed before any protocol-specific assessments are performed unless otherwise specified. A separate ICF for the STFU and LTFU Periods will be signed at the M24/PTFD Visit.
b. Baseline assessments must be conducted within 72 hours (3 days) of LDC. Baseline procedures may also occur on the day of LDC.
c. For KRAS and HLA testing, samples should be saved for potential diagnostics development
d. Enrollment evaluations must be conducted within 30 days of leukapheresis unless otherwise specified. Radiologic imaging within 45 days of leukapheresis is acceptable.
e. Subjects will record daily oral temperature at home twice daily (approximately 8 hours apart) for 14 days following AFNT-212 infusion and once daily through 28 days following AFNT-212 infusion. When subjects are in the clinic, temperature will be measured/recorded by the study staff. When not in clinic, temperature will be measured and recorded in the temperature diary by the subject or their dedicated caregiver.
f. Dedicated caregiver will be required to complete ICE assessment during the DLT period on days that the subject will not visit the study site.
g. Vital signs (including O₂ saturation) on day of AFNT-212 infusion (D1) must be obtained before start of infusion, every 15 minutes during infusion, within 5 minutes after the end of infusion, and approximately hourly for 2 hours after the end of infusion.
h. MUGA/ECHO and/or pulmonary function testing required if indicated (ie, history of pulmonary dysfunction or cardiovascular abnormalities). For subjects who received potentially cardiotoxic bridging therapy, a repeat ECHO or MUGA is required within 2 weeks of LDC.
i. Serum or urine pregnancy test for SOCBP within 72 hours prior to planned leukapheresis. A serum pregnancy test is also required within 72 hours prior to LDC.
j. If standard of care laboratory tests were performed per protocol requirements, they may be used to assess subject eligibility if collected within the eligibility screening period. Blood draws for laboratory tests do not need to be repeated.
k. CEA, CA-125, CA-19-9, CA-242 as clinically relevant per SOC
l. Inflammatory markers will be assessed as clinically indicated (e.g., if the subject becomes febrile or develops symptoms of CRS or TLS).
m. If >7 days from screening assessments to leukapheresis, relevant screening assessments must be repeated and within defined ranges prior to leukapheresis.
n. An MRI of the brain will be required at Screening. The disease will be assessed per RECIST 1.1 based on CT scans with contrast to include chest, abdomen and pelvis. For subjects/investigational sites unable to undergo/utilize CT scans, MRI or PET/CT scans may be used. The same modality used for imaging at Baseline should be used at each subsequent timepoint. Scans will be collected for review by the central imaging vendor.
o. Standard of care images that were taken before study entry may be used to determine subject eligibility for this study. Standard of care images used to assess eligibility must be obtained within 45 days of leukapheresis.
p. Imaging for Baseline must be within 28 days prior to the start of first planned AFNT-212 infusion. Subjects must be re-imaged if they receive bridging therapy.
q. Imaging will be obtained until M24/PTFD or PD, whichever occurs first. During STFU, imaging will only be collected if subjects did not have a PD event.
r. At the time of PTFD and/or documented PD, subjects will be asked to continue in the STFU/LTFU to be followed for up to 15 years from the first AFNT-212 infusion. Subsequent anticancer therapies and response for each line regimen will be collected. Subjects who do not consent to participate in the STFU/LTFU will be followed every 3 months for survival status, or through public records.
s. Relevant assessments may be conducted if subjects become febrile or develop signs of toxicity at any time.
t. To confirm PD.

TABLE 13
Schedule of Pharmacokinetic and Biomarker Activities

| Study Periods | | | | | | | | | | | | |
|---|---|---|---|---|---|---|---|---|---|---|---|---|
| Pretreatment | | | | Treatment | | | | | Post-Treatment | | | |
| Study Day/Month | | | | | | | | | | | | |
| | | | | | | | | | Post-treatment Follow-up | | | |
| | | | | Lymphodepleting | | | | KRAS G12D | Safety Monitoring/DLT | | | |
| KRAS-HLA | Eligibility | Eligibility/ | | Chemotherapy | | | | TCR T cells | Month 1 | | | |
| Screening | Screening^{a} | Leukapheresis | Baseline | D-6 | D-5 | D-4 | D-3 | D 1 | D 3 | D 8 | D 10 | D 15 |

Visit Window (Days)

| | |
|---|---|
| ±3 | ±3 |

Assessments
PHARMACOKINETICS AND BIOMARKERS

| | | | | | | | | | | | | | |
|---|---|---|---|---|---|---|---|---|---|---|---|---|---|
| Tumor | — | | — | X^{b} | — | — | — | — | — | — | — | — | — |
| biopsy^{a}/ | | | | | | | | | | | | | |
| archival | | | | | | | | | | | | | |
| tissue | | | | | | | | | | | | | |
| (optional) | | | | | | | | | | | | | |
| Buccal | — | — | — | X | — | — | — | — | — | — | — | — | — |
| sample for | | | | | | | | | | | | | |
| normal | | | | | | | | | | | | | |
| control^{c} | | | | | | | | | | | | | |
| Blood for | — | — | — | X | — | — | — | — | — | — | — | — | — |
| normal | | | | | | | | | | | | | |
| control^{c} | | | | | | | | | | | | | |
| Blood for | — | — | — | X | — | — | — | — | X | X | X | X | X |
| molecular | | | | | | | | | | | | | |
| PK^{d, e, g} | | | | | | | | | | | | | |
| Blood for | — | — | — | X | — | — | — | — | X | X | X | X | X |
| cellular | | | | | | | | | | | | | |
| PK^{d, e} | | | | | | | | | | | | | |
| Blood for | — | — | — | X | — | — | — | — | X | X | X | X | X |
| cytokine | | | | | | | | | | | | | |
| levels^{d, e} | | | | | | | | | | | | | |
| Blood for | — | — | — | X | — | — | — | — | X | — | — | — | X |
| ctDNA^{f} | | | | | | | | | | | | | |
| Blood for | — | — | — | X | X | X | X | X | X | — | — | — | X |
| immune | | | | | | | | | | | | | |
| profiling^{d, i} | | | | | | | | | | | | | |
| Blood for | — | — | — | X | X | X | X | X | X | — | — | — | X |
| serum | | | | | | | | | | | | | |
| profiling^{d, g, h} | | | | | | | | | | | | | |

| | Study Periods | | | | | | | | | | | | |
|---|---|---|---|---|---|---|---|---|---|---|---|---|---|
| | Post-Treatment | | | | | | | | | | | | |
| | Study Day/Month | | | | | | | | | | | | |
| | Post-treatment Follow-up | | | | | | | | | | | | |
| | Safety Monitoring/DLT | | Post-treatment Follow-up | | | | | | | | | | |
| | Month 1 | | Months 3-24 | | | | | | | | | | |
| | D 22 | D 29 | M 3 | M 5 | M 6 | M 7 | M 9 | M 12 | M 15 | M 18 | M 21 | M 24/PTFD | PD |
| | Visit Window (Days) | | | | | | | | | | | | |
| | ±3 | ±3 | ±7 | ±7 | ±7 | ±7 | ±7 | ±14 | ±14 | ±14 | ±14 | ±14 | |
| | Assessments | | | | | | | | | | | | |
| | PHARMACOKINETICS AND BIOMARKERS | | | | | | | | | | | | |
| Tumor | — | X | — | X | — | — | — | — | — | — | — | — | X |
| biopsy^{a}/ | | | | | | | | | | | | | |
| archival | | | | | | | | | | | | | |
| tissue | | | | | | | | | | | | | |
| (optional) | | | | | | | | | | | | | |
| Buccal | — | — | — | — | — | — | — | — | — | — | — | — | — |
| sample for | | | | | | | | | | | | | |
| normal | | | | | | | | | | | | | |
| control^{c} | | | | | | | | | | | | | |
| Blood for | — | — | — | — | — | — | — | — | — | — | — | — | — |
| normal | | | | | | | | | | | | | |
| control^{c} | | | | | | | | | | | | | |
| Blood for | X | X | X | X | — | X | X | X | — | X | — | X | X |
| molecular | | | | | | | | | | | | | |
| PK^{d, e, g} | | | | | | | | | | | | | |
| Blood for | X | X | X | X | — | X | — | X | — | X | — | X | X |
| cellular | | | | | | | | | | | | | |
| PK^{d, e} | | | | | | | | | | | | | |
| Blood for | X | X | X | X | X | X | X | X | — | — | — | — | X |
| cytokine | | | | | | | | | | | | | |
| levels^{d, e} | | | | | | | | | | | | | |
| Blood for | — | X | X | X | — | X | X | X | X | X | X | X | X |
| ctDNA^{f} | | | | | | | | | | | | | |
| Blood for | — | X | X | X | — | X | — | X | — | X | — | X | X |
| immune | | | | | | | | | | | | | |
| profiling^{d, i} | | | | | | | | | | | | | |
| Blood for | — | X | X | X | — | X | — | X | — | X | — | X | |
| serum | | | | | | | | | | | | | |
| profiling^{d, g, h} | | | | | | | | | | | | | |

Abbreviations: AESI=adverse event of special interest; CRS=cytokine release syndrome; D=day; ctDNA=circulating tumor DNA; DLT=dose-limiting toxicity; EoT=end of treatment; KRAS=Kirsten rat sarcoma viral oncogene homolog; LDC=lymphodepleting chemotherapy; LTFU=long-term follow-up; M=month; PD=progressive disease; PK=pharmacokinetics; PTFD=post-treatment follow-up discontinuation; RCL=replication-competent lentivirus; scRNAseq=single cell RNA sequencing; follow-up; TCR=T cell receptor; TLS=tumor lysis syndrome
a. Refer to laboratory manual. If no tumor is amenable to biopsy or biopsy is not safe and/or feasible, request at subsequent visits.
b. One core or equivalent of baseline biopsy should be saved for potential KRAS diagnostics development.
c. The first research biopsy should be supplemented with a blood sample or alternatively buccal swab for normal tissue control.
d. If the subject presents with AESI (s), or if such testing is clinically indicated) or clinically appropriate, the assessments may be performed at additional times.
e. Blood for Molecular PK (Persistence), Cellular PK, and Cytokine levels will be collected pre-dose and 120 min (+30 min) post-dose (ie, after the end of infusion). Tests for persistence (molecular PK) will be collected at Months 3, 6, 9, and 12, then every 6 months for up to 5 years. If persistence is indicated after 5 years, tests will be conducted annually until undetectable. If all samples are negative during the first year, the remaining samples can be archived.
f. If an autopsy is performed in the event of subject death, blood and tissue samples should be requested to potentially test vector persistence, transgene expression and other related analysis at the molecular, cellular or tissue level if there are deaths during the STFU and LTFU observation period.
g. Serum collected during LDC period may be used for pharmacokinetic assessment of fludarabine and cyclophosphamide and/or other molecular species
h. Blood collected for immune profiling may be used to assess frequencies of cell populations in the periphery and/or their phenotype

### Example 16: Large-Scale Manufacturing Process for KRAS G12D TCR T Cells

Peripheral blood mononuclear cells (PBMCs) were first isolated. The resulting isolate was enriched for CD4/CD8+ cells using CliniMACS. The enriched isolate was next transferred to a sterile culture system (GRex 500M) for activation.

Cells were activated in activation buffer comprising TGF-β, human platelet lysate, and cytokines (e.g., IL-2, IL-7, IL-15, IL-21).

DNA for knock-in was next prepared (a Nanoplasmid® construct including a sequence encoding a KRAS G12D TCR), and post-activation cells were contacted with the DNA during electroporation (Xenon) for knock-in using homology-directed repair (HDR). In short, electroporation was used to transduce the activated cells with an sgRNA, a nuclease, and the DNA. Once inside the cell, the sgRNA was used to guide the DNA to the TRAC site for knock-in with a MG29-1 nuclease.

Electroporation was conducted in an isotonic electroporation buffer. Multiple sets of electroporation were conducted (e.g., 4 sets) each followed by an extended rest period (e.g., 10-40 minutes or more). After electroporation, cells were transferred into a culture vessel for expansion with an expansion buffer which included human platelet lysate.

On day 8 after initiating activation, cells were fed additional cytokines (e.g., IL-2, IL-7, IL-15, IL-21).

Cells were subsequently harvested for storage or cryopreservation.

The results indicated that there were significant advantages to using the above process. Without intending to be bound by theory, the addition of TGF-β was found to advantageously upregulate various classical tissue residency (T_{RM}) markers, including CD103 and CD39 as compared to buffers lacking TGF-β, and the addition of human platelet lysate during the activation stage, and during the expansion stage, was shown to improve cell proliferation and yield as compared to buffers lacking human platelet lysate. Use of an isotonic electroporation buffer during electroporation was shown to significantly increase both knock-in frequency and expansion potential, as compared to use of standard electroporation buffer (Thermo Fisher). The extended post-electroporation rest period was shown to significantly increase knock-in efficiency and final yield of transduced T cells. The cytokine feeding regimen timing also was shown to significantly improve knock-in efficiency and cell yield.

### OTHER EMBODIMENTS

While embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. All publications, patents, and patent applications mentioned in this specification are herein incorporated by reference to the same extent as if each individual publication, patent, or patent application was specifically and individually indicated to be incorporated by reference.

What is claimed is:

1. A host cell comprising:
a heterologous extracellular binding protein, wherein the extracellular binding protein is capable of binding to a peptide:HLA complex, wherein the peptide comprises a KRAS G12D mutant peptide; and
a genomic mutation that decreases expression of an endogenous T cell receptor α constant (TRAC), T cell receptor β constant 1 (TRBC1), or T cell receptor β constant 2 (TRBC2).

2. The host cell of claim 1, wherein the peptide:HLA complex comprises an HLA protein encoded by an HLA-A*11 allele or an HLA protein encoded by an HLA-A*11:01 allele.

3. The host cell of claim 1, wherein the extracellular binding protein comprises a T cell receptor (TCR) α chain variable (Vα) region, a TCR β chain variable (Vβ) region, a T cell receptor (TCR) α chain constant (Cα) region, or a T cell receptor (TCR) β chain constant (Cβ) region.

4. The host cell of claim 1, wherein the KRAS G12D mutant peptide comprises an amino acid sequence of VVVGADGVGK (SEQ ID NO: 295).

5. The host cell of claim 1, wherein the Vα domain, the Vβ domain, or
extracellular binding protein are human, humanized, or chimeric.

6. The host cell of claim 5, wherein the extracellular binding protein comprises:
a TCR α chain variable (Vα) domain comprising an amino acid sequence with at least 80% sequence identity to any one of SEQ ID NOs: 9, 25, 41, 57, 73, 89, 105, 123, 139, 155, 171, 187, 229, 230, 239, 240, 249, 250, 259, 260, 269, and 270;
a TCR β chain variable (Vβ) domain comprising an amino acid sequence with at least 80% sequence identity to any one of SEQ ID NOs: 1, 17, 33, 49, 65, 81, 97, 131, 147, 163, 179, 195, 234, 235, 244, 245, 254, 255, 264, 265, 274, and 275; or
a TCRα FR1, CDR1, FR2, CDR2, FR3, CDR3, or FR4 region or a TCRβ FR1, CDR1, FR2, CDR2, FR3, CDR3, or FR4 region comprising an amino acid sequence with at least 80% sequence identity to any one of SEQ ID NOs: 2-8, 10-16, 18-24, 26-32,34-40, 42-48, 50-56, 58-64, 66-72, 74-80, 82-88, 90-96, 98-104, 106-112, 124-130, 132-138, 140-146, 148-154, 156-162, 164-170, 172-178, 180-186, 188-194, 196-202, 231-233, 236-238, 241-243, 246-248, 251-253, 25-258, 261-263, 266-268, 271-273, and 276-278.

7. The host cell of claim 1, wherein the extracellular binding protein specifically binds the KRAS G12D mutant peptide.

8. The host cell of claim 1, wherein the host cell further comprises (i) a transgenic polynucleotide encoding a polypeptide that comprises an extracellular portion of a CD8 co-receptor α (CD8α) chain or (ii) a transgenic polynucleotide encoding a polypeptide that comprises an extracellular portion of a CD8 co-receptor β (CD8β) chain.

9. The host cell of claim 1, wherein the genomic mutation that causes or contributes to decreased expression of the endogenous T cell receptor α constant (TRAC), T cell receptor β constant 1 (TRBC1), and/or a T cell receptor β constant 2 (TRBC2) comprises one or more of the following:
an indel in the TRAC, TRBC1, or TRBC2 locus;
a missense mutation that causes or contributes to reduced function or stability of a T cell receptor α or T cell receptor β polypeptide encoded by a genome of the cell;
premature termination of a T cell receptor α or T cell receptor β polypeptide encoded by the endogenous TRAC, TRBC1, or TRBC2.

10. The host cell of claim 9, wherein the host cell comprises a T cell, a NK cell, a NK-T cell, a dendritic cell, a macrophage, a monocyte, a precursor thereof, or any combination thereof.

11. The host cell of claim 10, wherein the T cell comprises a CD4⁺ T cell, a CD8⁺ T cell, a CD4⁻ CD8⁻ double negative T cell, a γδ T cell, or any combination thereof.

12. A host cell comprising a polynucleotide encoding a heterologous extracellular binding protein inserted at a TRAC, TRBC1, or TRBC2 locus, wherein the extracellular binding protein is capable of binding to a peptide:HLA complex, wherein the peptide comprises a KRAS G12D mutant peptide, and wherein the host cell has decreased expression of TRAC, TRBC1, or TRBC2.

13. A polynucleotide encoding an extracellular binding protein, wherein the extracellular binding protein comprises:
a TCR α chain variable (Vα) domain comprising an amino acid sequence with at least 80% sequence identity to any one of SEQ ID NOs: 9, 25, 41, 57, 73, 89, 105, 123, 139, 155, 171, 187, 229, 230, 239, 240, 249, 250, 259, 260, 269, and 270;
a TCR β chain variable (Vβ) domain comprising an amino acid sequence with at least 80% sequence identity to any one of SEQ ID NOs: 1, 17, 33, 49, 65, 81, 97, 131, 147, 163, 179, 195, 234, 235, 244, 245, 254, 255, 264, 265, 274, and 275; or
a TCRα FR1, CDR1, FR2, CDR2, FR3, CDR3, or FR4 region or a TCRβ FR1, CDR1, FR2, CDR2, FR3, CDR3, or FR4 region comprising an amino acid sequence with at least 80% sequence identity to any one of SEQ ID NOs: 2-8, 10-16, 18-24, 26-32,34-40, 42-48, 50-56, 58-64, 66-72, 74-80, 82-88, 90-96, 98-104, 106-112, 124-130, 132-138, 140-146, 148-154, 156-162, 164-170, 172-178, 180-186, 188-194, 196-202, 231-233, 236-238, 241-243, 246-248, 251-253, 25-258, 261-263, 266-268, 271-273, and 276-278.

14. A vector comprising the polynucleotide of claim 13.

15. A cell comprising the polynucleotide of claim 13.

16. A pharmaceutical composition comprising the host cell of claim 1 and a pharmaceutically acceptable carrier, excipient, or diluent.

17. A method of treating a disease or disorder associated with a KRAS G12 mutation in a subject, the method comprising administering to the subject an effective amount of the host cell of claim 1.

18. The method of claim 17, wherein the subject is positive for an HLA-A*11 allele.

19. The method of claim 17, wherein the KRAS G12 mutation is a KRAS G12D mutation.

20. The method of claim 17, wherein the disease or disorder comprises a cancer.


SEQUENCE LISTING (307 sequences)

SEQ ID NO: 1 - protein, 313 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSTGRVYQPQHFGDGTRLSILEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGGSG

SEQ ID NO: 2 - protein, 26 aa, Homo sapiens
DAMVIQNPRYQVTQFGKPVTLSCSQT

SEQ ID NO: 3 - protein, 5 aa, Homo sapiens
LNHNV

SEQ ID NO: 4 - protein, 17 aa, Homo sapiens
MYWYQQKSSQAPKLLFH

SEQ ID NO: 5 - protein, 6 aa, Homo sapiens
YYDKDF

SEQ ID NO: 6 - protein, 37 aa, Homo sapiens
NNEADTPDNFQSRRPNTSFCFLDIRSPGLGDAAMYLC

SEQ ID NO: 7 - protein, 12 aa, Homo sapiens
ATSTGRVYQPQH

SEQ ID NO: 8 - protein, 10 aa, Homo sapiens
FGDGTRLSIL

SEQ ID NO: 9 - protein, 278 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALNEGGGSTLGRLYFGRGTQLTVWPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 26 aa, Homo sapiens
AQKVTQAQTEISVVEKEDVTLDCVYE

SEQ ID NO: 11 - protein, 7 aa, Homo sapiens
TRDTTYY

SEQ ID NO: 12 - protein, 17 aa, Homo sapiens
LFWYKQPPSGELVFLIR

SEQ ID NO: 13 - protein, 8 aa, Homo sapiens
RNSFDEQN

SEQ ID NO: 14 - protein, 34 aa, Homo sapiens
EISGRYSWNFQKSTSSFNFTITASQVVDSAVYFC

SEQ ID NO: 15 - protein, 14 aa, Homo sapiens
ALNEGGGSTLGRLY

SEQ ID NO: 16 - protein, 11 aa, Homo sapiens
FGRGTQLTVWP

SEQ ID NO: 17 - protein, 314 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSGVDRAYGYTFGSGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGGSG

SEQ ID NO: 18 - protein, 26 aa, Homo sapiens
EPEVTQTPSHQVTQMGQEVILRCVPI

SEQ ID NO: 19 - protein, 5 aa, Homo sapiens
SNHLY

SEQ ID NO: 20 - protein, 17 aa, Homo sapiens
FYWYRQILGQKVEFLVS

SEQ ID NO: 21 - protein, 6 aa, Homo sapiens
FYNNEI

SEQ ID NO: 22 - protein, 38 aa, Homo sapiens
SEKSEIFDDQFSVERPDGSNFTLKIRSTKLEDSAMYFC

SEQ ID NO: 23 - protein, 12 aa, Homo sapiens
ASSGVDRAYGYT

SEQ ID NO: 24 - protein, 10 aa, Homo sapiens
FGSGTRLTVV

SEQ ID NO: 25 - protein, 275 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCASTLSSGDKLTFGTGTRLAVRPNIQKPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 26 aa, Homo sapiens
AQTVTQSQPEMSVQEAETVTLSCTYD

SEQ ID NO: 27 - protein, 7 aa, Homo sapiens
TSESDYY

SEQ ID NO: 28 - protein, 17 aa, Homo sapiens
LFWYKQPPSRQMILVIR

SEQ ID NO: 29 - protein, 8 aa, Homo sapiens
QEAYKQQN

SEQ ID NO: 30 - protein, 34 aa, Homo sapiens
ATENRFSVNFQKAAKSFSLKISDSQLGDAAMYFC

SEQ ID NO: 31 - protein, 11 aa, Homo sapiens
ASTLSSGDKLT

SEQ ID NO: 32 - protein, 11 aa, Homo sapiens
FGTGTRLAVRP

SEQ ID NO: 33 - protein, 313 aa, Homo sapiens
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSDRGWNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSG

SEQ ID NO: 34 - protein, 26 aa, Homo sapiens
ETGVTQSPTHLIKTRGQQVTLRCSSQ

SEQ ID NO: 35 - protein, 5 aa, Homo sapiens
SGHNT

SEQ ID NO: 36 - protein, 17 aa, Homo sapiens
VSWYQQALGQGPQFIFQ

SEQ ID NO: 37 - protein, 6 aa, Homo sapiens
YYREEE

SEQ ID NO: 38 - protein, 37 aa, Homo sapiens
NGRGNFPPRFSGLQFPNYSSELNVNALELDDSALYLC

SEQ ID NO: 39 - protein, 11 aa, Homo sapiens
ASSDRGWNEQF

SEQ ID NO: 40 - protein, 10 aa, Homo sapiens
FGPGTRLTVL

SEQ ID NO: 41 - protein, 279 aa, Homo sapiens
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMTPITQGGSEKLVFGKGTKLTVNPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 42 - protein, 26 aa, Homo sapiens
AQTVTQSQPEMSVQEAETVTLSCTYD

SEQ ID NO: 43 - protein, 7 aa, Homo sapiens
TSENNYY

SEQ ID NO: 44 - protein, 17 aa, Homo sapiens
LFWYKQPPSRQMILVIR

SEQ ID NO: 45 - protein, 8 aa, Homo sapiens
QEAYKQQN

SEQ ID NO: 46 - protein, 34 aa, Homo sapiens
ATENRFSVNFQKAAKSFSLKISDSQLGDTAMYFC

SEQ ID NO: 47 - protein, 15 aa, Homo sapiens
AFMTPITQGGSEKLV

SEQ ID NO: 48 - protein, 11 aa, Homo sapiens
FGKGTKLTVNP

SEQ ID NO: 49 - protein, 309 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSEWAGTEAFFGQGTRLTVVEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 50 - protein, 26 aa, Homo sapiens
EPEVTQTPSHQVTQMGQEVILRCVPI

SEQ ID NO: 51 - protein, 5 aa, Homo sapiens
SNHLY

SEQ ID NO: 52 - protein, 17 aa, Homo sapiens
FYWYRQILGQKVEFLVS

SEQ ID NO: 53 - protein, 6 aa, Homo sapiens
FYNNEI

SEQ ID NO: 54 - protein, 38 aa, Homo sapiens; identical to SEQ ID NO: 22

SEQ ID NO: 55 - protein, 11 aa, Homo sapiens
ASSEWAGTEAF

SEQ ID NO: 56 - protein, 10 aa, Homo sapiens
FGQGTRLTVV

SEQ ID NO: 57 - protein, 271 aa, Homo sapiens
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGKTTDSWGKLQFGAGTQVVVTPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 26 aa, Homo sapiens
TQLLEQSPQFLSIQEGENLTVYCNSS

SEQ ID NO: 59 - protein, 5 aa, Homo sapiens
SVFSS

SEQ ID NO: 60 - protein, 17 aa, Homo sapiens
LQWYRQEPGEGPVLLVT

SEQ ID NO: 61 - protein, 7 aa, Homo sapiens
VVTGGEV

SEQ ID NO: 62 - protein, 34 aa, Homo sapiens
KKLKRLTFQFGDARKDSSLHITAAQPGDTGLYLC

SEQ ID NO: 63 - protein, 12 aa, Homo sapiens
AGKTTDSWGKLQ

SEQ ID NO: 64 - protein, 11 aa, Homo sapiens
FGAGTQVVVTP

SEQ ID NO: 65 - protein, 315 aa, Homo sapiens
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCATPLGRTNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGSG

SEQ ID NO: 66 - protein, 26 aa, Homo sapiens
DTGVSQDPRHKITKRGQNVTFRCDPI

SEQ ID NO: 67 - protein, 5 aa, Homo sapiens
SEHNR

SEQ ID NO: 68 - protein, 17 aa, Homo sapiens
LYWYRQTLGQGPEFLTY

SEQ ID NO: 69 - protein, 6 aa, Homo sapiens
FQNEAQ

SEQ ID NO: 70 - protein, 38 aa, Homo sapiens
LEKSRLLSDRFSAERPKGSFSTLEIQRTEQGDSAMYLC

SEQ ID NO: 71 - protein, 11 aa, Homo sapiens
ATPLGRTNEQF

SEQ ID NO: 72 - protein, 10 aa, Homo sapiens
FGPGTRLTVL

SEQ ID NO: 73 - protein, 276 aa, Homo sapiens
MKSLRVLLVILWLQLSWVWSQQKEVEQNSGPLSVPEGAIASLNCTYSDRVSQSFFWYRQYSGKSPELIMSIYSNGDKEDGRFTAQLNKASQYVSLLIRDSQPSDSATYLCAVRWAGGTSYGKLTFGQGTILTVHPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 74 - protein, 26 aa, Homo sapiens
QKEVEQNSGPLSVPEGAIASLNCTYS

SEQ ID NO: 75 - protein, 6 aa, Homo sapiens
DRVSQS

SEQ ID NO: 76 - protein, 17 aa, Homo sapiens
FFWYRQYSGKSPELIMS

SEQ ID NO: 77 - protein, 6 aa, Homo sapiens
IYSNGD

SEQ ID NO: 78 - protein, 34 aa, Homo sapiens
KEDGRFTAQLNKASQYVSLLIRDSQPSDSATYLC

SEQ ID NO: 79 - protein, 14 aa, Homo sapiens
AVRWAGGTSYGKLT

SEQ ID NO: 80 - protein, 11 aa, Homo sapiens
FGQGTILTVHP

SEQ ID NO: 81 - protein, 317 aa, Homo sapiens
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASSFRLREGLGEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGGSG

SEQ ID NO: 82 - protein, 26 aa, Homo sapiens
DVKVTQSSRYLVKRTGEKVFLECVQD

SEQ ID NO: 83 - protein, 5 aa, Homo sapiens
MDHEN

SEQ ID NO: 84 - protein, 17 aa, Homo sapiens
MFWYRQDPGLGLRLIYF

SEQ ID NO: 85 - protein, 6 aa, Homo sapiens
SYDVKM

SEQ ID NO: 86 - protein, 37 aa, Homo sapiens
KEKGDIPEGYSVSREKKERFSLILESASTNQTSMYLC

SEQ ID NO: 87 - protein, 14 aa, Homo sapiens
ASSFRLREGLGEQF

SEQ ID NO: 88 - protein, 10 aa, Homo sapiens
FGPGTRLTVL

SEQ ID NO: 89 - protein, 275 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYCNPNAGKSTFGDGTTLTVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 26 aa, Homo sapiens
AQTVTQSQPEMSVQEAETVTLSCTYD

SEQ ID NO: 91 - protein, 7 aa, Homo sapiens
TSESDYY

SEQ ID NO: 92 - protein, 17 aa, Homo sapiens
LFWYKQPPSRQMILVIR

SEQ ID NO: 93 - protein, 8 aa, Homo sapiens
QEAYKQQN

SEQ ID NO: 94 - protein, 34 aa, Homo sapiens; identical to SEQ ID NO: 30

SEQ ID NO: 95 - protein, 11 aa, Homo sapiens
AYCNPNAGKST

SEQ ID NO: 96 - protein, 11 aa, Homo sapiens
FGDGTTLTVKP

SEQ ID NO: 97 - protein, 313 aa, Homo sapiens
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSTGRVYQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGGSG

SEQ ID NO: 98 - protein, 26 aa, Homo sapiens
DAMVIQNPRYQVTQFGKPVTLSCSQT

SEQ ID NO: 99 - protein, 5 aa, Homo sapiens
LNHNV

SEQ ID NO: 100 - protein, 17 aa, Homo sapiens
MYWYQQKSSQAPKLLFH

SEQ ID NO: 101 - protein, 6 aa, Homo sapiens
YYDKDF

SEQ ID NO: 102 - protein, 37 aa, Homo sapiens; identical to SEQ ID NO: 6

SEQ ID NO: 103 - protein, 12 aa, Homo sapiens
ATSTGRVYQPQH

SEQ ID NO: 104 - protein, 10 aa, Homo sapiens
FGDGTRLSIL

SEQ ID NO: 105 - protein, 278 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALNEGGGSTLGRLYFGRGTQLTVWPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 106 - protein, 26 aa, Homo sapiens
AQKVTQAQTEISVVEKEDVTLDCVYE

SEQ ID NO: 107 - protein, 7 aa, Homo sapiens
TRDTTYY

SEQ ID NO: 108 - protein, 17 aa, Homo sapiens
LFWYKQPPSGELVFLIR

SEQ ID NO: 109 - protein, 8 aa, Homo sapiens
RNSFDEQN

SEQ ID NO: 110 - protein, 34 aa, Homo sapiens; identical to SEQ ID NO: 14

SEQ ID NO: 111 - protein, 14 aa, Homo sapiens
ALNEGGGSTLGRLY

SEQ ID NO: 112 - protein, 11 aa, Homo sapiens
FGRGTQLTVWP

SEQ ID NO: 113-116 - skipped (4 entries)

SEQ ID NO: 117 - protein, 1280 aa, Homo sapiens
MFNNFIKKYSLQKTLRFELKPVGETADYIEDFKSEYLKDTVLKDEQRAKDYQEIKTLIDDYHREYIEECLREPVDKKTGEILDFTQDLEDAFSYYQKLKENPTENRVGWEKEQESLRKKLVTSFVGNDGLFKKEFITRDLPEWLQKKGLWGEYKDTVENFKKFTTYFSGFHENRKNMYTAEAQSTAIANRLMNDNLPKFFNNYLAYQTIKEKHPDLVFRLDDALLQAAGVEHLDEAFQPRYFSRLFAQSGITAFNELIGGRTTENGEKIQGLNEQINLYRQQNPEKAKGFPRFMPLFKQILSDRETHSFLPDAFENDKELLQALRDYVDAATSEEGMISQLNKAMNQFVTADLKRVYIKSAALTSLSQELFHFFGVISDAIAWYAEKRLSPKKAQESFLKQEVYAIEELNQAVVGYIDQLEDQSELQQLLVDLPDPQKPVSSFILTHWQKSQEPLQAVIAKVEPLFELEELSKNKRAPKHDKDQGGEGFQQVDAIKNMLDAFMEVSHAIKPLYLVKGRKAIDMPDVDTGFYADFAEAYSAYEQVTVSLYNKTRNHLSKKPFSKDKIKINFDAPTLLNGWDLNKESDNKSIILRKDGNFYLAIMHPKHTKVFDCYSASEAAGKCYEKMNYKLLSGANKMLPKVFFSKKGIETFSPPQEILDLYKNNEHKKGATFKLESCHKLIDFFKRNIPKYKVHPTDNFGWDVFGFHFSPTSSYGDLSGFYREVEAQGYKLWFSDVSEAYINKCVEEGKLFLFQIYNKDFSPNSTGKPNLHTLYWKGLFEPENLKDVVLKLNGEAEIFYRKHSIKHEDKTIHRAKDPIANKNADNPKKQSVFDYDIIKDKRYTQDKFFFHVPISLNFKSQGVVRFNDKINGLLAAQDDVHVIGIDRGERHLLYYTVVNGKGEVVEQGSLNQVATDQGYVVDYQQKLHAKEKERDQARKNWSTIENIKELKAGYLSQVVHKLAQLIVKHNAIVCLEDLNFGFKRGRFKVEKQVYQKFEKALIDKLNYLVFKERGATQAGGYLNAYQLAAPFESFEKLGKQTGILYYVRSDYTSKIDPATGFVDFLKPKYESMAKSKVFFESFERIQWNQAKGYFEFEFDYKKMCPSRKFGDYRTRWVVCTFGDTRYQNRRNKSSGQWETETIDVTAQLKALFAAYGITYNQEDNIKDAIAAVKYTKFYKQLYWLLRLTLSLRHSVTGTDEDFILSPVADENGVFFDSRKATDKQPKDADANGAYHIALKGLWNLQQIRQHDWNVEKPKKLNLAMKNEEWFGFAQKKKFRA

SEQ ID NO: 118 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 119 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 120 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 121 - protein, 189 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 122 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 123 - protein, 273 aa, Homo sapiens
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDRVGGARLMFGDGTQLVVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 124 - protein, 26 aa, Homo sapiens
GDSVTQMEGPVTLSEEAFLTINCTYT

SEQ ID NO: 125 - protein, 6 aa, Homo sapiens
ATGYPS

SEQ ID NO: 126 - protein, 17 aa, Homo sapiens
LFWYVQYPGEGLQLLLK

SEQ ID NO: 127 - protein, 7 aa, Homo sapiens
ATKADDK

SEQ ID NO: 128 - protein, 34 aa, Homo sapiens
GSNKGFEATYRKETTSFHLEKGSVQVSDSAVYFC

SEQ ID NO: 129 - protein, 12 aa, Homo sapiens
ALSDRVGGARLM

SEQ ID NO: 130 - protein, 11 aa, Homo sapiens
FGDGTQLVVKP

SEQ ID NO: 131 - protein, 310 aa, Homo sapiens
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEGLYNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 132 - protein, 26 aa, Homo sapiens
EADIYQTPRYLVIGTGKKITLECSQT

SEQ ID NO: 133 - protein, 5 aa, Homo sapiens
MGHDK

SEQ ID NO: 134 - protein, 17 aa, Homo sapiens
MYWYQQDPGMELHLIHY

SEQ ID NO: 135 - protein, 6 aa, Homo sapiens
SYGVNS

SEQ ID NO: 136 - protein, 37 aa, Homo sapiens
TEKGDLSSESTVSRIRTEHFPLTLESARPSHTSQYLC

SEQ ID NO: 137 - protein, 11 aa, Homo sapiens
ASSEGLYNEQF

SEQ ID NO: 138 - protein, 10 aa, Homo sapiens
FGPGTRLTVL

SEQ ID NO: 139 - protein, 278 aa, Homo sapiens
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMYPSQGGSEKLVFGKGMKLTVNPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 140 - protein, 26 aa, Homo sapiens
AQTVTQSQPEMSVQEAETVTLSCTYD

SEQ ID NO: 141 - protein, 7 aa, Homo sapiens
TSENNYY

SEQ ID NO: 142 - protein, 17 aa, Homo sapiens
LFWYKQPPSRQMILVIR

SEQ ID NO: 143 - protein, 8 aa, Homo sapiens
QEAYKQQN

SEQ ID NO: 144 - protein, 34 aa, Homo sapiens; identical to SEQ ID NO: 46

SEQ ID NO: 145 - protein, 14 aa, Homo sapiens
AFMYPSQGGSEKLV

SEQ ID NO: 146 - protein, 11 aa, Homo sapiens
FGKGMKLTVNP

SEQ ID NO: 147 - protein, 312 aa, Homo sapiens
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSSPGFRSYGYTFGSGTRLTVVEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 148 - protein, 26 aa, Homo sapiens
ETGVTQSPTHLIKTRGQQVTLRCSSQ

SEQ ID NO: 149 - protein, 5 aa, Homo sapiens
SGHNT

SEQ ID NO: 150 - protein, 17 aa, Homo sapiens
VSWYQQALGQGPQFIFQ

SEQ ID NO: 151 - protein, 6 aa, Homo sapiens
YYREEE

SEQ ID NO: 152 - protein, 37 aa, Homo sapiens; identical to SEQ ID NO: 38

SEQ ID NO: 153 - protein, 13 aa, Homo sapiens
ASSSPGFRSYGYT

SEQ ID NO: 154 - protein, 10 aa, Homo sapiens
FGSGTRLTVV

SEQ ID NO: 155 - protein, 277 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSDGGATNKLIFGTGTLLAVQPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 156 - protein, 26 aa, Homo sapiens
AQTVTQSQPEMSVQEAETVTLSCTYD

SEQ ID NO: 157 - protein, 7 aa, Homo sapiens
TSESDYY

SEQ ID NO: 158 - protein, 17 aa, Homo sapiens
LFWYKQPPSRQMILVIR

SEQ ID NO: 159 - protein, 8 aa, Homo sapiens
QEAYKQQN

SEQ ID NO: 160 - protein, 34 aa, Homo sapiens; identical to SEQ ID NO: 30

SEQ ID NO: 161 - protein, 13 aa, Homo sapiens
AYRSDGGATNKLI

SEQ ID NO: 162 - protein, 11 aa, Homo sapiens
FGTGTLLAVQP

SEQ ID NO: 163 - protein, 310 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGAGGYNSPLHFGNGTRLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 164 - protein, 26 aa, Homo sapiens
EAQVTQNPRYLITVTGKKLTVTCSQN

SEQ ID NO: 165 - protein, 5 aa, Homo sapiens
MNHEY

SEQ ID NO: 166 - protein, 17 aa, Homo sapiens
MSWYRQDPGLGLRQIYY

SEQ ID NO: 167 - protein, 6 aa, Homo sapiens
SMNVEV

SEQ ID NO: 168 - protein, 37 aa, Homo sapiens
TDKGDVPEGYKVSRKEKRNFPLILESPSPNQTSLYFC

SEQ ID NO: 169 - protein, 14 aa, Homo sapiens
ASSLGAGGYNSPLH

SEQ ID NO: 170 - protein, 7 aa, Homo sapiens
FGNGTRL

SEQ ID NO: 171 - protein, 278 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMGALNSGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 172 - protein, 26 aa, Homo sapiens
QKEVEQDPGPLSVPEGAIVSLNCTYS

SEQ ID NO: 173 - protein, 6 aa, Homo sapiens
NSAFQY

SEQ ID NO: 174 - protein, 17 aa, Homo sapiens
FMWYRQYSRKGPELLMY

SEQ ID NO: 175 - protein, 6 aa, Homo sapiens
TYSSGN

SEQ ID NO: 176 - protein, 34 aa, Homo sapiens
KEDGRFTAQVDKSSKYISLFIRDSQPSDSATYLC

SEQ ID NO: 177 - protein, 15 aa, Homo sapiens
AMGALNSGAGSYQLT

SEQ ID NO: 178 - protein, 11 aa, Homo sapiens
FGKGTKLSVIP

SEQ ID NO: 179 - protein, 311 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLSSGTGTEAFFGQGTRLTVEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 180 - protein, 26 aa, Homo sapiens
KAGVTQTPRYLIKTRGQQVTLSCSPI

SEQ ID NO: 181 - protein, 5 aa, Homo sapiens
SGHRS

SEQ ID NO: 182 - protein, 17 aa, Homo sapiens
VSWYQQTPGQGLQFLFE

SEQ ID NO: 183 - protein, 6 aa, Homo sapiens
YFSETQ

SEQ ID NO: 184 - protein, 37 aa, Homo sapiens
RNKGNFPGRFSGRQFSNSRSEMNVSTLELGDSALYLC

SEQ ID NO: 185 - protein, 13 aa, Homo sapiens
ASSLSSGTGTEAF

SEQ ID NO: 186 - protein, 9 aa, Homo sapiens
FGQGTRLTV

SEQ ID NO: 187 - protein, 274 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAERDAGNNRKLIWGLGTSLAVNPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 188 - protein, 25 aa, Homo sapiens
GEDVEQSLFLSVREGDSSVINCTYT

SEQ ID NO: 189 - protein, 6 aa, Homo sapiens
DSSSTY

SEQ ID NO: 190 - protein, 17 aa, Homo sapiens
LYWYKQEPGAGLQLLTY

SEQ ID NO: 191 - protein, 7 aa, Homo sapiens
IFSNMDM

SEQ ID NO: 192 - protein, 34 aa, Homo sapiens
KQDQRLTVLLNKKDKHLSLRIADTQTGDSAIYFC

SEQ ID NO: 193 - protein, 14 aa, Homo sapiens
AERDAGNNRKLIWG

SEQ ID NO: 194 - protein, 9 aa, Homo sapiens
LGTSLAVNP

SEQ ID NO: 195 - protein, 311 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLEGGDTQYFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 196 - protein, 26 aa, Homo sapiens
GAGVSQSPRYKVAKRGQDVALRCDPI

SEQ ID NO: 197 - protein, 5 aa, Homo sapiens
SGHVS

SEQ ID NO: 198 - protein, 17 aa, Homo sapiens
LFWYQQALGQGPEFLTY

SEQ ID NO: 199 - protein, 6 aa, Homo sapiens
FQNEAQ

SEQ ID NO: 200 - protein, 38 aa, Homo sapiens
LDKSGLPSDRFFAERPEGSVSTLKIQRTQQEDSAVYLC

SEQ ID NO: 201 - protein, 11 aa, Homo sapiens
ASSLEGGDTQY

SEQ ID NO: 202 - protein, 10 aa, Homo sapiens
FGPGTRLTVL

SEQ ID NO: 203 - protein, 270 aa, Homo sapiens
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGRREGAQKLVFGQGTRLTINPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 204 - protein, 26 aa, Homo sapiens
GQQLNQSPQSMFIQEGEDVSMNCTSS

SEQ ID NO: 205 - protein, 5 aa, Homo sapiens
SIFNT

SEQ ID NO: 206 - protein, 17 aa, Homo sapiens
WLWYKQDPGEGPVLLIA

SEQ ID NO: 207 - protein, 7 aa, Homo sapiens
LYKAGEL

SEQ ID NO: 208 - protein, 34 aa, Homo sapiens
TSNGRLTAQFGITRKDSFLNISASIPSDVGIYFC

SEQ ID NO: 209 - protein, 11 aa, Homo sapiens
AGRREGAQKLV

SEQ ID NO: 210 - protein, 11 aa, Homo sapiens
FGQGTRLTINP

SEQ ID NO: 211 - protein, 313 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSSTLMGVNIQYFGAGTRLSVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 212 - protein, 26 aa, Homo sapiens
GAGVSQSPRYKVAKRGQDVALRCDPI

SEQ ID NO: 213 - protein, 5 aa, Homo sapiens
SGHVS

SEQ ID NO: 214 - protein, 17 aa, Homo sapiens
LFWYQQALGQGPEFLTY

SEQ ID NO: 215 - protein, 6 aa, Homo sapiens
FQNEAQ

SEQ ID NO: 216 - protein, 38 aa, Homo sapiens; identical to SEQ ID NO: 200

SEQ ID NO: 217 - protein, 13 aa, Homo sapiens
ASSSTLMGVNIQY

SEQ ID NO: 218 - protein, 10 aa, Homo sapiens
FGAGTRLSVL

SEQ ID NO: 219 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 220 - protein, 210 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 221 - protein, 232 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRGRKKLLYIFKQPFMRPVQTTQEEDGCSCRFPEEEEGGCEL

SEQ ID NO: 222 - protein, 513 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 223 - protein, 441 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRPILLTCPTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSG

SEQ ID NO: 224 - protein, 195 aa, Homo sapiens
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 225 - protein, 25 aa, Homo sapiens
PILLTISILSFFSVALLVILACVLW

SEQ ID NO: 226 - protein, 242 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDN

SEQ ID NO: 227 - protein, 215 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHR

SEQ ID NO: 228 - protein, 290 aa, Homo sapiens
MLVRRGARAGPRMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 229 - protein, 273 aa, Homo sapiens
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDRVGGARLMFGDGTQLVVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 230 - protein, 132 aa, Homo sapiens
MNYSPGLVSLILLLLGRTRGDSVTQMEGPVTLSEEAFLTINCTYTATGYPSLFWYVQYPGEGLQLLLKATKADDKGSNKGFEATYRKETTSFHLEKGSVQVSDSAVYFCALSDRVGGARLMFGDGTQLVVKP

SEQ ID NO: 231 - protein, 7 aa, Homo sapiens
TATGYPS

SEQ ID NO: 232 - protein, 7 aa, Homo sapiens
ATKADDK

SEQ ID NO: 233 - protein, 14 aa, Homo sapiens
CALSDRVGGARLMF

SEQ ID NO: 234 - protein, 310 aa, Homo sapiens
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEGLYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 235 - protein, 131 aa, Homo sapiens
MTIRLLCYMGFYFLGAGLMEADIYQTPRYLVIGTGKKITLECSQTMGHDKMYWYQQDPGMELHLIHYSYGVNSTEKGDLSSESTVSRIRTEHFPLTLESARPSHTSQYLCASSEGLYNEQFFGPGTRLTVL

SEQ ID NO: 236 - protein, 5 aa, Homo sapiens
MGHDK

SEQ ID NO: 237 - protein, 6 aa, Homo sapiens
SYGVNS

SEQ ID NO: 238 - protein, 13 aa, Homo sapiens
CASSEGLYNEQFF

SEQ ID NO: 239 - protein, 278 aa, Homo sapiens
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMYPSQGGSEKLVFGKGMKLTVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 240 - protein, 137 aa, Homo sapiens
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFMYPSQGGSEKLVFGKGMKLTVNP

SEQ ID NO: 241 - protein, 7 aa, Homo sapiens
TSENNYY

SEQ ID NO: 242 - protein, 8 aa, Homo sapiens
QEAYKQQN

SEQ ID NO: 243 - protein, 16 aa, Homo sapiens
CAFMYPSQGGSEKLVF

SEQ ID NO: 244 - protein, 310 aa, Homo sapiens
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSSPGFRSYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 245 - protein, 133 aa, Homo sapiens
MGPGLLCWVLLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSSPGFRSYGYTFGSGTRLTVV

SEQ ID NO: 246 - protein, 5 aa, Homo sapiens
SGHNT

SEQ ID NO: 247 - protein, 6 aa, Homo sapiens
YYREEE

SEQ ID NO: 248 - protein, 15 aa, Homo sapiens
CASSSPGFRSYGYTF

SEQ ID NO: 249 - protein, 277 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSDGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 250 - protein, 136 aa, Homo sapiens
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSDGGATNKLIFGTGTLLAVQP

SEQ ID NO: 251 - protein, 7 aa, Homo sapiens
TSESDYY

SEQ ID NO: 252 - protein, 8 aa, Homo sapiens
QEAYKQQN

SEQ ID NO: 253 - protein, 15 aa, Homo sapiens
CAYRSDGGATNKLIF

SEQ ID NO: 254 - protein, 311 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGAGGYNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 255 - protein, 134 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLGAGGYNSPLHFGNGTRLTVT

SEQ ID NO: 256 - protein, 5 aa, Homo sapiens
MNHEY

SEQ ID NO: 257 - protein, 6 aa, Homo sapiens
SMNVEV

SEQ ID NO: 258 - protein, 16 aa, Homo sapiens
CASSLGAGGYNSPLHF

SEQ ID NO: 259 - protein, 278 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMGALNSGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 260 - protein, 137 aa, Homo sapiens
MMKSLRVLLVILWLQLSWVWSQQKEVEQDPGPLSVPEGAIVSLNCTYSNSAFQYFMWYRQYSRKGPELLMYTYSSGNKEDGRFTAQVDKSSKYISLFIRDSQPSDSATYLCAMGALNSGAGSYQLTFGKGTKLSVIP

SEQ ID NO: 261 - protein, 6 aa, Homo sapiens
NSAFQY

SEQ ID NO: 262 - protein, 6 aa, Homo sapiens
TYSSGN

SEQ ID NO: 263 - protein, 17 aa, Homo sapiens
CAMGALNSGAGSYQLTF

SEQ ID NO: 264 - protein, 310 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLSSGTGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 265 - protein, 133 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLSSGTGTEAFFGQGTRLTVV

SEQ ID NO: 266 - protein, 5 aa, Homo sapiens
SGHRS

SEQ ID NO: 267 - protein, 6 aa, Homo sapiens
YFSETQ

SEQ ID NO: 268 - protein, 15 aa, Homo sapiens
CASSLSSGTGTEAFF

SEQ ID NO: 269 - protein, 274 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAERDAGNNRKLIWGLGTSLAVNPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 270 - protein, 133 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAERDAGNNRKLIWGLGTSLAVNP

SEQ ID NO: 271 - protein, 6 aa, Homo sapiens
DSSSTY

SEQ ID NO: 272 - protein, 7 aa, Homo sapiens
IFSNMDM

SEQ ID NO: 273 - protein, 14 aa, Homo sapiens
CAERDAGNNRKLIW

SEQ ID NO: 274 - protein, 311 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLEGGDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 275 - protein, 132 aa, Homo sapiens
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLEGGDTQYFGPGTRLTVL

SEQ ID NO: 276 - protein, 5 aa, Homo sapiens
SGHVS

SEQ ID NO: 277 - protein, 6 aa, Homo sapiens
FQNEAQ

SEQ ID NO: 278 - protein, 13 aa, Homo sapiens
CASSLEGGDTQYF

SEQ ID NO: 279 - DNA, 1178 bp, Homo sapiens
  source 1..1178: mol_type = genomic DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 280 - DNA, 72 bp, Homo sapiens
  source 1..72: mol_type = genomic DNA
ggtgtggaaagtccccaggctccccagcaggcagaagtatgcaaagcatgcatctcaattagtcagcaacca

SEQ ID NO: 281 - protein, 22 aa, Homo sapiens
GSGATNFSLLKQAGDVEENPGP

SEQ ID NO: 282 - DNA, 66 bp, Homo sapiens
  source 1..66: mol_type = genomic DNA
ggaagcggcgccacaaacttctcactgctgaaacaggccggcgacgtggaagagaatcccggacct

SEQ ID NO: 283 - DNA, 66 bp, Homo sapiens
  source 1..66: mol_type = genomic DNA
ggttccggagccacgaacttctctctgttaaagcaagcaggagacgtggaagaaaaccccggtccc

SEQ ID NO: 284 - protein, 25 aa, Homo sapiens
RAKRGSGEGRGSLLTCGDVEENPGP

SEQ ID NO: 285 - DNA, 75 bp, Homo sapiens
  source 1..75: mol_type = genomic DNA
agagccaaaagaggatctggcgaaggcagaggctctctgctgacatgtggcgacgtcgaagaaaaccctgggcca

SEQ ID NO: 286 - protein, 21 aa, Homo sapiens
GSGEGRGSLLTCGDVEENPGP

SEQ ID NO: 287 - DNA, 63 bp, Homo sapiens
  source 1..63: mol_type = genomic DNA
ggcagcggagaaggcagaggctccctgcttacatgcggcgacgtggaagagaaccccggacct

SEQ ID NO: 288 - DNA, 834 bp, Homo sapiens
  source 1..834: mol_type = genomic DNA
atgaccagagtgtccctgctgtgggccgtcgtggtgtctacatgtctggaatctggcatggcccagacagtgacccagagccagcctgagatgtctgtgcaagaggccgaaaccgtgacactgagctgcacctacgacaccagcgagaacaactactacctgttctggtacaagcagcctcctagccggcagatgatcctggtcatcagacaagaggcctataagcagcagaacgccaccgagaacaggttcagcgtgaacttccagaaggccgccaagagcttcagcctgaagatcagcgatagccagctgggcgacaccgccatgtacttttgcgccttcatgtaccccagccaaggcggctctgagaaactggtgtttggcaagggcatgaagctgaccgtgaatcccaacattcagaaccccgatcctgccgtgtatcagctgcgggatagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagtccagctgcgacgtgaagctggtggaaaagagcttcgagacagacaccaacctgaatttccagaacctgagcgtgatcggcttccgcatcctgctgctgaaagtggccggattcaacctgctgatgaccctgcgactgtggtctagc

SEQ ID NO: 289 - DNA, 936 bp, Homo sapiens
  source 1..936: mol_type = genomic DNA
atgggacctggattgctttgttgggtgctgctgtgtctgctcggagccggatctgtggaaacaggcgtgacacagagccccacacacctgatcaagaccagaggccagcaagtgaccctgagatgcagctctcagagcggccacaataccgtgtcctggtatcagcaggccctcggacagggccctcagttcatcttccagtactacagagaggaagagaacggcagaggcaacttcccacctagattcagcggcctgcagttccccaactacagcagcgagctgaacgtgaacgccctggaactggatgacagcgccctgtacctgtgtgcctctagcagccctggcttcagaagctacggctacacatttggcagcggcaccagactgaccgtggtggaagatctgaagaacgtgttcccacctgaggtggccgtgttcgaaccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgactcagatcgtgtctgccgaagcctggggcagagccgattgtggctttaccagcgagtcttaccagcagggcgtgctgtctgccaccatcctgtatgagatcctgctgggcaaagccactctgtacgccgtgctggtttctgccctggtgctgatggccatggtcaagcggaaggatagcagaggc

SEQ ID NO: 290 - DNA, 1539 bp, Homo sapiens
  source 1..1539: mol_type = genomic DNA
atgctcgtcagaagaggcgctagagccggacctagaatgcccagaggatggacagccctctgcctcctgtctctgctgcctagcggcttcatgagcctggacaacaacggcacagccacacctgagctgcctacacagggcaccttcagcaatgtgtccacaaacgtgtcctaccaagagacaaccacacctagcacactgggcagcacatctctgcaccctgtgtctcagcacggcaatgaggccaccaccaatatcaccgagacaaccgtgaagttcaccagcaccagcgtgatcacctccgtgtacggcaacaccaacagcagcgtgcagagccagacctccgtgatcagcaccgtgtttacaacccctgccaatgtgtctacccctgagacaactctgaagcccagcctgtctcctgggaacgtgtccgatctgagcaccacctctaccagcctggccacctctcctacaaagccctacacaagcagcagccccatcctgagcgatatcaaggccgaaatcaagtgcagcggcatccgggaagtgaaactgacccagggcatctgcctggaacagaacaagaccagcagctgcgccgagttcaagaaggacagaggcgaaggactggccagagtgctgtgtggcgaagaacaggccgatgctgatgctggcgctcaagtctgttcactgctgctggcccagtctgaagtgcggcctcaatgtctgcttctggtcctggccaaccggaccgagatctctagcaaactgcagctgatgaagaagcaccagagcgacctgaagaagctgggcatcctggacttcaccgagcaggatgtggccagccaccagagctacagccagaaaacacctatcctgctgacctgtccgacaatcagcatcctgtcctttttcagcgtggccctgctcgtgatcctggcctgtgtgctgtggaagaagcggatcaagcccatcgtgtggcctagcctgcctgaccacaaaaagaccctggaacacctgtgcaaaaagccccggaagaacctgaatgtgtctttcaaccccgagagcttcctggactgccagatccacagagtggacgacatccaggccagagatgaggtggaaggctttctgcaggacacctttccgcagcagctggaagagtccgagaagcagagactcggcggagatgtgcagtcccctaattgccctagcgaggacgtggtcatcacccctgagtccttcggaagagactccagcctgacttgtctggccggaaatgtgtctgcctgcgacgcccctatcctgtccagctctagaagcctggactgtagagagagcggcaagaacggccctcatgtgtaccaggatctgctgctgtccctgggcaccaccaactctacactgcctccaccattcagcctgcagtccggcatcctgacactgaatcctgtggctcagggccagccaatcctgacaagcctgggctccaatcaagaagaggcttacgtcaccatgagcagcttctaccagaatcaa

SEQ ID NO: 291 - DNA, 705 bp, Homo sapiens
  source 1..705: mol_type = genomic DNA
atggctctgcctgtgacagctctgctgctgcctctggctctgcttctgcatgccgctagacccagccagttcagagtgtcccctctggacagaacctggaacctgggcgagacagtggaactgaagtgccaggtgctgctgagcaatcctaccagcggctgcagctggctgtttcagcctagaggtgctgccgcctctcctacctttctgctgtacctgagccagaacaagcccaaggccgccgaaggactggacacccagagattcagcggcaagagactgggcgacaccttcgtgctgaccctgagcgacttcagaagagagaacgagggctactacttctgcagcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgctggcggagccgtgcatacaagaggactggatttcgcctgcgacatctacatctgggcccctctggctggaacatgtggcgtgctgctgctgtccctggtcatcaccctgtactgcaaccaccggaacaggcggagagtgtgcaagtgccctagacctgtggtcaagagcggcgacaagcctagcctgagcgccagatatgtt

SEQ ID NO: 292 - DNA, 630 bp, Homo sapiens
  source 1..630: mol_type = genomic DNA
atgaggcctagactgtggctgcttctggctgcccagctgacagtgctgcacggcaattctgtcctgcagcagacccctgcctacatcaaggtgcagaccaacaagatggtcatgctgagctgcgaggccaagatcagcctgtccaacatgcggatctactggctgcggcagagacaggcccctagctctgatagccaccacgagtttctggccctgtgggattctgccaagggcaccattcacggcgaggaagtggaacaagagaagatcgccgtgttccgggacgccagcagattcatcctgaacctgaccagcgtgaagcccgaggacagcggcatctatttctgcatgatcgtgggcagccccgagctgacatttggcaagggaacacagctgagcgtggtggacttcctgcctactacagcccagcctaccaagaagtctaccctgaagaaacgcgtgtgcagactgcccaggcctgagacacaaaagggccctctgtgcagccctatcacactgggattgctggtggctggcgttctggtcctgctggtgtctctgggagttgccatccacctgtgctgtagaagaaggcgggccagactgcggttcatgaagcagttctacaaa

SEQ ID NO: 293 - DNA, 834 bp, Homo sapiens
  source 1..834: mol_type = genomic DNA
atgatgaagtccctgagagtgctgctggtcatcctgtggctgcagctgtcttgggtctggtcccagcagaaagaggtggaacaggaccctggacctctgtctgttcctgagggcgccatcgtgtccctgaattgcacctacagcaactccgccttccagtacttcatgtggtacagacagtacagccggaagggccccgagctgctgatgtacacatacagcagcggcaacaaagaggacggccggtttacagcccaggtggacaagagcagcaagtacatctccctgttcatccgggacagccagcctagcgatagcgccacatacctgtgtgctatgggcgccctgaattctggcgccggaagctatcagctgaccttcggcaagggcaccaagctgagcgtgatccccaacattcagaaccccgatcctgccgtgtatcagctgcgggatagcaagtccagcgacaagagcgtgtgcctgttcaccgacttcgacagccagaccaacgtgtcccagagcaaggacagcgacgtgtacattaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagtccagctgcgacgtgaagctggtggaaaagagcttcgagacagacaccaacctgaacttccagaacctgtccgtgatcggcttcagaatcctgctgctgaaggtggccggattcaacctgctgatgaccctcagactgtggtctagc

SEQ ID NO: 294 - DNA, 933 bp, Homo sapiens
  source 1..933: mol_type = genomic DNA
atgggatctagattgctttgttgggtgctgctgtgcctgctcggagccggacctgtgaaagctggcgttacccagacacctagatacctgatcaagaccagaggccagcaagtgaccctgagctgctctcctatcagcggccacagaagcgtgtcctggtatcagcagacacctggacagggcctgcagttcctgttcgagtacttcagcgagacacagcggaacaagggcaacttccccggcagattttccggcagacagttcagcaacagccgcagcgagatgaacgtgtccacactggaactgggcgacagcgccctgtatctgtgtgcctcttctctgtcctccggaaccggcaccgaggcctttttcggacaaggcaccagactgaccgtggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtttctgccctggtgctgatggccatggtcaagcggaaggatagcagaggc

SEQ ID NO: 295 - protein, 10 aa, synthetic construct
VVVGADGVGK

SEQ ID NO: 296 - protein, 198 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAGNRRRVCKCPRPVVKSGDKPSLSARYV

SEQ ID NO: 297 - protein, 246 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQMSREYGFGVLLQKDIGQ

SEQ ID NO: 298 - protein, 243 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQPQGEGISGTFVPQCLHGYYSNTTTSQKLLNPWILKT

SEQ ID NO: 299 - protein, 221 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 300 - protein, 213 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGRRRRARLRFMKQPQGEGISGTFVPQCLHGYYSNTTTSQKLLNPWILKT

SEQ ID NO: 301 - protein, 198 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGLKGKVYQEPLSPNACMDTTAILQPHRSCLTHGS

SEQ ID NO: 302 - protein, 8 aa, unidentified
VWPSLPDH

SEQ ID NO: 303 - protein, 7 aa, unidentified
KKRIKPI

SEQ ID NO: 304 - protein, 13 aa, unidentified
KKTLEHLCKKPRK

SEQ ID NO: 305 - protein, 4 aa, unidentified
RAKR

SEQ ID NO: 306 - protein, 34 aa, synthetic construct
VACIMTVVANDGWGATVDNCGAVAGHIFSTYGKR

SEQ ID NO: 307 - protein, 31 aa, synthetic construct
VAIMVVADGWGATVDNCGVVAHIFSTYGKAR